SEQUENCE LISTING (126 sequences)

SEQ ID NO: 1 - protein, 5 aa, Artificial Sequence
  Synthetic Sequence
  REPEAT (1)..(1): A spacer amino acid such as serine or glycine
  REPEAT (2)..(2): A positively charged amino acid such as arginine, histidine or lysine
  REPEAT (3)..(3): A spacer amino acid such as serine or glycine
  REPEAT (4)..(4): A positively charged amino acid such as arginine, histidine or lysine
  REPEAT (5)..(5): A spacer amino acid such as serine or glycine
XXXXX

SEQ ID NO: 2 - protein, 5 aa, Artificial Sequence
  Synthetic Sequence
  REPEAT (1)..(1): A spacer amino acid such as serine or glycine
  REPEAT (2)..(2): A negatively charged amino acid such as aspartic acid or glutamic acid
  REPEAT (3)..(3): A spacer amino acid such as serine or glycine
  REPEAT (4)..(4): A negatively charged amino acid such as aspartic acid or glutamic acid
  REPEAT (5)..(5): A spacer amino acid such as serine or glycine
XXXXX

SEQ ID NO: 3 - protein, 10 aa, Artificial Sequence
  Synthetic Sequence
  MISC_FEATURE (1)..(1): A spacer amino acid such as serine or glycine
  REPEAT (2)..(2): A spacer amino acid such as serine or glycine
  MISC_FEATURE (3)..(3): A positively charged amino acid such as arginine, histidine or lysine
  REPEAT (4)..(4): A positively charged amino acid such as arginine, histidine or lysine
  MISC_FEATURE (5)..(5): A spacer amino acid such as serine or glycine
  REPEAT (6)..(6): A spacer amino acid such as serine or glycine
  MISC_FEATURE (7)..(7): A positively charged amino acid such as arginine, histidine or lysine
  REPEAT (8)..(8): A positively charged amino acid such as arginine, histidine or lysine
  MISC_FEATURE (9)..(9): A spacer amino acid such as serine or glycine
  REPEAT (10)..(10): A spacer amino acid such as serine or glycine
XXXXXXXXXX

SEQ ID NO: 4 - protein, 10 aa, Artificial Sequence
  Synthetic Sequence
  MISC_FEATURE (1)..(1): A spacer amino acid such as serine or glycine
  REPEAT (2)..(2): A spacer amino acid such as serine or glycine
  MISC_FEATURE (3)..(3): A negatively charged amino acid such as aspartic acid or glutamic acid
  REPEAT (4)..(4): A negatively charged amino acid such as aspartic acid or glutamic acid
  MISC_FEATURE (5)..(5): A spacer amino acid such as serine or glycine
  REPEAT (6)..(6): A spacer amino acid such as serine or glycine
  MISC_FEATURE (7)..(7): A negatively charged amino acid such as aspartic acid or glutamic acid
  REPEAT (8)..(8): A negatively charged amino acid such as aspartic acid or glutamic acid
  MISC_FEATURE (9)..(9): A spacer amino acid such as serine or glycine
  REPEAT (10)..(10): A spacer amino acid such as serine or glycine
XXXXXXXXXX

SEQ ID NO: 5 - protein, 6 aa, Artificial Sequence
  Synthetic Sequence
  REPEAT (1)..(1): A spacer amino acid such as serine or glycine
  REPEAT (2)..(2): A positively charged amino acid such as arginine, histidine or lysine
  REPEAT (4)..(4): A spacer amino acid such as serine or glycine
  REPEAT (5)..(5): A positively charged amino acid such as arginine, histidine or lysine
  REPEAT (6)..(6): A spacer amino acid such as serine or glycine
XXCXXX

SEQ ID NO: 6 - protein, 6 aa, Artificial Sequence
  Synthetic Sequence
  REPEAT (1)..(1): A spacer amino acid such as serine or glycine
  REPEAT (2)..(2): A negatively charged amino acid such as aspartic acid or glutamic acid
  REPEAT (4)..(4): A spacer amino acid such as serine or glycine
  REPEAT (5)..(5): A negatively charged amino acid such as aspartic acid or glutamic acid
  REPEAT (6)..(6): A spacer amino acid such as serine or glycine
XXCXXX

SEQ ID NO: 7 - protein, 12 aa, Artificial Sequence
  Synthetic Sequence
GSGSRKGGKRGS

SEQ ID NO: 8 - protein, 12 aa, Artificial Sequence
  Synthetic Sequence
GSGSRKCGKRGS

SEQ ID NO: 9 - protein, 12 aa, Artificial Sequence
  Synthetic Sequence
GSGSDEGGEDGS

SEQ ID NO: 10 - protein, 12 aa, Artificial Sequence
  Synthetic Sequence
GSGSDECGEDGS

SEQ ID NO: 11 - protein, 6 aa, Artificial Sequence
  Synthetic polymer.
RKGGKR

SEQ ID NO: 12 - protein, 12 aa, Artificial Sequence
  Synthetic polymer.
GSGSRKGGKRGS

SEQ ID NO: 13 - protein, 6 aa, Artificial Sequence
  Synthetic polymer.
DEGGED

SEQ ID NO: 14 - protein, 12 aa, Artificial Sequence
  Synthetic polymer.
GSGSDEGGEDGS

SEQ ID NO: 15 - protein, 234 aa, Artificial Sequence
  Synthetic polymer.
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 16 - protein, 249 aa, Artificial Sequence
  Synthetic polymer.
GSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGS

SEQ ID NO: 17 - protein, 249 aa, Artificial Sequence
  Synthetic polymer.
GSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGS

SEQ ID NO: 18 - protein, 19 aa, Artificial Sequence
  Synthetic polymer.
MEFGLSWVFLVAIIKGVQC

SEQ ID NO: 19 - protein, 225 aa, Artificial Sequence
  Synthetic polymer.
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAG

SEQ ID NO: 20 - protein, 111 aa, Artificial Sequence
  Synthetic polymer.
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 21 - protein, 109 aa, Artificial Sequence
  Synthetic polymer.
EVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTV

SEQ ID NO: 22 - protein, 250 aa, Artificial Sequence
  Synthetic polymer.
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 23 - protein, 239 aa, Artificial Sequence
  Synthetic polymer.
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 24 - protein, 238 aa, Artificial Sequence
  Synthetic polymer.
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELK

SEQ ID NO: 25 - protein, 238 aa, Artificial Sequence
  Synthetic polymer.
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 26 - protein, 120 aa, Artificial Sequence
  Synthetic polymer.
RKGGKRGSGSGQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 27 - protein, 166 aa, Artificial Sequence
  Synthetic polymer.
GTDFTLKISRVEAEDLGVYFCSQSTHVPPLTFGAGTKLELKGGGSGGGSGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMKYWGQGTSVTVSS

SEQ ID NO: 28 - protein, 222 aa, Artificial Sequence
  Synthetic polymer.
CPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 29 - protein, 232 aa, Artificial Sequence
  Synthetic polymer.
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 232 aa, Artificial Sequence
  Synthetic polymer.
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 31 - protein, 227 aa, Artificial Sequence
  Synthetic polymer.
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 32 - protein, 222 aa, Artificial Sequence; identical to SEQ ID NO: 28
  Synthetic polymer.

SEQ ID NO: 33 - protein, 741 aa, Artificial Sequence
  Synthetic polymer.
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 34 - protein, 730 aa, Artificial Sequence
  Synthetic polymer.
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 35 - protein, 220 aa, Artificial Sequence
  Synthetic polymer.
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCA

SEQ ID NO: 36 - protein, 220 aa, Artificial Sequence; identical to SEQ ID NO: 35
  Synthetic polymer.

SEQ ID NO: 37 - protein, 725 aa, Artificial Sequence
  Synthetic polymer.
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 38 - protein, 716 aa, Artificial Sequence
  Synthetic polymer.
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 39 - protein, 716 aa, Artificial Sequence
  Synthetic polymer.
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 40 - protein, 732 aa, Artificial Sequence
  Synthetic polymer.
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 41 - protein, 720 aa, Artificial Sequence
  Synthetic polymer.
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 720 aa, Artificial Sequence
  Synthetic polymer.
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 43 - protein, 747 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGSGGSGSGGSGGSGAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 752 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGSGGSGSGGSGGSGAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 45 - protein, 225 aa, Artificial Sequence; identical to SEQ ID NO: 19
  Synthetic polymer.

SEQ ID NO: 46 - DNA, 2178 bp, Artificial Sequence
  Synthetic polymer.
atggagttcggcctgagctgggtgtttctggtggccatcatcaagggcgtgcagtgccagttcatcgtggtgggccctaccgacccaatcctggccacagtgggcgagaacaccacactgaggtgtcacctgtccccagagaagaatgccgaggatatggaggtgcggtggttcagatctcagtttagccccgccgtgttcgtgtataagggcggccgggagagaaccgaggagcagatggaggagtacaggggccgcaccacatttgtgagcaaggacatctcccgcggctctgtggccctggtcatccacaacatcaccgcccaggagaatggcacatatcggtgctactttcaggagggcagatcctacgatgaggccatcctgcacctggtggtggcaggcctgggatctaagcccctgatcagcatgaggggacacgaggacggaggaatcaggctggagtgtatcagcagaggctggtatcccaagcctctgaccgtgtggagagatccctacggaggagtggcacctgccctgaaggaggtgtccatgccagacgccgatggcctgttcatggtgaccacagccgtgatcatccgggacaagtctgtgagaaatatgtcttgcagcatcaacaatacactgctgggccagaagaaggagagcgtgatcttcatccccgagtcctttatgccatccgtgtctccatgtgcaggaagcggctccaggaagggaggcaagaggggaagcaagtatggaccaccttgcccaccatgtccagcaccagagtttctgggaggaccatccgtgttcctgtttcctccaaagcccaaggaccagctgatgatctccaggaccccagaggtgacatgcgtggtggtggacgtgtctcaggaggatcctgaggtgcagttcaactggtacgtggacggcgtggaggtgcacaatgccaagaccaagcccagggaggagcagtttaactccacctatcgcgtggtgtctgtgctgacagtgctgcaccaggactggctgagcggcaaggagtacaagtgcaaggtgagctccaagggcctgccttctagcatcgagaagaccatctccaacgccacaggccagcccagagagcctcaggtgtataccctgccccctagccaggaggagatgaccaagaatcaggtgtccctgacatgtctggtgaagggcttctacccttccgatatcgccgtggagtgggagtctaacggccagccagagaacaattataagaccacaccacccgtgctggacagcgatggctccttctttctgtactctaggctgaccgtggacaagagccgctggcaggagggcaacgtgttttcttgcagcgtgctgcacgaggccctgcacaatcactacacacagaagtccctgtctctgagcctgggcaaggatgagggaggagaggacggaagcgatatccagatgacccagaccacatcctctctgtccgcctctctgggcgacagggtgacaatctcctgtcgcgcctctcaggatatcagcaagtatctgaattggtatcagcagaagcctgacggcaccgtgaagctgctgatctatcacacatcccggctgcactctggcgtgccaagcagattcagcggatccggatctggcaccgactactccctgacaatctctaacctggagcaggaggatatcgccacctatttctgccagcagggcaataccctgccttacacatttggcggcggcaccaagctggagatcacaggcggaggaagcggaggaggatccggaggaggatctgaggtgaagctgcaggagagcggacctggcctggtggcaccaagccagtccctgtctgtgacctgtacagtgtctggcgtgagcctgcccgattacggcgtgtcttggatcaggcagcctccaaggaagggcctggagtggctgggcgtgatctggggcagcgagacaacatactataacagcgccctgaagtcccggctgaccatcatcaaggacaacagcaagtcccaggtgttcctgaagatgaattccctgcagaccgacgatacagccatctactattgtgccaagcactactattacggcggctcttatgccatggattactggggccagggcaccagcgtgacagtgagctcctga

SEQ ID NO: 47 - protein, 2151 aa, Artificial Sequence
  Synthetic polymer.
ATGGAGTTTGGCCTGTCTTGGGTGTTTCTGGTGGCTATCATCAAGGGAGTGCAGTGTCAGTTCATCGTTGTGGGACCTACCGATCCTATCTTGGCTACAGTGGGCGAGAATACAACCCTGAGATGTCACTTGTCTCCTGAGAAGAACGCCGAGGATATGGAGGTTAGGTGGTTCAGATCCCAGTTCTCTCCTGCCGTGTTTGTGTATAAGGGAGGCAGAGAGAGAACAGAAGAGCAGATGGAGGAGTACAGAGGAAGAACCACCTTCGTGTCTAAGGACATCAGCAGAGGCTCTGTGGCTCTGGTGATCCACAATATCACAGCTCAGGAGAATGGCACCTACAGATGCTACTTTCAGGAGGGCAGGTCCTACGATGAGGCTATTTTGCATCTGGTGGTTGCTGGACTGGGATCTAAACCTCTGATCAGCATGAGGGGACACGAGGATGGAGGAATTAGACTGGAGTGCATCTCTAGAGGCTGGTATCCTAAACCACTGACAGTGTGGAGAGACCCTTATGGAGGAGTTGCTCCTGCTCTGAAAGAGGTGTCTATGCCTGATGCTGATGGCCTGTTTATGGTGACAACAGCCGTGATCATCCGGGACAAATCCGTGAGGAACATGTCTTGCTCCATCAACAACACACTGTTGGGACAGAAGAAGGAGAGCGTGATCTTCATCCCCGAGAGCTTCATGCCTAGCGTTTCTCCTTGTGCTGAACCTAAGTCTTGCGACAAGACCCATACATGCCCTCCTTGTCCTGCTCCTGAAGCTGCTGGAGGACCTTCTGTGTTTTTGTTTCCTCCTAAGCCTAAGGATACCCTGATGATCTCCAGAACCCCCGAGGTGACCTGTGTGGTGGTTGATGTTTCTCATGAGGATCCTGAAGTGAAGTTCAACTGGTACGTGGACGGCGTGGAGGTGCACAACGCTAAGACAAAACCTAGAGAAGAGCAGTACAACTCTACCTACAGAGTGGTGTCCGTGCTGACCGTGCTGCACCAAGATTGGTTGAATGGAAAGGAGTACAAGTGCAAGGTGTCCAACAAGGCTCTGCCTGCCCCTATCGAGAAGACCATCTCTAAGGCTAAAGGACAACCTAGAGAACCTCAGGTGTATACCCTGCCTCCCTGCAGAGATGAGCTGACCAAGAATCAGGTTTCTCTGTGGTGTCTGGTGAAGGGCTTTTACCCTAGCGACATCGCTGTGGAGTGGGAGTCTAATGGACAACCTGAGAACAACTACAAGACCACACCTCCTGTGCTGGACTCTGATGGCTCCTTCTTCCTGTACTCTAAGCTGACCGTGGATAAGTCTAGATGGCAACAGGGCAACGTGTTCTCCTGCTCCGTGATGCATGAAGCTCTGCACAACCACTATACACAGAAGTCTCTGAGCCTGTCTCCTGGCAAGATCGAGGGCAGAATGGACGATATCCAGATGACACAGACAACCTCTTCTCTGTCTGCTTCTCTGGGCGATAGAGTGACCATCAGCTGCAGAGCTTCTCAGGACATCAGCAAGTATCTGAACTGGTATCAGCAGAAGCCTGATGGCACCGTGAAGCTGCTGATCTACCACACCTCCAGATTGCATTCTGGAGTTCCTTCCAGATTTTCTGGCTCTGGCTCTGGCACCGACTATTCTCTGACCATCAGCAATCTGGAACAGGAGGACATCGCTACCTACTTTTGCCAGCAGGGCAACACACTGCCTTACACATTTGGAGGAGGAACAAAGCTGGAGATCACAGGAGGAGGATCTGGAGGAGGATCTGGAGGAGGATCTGAAGTTAAACTGCAGGAATCTGGACCAGGATTAGTGGCCCCATCTCAGTCTCTGTCTGTGACCTGTACCGTTTCTGGAGTTTCTTTGCCTGATTACGGAGTGTCCTGGATCAGACAGCCCCCTAGAAAGGGACTGGAATGGTTAGGAGTGATTTGGGGATCTGAGACCACCTACTACAACTCTGCCCTGAAGAGCAGACTGACCATCATCAAGGACAACAGCAAGTCTCAGGTGTTCCTGAAGATGAACTCCCTGCAGACCGACGATACCGCCATCTACTACTGTGCTAAGCACTACTACTATGGCGGCTCTTATGCCATGGACTATTGGGGACAGGGCACCTCTGTGACAGTGTCTTCTTAA

SEQ ID NO: 48 - DNA, 2151 bp, Artificial Sequence
  Synthetic polymer.
atggagtttggcctgtcttgggtgtttctggtggctatcatcaagggagtgcagtgtcagttcatcgttgtgggacctaccgatcctatcttggctacagtgggcgagaatacaaccctgagatgtcacttgtctcctgagaagaacgccgaggatatggaggttaggtggttcagatcccagttctctcctgccgtgtttgtgtataagggaggcagagagagaacagaagagcagatggaggagtacagaggaagaaccaccttcgtgtctaaggacatcagcagaggctctgtggctctggtgatccacaatatcacagctcaggagaatggcacctacagatgctactttcaggagggcaggtcctacgatgaggctattttgcatctggtggttgctggactgggatctaaacctctgatcagcatgaggggacacgaggatggaggaattagactggagtgcatctctagaggctggtatcctaaaccactgacagtgtggagagacccttatggaggagttgctcctgctctgaaagaggtgtctatgcctgatgctgatggcctgtttatggtgacaacagccgtgatcatccgggacaaatccgtgaggaacatgtcttgctccatcaacaacacactgttgggacagaagaaggagagcgtgatcttcatccccgagagcttcatgcctagcgtttctccttgtgctgaacctaagtcttgcgacaagacccatacatgccctccttgtcctgctcctgaagctgctggaggaccttctgtgtttttgtttcctcctaagcctaaggataccctgatgatctccagaacccccgaggtgacctgtgtggtggttgatgtttctcatgaggatcctgaagtgaagttcaactggtacgtggacggcgtggaggtgcacaacgctaagacaaaacctagagaagagcagtacaactctacctacagagtggtgtccgtgctgaccgtgctgcaccaagattggttgaatggaaaggagtacaagtgcaaggtgtccaacaaggctctgcctgcccctatcgagaagaccatctctaaggctaaaggacaacctagagaacctcaggtgtataccctgcctccctgcagagatgagctgaccaagaatcaggtttctctgtggtgtctggtgaagggcttttaccctagcgacatcgctgtggagtgggagtctaatggacaacctgagaacaactacaagaccacacctcctgtgctggactctgatggctccttcttcctgtactctaagctgaccgtggataagtctagatggcaacagggcaacgtgttctcctgctctgtgctgcatgaagctctgcactctcactatacacagaagtctctgtccctgtctcctggcaagatcgagggcagaatggacgatatccagatgacacagacaacctcttctctgtctgcttctctgggcgatagagtgaccatcagctgcagagcttctcaggacatcagcaagtatctgaactggtatcagcagaagcctgatggcaccgtgaagctgctgatctaccacacctccagattgcattctggagttccttccagattttctggctctggctctggcaccgactattctctgaccatcagcaatctggaacaggaggacatcgctacctacttttgccagcagggcaacacactgccttacacatttggaggaggaacaaagctggagatcacaggaggaggatctggaggaggatctggaggaggatctgaagttaaactgcaggaatctggaccaggattagtggccccatctcagtctctgtctgtgacctgtaccgtttctggagtttctttgcctgattacggagtgtcctggatcagacagccccctagaaagggactggaatggttaggagtgatttggggatctgagaccacctactacaactctgccctgaagagcagactgaccatcatcaaggacaacagcaagtctcaggtgttcctgaagatgaactccctgcagaccgacgataccgccatctactactgtgctaagcactactactatggcggctcttatgccatggactattggggacagggcacctctgtgacagtgtcttcttaa

SEQ ID NO: 49 - DNA, 2199 bp, Artificial Sequence
  Synthetic polymer.
atggagttcggcctgtcctgggtgtttctggtggccatcatcaagggcgtgcagtgccagttctccgtgctgggcccttctggcccaatcctggcaatggtgggagaggacgcagatctgccatgtcacctgtttcccaccatgagcgccgagacaatggagctgaagtgggtgagctcctctctgcggcaggtggtgaacgtgtacgccgacggcaaggaggtggaggatagacagtccgccccctaccggggcagaacctctatcctgagggacggaatcacagcaggcaaggccgccctgagaatccacaacgtgaccgccagcgattccggcaagtatctgtgctacttccaggacggcgacttctacgagaaggccctggtggagctgaaggtggccgccctgggaagcgacctgcatgtggatgtgaagggctataaggacggcggcatccacctggagtgtcggagcaccggctggtatccccagcctcagatccagtggtccaacaataagggcgagaatatccctacagtggaggccccagtggtggcagatggagtgggcctgtacgcagtggccgcctctgtgatcatgaggggaagctccggagagggcgtgagctgcaccatccgctctagcctgctgggcctggagaagacagcctctatcagcatcgccgaccccttctttaggagcgcccagcggtggatcgccgccctggcaggcggctccggctctgacgagggcggcgaggatggctccaagtatggaccaccttgcccaccatgtccagcaccagagttcctgggaggaccaagcgtgttcctgtttcctccaaagcccaaggaccagctgatgatctccaggaccccagaggtgacctgcgtggtggtggacgtgtctcaggaggatcctgaggtgcagttcaactggtacgtggatggcgtggaggtgcacaatgccaagaccaagcctagggaggagcagtttaacagcacctatcgcgtggtgtccgtgctgacagtgctgcaccaggattggctgtccggcaaggagtacaagtgcaaggtgtcctctaagggcctgccaagctccatcgagaagaccatcagcaacgcaacaggccagccccgcgagcctcaggtgtataccctgcccccttctcaggaggagatgaccaagaatcaggtgagcctgacatgtctggtgaagggcttctaccctagcgacatcgcagtggagtgggagtccaacggacagccagagaacaattataagaccacaccacccgtgctggactctgatggcagcttctttctgtactctaggctgaccgtggataagagccgctggcaggagggcaacgtgtttagctgctccgtgctgcacgaggccctgcacaatcactacacacagaagtctctgagcctgtccctgggcaagaggaagggaggcaagaggggatctggaagcgacatccagatgacccagaccacatctagcctgtccgcctctctgggcgaccgggtgacaatcagctgtagagcctcccaggatatctctaagtatctgaattggtatcagcagaagccagatggcaccgtgaagctgctgatctatcacacaagcaggctgcactccggcgtgccctctagattcagcggatccggatctggcaccgactacagcctgacaatctccaacctggagcaggaggatatcgccacctatttctgccagcagggcaataccctgccctacacatttggcggcggcaccaagctggagatcacaggcggaggatctggaggaggaagcggaggaggctccgaggtgaagctgcaggagtccggaccaggcctggtggcacctagccagtccctgtctgtgacctgtacagtgtccggcgtgtctctgcctgactacggcgtgtcctggatccggcagcctccaagaaagggcctggagtggctgggcgtgatctggggcagcgagacaacatactataactctgccctgaagagcagactgaccatcatcaaggacaacagcaagtcccaggtgtttctgaagatgaatagcctgcagaccgacgatacagccatctactattgcgccaagcactactattacggcggctcctatgccatggattactggggccagggcacctctgtgacagtgtcctcttga

SEQ ID NO: 50 - DNA, 2163 bp, Artificial Sequence
  Synthetic polymer.
atggagtttggcctgtcttgggtgtttctggtggctatcatcaagggagtgcagtgtcagttctctgttctgggaccttctggacctatcttggctatggtgggagaagatgctgatctgccttgtcacctgtttcctaccatgtctgctgagaccatggagctgaaatgggtgtcctcttctctgagacaggtggtgaatgtgtacgctgatggaaaggaggtggaggacagacaatctgccccttatagaggaagaaccagcatcctgagagatggcatcacagctggaaaggctgctctgagaatccacaatgtgaccgcttctgattctggcaagtacctgtgctacttccaggatggcgacttctacgagaaggctctggttgagctgaaagttgctgctttgggctctgatctgcatgttgacgtgaagggctacaaggatggaggcatccatctggaatgtagatctaccggctggtatcctcaacctcagattcagtggagcaacaacaagggcgagaacatccctacagttgaagcccctgttgtggctgatggagttggactgtatgctgttgctgctagcgtgatcatgagaggatcttctggagaaggcgtgtcttgcaccatcagatcttctctgttgggactggagaagaccgctagcatctctatcgctgaccccttcttcagatctgctcaaagatggattgctgctctggctgagcctaagtcttgcgataagacccacacctgtcctccttgtcctgctcctgaagctgctggaggaccttctgtgtttttgtttcctcctaagcctaaggataccctgatgatctccagaacccccgaggtgacctgtgtggtggttgatgtttctcatgaggatcctgaagtgaagttcaactggtacgtggacggcgtggaggtgcacaacgctaagacaaaacctagagaagagcagtacaactctacctacagagtggtgtccgtgctgaccgtgctgcaccaagattggttgaatggaaaggagtacaagtgcaaggtgtccaacaaggctctgcctgcccctatcgagaagaccatctctaaggctaaaggacaacctagagaacctcaggtgtgtacactgcccccctctagagatgagctgaccaagaatcaggtttctctgtcttgtgctgtgaagggcttttacccctccgacatcgctgtggaatgggagtctaatggacaacctgagaacaactacaagaccacacctcctgtgctggactctgacggctccttctttctggtgtctaagctgacagtggataagtctagatggcaacagggcaacgtgttcagctgctccgtgatgcatgaagctctgcacaaccactatacacagaagtctctgagcctgtctcctggcaagatcgagggcagaatggacgatatccagatgacacagacaacctcttctctgtctgcttctctgggcgatagagtgaccatcagctgcagagcttctcaggacatcagcaagtatctgaactggtatcagcagaagcctgatggcaccgtgaagctgctgatctaccacacctccagattgcattctggagttccttccagattttctggctctggctctggcaccgactattctctgaccatcagcaatctggaacaggaggacatcgctacctacttttgccagcagggcaacacactgccttacacatttggaggaggaacaaagctggagatcacaggaggaggatctggaggaggatctggaggaggatctgaagttaaactgcaggaatctggaccaggattagtggccccatctcagtctctgtctgtgacctgtaccgtttctggagtttctttgcctgattacggagtgtcctggatcagacagccccctagaaagggactggaatggttaggagtgatttggggatctgagaccacctactacaactctgccctgaagagcagactgaccatcatcaaggacaacagcaagtctcaggtgttcctgaagatgaactccctgcagaccgacgataccgccatctactactgtgctaagcactactactatggcggctcttatgccatggactattggggacagggcacctctgtgacagtgtcttcttaa

SEQ ID NO: 51 - DNA, 2163 bp, Artificial Sequence
  Synthetic polymer.
atggagtttggcctgtcttgggtgtttctggtggctatcatcaagggagtgcagtgtcagttctctgttctgggaccttctggacctatcttggctatggtgggagaagatgctgatctgccttgtcacctgtttcctaccatgtctgctgagaccatggagctgaaatgggtgtcctcttctctgagacaggtggtgaatgtgtacgctgatggaaaggaggtggaggacagacaatctgccccttatagaggaagaaccagcatcctgagagatggcatcacagctggaaaggctgctctgagaatccacaatgtgaccgcttctgattctggcaagtacctgtgctacttccaggatggcgacttctacgagaaggctctggttgagctgaaagttgctgctttgggctctgatctgcatgttgacgtgaagggctacaaggatggaggcatccatctggaatgtagatctaccggctggtatcctcaacctcagattcagtggagcaacaacaagggcgagaacatccctacagttgaagcccctgttgtggctgatggagttggactgtatgctgttgctgctagcgtgatcatgagaggatcttctggagaaggcgtgtcttgcaccatcagatcttctctgttgggactggagaagaccgctagcatctctatcgctgaccccttcttcagatctgctcaaagatggattgctgctctggctgagcctaagtcttgcgataagacccacacctgtcctccttgtcctgctcctgaagctgctggaggaccttctgtgtttttgtttcctcctaagcctaaggataccctgatgatctccagaacccccgaggtgacctgtgtggtggttgatgtttctcatgaggatcctgaagtgaagttcaactggtacgtggacggcgtggaggtgcacaacgctaagacaaaacctagagaagagcagtacaactctacctacagagtggtgtccgtgctgaccgtgctgcaccaagattggttgaatggaaaggagtacaagtgcaaggtgtccaacaaggctctgcctgcccctatcgagaagaccatctctaaggctaaaggacaacctagagaacctcaggtgtgtacactgcccccctctagagatgagctgaccaagaatcaggtttctctgtcttgtgctgtgaagggcttttacccctccgacatcgctgtggaatgggagtctaatggacaacctgagaacaactacaagaccacacctcctgtgctggactctgacggctccttctttctggtgtctaagctgacagtggataagtctagatggcaacagggcaacgtgttcagctgcagcgttctgcatgaagctctgcattcccactatacacagaagtctctgtccctgtctcctggcaagatcgagggcagaatggatgacatccagatgacacagacaacctcttctctgtctgcttctctgggcgatagagtgaccatcagctgcagagcttctcaggacatcagcaagtatctgaactggtatcagcagaagcctgatggcaccgtgaagctgctgatctaccacacctccagattgcattctggagttccttccagattttctggctctggctctggcaccgactattctctgaccatcagcaatctggaacaggaggacatcgctacctacttttgccagcagggcaacacactgccttacacatttggaggaggaacaaagctggagatcacaggaggaggatctggaggaggatctggaggaggatctgaagttaaactgcaggaatctggaccaggattagtggccccatctcagtctctgtctgtgacctgtaccgtttctggagtttctttgcctgattacggagtgtcctggatcagacagccccctagaaagggactggaatggttaggagtgatttggggatctgagaccacctactacaactctgccctgaagagcagactgaccatcatcaaggacaacagcaagtctcaggtgttcctgaagatgaactccctgcagaccgacgataccgccatctactactgtgctaagcactactactatggcggctcttatgccatggactattggggacagggcacctctgtgacagtgtcttcttaa

SEQ ID NO: 52 - protein, 238 aa, Artificial Sequence
  Synthetic polymer.
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKIEGRMD

SEQ ID NO: 53 - protein, 238 aa, Artificial Sequence
  Synthetic polymer.
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKIEGRMD

SEQ ID NO: 54 - protein, 238 aa, Artificial Sequence
  Synthetic polymer.
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGKIEGRMD

SEQ ID NO: 55 - protein, 238 aa, Artificial Sequence
  Synthetic polymer.
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGKIEGRMD

SEQ ID NO: 56 - protein, 738 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 57 - protein, 742 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 58 - protein, 747 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 59 - protein, 742 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 60 - protein, 746 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 61 - protein, 751 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 62 - protein, 746 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 63 - protein, 750 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 64 - protein, 755 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 65 - protein, 739 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 66 - protein, 743 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 67 - protein, 748 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 68 - protein, 749 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 69 - protein, 753 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 70 - protein, 758 aa, Artificial Sequence
  Synthetic polymer.
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 71 - protein, 113 aa, Artificial Sequence
  Synthetic polymer.
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLV

SEQ ID NO: 72 - protein, 116 aa, Artificial Sequence
  Synthetic polymer.
AQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVA

SEQ ID NO: 73 - protein, 9 aa, Artificial Sequence
  Synthetic polymer.
GGGGSGGGS

SEQ ID NO: 74 - protein, 15 aa, Artificial Sequence
  Synthetic polymer.
GGGGSGGGGSGGGGS

SEQ ID NO: 75 - protein, 13 aa, Artificial Sequence
  Synthetic polymer.
GGGGSGGGSGGGS

SEQ ID NO: 76 - protein, 16 aa, Artificial Sequence
  Synthetic polymer.
GGGSGGGSGGGSGGGS

SEQ ID NO: 77 - protein, 17 aa, Artificial Sequence
  Synthetic polymer.
GGGGSGGGSGGGSGGGS

SEQ ID NO: 78 - protein, 10 aa, Artificial Sequence
  Synthetic polymer.
GGGGSGGGGS

SEQ ID NO: 79 - protein, 20 aa, Artificial Sequence
  Synthetic polymer.
GGGGSGGGGSGGGGSGGGGS

SEQ ID NO: 80 - protein, 220 aa, Artificial Sequence
  Synthetic polymer.
QWQVTGPGKFVQALVGEDAVFSCSLFPETSAEAMEVRFFRNQFHAVVHLYRDGEDWESKQMPQYRGRTEFVKDSIAGGRVSLRLKNITPSDIGLYGCWFSSQIYDEEATWELRVAALGSLPLISIVGYVDGGIQLLCLSSGWFPQPTAKWKGPQGQDLSSDSRANADGYSLYDVEISIIVQENAGSILCSIHLAEQSHEVESKVLIGETFFQPSPWRLAS

SEQ ID NO: 81 - protein, 219 aa, Artificial Sequence
  Synthetic polymer.
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPW

SEQ ID NO: 82 - protein, 221 aa, Artificial Sequence
  Synthetic polymer.
QWQVFGPDKPVQALVGEDAAFSCFLSPKTNAEAMEVRFFRGQFSSVVHLYRDGKDQPFMQMPQYQGRTKLVKDSIAEGRISLRLENITVLDAGLYGCRISSQSYYQKAIWELQVSALGSVPLISITGYVDRDIQLLCQSSGWFPRPTAKWKGPQGQDLSTDSRTNRDMHGLFDVEISLTVQENAGSISCSMRHAHLSREVESRVQIGDTFFEPISWHLATK

SEQ ID NO: 83 - protein, 216 aa, Artificial Sequence
  Synthetic polymer.
APFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPR

SEQ ID NO: 84 - protein, 215 aa, Artificial Sequence
  Synthetic polymer.
APFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLP

SEQ ID NO: 85 - protein, 432 aa, Artificial Sequence
  Synthetic polymer.
KQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGW

SEQ ID NO: 86 - protein, 233 aa, Artificial Sequence
  Synthetic polymer.
QFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIFIPESFMPSASPWMVALAVILTASPWM

SEQ ID NO: 87 - protein, 219 aa, Artificial Sequence
  Synthetic polymer.
QVTVVGPTDPILAMVGENTTLRCCLSPEENAEDMEVRWFQSQFSPAVFVYKGGRERTEEQKEEYRGRTTFVSKDSRGSVALIIHNVTAEDNGIYQCYFQEGRSCNEAILHLVVAGLDSEPVIEMRDHEDGGIQLECISGGWYPKPLTVWRDPYGEVVPALKEVSTPDADSLFMVTTAVIIRDKSVRNVSCSINDTLLGQKKESVIFIPESFMPSRSPCV

SEQ ID NO: 88 - protein, 220 aa, Artificial Sequence; identical to SEQ ID NO: 80
  Synthetic polymer.

SEQ ID NO: 89 - protein, 219 aa, Artificial Sequence; identical to SEQ ID NO: 81
  Synthetic polymer.

SEQ ID NO: 90 - protein, 219 aa, Artificial Sequence
  Synthetic polymer.
QFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPW

SEQ ID NO: 91 - protein, 221 aa, Artificial Sequence; identical to SEQ ID NO: 82
  Synthetic polymer.

SEQ ID NO: 92 - protein, 222 aa, Artificial Sequence
  Synthetic polymer.
SSEVKVLGPEYPILALVGEEVEFPCHLWPQLDAQQMEIRWFRSQTFNVVHLYQEQQELPGRQMPAFRNRTKLVKDDIAYGSVVLQLHSIIPSDKGTYGCRFHSDNFSGEALWELEVAGLGSDPHLSLEGFKEGGIQLRLRSSGWYPKPKVQWRDHQGQCLPPEFEAIVWDAQDLFSLETSVVVRAGALSNVSVSIQNLLLSQKKELVVQIADVFVPGASAWK

SEQ ID NO: 93 - protein, 228 aa, Artificial Sequence
  Synthetic polymer.
SIWKADFDVTGPHAPILAMAGGHVELQCQLFPNISAEDMELRWYRCQPSLAVHMHERGMDMDGEQKWQYRGRTTFMSDHVARGKAMVRSHRVTTFDNRTYCCRFKDGVKFGEATVQVQVAGLGREPRIQVTDQQDGVRAECTSAGCFPKSWVERRDFRGQARPAVTNLSASATTRLWAVASSLTLWDRAVEGLSCSISSPLLPERRKVAESHLPATFSRSSQFTAWKA

SEQ ID NO: 94 - protein, 249 aa, Artificial Sequence
  Synthetic polymer.
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKEDTIRGPNYYYYGMDVWGQGTTVTVSSASGGGGSGGGGSGGGGSETTLTQSPSSVSASVGDRVSITCRASQDIDTWLAWYQLKPGKAPKLLMYAASNLQGGVPSRFSGSGSGTDFILTISSLQPEDFATYYCQQASIFPPTFGGGTKVDIK

SEQ ID NO: 95 - protein, 250 aa, Artificial Sequence
  Synthetic polymer.
QVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 96 - protein, 240 aa, Artificial Sequence
  Synthetic polymer.
QVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 97 - protein, 248 aa, Artificial Sequence
  Synthetic polymer.
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 98 - protein, 245 aa, Artificial Sequence
  Synthetic polymer.
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSSASGGGGSGGGGSGGGGSDIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 99 - protein, 244 aa, Artificial Sequence
  Synthetic polymer.
QVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSSASGGGGSGGGGSGGGGSDIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 100 - protein, 244 aa, Artificial Sequence
  Synthetic polymer.
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 101 - protein, 255 aa, Artificial Sequence
  Synthetic polymer.
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSGGGGSGGGGSGGGGSGGGGSAEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 102 - protein, 247 aa, Artificial Sequence
  Synthetic polymer.
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSGGGGSGGGGSGGGGSGGGGSEIVLTQSPGTLSLSPGERATLSCRASSSVPYIHWYQQKPGQAPRLLIYATSALASGIPDRFSGSGSGTDFTLTISRLEPEDFAVYYCQQWLSHPPTFGQGTKLEIK

SEQ ID NO: 103 - protein, 247 aa, Artificial Sequence
  Synthetic polymer.
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSGGGGSGGGGSGGGGSGGGGSQIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 104 - protein, 249 aa, Artificial Sequence
  Synthetic polymer.
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 105 - protein, 250 aa, Artificial Sequence
  Synthetic polymer.
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 106 - protein, 248 aa, Artificial Sequence
  Synthetic polymer.
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 107 - protein, 244 aa, Artificial Sequence
  Synthetic polymer.
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 108 - protein, 244 aa, Artificial Sequence
  Synthetic polymer.
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 109 - protein, 243 aa, Artificial Sequence
  Synthetic polymer.
QIQLVQSGPELKKPGETVKISCKASGYTFTNYGMNWVKQAPGKGLKWMGWINTKTGEPTYAEEFKGRFAFSLETSASTAYLQINNLKKEDTATYFCGRGGYGSSYWYFDVWGAGTTVTVSSGGGGSGGGGSGGGGSDIVMTQSHKFMSTSVGDRVSITCKASQDVSIAVAWYQQKPGQSPKVLIYSASYRYTGVPDRFTGSGSGTDFTFTISRVQAEDLAVYYCQQHYITPLTFGAGTKLELK

SEQ ID NO: 110 - protein, 243 aa, Artificial Sequence
  Synthetic polymer.
DIVMTQSHKFMSTSVGDRVSITCKASQDVSIAVAWYQQKPGQSPKVLIYSASYRYTGVPDRFTGSGSGTDFTFTISRVQAEDLAVYYCQQHYITPLTFGAGTKLELKGGGGSGGGGSGGGGSQIQLVQSGPELKKPGETVKISCKASGYTFTNYGMNWVKQAPGKGLKWMGWINTKTGEPTYAEEFKGRFAFSLETSASTAYLQINNLKKEDTATYFCGRGGYGSSYWYFDVWGAGTTVTVSS

SEQ ID NO: 111 - protein, 241 aa, Artificial Sequence
  Synthetic polymer.
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSGGGGSGGGGSGGGGSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKR

SEQ ID NO: 112 - protein, 241 aa, Artificial Sequence
  Synthetic polymer.
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGGGSGGGGSGGGGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 113 - protein, 242 aa, Artificial Sequence
  Synthetic polymer.
EVQLQESGPGLVKPGGSLSLSCAASGFVFSSYDMSWVRQTPERRLEWVAYISSGGGITYFPSTVKGRFTVSRDNAKNTLYLQMNSLTSEDTAIYYCAAHYFGSSGPFAYWGQGTLVTVSAGGGGSGGGGSGGGGSDIQMTQSPASLSASVGDTVTITCRASENIFSYLAWYQQKPGKSPKLLVYNTKTLAEGVPSRFSGSGSGTQFSLTISSLQPEDFGSYYCQHHYGTPFTFGSGTKLEIK

SEQ ID NO: 114 - protein, 241 aa, Artificial Sequence
  Synthetic polymer.
DIQMTQSPASLSASVGDTVTITCRASENIFSYLAWYQQKPGKSPKLLVYNTKTLAEGVPSRFSGSGSGTQFSLTISSLQPEDFGSYYCQHHYGTPFTFGSGTKLEIKGGGGSGGGGSGGGGSVQLQESGPGLVKPGGSLSLSCAASGFVFSSYDMSWVRQTPERRLEWVAYISSGGGITYFPSTVKGRFTVSRDNAKNTLYLQMNSLTSEDTAIYYCAAHYFGSSGPFAYWGQGTLVTVSA

SEQ ID NO: 115 - protein, 252 aa, Artificial Sequence
  Synthetic polymer.
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 116 - protein, 252 aa, Artificial Sequence
  Synthetic polymer.
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 117 - protein, 240 aa, Artificial Sequence
  Synthetic polymer.
QVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 118 - protein, 240 aa, Artificial Sequence
  Synthetic polymer.
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKGGGGSGGGGSGGGGSQVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 119 - protein, 245 aa, Artificial Sequence
  Synthetic polymer.
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKR

SEQ ID NO: 120 - protein, 245 aa, Artificial Sequence
  Synthetic polymer.
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 121 - protein, 240 aa, Artificial Sequence
  Synthetic polymer.
QVQLVQSGAEVKKPGASVKVSCKASGYTFTSYYMHWVRQAPGQGLEWMGIINPNGGSTSYAQKFQGRVTMTRDTSTSTVYMELSSLRSEDTAVYYCARDSSYDAFDIWGQGTMVTVSSGGGGSGGGGSGGGGSAIQLTQSPSTLSASVGDRVTITCQASQDISNYLNWYQQKPGKAPKLLINDASYLETGVPSRFSGSGSGTDFTLTISSLQPEDIATYYCQQYESLPYTFGQGTKLEIK

SEQ ID NO: 122 - protein, 240 aa, Artificial Sequence
  Synthetic polymer.
AIQLTQSPSTLSASVGDRVTITCQASQDISNYLNWYQQKPGKAPKLLINDASYLETGVPSRFSGSGSGTDFTLTISSLQPEDIATYYCQQYESLPYTFGQGTKLEIKGGGGSGGGGSGGGGSQVQLVQSGAEVKKPGASVKVSCKASGYTFTSYYMHWVRQAPGQGLEWMGIINPNGGSTSYAQKFQGRVTMTRDTSTSTVYMELSSLRSEDTAVYYCARDSSYDAFDIWGQGTMVTVSS

SEQ ID NO: 123 - protein, 245 aa, Artificial Sequence
  Synthetic polymer.
QVTLKESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARDFGRWSYYFDYWSQGTLVTVSSGGGGSGGGGSGGGGSQSVLTQPSSVSGTPGQRVTISCSGSSSNIGSNYVYWYQQLPGTAPKWYRNNQRPSGVPDRFSGSKSGTSASLAISGLRSEDEADYYCAAWDDSLSGVVFGGGTKLTVL

SEQ ID NO: 124 - protein, 245 aa, Artificial Sequence
  Synthetic polymer.
QSVLTQPSSVSGTPGQRVTISCSGSSSNIGSNYVYWYQQLPGTAPKWYRNNQRPSGVPDRFSGSKSGTSASLAISGLRSEDEADYYCAAWDDSLSGVVFGGGTKLTVLGGGGSGGGGSGGGGSQVTLKESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARDFGRWSYYFDYWSQGTLVTVSS

SEQ ID NO: 125 - protein, 241 aa, Artificial Sequence
  Synthetic polymer.
EVQLVESGGGLVQPGRSLRLSCTASGFTFGDYAMSWVRQAPGKGLEWVSSISGSGRSTDHADYVKGRFTISRDNSKNTVYLQMNRLRAEDTAVYYCAKVSNYEYYFDYWAQGTLTVSSGGGGSGGGGSGGGGSEIVLTQSPSVSVAPGQTARITCGGSNIGSESVNWYQWKSGQVPVLVVSDTTDPRSGIPGRFTGTRSGTTATLTISGVEAGDEADYHCQVWDDTGDHPVFGGGTKLTVL

SEQ ID NO: 126 - protein, 241 aa, Artificial Sequence
  Synthetic polymer.
EIVLTQSPSVSVAPGQTARITCGGSNIGSESVNWYQWKSGQVPVLVVSDTTDPRSGIPGRFTGTRSGTTATLTISGVEAGDEADYHCQVWDDTGDHPVFGGGTKLTVLGGGGSGGGGSGGGGSEVQLVESGGGLVQPGRSLRLSCTASGFTFGDYAMSWVRQAPGKGLEWVSSISGSGRSTDHADYVKGRFTISRDNSKNTVYLQMNRLRAEDTAVYYCAKVSNYEYYFDYWAQGTLTVSS

(19) United States
(12) Patent Application Publication
(10) Pub. No.: US 2023/0416333 A1
(43) Pub. Date: Dec. 28, 2023

(54) HOMODIMERIC AND HETERODIMERIC PROTEINS COMPRISING BUTYROPHILIN

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US)

(21) Appl. No.: 18/033,403

(22) PCT Filed: Apr. 14, 2021
(86) PCT No.: PCT/US21/27294

§ 371 Date: Apr. 24, 2023

Related U.S. Application Data
(60) Provisional application No. 63/105,744, filed on Oct. 26, 2020.

(51) Int. Cl. C07K 14/705; C07K 16/28; C12N 15/63; A61P 37/04

(52) U.S. Cl. CPC C07K 14/70503; C07K 16/2803; C12N 15/63; A61P 37/04; C07K 2317/524; C07K 2317/526; C07K 2317/53

(57) ABSTRACT

The present disclosure relates, inter alia, to compositions and methods, including heterodimeric proteins and chimeric proteins comprising portions of butyrophilin family of proteins that find use in the treatment of disease, such as immunotherapies for cancer and autoimmunity.

### PRIORITY

This application claims the benefit of, and priority to, U.S. Application No. 63/105,744, filed Oct. 26, 2020, which is hereby incorporated by reference in its entirety.

### TECHNICAL FIELD

The current disclosure relates to heterodimeric proteins that find use in the treatment of diseases, such as immunotherapies for cancer and autoimmunity.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SHK-033PC_116981-5033_ST25; date created: Apr. 13, 2021; file size: 354,746 bytes).

### BACKGROUND

Gamma delta T cells amount to up to 5% of all T cells in a human, but they play an important role against cancer. Recent research has indicated that the amount of gamma delta T cells that infiltrate a tumor is an excellent predictor of a favorable outcome for the patient. Further, unlike the alpha beta T cells commonly used in CAR-T therapy, gamma delta T cells play a role in the innate immune response. The prognostic significance of gamma delta T cells in cancer has prompted an effort to manipulate gamma delta T cells as a therapeutic strategy for cancer. Current approaches are limited to ex vivo strategies, where a patients gamma delta T cells are either harvested and modified to express a chimeric antigen receptor, and/or expanded to greater numbers in cell culture, followed by infusion of the modified gamma delta T cells back into the cancer patient (Front Immunol. 2018 Jun. 26; 9:1409). Strategies to manipulate gamma delta T cells directly in cancer patients have been hampered by an inability to conclusively identify the molecular entities directly recognized by the gamma delta T cell receptor (Nat Immunol. 20(2):121-128 (2019)). In fact, the most widely accepted activators of gamma delta T cells include largely intracellular molecules such as heat shock proteins, intermediates of the non-mevalonate pathway of isopentyl pyrophosphate (IPP) biosynthesis (including HMB-PP), intracellular bacteria (eg. mycobacteria and listeria), viruses (eg. cytomegalovirus), and other lipid antigens.

Accordingly, there remains a need for novel compositions and methods gamma-delta T cell engagement that do not require use of the above molecules.

### SUMMARY

Accordingly, in one aspect, the current disclosure provides a heterodimeric protein comprising (a) a first domain comprising BTN2A1 and/or BTN3A1 butyrophilin family proteins, or fragments thereof; (b) a second domain comprising a targeting domain that specifically binds to CD19; and (c) a linker that adjoins the first and second domain and which facilitates heterodimerization.

In one aspect, the current disclosure relates to a heterodimeric protein comprising an alpha chain and a beta chain, wherein the alpha chain comprises: (a) a first domain comprising a BTN2A1 protein, or a fragment thereof; (b) a second domain comprising a targeting domain that specifically binds to CD19; and (c) a linker that adjoins the first and second domain; and wherein the beta chain comprises: (a) a first domain comprising a BTN3A1 protein, or a fragment thereof; (b) a second domain comprising a targeting domain that specifically binds to CD19; and (c) a linker that adjoins the first and second domains.

In one aspect, the current disclosure relates to a heterodimeric protein comprising an alpha chain and a beta chain, wherein the alpha chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domain; and wherein the beta chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence.

In one aspect, the current disclosure relates to a heterodimeric protein comprising: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence. In embodiments, two of the heterodimeric proteins associate to form a heterodimer.

In embodiments, the targeting domain is capable of binding CD19 on the surface of a cancer cell. In embodiments, the targeting domain is an antibody-like molecule, or antigen binding fragment thereof. In embodiments, the antibody-like molecule is an scFv.

In embodiments, the heterodimeric protein is capable of engaging gamma-delta T cells. In embodiments, the gamma delta T cell are Vγ9δ2 T cells.

In embodiments, the protein modulates the function of gamma delta T cells. In embodiments, the gamma delta T cell are Vγ9δ2 T cells.

In embodiments the alpha chain and the beta chain self-associate to form the heterodimer.

In various aspects, the heterodimeric protein of the current disclosure is used for contemporaneous activation and targeting of gamma delta T cells to tumor cells, modulating a patient's immune response, and/or stimulating proliferation of gamma delta T cells in vivo. Accordingly, in various aspects, the heterodimeric protein of the current disclosure is used in a method for treating cancer, infectious, or autoimmune diseases comprising administering an effective amount of a pharmaceutical composition comprising the heterodimeric protein to a patient in need thereof.

In various aspects, the heterodimeric protein of the current disclosure is used for stimulating proliferation of gamma delta T cells by administering an effective amount of a pharmaceutical composition of the current disclosure to a subject in need thereof thereby causing an in vivo proliferation of gamma delta T cells and/or contacting an effective amount of a pharmaceutical composition of the current disclosure with a cell derived from a subject in need thereof thereby causing an ex vivo proliferation of gamma delta T cells.

In various aspects, the heterodimeric protein of the current disclosure is used for stimulating proliferation of gamma delta T cells in the absence of heat shock proteins, intermediates of the non-mevalonate pathway of isopentyl pyrophosphate (IPP) biosynthesis (including HMB-PP), intracellular bacteria (eg. mycobacteria and listeria), viruses (eg. cytomegalovirus), and other lipid antigens.

Also in various aspects, the present heterodimeric protein is used in a method for treating autoimmune diseases comprising administering an effective amount of a pharmaceutical composition comprising the heterodimeric protein to a patient in need thereof. In further aspects, the present heterodimeric protein is used in a method for treating infections, including without limitation, viral infections or other intracellular pathogens. In still further aspects, the present heterodimeric protein is used in a method for treating cancers.

Also provided in various aspects are pharmaceutical compositions comprising the heterodimeric protein of any of the embodiments disclosed herein, expression vectors comprising a nucleic acids encoding the heterodimeric protein of any of the embodiments disclosed herein, or host cells comprising expression vectors comprising a nucleic acids encoding the heterodimeric protein of any of the embodiments disclosed herein. Any aspect or embodiment disclosed herein can be combined with any other aspect or embodiment as disclosed herein.

In one aspect, the current disclosure provides heterodimeric protein: (a) a first domain comprising (i) a BTN2A1 protein, or a fragment thereof, and (ii) a BTN3A1 protein, or a fragment thereof; (b) a second domain comprising a targeting domain that specifically binds to CD19; and (c) a linker that adjoins the first and second domain, wherein the BTN2A1 protein, or the fragment thereof, and the BTN3A1 protein, or the fragment thereof are adjoined by a second linker. In embodiments, the second linker is a flexible amino acid sequence.

In one aspect, the current disclosure provides a heterodimeric protein comprising an alpha chain and a beta chain, wherein the alpha chain comprises: (a) a first domain comprising (i) a BTN2A1 protein, or a fragment thereof, and (ii) a BTN3A1 protein, or a fragment thereof; (b) a second domain comprising a targeting domain that specifically binds to CD19; and (c) a linker that adjoins the first and second domain; and wherein the beta chain comprises: (a) a first domain (i) a BTN2A1 protein, or a fragment thereof, and (ii) a BTN3A1 protein, or a fragment thereof; (b) a second domain comprising a targeting domain that specifically binds to CD19; and (c) a linker that adjoins the first and second domains. In embodiments, the second linker is a flexible amino acid sequence.

In one aspect, the current disclosure relates to a chimeric protein of a general structure of: N terminus-(a)-(b)-(c)-C terminus, wherein: (a) is the first domain comprising the general structure of (a1)-SL-(a2), wherein (a1) is an extracellular domain (ECD) of a butyrophilin family protein, or a fragment thereof, (a2) is an extracellular domain (ECD) of a butyrophilin family protein, or a fragment thereof, and SL is a second linker adjoins (a1) and (a2) comprising a flexible amino acid sequence of about 4 to about 50 amino acids length, and (c) is a second domain comprising a targeting domain, the targeting domain being selected from (i) an antibody, antibody-like molecule, or antigen binding fragment thereof, and (ii) an extracellular domain of a membrane protein. (b) is linker that adjoins the first and second domains, wherein the a linker comprises at least one cysteine residue capable of forming a disulfide bond.

In embodiments, the (a1) and (a2) are two of the same butyrophilin family proteins. In embodiments, the (a1) and (a2) are different butyrophilin family proteins. In embodiments, the (a1) and/or (a2) is a fragment of the butyrophilin family protein comprising a variable domain. In embodiments, the (a1) and (a2) comprise butyrophilin family proteins independently selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the butyrophilin family proteins are independently selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In embodiments, the targeting domain is capable of binding an antigen on the surface of a cancer cell. In embodiments, the targeting domain comprises an extracellular domain of a membrane protein selected from LAG-3, PD-1, TIGIT, CD19, or PSMA.

In embodiments, the targeting domain is an antibody, or an antigen binding fragment thereof. In embodiments, the binding fragment comprises an Fv domain. In embodiments, the targeting domain is an antibody-like molecule, or antigen binding fragment thereof. In embodiments, the binding fragment comprises an scFv domain.

In embodiments, the targeting domain specifically binds one of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, and GD3. In embodiments, the targeting domain specifically binds CD19. In embodiments, the targeting domain specifically binds PSMA. In embodiments, the targeting domain specifically binds CD33. In embodiments, the targeting domain specifically binds CLL-1.

In embodiments, the linker comprises the hinge-CH2-CH3 Fc domain. In embodiments, he hinge-CH2-CH3 Fc domain is derived from IgG1, optionally human IgG1. In embodiments, the hinge-CH2-CH3 Fc domain is derived from IgG4, optionally human IgG4.

In embodiments, the chimeric protein is a homodimer.

In one aspect, the current disclosure relates to a pharmaceutical composition, comprising the chimeric protein of any of the embodiments disclosed herein.

In one aspect, the current disclosure relates to an expression vector, comprising a nucleic acid encoding the first and/or second polypeptide chains of the chimeric protein of any of the embodiments disclosed herein. In embodiments, the expression vector is a mammalian expression vector. In embodiments, the expression vector comprises DNA or RNA.

In one aspect, the current disclosure relates to a host cell, comprising the expression vector of any of the embodiments disclosed herein.

In one aspect, the current disclosure relates to a method of contemporaneous activation and targeting of gamma delta T cells to tumor cells comprising administering to a subject in need thereof an effective amount of a pharmaceutical composition of any of the embodiments disclosed herein to a subject in need thereof.

In one aspect, the current disclosure relates to a method of modulating a patient's immune response, comprising administering an effective amount of a pharmaceutical composition of any of the embodiments disclosed herein to a subject in need thereof.

In one aspect, the current disclosure relates to a method of stimulating proliferation of gamma delta T cells, comprising: administering an effective amount of a pharmaceutical composition of any of the embodiments disclosed herein to a subject in need thereof thereby causing an in vivo proliferation of gamma delta T cells and/or contacting an effective amount of a pharmaceutical composition of any of the embodiments disclosed herein with a cell derived from a subject in need thereof thereby causing an ex vivo proliferation of gamma delta T cells.

In embodiments, the subject's T cells are activated by the first domain. In embodiments, the subject has a tumor and the gamma delta T cells modulate cells of the tumor.

In one aspect, the current disclosure relates to a method of treating cancer, comprising administering to a subject in need thereof an effective amount of a pharmaceutical composition of any of the embodiments disclosed herein to a subject in need thereof. In embodiments, the cancer is a lymphoma. In embodiments, the cancer is a leukemia.

### BRIEF DESCRIPTION OF THE FIGURES

FIG. 1A shows a non-limiting schematic representation of a BTN2A1/3A1-Fc-CD19scFv heterodimeric protein, which comprises a heterodimer of i) a human butyrophilin BTN2A1 adjoined to a human CD19-specific scFv via a linker, and ii) a human butyrophilin BTN3A1 adjoined to a human CD19-specific scFv. This GAmma DELta T cell ENgager construct also is referred to herein as the BTN2A1/3A1-Fc-CD19scFv ‘GADLEN’ protein. FIG. 1B shows an illustrative chromatograph for the purified BTN2A1/3A1-Fc-CD19scFv GADLEN protein using FcXL chromatography. The protein was generated by dual-transfection of ExpiCHO or Expi293 cells with both a BTN2A1-Fc-CD19scFv (‘alpha’, chain) and a BTN3A1-Fc-CD19scFv (‘beta’ chain) construct, in which the so-called alpha and beta constructs contained charged polarized linker domains which facilitated heterodimerization of the desired BTN2A1/3A1-Fc-CD19scFv GADLEN protein.

FIG. 2A to FIG. 2C show the gel electrophoresis and western blot analysis of a purified BTN2A1/3A1-Fc-CD19scFv GADLEN protein. FIG. 2A shows an image of a SDS-PAGE gel of the BTN2A1/3A1-Fc-CD19scFv GADLEN protein stained with Coomassie blue indicating >90% purity. FIG. 2B shows the western blot analysis of a purified BTN2A1/3A1-Fc-CD19scFv GADLEN protein. The purified protein was analyzed by Western blot using non-reduced (lane “NR”), reduced (lane “R”) and both reduced and deglycosylated (lane “DG”) conditions, following detection with an anti-human BTN2A1 antibody, an anti-human BTN3A1 antibody, or an anti-mouse Fc antibody. The results indicate the presence of a disulfide-linked protein that reduces to two individual proteins (following disruption of the interchain disulfide bonds with R-mercaptoethanol) with molecular weights consistent with the predicted molecular weights for the alpha and beta chains. Based on the similarity between the reduced and both reduced and deglycosylated lanes, the BTN2A1/3A1-Fc-CD19scFv GADLEN construct appears to have few glycosylations. FIG. 2C shows the dual color western blot analysis of a purified BTN2A1/3A1-Fc-CD19scFv GADLEN protein. The purified protein was analyzed by Western blot using non-reduced (lane “NR”), reduced (lane “R”) and both reduced and deglycosylated (lane “DG”) conditions, following detection with an anti-human BTN2A1 antibody conjugated with Starbright Blue 520 and anti-human BTN3A1 antibody conjugated with Dylite800. The dual color western blot indicated the presence of BTN2A1-alpha and BTN3A1-beta chains.

FIG. 3 shows the binding kinetics of the BTN2A1/3A1-Fc-CD19scFv GADLEN protein to recombinant CD19-His protein as determined using the Octet system (ForteBio). Recombinant CD19-His protein was immobilized and detected using the BTN2A1/3A1-Fc-CD19scFv GADLEN protein. A heterodimer lacking CD19scFv was used as a negative control. As shown, the BTN2A1/3A1-Fc-CD19scFv GADLEN protein bound to CD19-His protein.

FIG. 4A and FIG. 4B show the results of Meso Scale Discovery (MSD) ELISA assays illustrating contemporaneous binding to anti-BTN2A1/3A1 antibody and CD19 by the BTN2A1/3A1-Fc-CD19scFv GADLEN protein. Recombinant CD19 protein was coated on plates and increasing amounts of the BTN2A1/3A1-Fc-CD19scFv GADLEN protein or a heterodimer lacking CD19scFv were added to the plates for capture by the plate-bound recombinant CD19 protein. The binding was detected using an anti-BTN2A1 antibody (FIG. 4A) or an anti-BTN3A1 antibody (FIG. 4B) using a electrochemiluminescence (ECL) readout.

FIG. 5A to FIG. 5C show the results of an MSD ELISA assays illustrating contemporaneous binding by the BTN2A1/3A1-Fc-CD19scFv GADLEN protein to anti-BTN2A1 and anti-BTN3A1 antibodies. FIG. 5A shows a schematic representation of the MSD ELISA assay used in FIG. 5B. FIG. 5B shows the assay performed with capture with an anti-BTN2A1 antibody and detection with an anti-BTN3A1 antibody. An anti-BTN2A1 antibody was coated on plates and increasing amounts of the BTN2A1/3A1-Fc-CD19scFv GADLEN protein were added to the plates for capture by the plate-bound anti-BTN2A1 antibody. The binding was detected using an anti-BTN3A1 antibody. FIG. 5C) shows the assay performed with capture with an anti-BTN3A1 antibody and detection with an anti-BTN2A1 antibody. An anti-BTN3A1 antibody was coated on plates and increasing amounts of the BTN2A1/3A1-Fc-CD19scFv GADLEN protein were added to the plates for capture by the plate-bound anti-BTN3A1 antibody. The binding was detected using an anti-BTN2A1 antibody.

FIG. 6A and FIG. 6B show the cell surface binding by the BTN2A1/3A1-Fc-CD19scFv GADLEN protein in a CD19-dependent manner. FIG. 6A shows a graph showing the percentage of binding of the BTN2A1/3A1-Fc-CD19scFv GADLEN protein to HEK293 cells expressing CD19 on surface (HEK293-CD19 cells) as assayed by flow cytometry. A heterodimer lacking CD19scFv was used as a negative control for binding. FIG. 6B shows a graph showing the percentage of binding of the BTN2A1/3A1-Fc-CD19scFv GADLEN protein to HEK293 parental cells as assayed by flow cytometry. A heterodimer lacking CD19scFv was used as a negative control for binding.

FIG. 7A and FIG. 7B show the binding to Daudi cells by the GADLEN proteins disclosed herein in a CD19scFv-dependent manner. FIG. 7A shows flow cytometry profiles of Daudi cells stained with isotype control or an anti-CD19 antibody illustrating that Daudi cells are CD19+. FIG. 7B shows a graph showing to Daudi cells the percentage of binding of the BTN2A1/3A1-Fc-CD19scFv GADLEN protein or a human IgG control as assayed by flow cytometry.

FIG. 8A to FIG. 8E demonstrate that the human BTN2A1/3A1-Fc-CD19scFv GADLEN protein specifically binds to Vγ9+Vδ2+T-cells. FIG. 8A shows the cell surface binding to Vγ9+Vδ2+T-cells by the human BTN2A1/3A1-Fc-CD19scFv GADLEN protein. Vγ9+Vδ2+T-cells were isolated and expanded from peripheral blood mononuclear cells (PBMCs) from a healthy donor. The isolated Vγ9+Vδ2+T-cells were incubated with the human BTN2A1/3A1-Fc-CD19scFv GADLEN protein, a control heterodimer protein lacking BTN2A1, or human IgG control. Binding was detected by flow cytometry using an APC conjugated anti-hFc antibody that binds to the Fc-domain of the Heterodimer protein. FIG. 8B shows that the human BTN2A1/3A1-Fc-CD19scFv GADLEN protein does not bind to Vγ9+Vδ1+T-cells. Vγ9+Vδ1+T-cells were isolated and expanded from PBMCs from a healthy donor. The isolated Vγ9+Vδ1+T-cells were incubated with the human BTN2A1/3A1-Fc-CD19scFv GADLEN protein, or human IgG control. Binding was detected by flow cytometry using an APC conjugated anti-hFc antibody that binds to the Fc-domain of the Heterodimer protein. FIG. 8C shows the binding by the human BTN2A1/3A1-Fc-CD19scFv protein to human Vγ9+S2+T cells. Vγ9+Vδ2+T-cells were isolated and expanded from peripheral blood mononuclear cells (PBMCs) from a healthy donor. The isolated Vγ9+Vδ2+T-cells were incubated with the human BTN2A1/3A1-Fc-CD19scFv GADLEN or BTN3A1/3A2-Fc-CD19scFv GADLEN proteins. FIG. 8D shows that the human BTN2A1/3A1-Fc-CD19scFv GADLEN protein does not bind to Vγ9− T-cells. Vγ9− T-cells were isolated and expanded from PBMCs from a healthy donor. The isolated Vγ9− T-cells were incubated with the human BTN2A1/3A1-Fc-CD19scFv GADLEN or BTN3A1/3A2-Fc-CD19scFv GADLEN proteins. Binding was detected by flow cytometry using an APC conjugated anti-hFc antibody that binds to the Fc-domain of the Heterodimer protein. FIG. 8E shows a graph showing the binding of the human BTN2A1/3A1-Fc-CD19scFv GADLEN protein to human γδ T cells expressing the Vγ9δ2 T cell receptor (TCR), compared to a heterodimer lacking BTN2A1. Inset shows binding of the human BTN2A1/3A1-Fc-CD19scFv GADLEN protein to human γδ T cells expressing the Vγ9δ2 TCR compared to unstained cells as shown by flow cytometry.

FIG. 9A and FIG. 9B show the cell surface binding by the BTN2A1 protein to Vγ9+Vδ2+T-cells requires dimerization. FIG. 9A shows the % binding of BTN2A1-His protein, which exists as a monomer in solution, Vγ9+Vδ2+T-cells. SIRPα-His, which binds to CD47 on cells, served as a positive control. Binding was detected using flow cytometry-based on detection of the His tag. FIG. 9B shows the % binding of BTN2A1-Fc, BTN2A1-Fc proteins, the human BTN2A1/3A1-Fc-CD19scFv GADLEN protein or human IgG control to Vγ9+Vδ2+T-cells as measured by flow cytometry. The BTN2A1-Fc protein exists as a dimer in solution. These data suggest that BTN2A1 needs to homodimerize in order to interact with the Vγ9+Vδ2 T cell receptor.

FIG. 10A to FIG. 10E illustrate the cell line development (CLD) for the production of BTN2A1/3A1-Fc-CD19scFv heterodimeric constructs. FIG. 10A shows the co-transfection of 2 single gene vectors (SGV) expressing the alpha chain and beta chain separately. FIG. 10B shows the transfection using a dual gene vector (DGV) that expresses the alpha and beta chain under 2 separate promoters in a single vector. FIG. 10C shows the comparison of BTN2A1-alpha and BTN3A1-beta chains in SGV and DGV mini-pools as assayed by MSD-ELISA based titers of shake flask cultures on day 14 for constructs having charged polarized linkers. FIG. 10D shows the comparison of BTN2A1-alpha and BTN3A1-beta chains in SGV and DGV mini-pools as assayed by qRT-PCR assessment of alpha and beta chain expression in cells for constructs having charged polarized linkers. FIG. 10E shows the comparison of BTN2A1-alpha and BTN3A1-beta chains in DGV mini-pools for constructs having KIH mutations in Fc domain (KIH-Fc) and KIH mutations with FcRn mutations (KIH-FcRn).

FIG. 11 shows a schematic representation of the second version of GADLEN proteins: a homodimeric fusion proteins, without limitation, e.g., the BTN2A1V/3A1V-Fc-CD19scFv homodimeric fusion protein where the variable domains of BTN2A1 and BTN3A1 are strung together in tandem using different kinds of linkers, and fused to the CD19scFv sequence through the IgG4 Fc sequence. Two such chains would homodimerize to form the functional tetramer unit of BTN2A1 and BTN3A1 for Vγ9δ2 TCR activation.

FIG. 12A and FIG. 12B show western blot analysis of the homodimeric GADLEN proteins. The purified BTN2A1V/3A1V-Fc IgG4-CD19scFv (A); 2, BTN2A1V/3A1V-Fc IgG1-CD19scFv (A); and 3, BTN2A1V/3A1V-Fc IgG4-CD19scFv (A2) proteins were analyzed by Western blot using non-reduced (lane “NR”), reduced 15 (lane “R”) conditions, following detection with an anti-human BTN2A1 antibody (FIG. 12A) or an anti-human BTN3A1 antibody (FIG. 12B).

FIG. 13 demonstrates contemporaneous binding by the BTN2A1V/3A1V-Fc-CD19scFv GADLEN protein to CD19 and an anti-BTN3A1 antibody as measured using MSD ELISA assays. Recombinant CD19 protein was coated on plates and the indicated BTN2A1V/3A1V-Fc-CD19scFv GADLEN homodimeric proteins were added to the plates for capture by the plate-bound CD19 protein. The binding was detected using an anti-BTN3A1 antibody.

FIG. 14A and FIG. 14B show the activation of γδ T cells by the indicated BTN2A1V/3A1V-Fc-CD19scFv homodimeric protein (FIG. 14A) or the BTN2A1/3A1-Fc-CD19scFv homodimeric protein (FIG. 14B) in the presence of an anti-NKG2D antibody (Clone #149810) as assayed by flow cytometry. IgG was used as a negative control in the presence of the anti-NKG2D antibody.

FIG. 15A and FIG. 15B show the size exclusion chromatography (SEC) profiles of the BTN2A1/3A1-Fc-CD19scFv heterodimeric GADLEN proteins manufactured using two single gene vectors (SGV, FIG. 15A) and a dual gene vector (DGV, FIG. 15B) approaches.

FIG. 16 shows western blot analysis of the BTN2A1/3A1-Fc-CD19scFv heterodimeric GADLEN proteins manufactured using two single gene vectors (FIG. 15A) and a dual gene vector (FIG. 15B) approaches. The purified protein was processed under non-reduced (lane “NR”), reduced (lane “R”) and both reduced and deglycosylated (lane “D”) conditions, separated using SDS-PAGE and detected with an anti-human BTN2A1 antibody (blue bands, triangular arrowheads) or an anti-human BTN3A1 antibody (green bands, square arrowheads).

FIG. 17 shows a graph comparing the binding to CD19 expressed on a B-cell lymphoma cell line (Daudi) by the BTN2A1V/3A1V-Fc-CD19scFv GADLEN protein produced using two single gene vectors (SGV) and a dual gene vector (DGV) in comparison with a BTN2A1/3A1-Fc-CD19scFv heterodimeric protein reference material. A human IgG protein was used as a negative control and tested at the highest concentration of 6.25 μg/ml. Binding was measured using flow cytometry.

FIG. 18 shows a graph comparing the extent of activation of γδ T cells induced by 6.25 μg/ml of the BTN2A1/3A1-Fc-CD19scFv heterodimeric protein produced using two single gene vectors (SGV) and a dual gene vector (DGV) in comparison with a BTN2A1/3A1-Fc-CD19scFv heterodimeric protein reference material in the presence of an anti-NKG2D antibody (Clone #149810). Activation of γδ T cells was assayed in a plate-bound format and assayed by flow cytometry. IgG was used as a negative control in the presence of the anti-NKG2D antibody.

FIG. 19 shows schematic representations of charged polarized linkers and knob-in-hole (KIH) mutations as the domains that promote heterodimerization and disfavor homodimerization.

FIG. 20 shows a bar graph of the amounts of the BTN2A1-alpha and BTN3A1-beta chains as assayed using an ELISA assay in the culture supernatants of mini pools generated using the charged polarized linkers (CPL) approach and the KIH mutation approach.

FIG. 21A to FIG. 21C show western blot analysis of the BTN2A1/3A1-Fc-CD19scFv heterodimeric GADLEN proteins manufactured using the charged polarized linkers (CPL) approach (FIG. 21A), the KIH mutation approach (FIG. 21B), and the KIH mutation approach with FcRn mutations (KIH-FcRn; FIG. 21C). The purified protein was analyzed by Western blot using non-reduced (lane “NR”), reduced (lane “R”) and both reduced and deglycosylated (lane “D”) conditions, following detection with an anti-human BTN2A1 antibody or an anti-human BTN3A1 antibody.

FIG. 22A to FIG. 22C show graphs comparing the extent of activation of γδ T cells induced the BTN2A1/3A1-Fc-CD19scFv heterodimeric protein produced using the charged polarized linkers (CPL) approach, the KIH mutation approach, and the KIH mutation approach with FcRn mutations in comparison in the presence of an anti-NKG2D antibody (Clone #149810). Activation of γδ T cells was measured in a plate-bound format based on the expression of TNFα (FIG. 22A), IFNγ (FIG. 22B), and CD107a (FIG. 22C) as assayed by flow cytometry. IgG in was used as a negative control the presence of the anti-NKG2D antibody.

### DETAILED DESCRIPTION

The current disclosure is directed to novel chimeric proteins that have the ability to, inter alia, target gamma delta T cells and cause their activation, while also forming a synapse with, e.g., tumor cells. Thus, the present multifunctional chimeric proteins provide for unique means to modulate a subject's immune system for therapy.

The Heterodimeric Proteins of the Current Disclosure

In one aspect, the current disclosure relates to a heterodimeric protein comprising an alpha chain and a beta chain, wherein the alpha chain comprises: (a) a first domain comprising a BTN2A1 protein, or a fragment thereof; (b) a second domain comprising a targeting domain that specifically binds to CD19; and (c) a linker that adjoins the first and second domain; and wherein the beta chain comprises: (a) a first domain comprising a BTN3A1 protein, or a fragment thereof; (b) a second domain comprising a targeting domain that specifically binds to CD19; and (c) a linker that adjoins the first and second domains.

In one aspect, the current disclosure relates to a heterodimeric protein comprising an alpha chain and a beta chain, wherein the alpha chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domain; and wherein the beta chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domains. In embodiments, a second linker adjoins (i) the BTN2A1 protein, or the fragment thereof, and (ii) the BTN3A1 protein, or the fragment thereof.

In embodiments, the second linker is a flexible amino acid sequence. In embodiments, the alpha chain and the beta chain self-associate to form the heterodimer of alpha and beta chains, which comprise a BTN2A12-BTN3A12 tetramer.

In one aspect, the current disclosure relates to a heterodimeric protein comprising: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence. In embodiments, two of the heterodimeric proteins associate to form a heterodimer of two chains, which comprise a BTN2A12-BTN3A12 tetramer.

In one aspect, the current disclosure relates to a heterodimeric protein comprising an alpha chain and a beta chain, wherein the alpha chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) an alpha chain linker that adjoins the first and second domain; and wherein the beta chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a beta chain linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence. In embodiments, the alpha chain linker and the beta chain linker self-associate. In embodiments, the alpha chain and the beta chain self-associate to form the heterodimer of alpha and beta chains, which comprise a BTN2A12-BTN3A12 tetramer. In embodiments, the alpha chain linker and the beta chain linker are charged polarized linkers, wherein one of the alpha chain linker and the beta chain linker is positively charged and the other is negatively charged. In embodiments, the alpha chain linker and the beta chain linker comprise an Fc domain comprising knob-in-hole (KIH) mutations. In embodiments, the alpha chain linker and the beta chain linker comprise an Fc domain comprising KIH mutations and FcRn mutations.

In embodiments, the alpha chain and the beta chain self-associate to form the heterodimer.

In embodiments, the first domain of the alpha chain comprises the extracellular domain of BTN2A1 protein. In embodiments, the first domain of the alpha chain comprises a polypeptide having an amino acid sequence that has at least about 95% identity with the amino acid sequence of SEQ ID NO: 35 or SEQ ID NO: 71. In embodiments, the first domain of the alpha chain comprises a polypeptide having an amino acid sequence that is identical to the amino acid sequence of SEQ ID NO: 35 or SEQ ID NO: 71. In embodiments, the first domain of the beta chain comprises the extracellular domain of BTN3A1 protein. In embodiments, the first domain of the beta chain comprises a polypeptide having an amino acid sequence that has at least about 95% identity with the amino acid sequence of SEQ ID NO: 19 or SEQ ID NO: 72. In embodiments, the first domain of the beta chain comprises a polypeptide having an amino acid sequence that is identical to the amino acid sequence of SEQ ID NO: 19 or SEQ ID NO: 72.

In embodiments, the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains. In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally human IgG1. In embodiments, the linker comprises the hinge-CH2-CH3 Fc domain derived from IgG4, optionally human IgG4. In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains. In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12). In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where each n is independently an integer 0 to 4) (SEQ ID NO: 6).

In embodiments, the linker of alpha chain and/or beta chain comprises a polypeptide having an amino acid sequence that has at least about 95% identity with an amino acid sequence selected from SEQ ID NOs: 15-17, 28-32 and 52-55. In embodiments, the linker of alpha chain and/or beta chain comprises a polypeptide having an amino acid sequence that is identical to an amino acid sequence the amino acid sequence selected from SEQ ID NOs: 15-17, 28-32 and 52-55. In embodiments, the linker of alpha chain and/or beta chain comprises a polypeptide having an amino acid sequence that has at least about 95% identity with an amino acid sequence selected from SEQ ID NOs: 15-17 and 28-32. In embodiments, the linker of alpha chain and/or beta chain comprises a polypeptide having an amino acid sequence that is identical to an amino acid sequence the amino acid sequence selected from SEQ ID NOs: 15-17 and 28-32.

In embodiments, the second domain of the alpha chain and/or beta chain comprises a polypeptide having an amino acid sequence that has at least about 95% identity with an amino acid sequence selected from SEQ ID NOs: 20-23. In embodiments, the second domain of the alpha chain and/or beta chain comprises a polypeptide having an amino acid sequence that is identical to an amino acid sequence the amino acid sequence selected from SEQ ID NOs: 20-27 and 94-126.

In embodiments, the alpha chain comprises a polypeptide having an amino acid sequence that has at least about 95% identity with an amino acid sequence selected from SEQ ID NOs: 37-39. In embodiments, the alpha chain comprises a polypeptide having an amino acid sequence that is identical to an amino acid sequence the amino acid sequence selected from SEQ ID NOs: 37-39.

In embodiments, the beta chain comprises a polypeptide having an amino acid sequence that has at least about 95% identity with an amino acid sequence selected from SEQ ID NOs: 40-42. In embodiments, the beta chain comprises a polypeptide having an amino acid sequence that is identical to an amino acid sequence the amino acid sequence selected from SEQ ID NOs: 40-42. In embodiments, the heterodimeric chimeric protein comprises an amino acid sequence that is identical to an amino acid sequence the amino acid sequence of: (a) SEQ ID NO: 37 and SEQ ID NO: 40; (b) SEQ ID NO: 38 and SEQ ID NO: 41; or (c) SEQ ID NO: 39 and SEQ ID NO: 42.

The sequences of exemplary embodiments of GADLEN fusion proteins are provided in the Table below (Leader sequence is indicated by a double underlined font, extracellular domain of human BTN2A1 is shown in bold-underlined-italicized font, extracellular domain of human BTN3A1 is shown in bold-underlined font, a core domain of the linker is shown in a single underlined font, and anti-CD19 ScFv sequence is shown in a boldface font):

| Description | Sequence |
|---|---|
| Human | MEFGLSWVFLVAIIKGVQCQFSVLGPSGPILAMVGEDADLPCHLFPTMSA |
| BTN3A1-Alpha-scFvCD19 | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | RKGGKRGSKYGPPCPPCPAPEFLGGPSVFLFPPKPKDQLMISRTPEVTCV |
| | VVDVSQEDPEVQFNWYVDGVEVHNAKTKPREEQFNSTYRVVSVLTVLHQ |
| | DWLSGKEYKCKVSSKGLPSSIEKTISNATGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVLHEALHNHYTQKSLSLSLGKDEGGEDGSQVQLQ |
| | QSGAELVRPGSSVKISCKASGYAFSSYWMNWVKQRPGQGLEWIGQIWP |
| | GDGDTNYNGKFKGKATLTADESSSTAYMQLSSLASEDSAVYFCARRETT |
| | TVGRYYYAMDYWGQGTTVTVSSGGGGSGGGGSGGGGSDIQLTQSPAS |
| | LAVSLGQRATISCKASQSVDYDGDSYLNWYQQIPGQPPKLLIYDASNLVS |
| | GIPPRFSGSGSGTDFTLNIHPVEKVDAATYHCQQSTEDPWTFGGGTKLEI |
| | K (SEQ ID NO: 33) |
| Human | MEFGLSWVFLVAIIKGVQCQFSVLGPSGPILAMVGEDADLPCHLFPTMSA |
| BTN3A1-Alpha-19scFv3 | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | RKGGKRGSKYGPPCPPCPAPEFLGGPSVFLFPPKPKDQLMISRTPEVTCV |
| | VVDVSQEDPEVQFNWYVDGVEVHNAKTKPREEQFNSTYRVVSVLTVLHQ |
| | DWLSGKEYKCKVSSKGLPSSIEKTISNATGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVLHEALHNHYTQKSLSLSLGKDEGGEDGSDIQMTQ |
| | TTSSLSASLGDRVTISCRASQDISKYLNWYQQKPDGTVKLLIYHTSRLHS |
| | GVPSRFSGSGSGTDYSLTISNLEQEDIATYFCQQGNTLPYTFGGGTKLEIT |
| | GGGSGGGSGGGSEVKLQESGPGLVAPSQSLSVTCTVSGVSLPDYGVS |
| | WIRQPPRKGLEWLGVIWGSETTYYNSALKSRLTIIKDNSKSQVFLKMNSL |
| | QTDDTAIYYCAKHYYYGGSYAMDYWGQGTSVTVSS (SEQ ID NO: 34) |
| Human | MEFGLSWVFLVAIIKGVQC [symbol] [symbol] |
| BTN2A1-Alpha-scFvCD19 | [symbol] [symbol] |
| with a polarized linker | [symbol] [symbol] |
| | [symbol] [symbol] |
| | [symbol] [symbol] GSGSRKGGKRG |
| | SKYGPPCPPCPAPEFLGGPSVFLFPPKPKDQLMISRTPEVTCVVVDVSQE |
| | DPEVQFNWYVDGVEVHNAKTKPREEQFNSTYRVVSVLTVLHQDWLSGKE |
| | YKCKVSSKGLPSSIEKTISNATGQPREPQVYTLPPSQEEMTKNQVSLTCLV |
| | KGFYPSDIAVEWESNGQPENNYKTTPPVLDSDGSFFLYSRLTVDKSRWQE |
| | GNVFSCSVLHEALHNHYTQKSLSLSLGKDEGGEDGSDIQMTQTTSSLSA |
| | SLGDRVTISCRASQDISKYLNWYQQKPDGTVKLLIYHTSRLHSGVPSRFS |
| | GSGSGTDYSLTISNLEQEDIATYFCQQGNTLPYTFGGGTKLEITGGGSGG |
| | GSGGGSEVKLQESGPGLVAPSQSLSVTCTVSGVSLPDYGVSWIRQPPR |
| | KGLEWLGVIWGSETTYYNSALKSRLTIIKDNSKSQVFLKMNSLQTDDTAI |
| | YYCAKHYYYGGSYAMDYWGQGTSVTVSS (SEQ ID NO: 37) |
| | Nucleotide Sequence of Human BTN2A1-Alpha-scFvCD19 with a polarized |
| | linker: |
| | ATGGAGTTCGGCCTGAGCTGGGTGTTTCTGGTGGCCATCATCAAGGGC |
| | GTGCAGTGCCAGTTCATCGTGGTGGGCCCTACCGACCCAATCCTGGC |
| | CACAGTGGGCGAGAACACCACACTGAGGTGTCACCTGTCCCCAGAGA |
| | AGAATGCCGAGGATATGGAGGTGCGGTGGTTCAGATCTCAGTTTAGCC |
| | CCGCCGTGTTCGTGTATAAGGGCGGCCGGGAGAGAACCGAGGAGCAG |
| | ATGGAGGAGTACAGGGGCCGCACCACATTTGTGAGCAAGGACATCTCC |
| | CGCGGCTCTGTGGCCCTGGTCATCCACAACATCACCGCCCAGGAGAA |
| | TGGCACATATCGGTGCTACTTTCAGGAGGGCAGATCCTACGATGAGGC |
| | CATCCTGCACCTGGTGGTGGCAGGCCTGGGATCTAAGCCCCTGATCAG |
| | CATGAGGGGACACGAGGACGGAGGAATCAGGCTGGAGTGTATCAGCA |
| | GAGGCTGGTATCCCAAGCCTCTGACCGTGTGGAGAGATCCCTACGGA |
| | GGAGTGGCACCTGCCCTGAAGGAGGTGTCCATGCCAGACGCCGATGG |
| | CCTGTTCATGGTGACCACAGCCGTGATCATCCGGGACAAGTCTGTGAG |
| | AAATATGTCTTGCAGCATCAACAATACACTGCTGGGCCAGAAGAAGGAG |
| | AGCGTGATCTTCATCCCCGAGTCCTTTATGCCATCCGTGTCTCCATGTG |
| | CAGGAAGCGGCTCCAGGAAGGGAGGCAAGAGGGGAAGCAAGTATGG |
| | ACCACCTTGCCCACCATGTCCAGCACCAGAGTTTCTGGGAGGACCATC |
| | CGTGTTCCTGTTTCCTCCAAAGCCCAAGGACCAGCTGATGATCTCCAG |
| | GACCCCAGAGGTGACATGCGTGGTGGTGGACGTGTCTCAGGAGGATC |
| | CTGAGGTGCAGTTCAACTGGTACGTGGACGGCGTGGAGGTGCACAAT |
| | GCCAAGACCAAGCCCAGGGAGGAGCAGTTTAACTCCACCTATCGCGT |
| | GGTGTCTGTGCTGACAGTGCTGCACCAGGACTGGCTGAGCGGCAAGG |
| | AGTACAAGTGCAAGGTGAGCTCCAAGGGCCTGCCTTCTAGCATCGAGA |
| | AGACCATCTCCAACGCCACAGGCCAGCCCAGAGAGCCTCAGGTGTATA |
| | CCCTGCCCCCTAGCCAGGAGGAGATGACCAAGAATCAGGTGTCCCTG |
| | ACATGTCTGGTGAAGGGCTTCTACCCTTCCGATATCGCCGTGGAGTGG |
| | GAGTCTAACGGCCAGCCAGAGAACAATTATAAGACCACACCACCCGTG |
| | CTGGACAGCGATGGCTCCTTCTTTCTGTACTCTAGGCTGACCGTGGAC |
| | AAGAGCCGCTGGCAGGAGGGCAACGTGTTTTCTTGCAGCGTGCTGCA |
| | CGAGGCCCTGCACAATCACTACACACAGAAGTCCCTGTCTCTGAGCCT |
| | GGGCAAGGATGAGGGAGGAGAGGACGGAAGCGATATCCAGATGACCC |
| | AGACCACATCCTCTCTGTCCGCCTCTCTGGGCGACAGGGTGACAATCT |
| | CCTGTCGCGCCTCTCAGGATATCAGCAAGTATCTGAATTGGTATCAGCA |
| | GAAGCCTGACGGCACCGTGAAGCTGCTGATCTATCACACATCCCGGCT |
| | GCACTCTGGCGTGCCAAGCAGATTCAGCGGATCCGGATCTGGCACCG |
| | ACTACTCCCTGACAATCTCTAACCTGGAGCAGGAGGATATCGCCACCTA |
| | TTTCTGCCAGCAGGGCAATACCCTGCCTTACACATTTGGGGGGGGCAC |
| | CAAGCTGGAGATCACAGGCGGAGGAAGCGGAGGAGGATCCGGAGGA |
| | GGATCTGAGGTGAAGCTGCAGGAGAGCGGACCTGGCCTGGTGGCAC |
| | CAAGCCAGTCCCTGTCTGTGACCTGTACAGTGTCTGGCGTGAGCCTGC |
| | CCGATTACGGCGTGTCTTGGATCAGGCAGCCTCCAAGGAAGGGCCTG |
| | GAGTGGCTGGGCGTGATCTGGGGCAGCGAGACAACATACTATAACAGC |
| | GCCCTGAAGTCCCGGCTGACCATCATCAAGGACAACAGCAAGTCCCA |
| | GGTGTTCCTGAAGATGAATTCCCTGCAGACCGACGATACAGCCATCTAC |
| | TATTGTGCCAAGCACTACTATTACGGCGGCTCTTATGCCATGGATTACTG |
| | GGGCCAGGGCACCAGCGTGACAGTGAGCTCCTGA |
| | (SEQ ID NO: 46) |
| Human | MEFGLSWVFLVAIIKGVQC [symbol] [symbol] |
| BTN2A1-Alpha-scFvCD19 | [symbol] [symbol] |
| with linker having knob-in- | [symbol] [symbol] |
| hole mutations | [symbol] [symbol] |
| | [symbol] [symbol] EPKSCDKTHTCP |
| | PCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPSDI |
| | AVEWESNGQPENNYKTTPPVLDSDGSFFLYSKLTVDKSRWQQGNVFSCS |
| | VMHEALHNHYTQKSLSLSPGKIEGRMDDIQMTQTTSSLSASLGDRVTISC |
| | RASQDISKYLNWYQQKPDGTVKLLIYHTSRLHSGVPSRFSGSGSGTDYS |
| | LTISNLEQEDIATYFCQQGNTLPYTFGGGTKLEITGGGSGGGSGGGSEVK |
| | LQESGPGLVAPSQSLSVTCTVSGVSLPDYGVSWIRQPPRKGLEWLGVIW |
| | GSETTYYNSALKSRLTIIKDNSKSQVFLKMNSLQTDDTAIYYCAKHYYYG |
| | GSYAMDYWGQGTSVTVSS (SEQ ID NO: 38) |
| | Nucleotide sequence of human BTN2A1-Alpha-scFvCD19 with linker having |
| | knob-in-hole mutations: |
| | ATGGAGTTTGGCCTGTCTTGGGTGTTTCTGGTGGCTATCATCAAGGGA |
| | GTGCAGTGTCAGTTCATCGTTGTGGGACCTACCGATCCTATCTTGGCTA |
| | CAGTGGGCGAGAATACAACCCTGAGATGTCACTTGTCTCCTGAGAAGA |
| | ACGCCGAGGATATGGAGGTTAGGTGGTTCAGATCCCAGTTCTCTCCTG |
| | CCGTGTTTGTGTATAAGGGAGGCAGAGAGAGAACAGAAGAGCAGATG |
| | GAGGAGTACAGAGGAAGAACCACCTTCGTGTCTAAGGACATCAGCAGA |
| | GGCTCTGTGGCTCTGGTGATCCACAATATCACAGCTCAGGAGAATGGC |
| | ACCTACAGATGCTACTTTCAGGAGGGCAGGTCCTACGATGAGGCTATTT |
| | TGCATCTGGTGGTTGCTGGACTGGGATCTAAACCTCTGATCAGCATGA |
| | GGGGACACGAGGATGGAGGAATTAGACTGGAGTGCATCTCTAGAGGCT |
| | GGTATCCTAAACCACTGACAGTGTGGAGAGACCCTTATGGAGGAGTTG |
| | CTCCTGCTCTGAAAGAGGTGTCTATGCCTGATGCTGATGGCCTGTTTAT |
| | GGTGACAACAGCCGTGATCATCCGGGACAAATCCGTGAGGAACATGTC |
| | TTGCTCCATCAACAACACACTGTTGGGACAGAAGAAGGAGAGCGTGAT |
| | CTTCATCCCCGAGAGCTTCATGCCTAGCGTTTCTCCTTGTGCTGAACCT |
| | AAGTCTTGCGACAAGACCCATACATGCCCTCCTTGTCCTGCTCCTGAA |
| | GCTGCTGGAGGACCTTCTGTGTTTTTGTTTCCTCCTAAGCCTAAGGATA |
| | CCCTGATGATCTCCAGAACCCCCGAGGTGACCTGTGTGGTGGTTGATG |
| | TTTCTCATGAGGATCCTGAAGTGAAGTTCAACTGGTACGTGGACGGCG |
| | TGGAGGTGCACAACGCTAAGACAAAACCTAGAGAAGAGCAGTACAACT |
| | CTACCTACAGAGTGGTGTCCGTGCTGACCGTGCTGCACCAAGATTGGT |
| | TGAATGGAAAGGAGTACAAGTGCAAGGTGTCCAACAAGGCTCTGCCTG |
| | CCCCTATCGAGAAGACCATCTCTAAGGCTAAAGGACAACCTAGAGAAC |
| | CTCAGGTGTATACCCTGCCTCCCTGCAGAGATGAGCTGACCAAGAATC |
| | AGGTTTCTCTGTGGTGTCTGGTGAAGGGCTTTTACCCTAGCGACATCG |
| | CTGTGGAGTGGGAGTCTAATGGACAACCTGAGAACAACTACAAGACCA |
| | CACCTCCTGTGCTGGACTCTGATGGCTCCTTCTTCCTGTACTCTAAGCT |
| | GACCGTGGATAAGTCTAGATGGCAACAGGGCAACGTGTTCTCCTGCTC |
| | CGTGATGCATGAAGCTCTGCACAACCACTATACACAGAAGTCTCTGAGC |
| | CTGTCTCCTGGCAAGATCGAGGGCAGAATGGACGATATCCAGATGACA |
| | CAGACAACCTCTTCTCTGTCTGCTTCTCTGGGCGATAGAGTGACCATCA |
| | GCTGCAGAGCTTCTCAGGACATCAGCAAGTATCTGAACTGGTATCAGC |
| | AGAAGCCTGATGGCACCGTGAAGCTGCTGATCTACCACACCTCCAGAT |
| | TGCATTCTGGAGTTCCTTCCAGATTTTCTGGCTCTGGCTCTGGCACCG |
| | ACTATTCTCTGACCATCAGCAATCTGGAACAGGAGGACATCGCTACCTA |
| | CTTTTGCCAGCAGGGCAACACACTGCCTTACACATTTGGAGGAGGAAC |
| | AAAGCTGGAGATCACAGGAGGAGGATCTGGAGGAGGATCTGGAGGAG |
| | GATCTGAAGTTAAACTGCAGGAATCTGGACCAGGATTAGTGGCCCCAT |
| | CTCAGTCTCTGTCTGTGACCTGTACCGTTTCTGGAGTTTCTTTGCCTGA |
| | TTACGGAGTGTCCTGGATCAGACAGCCCCCTAGAAAGGGACTGGAATG |
| | GTTAGGAGTGATTTGGGGATCTGAGACCACCTACTACAACTCTGCCCT |
| | GAAGAGCAGACTGACCATCATCAAGGACAACAGCAAGTCTCAGGTGTT |
| | CCTGAAGATGAACTCCCTGCAGACCGACGATACCGCCATCTACTACTGT |
| | GCTAAGCACTACTACTATGGCGGCTCTTATGCCATGGACTATTGGGGAC |
| | AGGGCACCTCTGTGACAGTGTCTTCTTAA |
| | (SEQ ID NO: 47) |
| Human | MEFGLSWVFLVAIIKGVQC [symbol] [symbol] |
| BTN2A1-Alpha-scFvCD19 | [symbol] [symbol] |
| with linker having knob-in- | [symbol] [symbol] |
| hole mutations and FcRn | [symbol] [symbol] |
| mutations | [symbol] [symbol] EPKSCDKTHTCP |
| | PCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPSDI |
| | AVEWESNGQPENNYKTTPPVLDSDGSFFLYSKLTVDKSRWQQGNVFSCS |
| | VLHEALHSHYTQKSLSLSPGKIEGRMDDIQMTQTTSSLSASLGDRVTISCR |
| | ASQDISKYLNWYQQKPDGTVKLLIYHTSRLHSGVPSRFSGSGSGTDYSL |
| | TISNLEQEDIATYFCQQGNTLPYTFGGGTKLEITGGGSGGGSGGGSEVKL |
| | QESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYGG |
| | SYAMDYWGQGTSVTVSS (SEQ ID NO: 39) |
| | Nucleotide sequence of human BTN2A1-Alpha-scFvCD19 with linker having |
| | knob-in-hole mutations and FcRn mutations: |
| | ATGGAGTTTGGCCTGTCTTGGGTGTTTCTGGTGGCTATCATCAAGGGA |
| | GTGCAGTGTCAGTTCATCGTTGTGGGACCTACCGATCCTATCTTGGCTA |
| | CAGTGGGCGAGAATACAACCCTGAGATGTCACTTGTCTCCTGAGAAGA |
| | ACGCCGAGGATATGGAGGTTAGGTGGTTCAGATCCCAGTTCTCTCCTG |
| | CCGTGTTTGTGTATAAGGGAGGCAGAGAGAGAACAGAAGAGCAGATG |
| | GAGGAGTACAGAGGAAGAACCACCTTCGTGTCTAAGGACATCAGCAGA |
| | GGCTCTGTGGCTCTGGTGATCCACAATATCACAGCTCAGGAGAATGGC |
| | ACCTACAGATGCTACTTTCAGGAGGGCAGGTCCTACGATGAGGCTATTT |
| | TGCATCTGGTGGTTGCTGGACTGGGATCTAAACCTCTGATCAGCATGA |
| | GGGGACACGAGGATGGAGGAATTAGACTGGAGTGCATCTCTAGAGGCT |
| | GGTATCCTAAACCACTGACAGTGTGGAGAGACCCTTATGGAGGAGTTG |
| | CTCCTGCTCTGAAAGAGGTGTCTATGCCTGATGCTGATGGCCTGTTTAT |
| | GGTGACAACAGCCGTGATCATCCGGGACAAATCCGTGAGGAACATGTC |
| | TTGCTCCATCAACAACACACTGTTGGGACAGAAGAAGGAGAGCGTGAT |
| | CTTCATCCCCGAGAGCTTCATGCCTAGCGTTTCTCCTTGTGCTGAACCT |
| | AAGTCTTGCGACAAGACCCATACATGCCCTCCTTGTCCTGCTCCTGAA |
| | GCTGCTGGAGGACCTTCTGTGTTTTTGTTTCCTCCTAAGCCTAAGGATA |
| | CCCTGATGATCTCCAGAACCCCCGAGGTGACCTGTGTGGTGGTTGATG |
| | TTTCTCATGAGGATCCTGAAGTGAAGTTCAACTGGTACGTGGACGGCG |
| | TGGAGGTGCACAACGCTAAGACAAAACCTAGAGAAGAGCAGTACAACT |
| | CTACCTACAGAGTGGTGTCCGTGCTGACCGTGCTGCACCAAGATTGGT |
| | TGAATGGAAAGGAGTACAAGTGCAAGGTGTCCAACAAGGCTCTGCCTG |
| | CCCCTATCGAGAAGACCATCTCTAAGGCTAAAGGACAACCTAGAGAAC |
| | CTCAGGTGTATACCCTGCCTCCCTGCAGAGATGAGCTGACCAAGAATC |
| | AGGTTTCTCTGTGGTGTCTGGTGAAGGGCTTTTACCCTAGCGACATCG |
| | CTGTGGAGTGGGAGTCTAATGGACAACCTGAGAACAACTACAAGACCA |
| | CACCTCCTGTGCTGGACTCTGATGGCTCCTTCTTCCTGTACTCTAAGCT |
| | GACCGTGGATAAGTCTAGATGGCAACAGGGCAACGTGTTCTCCTGCTC |
| | TGTGCTGCATGAAGCTCTGCACTCTCACTATACACAGAAGTCTCTGTCC |
| | CTGTCTCCTGGCAAGATCGAGGGCAGAATGGACGATATCCAGATGACA |
| | CAGACAACCTCTTCTCTGTCTGCTTCTCTGGGCGATAGAGTGACCATCA |
| | GCTGCAGAGCTTCTCAGGACATCAGCAAGTATCTGAACTGGTATCAGC |
| | AGAAGCCTGATGGCACCGTGAAGCTGCTGATCTACCACACCTCCAGAT |
| | TGCATTCTGGAGTTCCTTCCAGATTTTCTGGCTCTGGCTCTGGCACCG |
| | ACTATTCTCTGACCATCAGCAATCTGGAACAGGAGGACATCGCTACCTA |
| | CTTTTGCCAGCAGGGCAACACACTGCCTTACACATTTGGAGGAGGAAC |
| | AAAGCTGGAGATCACAGGAGGAGGATCTGGAGGAGGATCTGGAGGAG |
| | GATCTGAAGTTAAACTGCAGGAATCTGGACCAGGATTAGTGGCCCCAT |
| | CTCAGTCTCTGTCTGTGACCTGTACCGTTTCTGGAGTTTCTTTGCCTGA |
| | TTACGGAGTGTCCTGGATCAGACAGCCCCCTAGAAAGGGACTGGAATG |
| | GTTAGGAGTGATTTGGGGATCTGAGACCACCTACTACAACTCTGCCCT |
| | GAAGAGCAGACTGACCATCATCAAGGACAACAGCAAGTCTCAGGTGTT |
| | CCTGAAGATGAACTCCCTGCAGACCGACGATACCGCCATCTACTACTGT |
| | GCTAAGCACTACTACTATGGCGGCTCTTATGCCATGGACTATTGGGGAC |
| | AGGGCACCTCTGTGACAGTGTCTTCTTAA (SEQ ID NO: 48) |
| Human | MEFGLSWVFLVAIIKGVQCQFSVLGPSGPILAMVGEDADLPCHLFPTMSA |
| BTN3A1-beta-scFvCD19 | ETMELKWVSSSLRQVVNVYADGKEVEDRQSAPYRGRTSILRDGITAGKA |
| with polarized linkers | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | DEGGEDGSKYGPPCPPCPAPEFLGGPSVFLFPPKPKDQLMISRTPEVTCV |
| | VVDVSQEDPEVQFNWYVDGVEVHNAKTKPREEQFNSTYRVVSVLTVLHQ |
| | DWLSGKEYKCKVSSKGLPSSIEKTISNATGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVLHEALHNHYTQKSLSLSLGKRKGGKRGSGSDIQM |
| | TQTTSSLSASLGDRVTISCRASQDISKYLNWYQQKPDGTVKLLIYHTSRL |
| | HSGVPSRFSGSGSGTDYSLTISNLEQEDIATYFCQQGNTLPYTFGGGTKL |
| | EITGGGSGGGSGGGSEVKLQESGPGLVAPSQSLSVTCTVSGVSLPDYGV |
| | SWIRQPPRKGLEWLGVIWGSETTYYNSALKSRLTIIKDNSKSQVFLKMNS |
| | LQTDDTAIYYCAKHYYYGGSYAMDYWGQGTSVTVSS (SEQ ID NO: 40) |
| | Nucleotide Sequence of Human BTN3A1-beta-scFvCD19 with polarized |
| | linkers: |
| | ATGGAGTTCGGCCTGTCCTGGGTGTTTCTGGTGGCCATCATCAAGGGC |
| | GTGCAGTGCCAGTTCTCCGTGCTGGGCCCTTCTGGCCCAATCCTGGC |
| | AATGGTGGGAGAGGACGCAGATCTGCCATGTCACCTGTTTCCCACCAT |
| | GAGCGCCGAGACAATGGAGCTGAAGTGGGTGAGCTCCTCTCTGCGGC |
| | AGGTGGTGAACGTGTACGCCGACGGCAAGGAGGTGGAGGATAGACAG |
| | TCCGCCCCCTACCGGGGCAGAACCTCTATCCTGAGGGACGGAATCACA |
| | GCAGGCAAGGCCGCCCTGAGAATCCACAACGTGACCGCCAGCGATTC |
| | CGGCAAGTATCTGTGCTACTTCCAGGACGGCGACTTCTACGAGAAGGC |
| | CCTGGTGGAGCTGAAGGTGGCCGCCCTGGGAAGCGACCTGCATGTG |
| | GATGTGAAGGGCTATAAGGACGGCGGCATCCACCTGGAGTGTCGGAG |
| | CACCGGCTGGTATCCCCAGCCTCAGATCCAGTGGTCCAACAATAAGGG |
| | CGAGAATATCCCTACAGTGGAGGCCCCAGTGGTGGCAGATGGAGTGG |
| | GCCTGTACGCAGTGGCCGCCTCTGTGATCATGAGGGGAAGCTCCGGA |
| | GAGGGCGTGAGCTGCACCATCCGCTCTAGCCTGCTGGGCCTGGAGAA |
| | GACAGCCTCTATCAGCATCGCCGACCCCTTCTTTAGGAGCGCCCAGCG |
| | GTGGATCGCCGCCCTGGCAGGCGGCTCCGGCTCTGACGAGGGCGGC |
| | GAGGATGGCTCCAAGTATGGACCACCTTGCCCACCATGTCCAGCACCA |
| | GAGTTCCTGGGAGGACCAAGCGTGTTCCTGTTTCCTCCAAAGCCCAAG |
| | GACCAGCTGATGATCTCCAGGACCCCAGAGGTGACCTGCGTGGTGGT |
| | GGACGTGTCTCAGGAGGATCCTGAGGTGCAGTTCAACTGGTACGTGG |
| | ATGGCGTGGAGGTGCACAATGCCAAGACCAAGCCTAGGGAGGAGCAG |
| | TTTAACAGCACCTATCGCGTGGTGTCCGTGCTGACAGTGCTGCACCAG |
| | GATTGGCTGTCCGGCAAGGAGTACAAGTGCAAGGTGTCCTCTAAGGG |
| | CCTGCCAAGCTCCATCGAGAAGACCATCAGCAACGCAACAGGCCAGC |
| | CCCGCGAGCCTCAGGTGTATACCCTGCCCCCTTCTCAGGAGGAGATGA |
| | CCAAGAATCAGGTGAGCCTGACATGTCTGGTGAAGGGCTTCTACCCTA |
| | GCGACATCGCAGTGGAGTGGGAGTCCAACGGACAGCCAGAGAACAAT |
| | TATAAGACCACACCACCCGTGCTGGACTCTGATGGCAGCTTCTTTCTGT |
| | ACTCTAGGCTGACCGTGGATAAGAGCCGCTGGCAGGAGGGCAACGTG |
| | TTTAGCTGCTCCGTGCTGCACGAGGCCCTGCACAATCACTACACACAG |
| | AAGTCTCTGAGCCTGTCCCTGGGCAAGAGGAAGGGAGGCAAGAGGG |
| | GATCTGGAAGCGACATCCAGATGACCCAGACCACATCTAGCCTGTCCG |
| | CCTCTCTGGGCGACCGGGTGACAATCAGCTGTAGAGCCTCCCAGGATA |
| | TCTCTAAGTATCTGAATTGGTATCAGCAGAAGCCAGATGGCACCGTGAA |
| | GCTGCTGATCTATCACACAAGCAGGCTGCACTCCGGCGTGCCCTCTAG |
| | ATTCAGCGGATCCGGATCTGGCACCGACTACAGCCTGACAATCTCCAA |
| | CCTGGAGCAGGAGGATATCGCCACCTATTTCTGCCAGCAGGGCAATAC |
| | CCTGCCCTACACATTTGGCGGCGGCACCAAGCTGGAGATCACAGGCG |
| | GAGGATCTGGAGGAGGAAGCGGAGGAGGCTCCGAGGTGAAGCTGCA |
| | GGAGTCCGGACCAGGCCTGGTGGCACCTAGCCAGTCCCTGTCTGTGA |
| | CCTGTACAGTGTCCGGCGTGTCTCTGCCTGACTACGGCGTGTCCTGGA |
| | TCCGGCAGCCTCCAAGAAAGGGCCTGGAGTGGCTGGGCGTGATCTGG |
| | GGCAGCGAGACAACATACTATAACTCTGCCCTGAAGAGCAGACTGACC |
| | ATCATCAAGGACAACAGCAAGTCCCAGGTGTTTCTGAAGATGAATAGCC |
| | TGCAGACCGACGATACAGCCATCTACTATTGCGCCAAGCACTACTATTA |
| | CGGCGGCTCCTATGCCATGGATTACTGGGGCCAGGGCACCTCTGTGA |
| | CAGTGTCCTCTTGA (SEQ ID NO: 49) |
| Human | MEFGLSWVFLVAIIKGVQCQFSVLGPSGPILAMVGEDADLPCHLFPTMSA |
| BTN3A1-beta-scFvCD19 | ETMELKWVSSSLRQVVNVYADGKEVEDRQSAPYRGRTSILRDGITAGKA |
| with linker having knob-in- | ALRIHNVTASDSGKYLCYFQDGDFYEKALVELKVAALGSDLHVDVKGYK |
| hole (KIH) mutations | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAEPKSC |
| | DKTHTCPPCPAPEAAGGPSVFLFPPKPKDTLMISRTPEVTCVWVDVSHED |
| | PEVKFNWYVDGVEVHNAKTKPREEQYNSTYRVVSVLTVLHQDWLNGKEY |
| | KCKVSNKALPAPIEKTISKAKGQPREPQVCTLPPSRDELTKNQVSLSCAVK |
| | GFYPSDIAVEWESNGQPENNYKTTPPVLDSDGSFFLVSKLTVDKSRWQQ |
| | GNVFSCSVMHEALHNHYTQKSLSLSPGKIEGRMDDIQMTQTTSSLSASL |
| | GDRVTISCRASQDISKYLNWYQQKPDGTVKLLIYHTSRLHSGVPSRFSGS |
| | GSGTDYSLTISNLEQEDIATYFCQQGNTLPYTFGGGTKLEITGGGSGGGS |
| | GGGSEVKLQESGPGLVAPSQSLSVTCTVSGVSLPDYGVSWIRQPPRKGL |
| | EWLGVIWGSETTYYNSALKSRLTIIKDNSKSQVFLKMNSLQTDDTAIYYC |
| | AKHYYYGGSYAMDYWGQGTSVTVSS (SEQ ID NO: 41) |
| | Nucleotide sequence of human BTN3A1-beta-scFvCD19 with linker having |
| | knob-in-hole (KIH) mutations: |
| | ATGGAGTTTGGCCTGTCTTGGGTGTTTCTGGTGGCTATCATCAAGGGA |
| | GTGCAGTGTCAGTTCTCTGTTCTGGGACCTTCTGGACCTATCTTGGCTA |
| | TGGTGGGAGAAGATGCTGATCTGCCTTGTCACCTGTTTCCTACCATGTC |
| | TGCTGAGACCATGGAGCTGAAATGGGTGTCCTCTTCTCTGAGACAGGT |
| | GGTGAATGTGTACGCTGATGGAAAGGAGGTGGAGGACAGACAATCTG |
| | CCCCTTATAGAGGAAGAACCAGCATCCTGAGAGATGGCATCACAGCTG |
| | GAAAGGCTGCTCTGAGAATCCACAATGTGACCGCTTCTGATTCTGGCA |
| | AGTACCTGTGCTACTTCCAGGATGGCGACTTCTACGAGAAGGCTCTGG |
| | TTGAGCTGAAAGTTGCTGCTTTGGGCTCTGATCTGCATGTTGACGTGA |
| | AGGGCTACAAGGATGGAGGCATCCATCTGGAATGTAGATCTACCGGCT |
| | GGTATCCTCAACCTCAGATTCAGTGGAGCAACAACAAGGGCGAGAACA |
| | TCCCTACAGTTGAAGCCCCTGTTGTGGCTGATGGAGTTGGACTGTATG |
| | CTGTTGCTGCTAGCGTGATCATGAGAGGATCTTCTGGAGAAGGCGTGT |
| | CTTGCACCATCAGATCTTCTCTGTTGGGACTGGAGAAGACCGCTAGCA |
| | TCTCTATCGCTGACCCCTTCTTCAGATCTGCTCAAAGATGGATTGCTGC |
| | TCTGGCTGAGCCTAAGTCTTGCGATAAGACCCACACCTGTCCTCCTTG |
| | TCCTGCTCCTGAAGCTGCTGGAGGACCTTCTGTGTTTTTGTTTCCTCCT |
| | AAGCCTAAGGATACCCTGATGATCTCCAGAACCCCCGAGGTGACCTGT |
| | GTGGTGGTTGATGTTTCTCATGAGGATCCTGAAGTGAAGTTCAACTGGT |
| | ACGTGGACGGCGTGGAGGTGCACAACGCTAAGACAAAACCTAGAGAA |
| | GAGCAGTACAACTCTACCTACAGAGTGGTGTCCGTGCTGACCGTGCTG |
| | CACCAAGATTGGTTGAATGGAAAGGAGTACAAGTGCAAGGTGTCCAAC |
| | AAGGCTCTGCCTGCCCCTATCGAGAAGACCATCTCTAAGGCTAAAGGA |
| | CAACCTAGAGAACCTCAGGTGTGTACACTGCCCCCCTCTAGAGATGAG |
| | CTGACCAAGAATCAGGTTTCTCTGTCTTGTGCTGTGAAGGGCTTTTACC |
| | CCTCCGACATCGCTGTGGAATGGGAGTCTAATGGACAACCTGAGAACA |
| | ACTACAAGACCACACCTCCTGTGCTGGACTCTGACGGCTCCTTCTTTC |
| | TGGTGTCTAAGCTGACAGTGGATAAGTCTAGATGGCAACAGGGCAACG |
| | TGTTCAGCTGCTCCGTGATGCATGAAGCTCTGCACAACCACTATACACA |
| | GAAGTCTCTGAGCCTGTCTCCTGGCAAGATCGAGGGCAGAATGGACG |
| | ATATCCAGATGACACAGACAACCTCTTCTCTGTCTGCTTCTCTGGGCGA |
| | TAGAGTGACCATCAGCTGCAGAGCTTCTCAGGACATCAGCAAGTATCT |
| | GAACTGGTATCAGCAGAAGCCTGATGGCACCGTGAAGCTGCTGATCTA |
| | CCACACCTCCAGATTGCATTCTGGAGTTCCTTCCAGATTTTCTGGCTCT |
| | GGCTCTGGCACCGACTATTCTCTGACCATCAGCAATCTGGAACAGGAG |
| | GACATCGCTACCTACTTTTGCCAGCAGGGCAACACACTGCCTTACACAT |
| | TTGGAGGAGGAACAAAGCTGGAGATCACAGGAGGAGGATCTGGAGGA |
| | GGATCTGGAGGAGGATCTGAAGTTAAACTGCAGGAATCTGGACCAGGA |
| | TTAGTGGCCCCATCTCAGTCTCTGTCTGTGACCTGTACCGTTTCTGGAG |
| | TTTCTTTGCCTGATTACGGAGTGTCCTGGATCAGACAGCCCCCTAGAAA |
| | GGGACTGGAATGGTTAGGAGTGATTTGGGGATCTGAGACCACCTACTA |
| | CAACTCTGCCCTGAAGAGCAGACTGACCATCATCAAGGACAACAGCAA |
| | GTCTCAGGTGTTCCTGAAGATGAACTCCCTGCAGACCGACGATACCGC |
| | CATCTACTACTGTGCTAAGCACTACTACTATGGCGGCTCTTATGCCATGG |
| | ACTATTGGGGACAGGGCACCTCTGTGACAGTGTCTTCTTAA (SEQ ID |
| | NO: 50) |
| Human | MEFGLSWVFLVAIIKGVQCQFSVLGPSGPILAMVGEDADLPCHLFPTMSA |
| BTN3A1-beta-scFvCD19 | ETMELKWVSSSLRQVVNVYADGKEVEDRQSAPYRGRTSILRDGITAGKA |
| with knob-in-hole (KIH) | ALRIHNVTASDSGKYLCYFQDGDFYEKALVELKVAALGSDLHVDVKGYK |
| mutation and FcRn | DGGIHLECRSTGWYPQPQIQWSNNKGENIPTVEAPVVADGVGLYAVAAS |
| mutations | VIMRGSSGEGVSCTIRSSLLGLEKTASISIADPFFRSAQRWIAALAEPKSC |
| | DKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGKEY |
| | KCKVSNKALPAPIEKTISKAKGQPREPQVCTLPPSRDELTKNQVSLSCAVK |
| | GFYPSDIAVEWESNGQPENNYKTTPPVLDSDGSFFLVSKLTVDKSRWQQ |
| | GNVFSCSVLHEALHSHYTQKSLSLSPGKIEGRMDDIQMTQTTSSLSASLG |
| | DRVTISCRASQDISKYLNWYQQKPDGTVKLLIYHTSRLHSGVPSRFSGSG |
| | SGTDYSLTISNLEQEDIATYFCQQGNTLPYTFGGGTKLEITGGGSGGGSG |
| | GGSEVKLQESGPGLVAPSQSLSVTCTVSGVSLPDYGVSWIRQPPRKGLE |
| | WLGVIWGSETTYYNSALKSRLTIIKDNSKSQVFLKMNSLQTDDTAIYYCA |
| | KHYYYGGSYAMDYWGQGTSVTVSS (SEQ ID NO: 42) |
| | Nucleotide sequence of human BTN3A1-beta-scFvCD19 with knob-in-hole |
| | (KIH) mutation and FcRn mutations: |
| | ATGGAGTTTGGCCTGTCTTGGGTGTTTCTGGTGGCTATCATCAAGGGA |
| | GTGCAGTGTCAGTTCTCTGTTCTGGGACCTTCTGGACCTATCTTGGCTA |
| | TGGTGGGAGAAGATGCTGATCTGCCTTGTCACCTGTTTCCTACCATGTC |
| | TGCTGAGACCATGGAGCTGAAATGGGTGTCCTCTTCTCTGAGACAGGT |
| | GGTGAATGTGTACGCTGATGGAAAGGAGGTGGAGGACAGACAATCTG |
| | CCCCTTATAGAGGAAGAACCAGCATCCTGAGAGATGGCATCACAGCTG |
| | GAAAGGCTGCTCTGAGAATCCACAATGTGACCGCTTCTGATTCTGGCA |
| | AGTACCTGTGCTACTTCCAGGATGGCGACTTCTACGAGAAGGCTCTGG |
| | TTGAGCTGAAAGTTGCTGCTTTGGGCTCTGATCTGCATGTTGACGTGA |
| | AGGGCTACAAGGATGGAGGCATCCATCTGGAATGTAGATCTACCGGCT |
| | GGTATCCTCAACCTCAGATTCAGTGGAGCAACAACAAGGGCGAGAACA |
| | TCCCTACAGTTGAAGCCCCTGTTGTGGCTGATGGAGTTGGACTGTATG |
| | CTGTTGCTGCTAGCGTGATCATGAGAGGATCTTCTGGAGAAGGCGTGT |
| | CTTGCACCATCAGATCTTCTCTGTTGGGACTGGAGAAGACCGCTAGCA |
| | TCTCTATCGCTGACCCCTTCTTCAGATCTGCTCAAAGATGGATTGCTGC |
| | TCTGGCTGAGCCTAAGTCTTGCGATAAGACCCACACCTGTCCTCCTTG |
| | TCCTGCTCCTGAAGCTGCTGGAGGACCTTCTGTGTTTTTGTTTCCTCCT |
| | AAGCCTAAGGATACCCTGATGATCTCCAGAACCCCCGAGGTGACCTGT |
| | GTGGTGGTTGATGTTTCTCATGAGGATCCTGAAGTGAAGTTCAACTGGT |
| | ACGTGGACGGCGTGGAGGTGCACAACGCTAAGACAAAACCTAGAGAA |
| | GAGCAGTACAACTCTACCTACAGAGTGGTGTCCGTGCTGACCGTGCTG |
| | CACCAAGATTGGTTGAATGGAAAGGAGTACAAGTGCAAGGTGTCCAAC |
| | AAGGCTCTGCCTGCCCCTATCGAGAAGACCATCTCTAAGGCTAAAGGA |
| | CAACCTAGAGAACCTCAGGTGTGTACACTGCCCCCCTCTAGAGATGAG |
| | CTGACCAAGAATCAGGTTTCTCTGTCTTGTGCTGTGAAGGGCTTTTACC |
| | CCTCCGACATCGCTGTGGAATGGGAGTCTAATGGACAACCTGAGAACA |
| | ACTACAAGACCACACCTCCTGTGCTGGACTCTGACGGCTCCTTCTTTC |
| | TGGTGTCTAAGCTGACAGTGGATAAGTCTAGATGGCAACAGGGCAACG |
| | TGTTCAGCTGCAGCGTTCTGCATGAAGCTCTGCATTCCCACTATACACA |
| | GAAGTCTCTGTCCCTGTCTCCTGGCAAGATCGAGGGCAGAATGGATGA |
| | CATCCAGATGACACAGACAACCTCTTCTCTGTCTGCTTCTCTGGGCGAT |
| | AGAGTGACCATCAGCTGCAGAGCTTCTCAGGACATCAGCAAGTATCTG |
| | AACTGGTATCAGCAGAAGCCTGATGGCACCGTGAAGCTGCTGATCTAC |
| | CACACCTCCAGATTGCATTCTGGAGTTCCTTCCAGATTTTCTGGCTCTG |
| | GCTCTGGCACCGACTATTCTCTGACCATCAGCAATCTGGAACAGGAGG |
| | ACATCGCTACCTACTTTTGCCAGCAGGGCAACACACTGCCTTACACATT |
| | TGGAGGAGGAACAAAGCTGGAGATCACAGGAGGAGGATCTGGAGGAG |
| | GATCTGGAGGAGGATCTGAAGTTAAACTGCAGGAATCTGGACCAGGAT |
| | TAGTGGCCCCATCTCAGTCTCTGTCTGTGACCTGTACCGTTTCTGGAGT |
| | TTCTTTGCCTGATTACGGAGTGTCCTGGATCAGACAGCCCCCTAGAAA |
| | GGGACTGGAATGGTTAGGAGTGATTTGGGGATCTGAGACCACCTACTA |
| | CAACTCTGCCCTGAAGAGCAGACTGACCATCATCAAGGACAACAGCAA |
| | GTCTCAGGTGTTCCTGAAGATGAACTCCCTGCAGACCGACGATACCGC |
| | CATCTACTACTGTGCTAAGCACTACTACTATGGCGGCTCTTATGCCATGG |
| | ACTATTGGGGACAGGGCACCTCTGTGACAGTGTCTTCTTAA (SEQ ID |
| | NO: 51) |
| Human | Domain Linker: GSGGSGSGGSGGSG |
| BTN2A1-BTN3A1-alpha- | Linker to Fc: SKYGPP |
| scFvCD19 | CCGCCACCMEFGLSWVFLVAIIKGVQC [symbol] |
| BTN2A1V-BTN3A1V-G4- | [symbol] [symbol] |
| 19scFv3_A (IgG4 S228P, | [symbol] [symbol] GSGGSGSG |
| T250Q, M428L) | GSGGSGAQFSVLGPSGPILAMVGEDADLPCHLFPTMSAETMELKWVSS |
| BTN2A1V-BTN3A1V-G1- | SLRQVVNVYADGKEVEDRQSAPYRGRTSILRDGITAGKAALRIHNVTAS |
| 19scFv3_A (IgG1, N297A) | DSGKYLCYFQDGDFYEKALVELKVAEEPKSCDKTHTCPPCPAPELLGGPS |
| | VFLFPPKPKDTLMISRTPEVTCVVVDVSHEDPEVKFNWYVDGVEVHNAKT |
| | KPREEQYASTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQ |
| | KSLSLSPGKIEGRMDDIQMTQTTSSLSASLGDRVTISCRASQDISKYLNW |
| | YQQKPDGTVKLLIYHTSRLHSGVPSRFSGSGSGTDYSLTISNLEQEDIAT |
| | YFCQQGNTLPYTFGGGTKLEITGGGSGGGSGGGSEVKLQESGPGLVAP |
| | SQSLSVTCTVSGVSLPDYGVSWIRQPPRKGLEWLGVIWGSETTYYNSAL |
| | KSRLTIIKDNSKSQVFLKMNSLQTDDTAIYYCAKHYYYGGSYAMDYWGQ |
| | GTSVTVSS* |
| | (SEQ ID NO: 43) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GSGGSGSG |
| Linker to Fc: | GSGGSGAQFSVLGPSGPILAMVGEDADLPCHLFPTMSAETMELKWVSS |
| GGGGSGGGGSGGGGS | SLRQVVNVYADGKEVEDRQSAPYRGRTSILRDGITAGKAALRIHNVTAS |
| BTN2A1V-BTN3A1V-G4- | DSGKYLCYFQDGDFYEKALVELKVAGGGGGGGGSGGGGSCPPCPAP |
| 19scFv3_A2 (IgG4 S228P, | EFLGGPSVFLFPPKPKQQLMISRTPEVTCVVVDVSQEDPEVQFNWYVDG |
| T250Q, M428L) | VEVHNAKTKPREEQFNSTYRVVSVLTVLHQDWLSGKEYKCKVSSKGLPS |
| | SIEKTISNATGQPREPQVYTLPPSQEEMTKNQVSLTCLVKGFYPSDIAVEW |
| | ESNGQPENNYKTTPPVLDSDGSFFLYSRLTVDKSRWQEGNVFSCSVLHE |
| | ALHNHYTQKSLSLSLGKIEGRMDDIQMTQTTSSLSASLGDRVTISCRASQ |
| | DISKYLNWYQQKPDGTVKLLIYHTSRLHSGVPSRFSGSGSGTDYSLTISN |
| | LEQEDIATYFCQQGNTLPYTFGGGTKLEITGGGSGGGSGGGSEVKLQES |
| | GPGLVAPSQSLSVTCTVSGVSLPDYGVSWIRQPPRKGLEWLGVIWGSET |
| | TYYNSALKSRLTIIKDNSKSQVFLKMNSLQTDDTAIYYCAKHYYYGGSYA |
| | MDYWGQGTSVTVSS* (SEQ ID NO: 44) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Domain Linker: G(G₃S)₂ | SAQFSVLGPSGPILAMVGEDADLPCHLFPTMSAETMELKWVSSSLRQVV |
| Linker to Fc: SKYGPP | NVYADGKEVEDRQSAPYRGRTSILRDGITAGKAALRIHNVTASDSGKYL |
| BTN2A1V-BTN3A1V-G4- | CYFQDGDFYEKALVELKVASKYGPPCPPCPAPEFLGGPSVFLFPPKPKD |
| 19scFv3_B (IgG4 S228P, | QLMISRTPEVTCVVVDVSQEDPEVQFNWYVDGVEVHNAKTKPREEQFNS |
| T250Q, M428L) | TYRVVSVLTVLHQDWLSGKEYKCKVSSKGLPSSIEKTISNATGQPREPQV |
| | YTLPPSQEEMTKNQVSLTCLVKGFYPSDIAVEWESNGQPENNYKTTPPVL |
| | DSDGSFFLYSRLTVDKSRWQEGNVFSCSVLHEALHNHYTQKSLSLSLGKI |
| | EGRMDDIQMTQTTSSLSASLGDRVTISCRASQDISKYLNWYQQKPDGTV |
| | KLLIYHTSRLHSGVPSRFSGSGSGTDYSLTISNLEQEDIATYFCQQGNTLP |
| | YTFGGGTKLEITGGGSGGGSGGGSEVKLQESGPGLVAPSQSLSVTCTV |
| | SGVSLPDYGVSWIRQPPRKGLEWLGVIWGSETTYYNSALKSRLTIIKDNS |
| | KSQVFLKMNSLQTDDTAIYYCAKHYYYGGSYAMDYWGQGTSVTVSS* |
| | (SEQ ID NO: 56) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| BTN2A1V-BTN3A1V-G1- | SAQFSVLGPSGPILAMVGEDADLPCHLFPTMSAETMELKWVSSSLRQVV |
| 19scFv3_B (IgG1, N297A) | NVYADGKEVEDRQSAPYRGRTSILRDGITAGKAALRIHNVTASDSGKYL |
| | CYFQDGDFYEKALVELKVAEPKSCDKTHTCPPCPAPELLGGPSVFLFPP |
| | KPKDTLMISRTPEVTCVVVDVSHEDPEVKFNWYVDGVEVHNAKTKPREE |
| | QYASTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSL |
| | SPGKIEGRMDDIQMTQTTSSLSASLGDRVTISCRASQDISKYLNWYQQKP |
| | DGTVKLLIYHTSRLHSGVPSRFSGSGSGTDYSLTISNLEQEDIATYFCQQ |
| | GNTLPYTFGGGTKLEITGGGSGGGSGGGSEVKLQESGPGLVAPSQSLS |
| | VTCTVSGVSLPDYGVSWIRQPPRKGLEWLGVIWGSETTYYNSALKSRLTI |
| | IKDNSKSQVFLKMNSLQTDDTAIYYCAKHYYYGGSYAMDYWGQGTSVT |
| | VSS* |
| | (SEQ ID NO: 57) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Linker to Fc: | SAQFSVLGPSGPILAMVGEDADLPCHLFPTMSAETMELKWVSSSLRQVV |
| GGGGSGGGGSGGGGS | NVYADGKEVEDRQSAPYRGRTSILRDGITAGKAALRIHNVTASDSGKYL |
| BTN2A1V-BTN3A1V-G4- | CYFQDGDFYEKALVELKVAGGGGSGGGGSGGGGSCPPCPAPEFLGGP |
| 19scFv3_B2 (IgG4 S228P, | SVFLFPPKPKDQLMISRTPEVTCVVVDVSQEDPEVQFNWYVDGVEVHNA |
| T250Q, M428L) | KTKPREEQFNSTYRVVSVLTVLHQDWLSGKEYKCKVSSKGLPSSIEKTISN |
| | ATGQPREPQVYTLPPSQEEMTKNQVSLTCLVKGFYPSDIAVEWESNGQP |
| | ENNYKTTPPVLDSDGSFFLYSRLTVDKSRWQEGNVFSCSVLHEALHNHY |
| | TQKSLSLSLGKIEGRMDDIQMTQTTSSLSASLGDRVTISCRASQDISKYLN |
| | WYQQKPDGTVKLLIYHTSRLHSGVPSRFSGSGSGTDYSLTISNLEQEDIA |
| | TYFCQQGNTLPYTFGGGTKLEITGGGSGGGSGGGSEVKLQESGPGLVA |
| | PSQSLSVTCTVSGVSLPDYGVSWIRQPPRKGLEWLGVIWGSETTYYNSA |
| | LKSRLTIIKDNSKSQVFLKMNSLQTDDTAIYYCAKHYYYGGSYAMDYWG |
| | QGTSVTVSS* |
| | (SEQ ID NO: 58) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Domain Linker: G(G₃S)₃ | SGGGSAQFSVLGPSGPILAMVGEDADLPCHLFPTMSAETMELKWVSSS |
| Linker to Fc: SKYGPP | LRQVVNVYADGKEVEDRQSAPYRGRTSILRDGITAGKAALRIHNVTASD |
| BTN2A1V-BTN3A1V-G4- | SGKYLCYFQDGDFYEKALVELKVASKYGPPCPPCPAPEFLGGPSVFLFP |
| 19scFv3_C (IgG4 S228P, | PKPKDQLMISRTPEVTCVVVDVSQEDPEVQFNWYVDGVEVHNAKTKPRE |
| T250Q, M428L) | EQFNSTYRVVSVLTVLHQDWLSGKEYKCKVSSKGLPSSIEKTISNATGQP |
| | REPQVYTLPPSQEEMTKNQVSLTCLVKGFYPSDIAVEWESNGQPENNYK |
| | TTPPVLDSDGSFFLYSRLTVDKSRWQEGNVFSCSVLHEALHNHYTQKSLS |
| | LSLGKIEGRMDDIQMTQTTSSLSASLGDRVTISCRASQDISKYLNWYQQK |
| | PDGTVKLLIYHTSRLHSGVPSRFSGSGSGTDYSLTISNLEQEDIATYFCQ |
| | QGNTLPYTFGGGTKLEITGGGSGGGSGGGSEVKLQESGPGLVAPSQSL |
| | SVTCTVSGVSLPDYGVSWIRQPPRKGLEWLGVIWGSETTYYNSALKSRL |
| | TIIKDNSKSQVFLKMNSLQTDDTAIYYCAKHYYYGGSYAMDYWGQGTSV |
| | TVSS* |
| | (SEQ ID NO: 59) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| | SGGGSAQFSVLGPSGPILAMVGEDADLPCHLFPTMSAETMELKWVSSS |
| | LRQVVNVYADGKEVEDRQSAPYRGRTSILRDGITAGKAALRIHNVTASD |
| BTN2A1V-BTN3A1V-G1- | SGKYLCYFQDGDFYEKALVELKVAEPKSCDKTHTCPPCPAPELLGGPSV |
| 19scFv3_C (IgG1, N297A) | FLFPPKPKDTLMISRTPEVTCVVVDVSHEDPEVKFNWYVDGVEVHNAKTK |
| | PREEQYASTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQ |
| | KSLSLSPGKIEGRMDDIQMTQTTSSLSASLGDRVTISCRASQDISKYLNW |
| | YQQKPDGTVKLLIYHTSRLHSGVPSRFSGSGSGTDYSLTISNLEQEDIAT |
| | YFCQQGNTLPYTFGGGTKLEITGGGSGGGSGGGSEVKLQESGPGLVAP |
| | SQSLSVTCTVSGVSLPDYGVSWIRQPPRKGLEWLGVIWGSETTYYNSAL |
| | KSRLTIIKDNSKSQVFLKMNSLQTDDTAIYYCAKHYYYGGSYAMDYWGQ |
| | GTSVTVSS* |
| | (SEQ ID NO: 60) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Linker to Fc: | SGGGSAQFSVLGPSGPILAMVGEDADLPCHLFPTMSAETMELKWVSSS |
| GGGGSGGGGSGGGGS | LRQVVNVYADGKEVEDRQSAPYRGRTSILRDGITAGKAALRIHNVTASD |
| BTN2A1V-BTN3A1V-G4- | SGKYLCYFQDGDFYEKALVELKVAGGGGSGGGGSGGGGSCPPCPAPE |
| 19scFv3_C2 (IgG4 S228P, | FLGGPSVFLFPPKPKDQLMISRTPEVTDVVVDVSQEDPEVQFNWYVDGV |
| T250Q, M428L) | EVHNAKTKPREEQFNSTYRVVSVLTVLHQDWLSGKEYKCKVSSKGLPSSI |
| | EKTISNATGQPREPQVYTLPPSQEEMTKNQVSLTCLVKGFYPSDIAVEWE |
| | SNGQPENNYKTTPPVLDSDGSFFLYSRLTVDKSRWQEGNVFSCSVLHEA |
| | LHNHYTQKSLSLSLGKIEGRMDDIQMTQTTSSLSASLGDRVTISCRASQDI |
| | SKYLNWYQQKPDGTVKLLIYHTSRLHSGVPSRFSGSGSGTDYSLTISNLE |
| | QEDIATYFCQQGNTLPYTFGGGTKLEITGGGSGGGSGGGSEVKLQESG |
| | PGLVAPSQSLSVTCTVSGVSLPDYGVSWIRQPPRKGLEWLGVIWGSETT |
| | YYNSALKSRLTIIKDNSKSQVFLKMNSLQTDDTAIYYCAKHYYYGGSYAM |
| | DYWGQGTSVTVSS (SEQ ID NO: 61) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Domain Linker: G(G₃S)₄ | SGGGSGGGSAQFSVLGPSGPILAMVGEDADLPCHLFPTMSAETMELKW |
| Linker to Fc: SKYGPP | VSSSLRQVVNVYADGKEVEDRQSAPYRGRTSILRDGITAGKAALRIHNVT |
| BTN2A1V-BTN3A1V-G4- | ASDSGKYLCYFQDGDFYEKALVELKVASKYGPPCPPCPAPEFLGGPSVF |
| 19scFv3_D (IgG4 S228P, | LFPPKPKDQLMISRTPEVTDVVVDVSQEDPEVQFNWYVDGVEVHNAKTK |
| T250Q, M428L) | PREEQFNSTYRVVSVLTVLHQDWLSGKEYKCKVSSKGLPSSIEKTISNAT |
| | GQPREPQVYTLPPSQEEMTKNQVSLTCLVKGFYPSDIAVEWESNGQPEN |
| | NYKTTPPVLDSDGSFFLYSRLTVDKSRWQEGNVFSCSVLHEALHNHYTQ |
| | KSLSLSLGKIEGRMDDIQMTQTTSSLSASLGDRVTISCRASQDISKYLNW |
| | YQQKPDGTVKLLIYHTSRLHSGVPSRFSGSGSGTDYSLTISNLEQEDIAT |
| | YFCQQGNTLPYTFGGGTKLEITGGGSGGGSGGGSEVKLQESGPGLVAP |
| | SQSLSVTCTVSGVSLPDYGVSWIRQPPRKGLEWLGVIWGSETTYYNSAL |
| | KSRLTIIKDNSKSQVFLKMNSLQTDDTAIYYCAKHYYYGGSYAMDYWGQ |
| | GTSVTVSS* |
| | (SEQ ID NO: 62) |
| Human | CCGCCACCMEFGLSWVFLVAIIKGVQC [symbol] |
| | [symbol] [symbol] |
| BTN2A1-BTN3A1-alpha- | [symbol] [symbol] GGGGSGGG |
| scFvCD19 | SGGGSGGGSAQFSVLGPSGPILAMVGEDADLPCHLFPTMSAETMELKW |
| | VSSSLRQVVNVYADGKEVEDRQSAPYRGRTSILRDGITAGKAALRIHNVT |
| BTN2A1V-BTN3A1V-G1- | ASDSGKYLCYFQDGDFYEKALVELKVAEPKSCDKTHTCPPCPAPELLGG |
| 19scFv3_D (IgG1, N297A) | PSVFLFPPKPKDTLMISRTPEVTCVVVDVSHEDPEVKFNWYVDGVEVHNA |
| | KTKPREEQYASTYRVVSVLTVLHQDWLNGKEYKCKVSNKALPAPIEKTISK |
| | AKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHY |
| | TQKSLSLSPGKIEGRMDDIQMTQTTSSLSASLGDRVTISCRASQDISKYLN |
| | WYQQKPDGTVKLLIYHTSRLHSGVPSRFSGSGSGTDYSLTISNLEQEDIA |
| | TYFCQQGNTLPYTFGGGTKLEITGGGSGGGSGGGSEVKLQESGPGLVA |
| | PSQSLSVTCTVSGVSLPDYGVSWIRQPPRKGLEWLGVIWGSETTYYNSA |
| | LKSRLTIIKDNSKSQVFLKMNSLQTDDTAIYYCAKHYYYGGSYAMDYWG |
| | QGTSVTVSS* |
| | (SEQ ID NO: 63) |
| Human | CCGCCACCMREGLSWVFLVAIIKGVQC [symbol] |
| BTN2A1-BTN3A1-alpha- | [symbol] [symbol] |
| scFvCD19 | [symbol] [symbol] GGGGSGGG |
| Linker to Fc: | SGGGSGGGSAQFSVLGPSGPILAMVGEDADLPCHLFPTMSAETMELKW |
| GGGGSGGGGSGGGGS | VSSSLRQVVNVYADGKEVEDRQSAPYRGRTSILRDGITAGKAALRIHNVT |
| BTN2A1V-BTN3A1V-G4- | ASDSGKYLCYFQDGDFYEKALVELKVAGGGGSGGGGSGGGGSCPPCP |
| 19scFv3_D2 (IgG4 S228P, | APEFLGGPSVFLFPPKPKDQLMISRTPEVTCVVVDVSQEDPEVQFNWYV |
| T250Q, M428L) | DGVEVHNAKTKPREEQFNSTYRVVSVLTVLHQDWLSGKEYKCKVSSKGL |
| | PSSIEKTISNATGQPREPQVYTLPPSQEEMTKNQVSLTCLVKGFYPSDIAV |
| | EWESNGQPENNYKTTPPVLDSDGSFFLYSRLTVDKSRWQEGNVFSCSVL |
| | HEALHNHYTQKSLSLSLGKIEGRMDDIQMTQTTSSLSASLGDRVTISCRA |
| | SQDISKYLNWYQQKPDGTVKLLIYHTSRLHSGVPSRFSGSGSGTDYSLTI |
| | SNLEQEDIATYFCQQGNTLPYTFGGGTKLEITGGGSGGGSGGGSEVKLQ |
| | ESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYGG |
| | SYAMDYWGQGTSVTVSS* |
| | (SEQ ID NO: 64) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Domain Linker: (G₄S)₂ | GSAQFSVLGPSGPILAMVGEDADLPCHLFPTMSAETMELKWVSSSLRQ |
| Linker to Fc: SKYGPP | VVNVYADGKEVEDRQSAPYRGRTSILRDGITAGKAALRIHNVTASDSGK |
| BTN2A1V-BTN3A1V-G4- | YLCYFQDGDFYEKALVELKVASKYGPPCPPCPAPEFLGGPSVFLFPPKP |
| 19scFv3_E (IgG4 S228P, | KDQLMISRTPEVTDVVVDVSQEDPEVQFNWYVDGVEVHNAKTKPREEQF |
| T250Q, M428L) | NSTYRVVSVLTVLHQDWLSGKEYKCKVSSKGLPSSIEKTISNATGQPREP |
| | QVYTLPPSQEEMTKNQVSLTCLVKGFYPSDIAVEWESNGQPENNYKTTP |
| | PVLDSDGSFFLYSRLTVDKSRWQEGNVFSCSVLHEALHNHYTQKSLSLSL |
| | GKIEGRMDDIQMTQTTSSLSASLGDRVTISCRASQDISKYLNWYQQKPD |
| | GTVKLLIYHTSRLHSGVPSRFSGSGSGTDYSLTISNLEQEDIATYFCQQG |
| | NTLPYTFGGGTKLEITGGGSGGGSGGGSEVKLQESGPGLVAPSQSLSV |
| | TCTVSGVSLPDYGVSWIRQPPRKGLEWLGVIWGSETTYYNSALKSRLTII |
| | KDNSKSQVFLKMNSLQTDDTAIYYCAKHYYYGGSYAMDYWGQGTSVTV |
| | SS* |
| | (SEQ ID NO: 65) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| BTN2A1V-BTN3A1V-G1- | GSAQFSVLGPSGPILAMVGEDADLPCHLFPTMSAETMELKWVSSSLRQ |
| 19scFv3_E (IgG1, N297A) | WVNVYADGKEVEDRQSAPYRGRTSILRDGITAGKAALRIHNVTASDSGK |
| | YLCYFQDGDFYEKALVELKVAEPKSCDKTHTCPPCPAPELLGGPSVFLFP |
| | PKPKDTLMISRTPEVTCVVVDVSHEDPEVKFNWYVDGVEVHNAKTKPRE |
| | EQYASTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLS |
| | LSPGKIEGRMDDIQMTQTTSSLSASLGDRVTISCRASQDISKYLNWYQQK |
| | PDGTVKLLIYHTSRLHSGVPSRFSGSGSGTDYSLTISNLEQEDIATYFCQ |
| | QGNTLPYTFGGGTKLEITGGGSGGGSGGGSEVKLQESGPGLVAPSQSL |
| | SVTCTVSGVSLPDYGVSWIRQPPRKGLEWLGVIWGSETTYYNSALKSRL |
| | TIIKDNSKSQVFLKMNSLQTDDTAIYYCAKHYYYGGSYAMDYWGQGTSV |
| | TVSS (SEQ ID NO: 66) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Linker to Fc: | GSAQFSVLGPSGPILAMVGEDADLPCHLFPTMSAETMELKWVSSSLRQ |
| GGGGSGGGGSGGGGS | WNVYADGKEVEDRQSAPYRGRTSILRDGITAGKAALRIHNVTASDSGK |
| BTN2A1V-BTN3A1V-G4- | YLCYFQDGDFYEKALVELKVAGGGGSGGGGSGGGGSCPPCPAPEFLG |
| 19scFv3_E2 (IgG4 S228P, | GPSVFLFPPKPKDQLMISRTPEVTCVVVDVSQEDPEVQFNQYVDGVEVH |
| T250Q, M428L) | NAKTKPREEQFNSTYRVVSVLTVLHQDWLSGKEYKCKVSSKGLPSSIEKTI |
| | SNATGQPREPQVYTLPPSQEEMTKNQVSLTCLVKGFYPSDIAVEWESNG |
| | QPENNYKTTPPVLDSDGSFFLYSRLTVDKSRWQEGNVFSCSVLHEALHN |
| | HYTQKSLSLSLGKIEGRMDDIQMTQTTSSLSASLGDRVTISCRASQDISKY |
| | LNWYQQKPDGTVKLLIYHTSRLHSGVPSRFSGSGSGTDYSLTISNLEQE |
| | DIATYFCQQGNTLPYTFGGGTKLEITGGGSGGGSGGGSEVKLQESGPGL |
| | VAPSQSLSVTCTVSGVSLPDYGVSWIRQPPRKGLEWLGVIWGSETTYYN |
| | SALKSRLTIIKDNSKSQVFLKMNSLQTDDTAIYYCAKHYYYGGSYAMDY |
| | WGQGTSVTVSS |
| | (SEQ ID NO: 67) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Domain Linker: (G₄S)₄ | GSGGGGSGGGGSAQFSVLGPSGPILAMVGEDADLPCHLFPTMSAETME |
| Linker to Fc: SKYGPP | LKWVSSSLRQVVNVYADGKEVEDRQSAPYRGRTSILRDGITAGKAALRI |
| BTN2A1V-BTN3A1V-G4- | HNVTASDSGKYLCYFQDGDFYEKALVELKVASKYGPPCPPCPAPEFLGG |
| 19scFv3_F (IgG4 S228P, | PSVFLFPPKPKDQLMISRTPEVTCVVVDVSQEDPEVQFNWYVDGVEVHN |
| T250Q, M428L) | AKTKPREEQFNSTYRVVSVLTVLHQDWLSGKEYKCKVSSKGLPSSIEKTIS |
| | NATGQPREPQVYTLPPSQEEMTKNQVSLTCLVKGFYPSDIAVEWESNGQ |
| | PENNYKTTPPVLDSDGSFFLYSRLTVDKSRWQEGNVFSCSVLHEALHNH |
| | YTQKSLSLSLGKIEGRMDDIQMTQTTSSLSASLGDRVTISCRASQDISKYL |
| | NWYQQKPDGTVKLLIYHTSRLHSGVPSRFSGSGSGTDYSLTISNLEQEDI |
| | ATYFCQQGNTLPYTFGGGTKLEITGGGSGGGSGGGSEVKLQESGPGLV |
| | APSQSLSVTCTVSGVSLPDYGVSWIRQPPRKGLEWLGVIWGSETTYYNS |
| | ALKSRLTIIKDNSKSQVFLKMNSLQTDDTAIYYCAKHYYYGGSYAMDYW |
| | GQGTSVTVSS (SEQ ID NO: 68) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| BTN2A1V-BTN3A1V-G1- | GSGGGGSGGGGSAQFSVLGPSGPILAMVGEDADLPCHLFPTMSAETME |
| 19scFv3_F (IgG1, N297A) | LKWVSSSLRQVVNVYADGKEVEDRQSAPYRGRTSILRDGITAGKAALRI |
| | HNVTASDSGKYLCYFQDGDFYEKALVELKVAEPKSCDKTHTCPPCPAPE |
| | LLGGPSVFLFPPKPKDTLMISRTPEVTCVVVDVSHEDPEVKFNWYVDGVE |
| | VHNAKTKPREEQYASTYRVVSVLTVLHQDWLNGKEYKCKVSNKALPAPIE |
| | KTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEAL |
| | HNHYTQKSLSLSPGKIEGRMDDIQMTQTTSSLSASLGDRVTISCRASQDI |
| | SKYLNWYQQKPDGTVKLLIYHTSRLHSGVPSRFSGSGSGTDYSLTISNLE |
| | QEDIATYFCQQGNTLPYTFGGGTKLEITGGGSGGGSGGGSEVKLQESG |
| | PGLVAPSQSLSVTCTVSGVSLPDYGVSWIRQPPRKGLEWLGVIWGSETT |
| | YYNSALKSRLTIIKDNSKSQVFLKMNSLQTDDTAIYYCAKHYYYGGSYAM |
| | DYWGQGTSVTVSS* |
| | (SEQ ID NO: 69) |
| Human | CCGCCACCMEFGLSWVFLVAIIKGVQC [symbol] |
| BTN2A1-BTN3A1-alpha- | [symbol] [symbol] |
| scFvCD19 | [symbol] [symbol] GGGGSGGG |
| Linker to Fc: | GSGGGGSGGGGSAQFSVLGPSGPILAMVGEDADLPCHLFPTMSAETME |
| GGGGSGGGGSGGGGS | LKWVSSSLRQVVNVYADGKEVEDRQSAPYRGRTSILRDGITAGKAALRI |
| BTN2A1V-BTN3A1V-G4- | HNVTASDSGKYLCYFQDGDFYEKALVELKVAGGGGSGGGGSGGGGSC |
| 19scFv3_F2 (IgG4 S228P, | PPCPAPEFLGGPSVFLFPPKPKDQLMISRTPEVTCVVVDVSQEDPEVQFN |
| T250Q, M428L) | WYVDGVEVHNAKTKPREEQFNSTYRVVSVLTVLHQDWLSGKEYKCKVSS |
| | KGLPSSIEKTISNATGQPREPQVYTLPPSQEEMTKNQVSLTCLVKGFYPS |
| | DIAVEWESNGQPENNYKTTPPVLDSDGSFFLYSRLTVDKSRWQEGNVFS |
| | CSVLHEALHNHYTQKSLSLSLGKIEGRMDDIQMTQTTSSLSASLGDRVTIS |
| | CRASQDISKYLNWYQQKPDGTVKLLIYHTSRLHSGVPSRFSGSGSGTDY |
| | SLTISNLEQEDIATYFCQQGNTLPYTFGGGTKLEITGGGSGGGSGGGSEV |
| | KLQESGPGLVAPSQSLSVTCTVSGVSLPDYGVSWIRQPPRKGLEWLGVI |
| | WGSETTYYNSALKSRLTIIKDNSKSQVFLKMNSLQTDDTAIYYCAKHYYY |
| | GGSYAMDYWGQGTSVTVSS |
| | (SEQ ID NO: 70) |

In one aspect, the current disclosure relates to heterodimeric proteins comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the heterodimeric protein of the invention comprises two polypeptide chains, wherein the first polypeptide chain and the second polypeptide chain comprise (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the heterodimeric protein comprises two individual polypeptide chains which self-associate. In embodiments, the first domain comprising one or more butyrophilin family proteins, or a fragment thereof of the first and the second polypeptide chain are the same. In embodiments, the second domain comprising a targeting domain of the first and the second polypeptide chain are the same. In embodiments, the linker that adjoins the first and second domain are the same.

In embodiments, the first domain comprises one or more butyrophilin family proteins, or a fragment thereof. In embodiments, the butyrophilin family proteins are selected from BTN2A1, BTN3A1, and a fragment thereof. In embodiments, the first domain comprises: (i) BTN2A1, BTN3A1, and a fragment thereof; and (i) BTN2A1, BTN3A1, and a fragment thereof.

In embodiments, the first domain comprises a fragment of butyrophilin family proteins, wherein the fragment is capable of binding a gamma delta T cell receptor and is optionally an extracellular domain, optionally comprising one or more of an immunoglobulin V (IgV)- and IgC-like domain. In embodiments, the first domain comprises a fragment of butyrophilin family proteins, wherein the fragment is capable of binding a Vγ9δ2 gamma delta T cell receptor.

In embodiments, the first domain and/or the heterodimeric protein modulates or is capable of modulating a γδ (gamma delta) T cell. In embodiments, the gamma delta T cell is Vγ9δ2 T cell. In embodiments, the modulation of a gamma delta T cell is activation of a gamma delta T cell. In embodiments, the heterodimeric protein is capable of forming a synapse between a gamma delta T cell and a tumor cell and/or the heterodimeric protein is capable of contemporaneous activation and targeting of gamma delta T cells to tumor cells.

In one aspect, the current disclosure relates to heterodimeric proteins comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the heterodimeric protein of the invention comprises two polypeptide chains, wherein the first polypeptide chain and the second polypeptide chain comprise (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the heterodimeric protein comprises two individual polypeptide chains which self-associate. In embodiments, the first domain comprising one or more butyrophilin family proteins, or a fragment thereof of the first and the second polypeptide chain are the same. In embodiments, the second domain comprising a targeting domain of the first and the second polypeptide chain are the same. In embodiments, the linker that adjoins the first and second domains are the same.

In one aspect, the current disclosure relates to a heterodimeric protein comprising an alpha chain and a beta chain, wherein the alpha chain comprises: (a) a first domain comprising (i) BTN2A1, BTN3A1, and a fragment thereof; and (ii) BTN2A1, BTN3A1, and a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains; and wherein the beta chain comprises: (a) (i) a first domain comprising a BTN2A1 protein, or a fragment thereof, and (ii) a BTN3A1 protein, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, a second linker adjoins (i) and (ii). In embodiments, the second linker is a flexible amino acid sequence of any of embodiments disclosed herein.

In one aspect, the current disclosure relates to a heterodimeric protein comprising: (a) a first domain comprising (i) BTN2A1, BTN3A1, and a fragment thereof; and (ii) BTN2A1, BTN3A1, and a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence. In embodiments, two of the heterodimeric proteins associate to form a heterodimer of two chains, which comprise a BTN2A12-BTN3A12 tetramer.

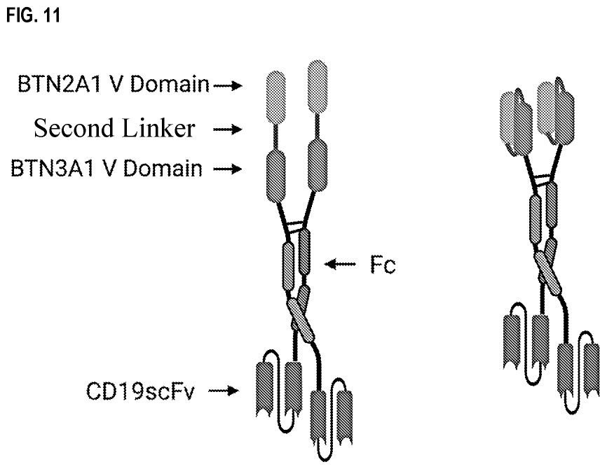

In embodiments, the present heterodimers associate to form a heterotetramer. In embodiments, the present molecules are in the form of FIG. 11.

In one aspect, the current disclosure relates to a tetrameric chimeric protein comprising two heterodimeric chimeric proteins of the heterodimeric protein of any embodiments disclosed herein, the tetramer comprises two protein chains which homodimerize to form a tetramer unit comprising BTN2A1 and BTN3A1. In embodiments, the tetramer unit is a BTN2A12-BTN3A12 tetramer unit. In embodiments, the tetrameric chimeric protein comprises a polypeptide having an amino acid sequence that has at least about 95% identity with an amino acid sequence selected from SEQ ID NOs: 43, 44 and 56-70. In embodiments, the tetrameric chimeric protein comprises a polypeptide having an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%, or at least about 99.2%, or at least about 99.4%, or at least about 99.6%, or at least about 99.8% sequence identity with an amino acid sequence selected from SEQ ID NOs: 43, 44 and 56-70. In embodiments, the tetrameric chimeric protein comprises a polypeptide having an amino acid sequence selected from SEQ ID NOs: 43, 44 and 56-70.

In embodiments, the tetrameric chimeric protein is as depicted in FIG. 11, optionally comprising a polypeptide 5 having an amino acid sequence that has at least about 95% identity with an amino acid sequence selected from SEQ ID NOs: 43, 44 and 56-70. In embodiments, the tetrameric chimeric protein is as depicted in FIG. 11, optionally comprising a polypeptide having an amino acid sequence that has an amino acid sequence selected from SEQ ID NOs: 43, 44 and 56-70.

The First Domain

In embodiments, the first domain comprises two of the same butyrophilin family proteins. In embodiments, wherein the first domain comprises two different butyrophilin family proteins. In embodiments, the butyrophilin family proteins comprise a V-type domain. Suitable butyrophilin family proteins or fragments thereof are derived from the native butyrophilin family proteins that comprise a B30.2 domain in the cytosolic tail of the full length protein.

In embodiments, the first domain is a portion of Butyrophilin subfamily 2 member A1 (BTN2A1). In embodiments, the first domain comprises substantially all the extracellular domain of BTN2A1. In embodiments, the first domain is capable of binding a gamma delta T cell receptor (e.g. Vγ9δ2). BTN2A1 is also known as BT2.1, BTF1. In embodiments, the portion of BTN2A1 is a portion of the extracellular domain of BTN2A1. In embodiments, the present chimeric protein further comprises a domain, e.g., the extracellular domain BTN2A1.

The amino acid sequence of extracellular domain of human BTN2A1, which is an illustrative amino acid sequence of human BTN2A1 suitable in the current disclosure is the following:

(SEQ ID NO: 35)
QFIVVGPTDPILATVGENTTLRCHLSPEKNAEDMEVRWFR
SQFSPAVFVYKGGRERTEEQMEEYRGRTTFVSKDISRGSV
ALVIHNITAQENGTYRCYFQEGRSYDEAILHLVVAGLGSK
PLISMRGHEDGGIRLECISRGWYPKPLTVWRDPYGGVAPA
LKEVSMPDADGLFMVTTAVIIRDKSVRNMSCSINNTLLGQ
KKESVIFIPESFMPSVSPCA

In some embodiments, the fragment of extracellular domain of human BTN2A1, which is an illustrative amino acid sequence of human BTN2A1 suitable in the current disclosure is the following:

(SEQ ID NO: 71)
QFIVVGPTDPILATVGENTTLRCHLSPEKNAEDMEVRWFR
SQFSPAVFVYKGGRERTEEQMEEYRGRTTFVSKDISRGSV
ALVIHNITAQENGTYRCYFQEGRSYDEAILHLV

In embodiments, the present chimeric protein comprises the extracellular domain of human BTN2A1 which has the amino acid sequence of SEQ ID NO: 35 or SEQ ID NO: 71. In embodiments, the present chimeric proteins may comprise the extracellular domain of BTN2A1 as described herein, or a variant or functional fragment thereof. For instance, the chimeric protein may comprise a sequence of the extracellular domain of BTN2A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of BTN2A1 as described herein.

BTN2A1 derivatives can be constructed from available structural data, including a homology model described by Karunakaran et al., Butyrophilin-2A1 Directly Binds Germline-Encoded Regions of the Vγ9Vδ2 TCR and Is Essential for Phosphoantigen Sensing, Immunity. 52(3): 487-498 (2020). Moreover, without wishing to be bound by theory, the protein structure homology-model of BTN2A1 is available at SWISS-MODEL repository. Bienert et al., “The SWISS-MODEL Repository—new features and functionality.” Nucleic Acids Research, 45(D1): D313-D319 (2017). Additional structural insight obtained from mutagenesis. Rigau et al., Butyrophilin 2A1 is essential for phosphoantigen reactivity by γδ T cells. Science 367(6478):eaay5516 (2020).

In embodiments, the first domain is a portion of Butyrophilin subfamily 3 member A1 (BTN3A1). In embodiments, the first domain comprises substantially all the extracellular domain of BTN3A1. In embodiments, the first domain is capable of binding a gamma delta T cell receptor (e.g. Vγ9δ2). BTN3A1 is also known as BTF5. In embodiments, the portion of BTN3A1 is a portion of the extracellular domain of BTN3A1. In embodiments, the present chimeric protein further comprises a domain, e.g., the extracellular domain BTN3A1.

The amino acid sequence of extracellular domain of human BTN3A1, which is an illustrative amino acid sequence of human BTN3A1 suitable in the current disclosure is the following:

(SEQ ID NO: 19)
QFSVLGPSGPILAMVGEDADLPCHLFPTMSAETMELKWVS
SSLRQVVNVYADGKEVEDRQSAPYRGRTSILRDGITAGKA
ALRIHNVTASDSGKYLCYFQDGDFYEKALVELKVAALGSD
LHVDVKGYKDGGIHLECRSTGWYPQPQIQWSNNKGENIPT
VEAPVVADGVGLYAVAASVIMRGSSGEGVSCTIRSSLLGL
EKTASISIADPFFRSAQRWIAALAG

In some embodiments, the fragment of extracellular domain of human BTN3A1, which is an illustrative amino acid sequence of human BTN2A1 suitable in the current disclosure is the following:

(SEQ ID NO: 72)
AQFSVLGPSGPILAMVGEDADLPCHLFPTMSAETMELKWV
SSSLRQVVNVYADGKEVEDRQSAPYRGRTSILRDGITAGK
AALRIHNVTASDSGKYLCYFQDGDFYEKALVELKVA

In embodiments, the present chimeric protein comprises the extracellular domain of human BTN3A1 which has the amino acid sequence of SEQ ID NO: 19 or SEQ ID NO: 72. In embodiments, the present chimeric proteins may comprise the extracellular domain of BTN3A1 as described herein, or a variant or functional fragment thereof. For instance, the chimeric protein may comprise a sequence of the extracellular domain of BTN3A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of BTN3A1 as described herein.

BTN3A1 derivatives can be constructed from available structural data, including the following: Palakodeti et al., The molecular basis for modulation of human V(gamma)9V(delta)2 T cell responses by CD277/Butyrophilin-3 (BTN3A)-specific antibodies, J Biol Chem 287: 32780-32790 (2012); Vavassori et al., Butyrophilin 3A1 binds phosphorylated antigens and stimulates human gamma delta T cells. Nat Immunol 14: 908-916 (2013); Sandstrom et al., The Intracellular B30.2 Domain of Butyrophilin 3A1 Binds Phosphoantigens to Mediate Activation of Human V gamma 9V delta 2 T Cells. Immunity 40: 490-500 (2014); Rhodes et al., Activation of Human Gammadelta T Cells by Cytosolic Interactions of Btn3A1 with Soluble Phosphoantigens and the Cytoskeletal Adaptor Periplakin. J Immunol 194: 2390 (2015); Gu et al., Phosphoantigen-induced conformational change of butyrophilin 3A1 (BTN3A1) and its implication on V gamma 9V delta 2 T cell activation. Proc Natl Acad Sci USA 114: E7311-E7320 (2017); Salim et al., BTN3A1 Discriminates gamma delta T Cell Phosphoantigens from Nonantigenic Small Molecules via a Conformational Sensor in Its B30.2 Domain. ACS Chem Biol 12: 2631-2643 (2017); Yang et al., A Structural Change in Butyrophilin upon Phosphoantigen Binding Underlies Phosphoantigen-Mediated V gamma 9V delta 2 T Cell Activation. Immunity 50: 1043 (2019).

In embodiments, the first domain comprises a portion of BTN2A1. In embodiments, the portion of BTN2A1 is an extracellular domain of BTN2A1, or a γδ T-cell receptor (e.g. γ9δ2)-binding fragment thereof.

In embodiments, the first domain comprises a portion of BTN3A1. In embodiments, the portion of BTN3A1 is an extracellular domain of BTN3A1, or a γδ T-cell receptor (e.g. γ9δ2)-binding fragment thereof.

In embodiments, the first domain comprises a portion of BTN2A1 and a portion of BTN3A1. In embodiments, the portion of BTN2A1 is an extracellular domain of BTN2A1, or a γδ T-cell receptor (e.g. γ9δ2)-binding fragment thereof. In embodiments, the portion of BTN3A1 is an extracellular domain of BTN3A1, or a γδ T-cell receptor (e.g. γ9δ2)-binding fragment thereof. In embodiments, a second linker adjoins (i) the BTN2A1 protein, or the fragment thereof, and (ii) the BTN3A1 protein, or the fragment thereof. In embodiments, the second linker is a flexible amino acid sequence. Exemplary second linkers are G(G₃S)ₘ, or GGGSₙ where m or n is 2-6, for example, GGGGSGGGS (SEQ ID NO: 73), GGGGSGGGGSGGGGS (SEQ ID NO: 74), GGGGSGGGSGGGS (SEQ ID NO: 75), GGGSGGGSGGGSGGGS (SEQ ID NO: 76), GGGGSGGGSGGGSGGGS (SEQ ID NO: 77), GGGGSGGGGS (SEQ ID NO: 78), and GGGGSGGGGSGGGGSGGGGS (SEQ ID NO: 79). In embodiments, two of the heterodimeric proteins associate to form a heterodimer of two chains, which comprise a BTN2A12-BTN3A12 tetramer.

The Second Domain Comprising a Targeting Domain

In one aspect, the current disclosure relates to a heterodimeric protein a second domain comprising a targeting domain that specifically binds to CD19.

The heterodimeric proteins of any of the embodiments disclosed herein comprise a second domain comprising a targeting domain. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv. In embodiments, the targeting domain is an extracellular domain. In embodiments, the targeting domain is capable of binding an antigen on the surface of a cancer cell. In embodiments, the targeting domain specifically binds one of CD19, PSMA, GD2, PSCA, BCMA, CD123, B7-H3, CD20, CD30, CD33, CD38, CEA, CLEC12A, DLL3, EGFRvIII, EpCAM, CD307, FLT3, GPC3, gpA33, HER2, MUC16, P-cadherin, SSTR2, and mesothelin. In embodiments, the targeting domain comprises a portion of the extracellular domain of LAG-3, PD-1, TIGIT, CD19, or PSMA. In embodiments, the targeting domain specifically binds PSMA. In embodiments, the targeting domain specifically binds CD19.

Illustrative sequences of second domain comprising a targeting domain are provided below:

An illustrative targeting domain is scFVh19, which is the heavy chain variable domain of an scFV specific to human CD19, and has the following sequence:

(SEQ ID NO: 20)
DIQLTQSPASLAVSLGQRATISCKASQSVDYDGDSYLNWY
QQIPGQPPKLLIYDASNLVSGIPPRFSGSGSGTDFTLNIH
PVEKVDAATYHCQQSTEDPWTFGGGTKLEIK

An illustrative targeting domain is scFVlh19, which is light chain variable domain of an scFV specific to human CD19, and has the following sequence:

(SEQ ID NO: 21)
EVQLVESGGGLVQPGGSLTLSCAASRFMISEYHMHWVRQA
PGKGLEWVSTINPAGTTDYAESVKGRFTISRDNAKNTLYL
QMNSLKPEDTAVYYCDSYGYRGQGTQVTV

An illustrative targeting domain is scFvCD19, which an scFV specific to human CD19, and has the following sequence:

(SEQ ID NO: 22)
QVQLQQSGAELVRPGSSVKISCKASGYAFSSYWMNWVKQR
PGQGLEWIGQIWPGDGDTNYNGKFKGKATLTADESSSTAY
MQLSSLASEDSAVYFCARRETTTVGRYYYAMDYWGQGTTV
TVSSGGGGSGGGGSGGGGSDIQLTQSPASLAVSLGQRATI
SCKASQSVDYDGDSYLNWYQQIPGQPPKLLIYDASNLVSG
IPPRFSGSGSGTDFTLNIHPVEKVDAATYHCQQSTEDPWT
FGGGTKLEIK

An illustrative targeting domain is 19scFv3, which an scFV specific to human CD19, and has the following sequence:

(SEQ ID NO: 23)
DIQMTQTTSSLSASLGDRVTISCRASQDISKYLNWYQQKP
DGTVKLLIYHTSRLHSGVPSRFSGSGSGTDYSLTISNLEQ
EDIATYFCQQGNTLPYTFGGGTKLEITGGGSGGGSGGGSE
VKLQESGPGLVAPSQSLSVTCTVSGVSLPDYGVSWIRQPP
RKGLEWLGVIWGSETTYYNSALKSRLTIIKDNSKSQVFLK
MNSLQTDDTAIYYCAKHYYYGGSYAMDYWGQGTSVTVSS

An illustrative targeting domain is scFvCD19VHVL, which an scFV specific to mouse CD19, and has the following sequence:

(SEQ ID NO: 24)
EVQLQQSGAELVRPGTSVKLSCKVSGDTITFYYMHFVKQR
PGQGLEWIGRIDPEDESTKYSEKFKNKATLTADTSSNTAY
LKLSSLTSEDTATYFCIYGGYYFDYWGQGVMVTVSSGGGG
SGGGGSGGGGSDIQMTQSPASLSTSLGETVTIQCQASEDI
YSGLAWYQQKPGKSPQLLIYGASDLQDGVPSRFSGSGSGT
QYSLKITSMQTEDEGVYFCQQGLTYPRTFGGGTKLELK

An illustrative targeting domain is scFvCD19VLVH, which an scFV specific to mouse CD19, and has the following sequence:

(SEQ ID NO: 25)
DIQMTQSPASLSTSLGETVTIQCQASEDIYSGLAWYQQKP
GKSPQLLIYGASDLQDGVPSRFSGSGSGTQYSLKITSMQT
EDEGVYFCQQGLTYPRTFGGGTKLELKGGGGSGGGGSGGG
GSEVQLQQSGAELVRPGTSVKLSCKVSGDTITFYYMHFVK
QRPGQGLEWIGRIDPEDESTKYSEKFKNKATLTADTSSNT
AYLKLSSLTSEDTATYFCIYGGYYFDYWGQGVMVTVSS

An illustrative targeting domain is scFVIPSMA, which is light chain variable domain of an scFV specific to human PSMA, and has the following sequence:

(SEQ ID NO: 26)
RKGGKRGSGSGQTVVTQEPSLTVSPGGTVTLTCASSTGAV
TSGNYPNWVQQKPGQAPRGLIGGTKFLVPGTPARFSGSLL
GGKAALTLSGVQPEDEAEYYCTLWYSNRWVFGGGTKLTVL

An illustrative targeting domain is GD2scFv3, which an scFV specific to human GD2, and has the following sequence

(SEQ ID NO: 27)
GTDFTLKISRVEAEDLGVYFCSQSTHVPPLTFGAGTKLEL
KGGGSGGGSGGGSEVQLLQSGPELEKPGASVMISCKASGS
SFTGYNMNWVRQNIGKSLEWIGAIDPYYGGTSYNQKFKGR
ATLTVDKSSSTAYMHLKSLTSEDSAVYYCVSGMKYWGQGT
SVTVSS

In embodiments, the second domain of the alpha chain and/or beta chain comprises a polypeptide having an amino acid sequence selected from SEQ ID NOs: 20-23 and 94-126. In embodiments, the second domain of the alpha chain and/or beta chain comprises a polypeptide having an amino acid sequence that has at least about 95% identity with an amino acid sequence selected from SEQ ID NOs: 20-23 and 94-126. In embodiments, the second domain of the alpha chain and/or beta chain comprises a polypeptide having an amino acid sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence an amino acid sequence selected from SEQ ID NOs: 20-23 and 94-126.

The Linker Domain that Adjoins the First and the Second Domain

In embodiments, the linker that adjoins the first and second domains comprises a charge polarized core domain. In various embodiments, each of the first and second charge polarized core domains comprises proteins having positively or negatively charged amino acid residues at the amino and carboxy terminus of the core domain. In an illustrative embodiment, the first charge polarized core domain may comprise a protein having positively charged amino acids at the amino terminus which are adjoined by a linker (e.g., a stabilizing domain) to a protein having negatively charged amino acid residues at the carboxy terminus. The second charge polarized core domain may comprise a protein having negatively charged amino acids at the amino terminus which are adjoined by a linker (e.g., a stabilizing domain) to a protein having positively charged amino acid residues at the carboxy terminus.

In another illustrative embodiment, the first charge polarized core domain may comprise a protein having negatively charged amino acids at the amino terminus which are adjoined by a linker (e.g., a stabilizing domain) to a protein having positively charged amino acid residues at the carboxy terminus. The second charge polarized core domain may comprise proteins having positively charged amino acids at the amino terminus which are adjoined by a linker (e.g., a stabilizing domain) to a protein having negatively charged amino acid residues at the carboxy terminus.

In various embodiments, formation of heterodimeric proteins is driven by electrostatic interactions between the positively charged and negatively charged amino acid residues located at the amino and carboxy termini of the first and second charge polarized core domains. Further, formation of homodimeric proteins is prevented by the repulsion between the positively charged amino acid residues or negatively charged amino acid residues located at the amino and carboxy termini of the first and second charge polarized core domains.

In various embodiments, the protein comprising positively and/or negatively charged amino acid residues at the amino or carboxy terminus of the charge polarized core domains is about 2 to about 50 amino acids long. For example, the protein comprising positively and/or negatively charged amino acid residues at either terminus of the charge polarized core domain may be about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the protein comprising positively charged amino acid residues may include one or more of amino acids selected from His, Lys, and Arg. In various embodiments, the protein comprising negatively charged amino acid residues may include one or more amino acids selected from Asp and Glu.

In various embodiments, each of the first and/or second charge polarized core domains may comprise a protein comprising an amino acid sequence as provided in the Table below or an amino acid sequence having at least 90%, or 93%, or 95%, or 97%, or 98%, or 99% identity thereto.

| SEQ ID | |
|---|---|
| NO. | Sequence |
| 1 | YₙXₙYₙXₙYₙ (where X is a positively charged amino acid such as arginine, histidine |
| | or lysine and Y is a spacer amino acid such as serine or glycine, and where each |
| | n is independently an integer 0 to 4) |
| 2 | YₙZₙYₙZₙYₙ (where Z is a negatively charged amino acid such as aspartic acid or |
| | glutamic acid and Y is a spacer amino acid such as serine or glycine, and where |
| | each n is independently an integer 0 to 4) |
| 3 | YYₙXXₙYYₙXXₙYYₙ (where X is a positively charged amino acid such as arginine, |
| | histidine or lysine and Y is a spacer amino acid such as serine or glycine, and |
| | where each n is independently an integer 0 to 4) |
| 4 | YYₙZZₙYYₙZZₙYYₙ (where Z is a negatively charged amino acid such as aspartic |
| | acid or glutamic acid and Y is a spacer amino acid such as serine or glycine, and |
| | where each n is independently an integer 0 to 4) |
| 5 | YₙXₙCYₙXₙYₙ (where X is a positively charged amino acid such as arginine, |
| | histidine or lysine and Y is a spacer amino acid such as serine or glycine, and |
| | where each n is independently an integer 0 to 4) |
| 6 | YₙZₙCYₙZₙYₙ (where Z is a negatively charged amino acid such as aspartic acid or |
| | glutamic acid and Y is a spacer amino acid such as serine or glycine, and where |
| | each n is independently an integer 0 to 4) |
| 7 | GSGSRKGGKRGS |
| 8 | GSGSRKCGKRGS |
| 9 | GSGSDEGGEDGS |
| 10 | GSGSDECGEDGS |

For example, in an embodiment, each of the first and second charge polarized core domains may comprise a peptide comprising the sequence YYₙXXₙYYₙXXₙYYₙ (where X is a positively charged amino acid such as arginine, histidine or lysine and Y is a spacer amino acid such as serine or glycine; SEQ ID NO: 3). Illustrative peptide sequences include, but are not limited to, RKGGKR (SEQ ID NO: 11) or GSGSRKGGKRGS (SEQ ID NO: 12).

In another illustrative embodiment, each of the first and second charge polarized core domains may comprise a peptide comprising the sequence YYₙZZₙYYₙZZₙYYₙ (where Z is a negatively charged amino acid such as aspartic acid or glutamic acid and Y is a spacer amino acid such as serine or glycine). Illustrative peptide sequences include, but are not limited to, DEGGED (SEQ ID NO: 13) or GSGSDEGGEDGS (SEQ ID NO: 14).

In one aspect, the current disclosure provides a heterodimeric protein comprising (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the heterodimeric protein comprises two individual polypeptide chains which self-associate. In embodiments, the linker facilitates heterodimerization. In embodiments, the heterodimeric protein comprises two of the same butyrophilin family proteins or two different butyrophilin family proteins. In embodiments, the butyrophilin family proteins comprise a V-type domain and/or a B30.2 domain. In embodiments, the first domain is a butyrophilin-like (BTNL) family protein, such as BTN2A1, BTN3A1, and a fragment thereof.

In embodiments, the first polypeptide chain and the second polypeptide chain heterodimers through electrostatic interactions between positively charged amino acid residues and negatively charged amino acid residues on the first and second charge polarized core domains. In embodiments, the positively charged amino acid residues may include one or more of amino acids selected from His, Lys, and Arg. In embodiments, the negatively charged amino acid residues may include one or more amino acids selected from Asp and Glu.

Accordingly, in embodiments, each of the first and/or second charge polarized core domains comprises proteins having positively or negatively charged amino acid residues at the amino and carboxy terminus of the core domain. In an illustrative embodiment, the first charge polarized core domain may comprise a protein having positively charged amino acids at the amino terminus which are adjoined by a linker (e.g., a stabilizing domain) to a protein having negatively charged amino acid residues at the carboxy terminus. In such an embodiment, the second charge polarized core domain may comprise a protein having negatively charged amino acids at the amino terminus which are adjoined by a linker (e.g., a stabilizing domain) to a protein having positively charged amino acid residues at the carboxy terminus. In another illustrative embodiment, the first charge polarized core domain may comprise a protein having negatively charged amino acids at the amino terminus which are adjoined by a linker (e.g., a stabilizing domain) to a protein having positively charged amino acid residues at the carboxy terminus. In such an embodiment, the second charge polarized core domain may comprise proteins having positively charged amino acids at the amino terminus which are adjoined by a linker (e.g., a stabilizing domain) to a protein having negatively charged amino acid residues at the carboxy terminus.

In various embodiments, each of the first and/or second charge polarized core domains further comprise a linker (e.g., a stabilizing domain) which adjoins the proteins having positively or negatively charged amino acids. In embodiments, the linker (e.g., a stabilizing domain) is optionally selected from a flexible amino acid sequence, IgG hinge region, or antibody sequence. In an embodiment, the linker (e.g., a stabilizing domain) comprises the hinge-CH2-CH3 Fc domain derived from IgG1, optionally human IgG1. In another embodiment, the linker (e.g., a stabilizing domain) comprises the hinge-CH2-CH3 Fc domain derived from IgG4, optionally human IgG4.

Illustrative sequences of linkers that adjoins the first and second domains, also referred to herein as a core domain are provided below:

In embodiments, the core domain has the following sequence:

(SEQ ID NO: 15)
SKYGPPCPPCPAPEFLGGPSVFLFPPKPKDQLMISRTPEVTCVVVDVSQE
DPEVQFNWYVDGVEVHNAKTKPREEQFNSTYRVVSVLTVLHQDWLSGKEY
KCKVSSKGLPSSIEKTISNATGQPREPQVYTLPPSQEEMTKNQVSLTCLV
KGFYPSDIAVEWESNGQPENNYKTTPPVLDSDGSFFLYSRLTVDKSRWQE
GNVFSCSVLHEALHNHYTQKSLSLSLGKIEGRMD.

In embodiments, the core domain has the following sequence:

(SEQ ID NO: 28)
CPPCPAPEFLGGPSVFLFPPKPKDQLMISRTPEVTCVVVDVSQEDPEVQF
NWYVDGVEVHNAKTKPREEQFNSTYRVVSVLTVLHQDWLSGKEYKCKVSS
KGLPSSIEKTISNATGQPREPQVYTLPPSQEEMTKNQVSLTCLVKGFYPS
DIAVEWESNGQPENNYKTTPPVLDSDGSFFLYSRLTVDKSRWQEGNVFSC
SVLHEALHNHYTQKSLSLSLGK.

In embodiments, the core domain is a KIHT22Y protein having the following sequence:

(SEQ ID NO: 29)
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
YCLVKGFYPSDIAVEWESNGQPENNYKTTPPVLDSDGSFFLYSKLTVDKS
RWQQGNVFSCSVMHEALHNHYTQKSLSLSPGK.

In embodiments, the core domain is a KIHY86T protein having the following sequence:

(SEQ ID NO: 30)
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLSKLTVDKSR
WQQGNVFSCSVMHEALHNHYTQKSLSLSPGK.

In embodiments, the core domain is a KIHY86T protein having the following sequence:

(SEQ ID NO: 31)
VPRDCGCKPCICTVPEVSSVFIFPPKPKDVITITUTPKVTCVWVDISKDD
PEVQFSWFVDDVEVHTAQTQPREEQFNSTFRSVSELPIMHQDWLNGKEFK
CRVNSAAFPAPIEKTISKTKGRPKAPQVYTIPPPKEOMAKDKVSLTCMIT
DFFPEDITVEWOWNGQPAENYKNTQPIMDTDGSYFVYSKLNVQKSNWEAG
NTFTCSVLHEGLHNHHTEKSLSHSPGI.

The sequence of an illustrative charge polarized core domain (positive-negative) is provided below:

(SEQ ID NO: 16)
GSGSRKGGKRGSKYGPP

[symbol]

[symbol]

[symbol]

[symbol]

[symbol]

[symbol]

[symbol]

[symbol]

[symbol]

[symbol]

DEGGEDGSGS.

The sequence of an illustrative charge polarized core domain (negative-positive) is provided below:

(SEQ ID NO: 17)
GSGSDEGGEDGSKYGPP

[symbol]

[symbol]

[symbol]

[symbol]

[symbol]

[symbol]

[symbol]

[symbol]

[symbol]

[symbol]

RKGGKRGSGS.

The sequence of an illustrative charge polarized core domain (negative-positive) is provided below:

(SEQ ID NO: 32)

[symbol]

[symbol]

[symbol]

[symbol]

[symbol]

[symbol]

[symbol]

.

The sequence of an illustrative Fc domains containing knob-in-hole (KIH) mutations are provided below:

(SEQ ID NO: 52)
EPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPCRDELTKNQVSL
WCLVKGFYPSDIAVEWESNGQPENNYKTTPPVLDSDGSFFLYSKLTVDKS
RWQQGNVFSCSVMHEALHNHYTQKSLSLSPGKIEGRMD.

The sequence of an illustrative Fc domains containing knob-in-hole (KIH) mutations are provided below:

(SEQ ID NO: 53)
EPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVCTLPPSRDELTKNQVSL
SCAVKGFYPSDIAVEWESNGQPENNYKTTPPVLDSDGSFFLVSKLTVDKS
RWQQGNVFSCSVMHEALHNHYTQKSLSLSPGKIEGRMD.

The sequence of an illustrative Fc domains containing knob-in-hole (KIH) mutations and FcRn mutations are provided below:

(SEQ ID NO: 54)
EPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPCRDELTKNQVSL
WCLVKGFYPSDIAVEWESNGQPENNYKTTPPVLDSDGSFFLYSKLTVDKS
RWQQGNVFSCSVLHEALHSHYTQKSLSLSPGKIEGRMD.

The sequence of an illustrative Fc domains containing knob-in-hole (KIH) mutations and FcRn mutations are provided below:

(SEQ ID NO: 55)
EPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVCTLPPSRDELTKNQVSL
SCAVKGFYPSDIAVEWESNGQPENNYKTTPPVLDSDGSFFLVSKLTVDKS
RWQQGNVFSCSVLHEALHSHYTQKSLSLSPGKIEGRMD.

In various embodiments, the protein comprising the charged amino acid residues may further comprise one or more cysteine residues to facilitate disulfide bonding between the electrostatically charged core domains as an additional method to stabilize the heterodimer.

In various embodiments, each of the first and second charge polarized core domains comprises a linker sequence which may optionally function as a stabilizing domain. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker (e.g., a stabilizing domain) is a synthetic linker such as PEG.

In other embodiments, the linker (e.g., a stabilizing domain) is a polypeptide. In embodiments, the linker (e.g., a stabilizing domain) is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker (e.g., a stabilizing domain) may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the linker (e.g., a stabilizing domain) is substantially comprised of glycine and serine residues (e.g., about 30%, or about 40%, or about 50%, or about 60%, or about 70%, or about 80%, or about 90%, or about 95%, or about 97% glycines and serines).

In various embodiments, the linker (e.g., a stabilizing domain) is a hinge region of an antibody (e.g., of IgG, IgA, IgD, and IgE, inclusive of subclasses (e.g., IgG1, IgG2, IgG3, and IgG4, and IgA1 and IgA2). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In other embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys which, when dimerized by disulfide bond formation, results in a cyclic octapeptide believed to act as a pivot, thus conferring flexibility. In various embodiments, the present linker (e.g., a stabilizing domain) comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker (e.g., a stabilizing domain) of the current disclosure comprises one or more glycosylation sites.

In various embodiments, the linker (e.g., a stabilizing domain) comprises an Fc domain of an antibody (e.g., of IgG, IgA, IgD, and IgE, inclusive of subclasses (e.g., IgG1, IgG2, IgG3, and IgG4, and IgA1 and IgA2)). In various embodiments, the linker (e.g., a stabilizing domain) comprises a hinge-CH2-CH3 Fc domain derived from a human IgG4 antibody. In various embodiments, the linker (e.g., a stabilizing domain) comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present heterodimeric proteins.

In embodiments, the Fc domain contains one or more amino acid substitutions at amino acid residue 250, 252, 254, 256, 308, 309, 311, 428, 433 or 434 (in accordance with Kabat numbering), or equivalents thereof. In an embodiment, the amino acid substitution at amino acid residue 250 is a substitution with glutamine. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, or threonine. In an embodiment, the amino acid substitution at amino acid residue 308 is a substitution with threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 428 is a substitution with leucine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In embodiments, the Fc domain (e.g., comprising an IgG constant region) comprises one or more mutations such as substitutions at amino acid residue 252, 254, 256, 433, 434, or 436 (in accordance with Kabat numbering). In an embodiment, the IgG constant region includes a triple M252Y/S254T/T256E mutation or YTE mutation. In another embodiment, the IgG constant region includes a triple H433K/N434F/Y436H mutation or KFH mutation. In a further embodiment, the IgG constant region includes an YTE and KFH mutation in combination.

In embodiments, the modified humanized antibodies of the invention comprise an IgG constant region that contains one or more mutations at amino acid residues 250, 253, 307, 310, 380, 428, 433, 434, and 435. Illustrative mutations include T250Q, M428L, T307A, E380A, I253A, H310A, M428L, H433K, N434A, N434F, N434S, and H435A. In an embodiment, the IgG constant region comprises a M428L/N434S mutation or LS mutation. In another embodiment, the IgG constant region comprises a T250Q/M428L mutation or QL mutation. In another embodiment, the IgG constant region comprises an N434A mutation. In another embodiment, the IgG constant region comprises a T307A/E380A/N434A mutation or AAA mutation. In another embodiment, the IgG constant region comprises an I253A/H310A/H435A mutation or IHH mutation. In another embodiment, the IgG constant region comprises a H433K/N434F mutation. In another embodiment, the IgG constant region comprises a M252Y/S254T/T256E and a H433K/N434F mutation in combination.

In various embodiments, mutations are introduced to increase stability and/or half-life of the Fc domain. An illustrative Fc stabilizing mutant is S228P. Additional illustrative Fc half-life extending mutants are T250Q, M428L, V308T, L309P, and Q311S and the present linkers (e.g., stabilizing domains) may comprise 1, or 2, or 3, or 4, or 5 of these mutants.

Additional illustrative mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al., Nature (2014) 514:642-645, Grevys et al., Journal of Immunology. (2015), 194(11):5497-508, and U.S. Pat. No. 7,083,784, the entire contents of which are hereby incorporated by reference.

In various embodiments, the linker may be flexible, including without limitation highly flexible. In various embodiments, the linker may be rigid, including without limitation a rigid alpha helix.

In various embodiments, the linker may be functional. For example, without limitation, the linker may function to improve the folding and/or stability, improve the expression, improve the pharmacokinetics, and/or improve the bioactivity of the present heterodimeric protein. In another example, the linker may function to target the heterodimeric protein to a particular cell type or location.

The Heterodimeric Proteins

In one aspect, the current disclosure provides a heterodimeric protein comprising: (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains.

In embodiments the heterodimeric protein is a complex of two polypeptide chains.

In embodiments the heterodimeric protein comprises an alpha chain and a beta chain wherein the alpha chain and the beta chain each independently comprise (a) a first domain comprising a butyrophilin family protein, or fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains.

In embodiments the alpha chain and the beta chain self-associate to form the heterodimer.

In embodiments, the first domain comprises two of the same butyrophilin family proteins. In embodiments, wherein the first domain comprises two different butyrophilin family proteins. In embodiments, the butyrophilin family proteins comprise a V-type domain. In embodiments, the butyrophilin family proteins or fragments thereof are derived from the native butyrophilin family proteins that comprise a B30.2 domain in the cytosolic tail.

In embodiments, the butyrophilin family proteins are selected from BTN2A1, BTN3A1, and a fragment thereof. In embodiments, the first domain comprises: (a) BTN2A1, BTN3A1, and a fragment thereof; and (b) BTN2A1, BTN3A1, and a fragment thereof.

In embodiments, the first domain comprises a fragment of butyrophilin family proteins, wherein the fragment is capable of binding a gamma delta T cell receptor and is optionally an extracellular domain, optionally comprising one or more of an immunoglobulin V (IgV)- and IgC-like domain. In embodiments, the first domain comprises a fragment of butyrophilin family proteins, wherein the fragment is capable of binding a Vγ9δ2 gamma delta T cell receptor.

In embodiments, the first domain comprises a polypeptide having an amino acid sequence of: (a) any one of SEQ ID NOs: 19, 35, or a fragment thereof; and (b) any one of SEQ ID NOs: 19, 35, or a fragment thereof. In embodiments, the first domain comprises a polypeptide having (a) an amino acid sequence having at least 90%, or 95%, or 97%, or 98%, or 99% identity with SEQ ID NO: 19 or SEQ ID NO: 72, and an amino acid sequence having at least 90%, or 95%, or 97%, or 98%, or 99% identity with SEQ ID NO: 35 or SEQ ID NO: 71.

Additionally, or alternatively, in any of the embodiments disclosed herein, in the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv. In embodiments, the targeting domain is an extracellular domain. In embodiments, the targeting domain is capable of binding an antigen on the surface of a cancer cell. In embodiments, the targeting domain specifically binds one of CD19, PSMA, GD2, PSCA, BCMA, CD123, B7-H3, CD20, CD30, CD33, CD38, CEA, CLEC12A, DLL3, EGFRvIII, EpCAM, CD307, FLT3, GPC3, gpA33, HER2, MUC16, P-cadherin, SSTR2, and mesothelin. In embodiments, the targeting domain comprises a portion of the extracellular domain of LAG-3, PD-1, TIGIT, CD19, or PSMA. In embodiments, the targeting domain specifically binds CD19. In embodiments, the targeting domain specifically binds PSMA. Additionally or alternatively, In embodiments, the targeting domain is a polypeptide having an amino acid sequence with at least 90%, or 95%, or 97%, or 98%, or 99% identity with a polypeptide selected from SEQ ID NOs: 20-27 and 94-126. In embodiments, the targeting domain is a polypeptide having an amino acid sequence of selected from SEQ ID NOs: 20-27 and 94-126.

Additionally or alternatively, In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains. In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally human IgG1. In embodiments, the linker comprises the hinge-CH2-CH3 Fc domain derived from IgG4, optionally human IgG4. In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains.

In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12). In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine) (SEQ ID NO: 6, and where each n is independently an integer 0 to 4). In embodiments, the peptide comprising negatively charged amino acid residues comprises the sequence DEGGED (SEQ ID NO: 13) or GSGSDEGGEDGS (SEQ ID NO: 14).

Additionally or alternatively, in embodiments, the first domain and/or the heterodimeric protein modulates or is capable of modulating a γδ (gamma delta) T cell. In embodiments, the gamma delta T cell is a Vγ9δ2 gamma delta T cell.

Additionally or alternatively, In embodiments, the heterodimeric protein is capable of forming a synapse between a gamma delta T cell and a tumor cell. In embodiments, the heterodimeric protein is capable of contemporaneous activation and targeting of gamma delta T cells to tumor cells.

In embodiments, the heterodimeric protein comprises an amino acid sequence having at least 90%, or at least 91%, or at least 92%, or at least 93%, or at least 94%, or at least 95%, or at least 96%, or at least 97%, or at least 98%, or at least 98%, or at least 99% sequence identity to SEQ ID NO: 19, 35, 71, or 72.

In embodiments, the second domain is a LAG-3 protein.

In embodiments, the second domain is a PD-1 protein.

In embodiments, the second domain is a TIGIT protein.

In embodiments, the second domain is a CD19 protein binding domain, such as an scFv, CDR3, or Fab. In embodiments, the second domain is a CD19 protein and the heterodimeric protein further comprise an antibody or fragment thereof (e.g., comprising a portion of the antigen-binding domain of an antibody) and which is capable of binding an antigen on the surface of a cancer cell.

In embodiments, the second domain is a PSMA protein binding domain, such as an scFv, CDR3, or Fab. In embodiments, the second domain is a PSMA protein and the heterodimeric protein further comprise an antibody or fragment thereof (e.g., comprising a portion of the antigen-binding domain of an antibody) and which is capable of binding an antigen on the surface of a cancer cell.

In an illustrative embodiment, the second domain is a receptor for EGP such as EGFR (ErbB1), ErbB2, ErbB3 and ErbB4.

In an illustrative embodiment, the second domain is a receptor for insulin or an insulin analog such as the insulin receptor and/or IGF1 or IGF2 receptor.

In an illustrative embodiment, the second domain is a receptor for EPO such as the EPO receptor (EPOR) receptor and/or the ephrin receptor (EphR)

In various embodiments, the heterodimeric protein may comprise a domain of a soluble (e.g., non-membrane associated) protein. In various embodiments, the heterodimeric protein may comprise a fragment of the soluble protein which is involved in signaling (e.g., a portion of the soluble protein which interacts with a receptor).

In various embodiments, the heterodimeric protein may comprise the extracellular domain of a transmembrane protein. In various embodiments, one of the extracellular domains transduces an immune inhibitory signal and one of the extracellular domains transduces an immune stimulatory signal.

In embodiments, an extracellular domain refers to a portion of a transmembrane protein which is capable of interacting with the extracellular environment. In various embodiments, an extracellular domain refers to a portion of a transmembrane protein which is sufficient to bind to a ligand or receptor and effective transmit a signal to a cell. In various embodiments, an extracellular domain is the entire amino acid sequence of a transmembrane protein which is external of a cell or the cell membrane. In various embodiments, an extracellular domain is the that portion of an amino acid sequence of a transmembrane protein which is external of a cell or the cell membrane and is needed for signal transduction and/or ligand binding as may be assayed using methods know in the art (e.g., in vitro ligand binding and/or cellular activation assays).

In various embodiments, the heterodimeric protein may comprise an antibody binding domain (e.g. CDR3, Fab, scFv domain, etc.). In various embodiments, one of the antibody binding domains transduces an immune inhibitory signal and one of the antibody binding domains transduces an immune stimulatory signal.

In embodiments, an immune inhibitory signal refers to a signal that diminishes or eliminates an immune response. For example, in the context of oncology, such signals may diminish or eliminate antitumor immunity. Under normal physiological conditions, inhibitory signals are useful in the maintenance of self-tolerance (e.g., prevention of autoimmunity) and also to protect tissues from damage when the immune system is responding to pathogenic infection. For instance, without limitation, immune inhibitory signal may be identified by detecting an increase in cellular proliferation, cytokine production, cell killing activity or phagocytic activity when such an inhibitory signal is blocked.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes. Specific examples include direct stimulation of cytokine receptors such as IL-2R, IL-7R, IL-15R, IL-17R or IL-21R using fusion proteins encoding the ligands for such receptors (IL-2, IL-7, IL-15, IL-17 or IL-21, respectively). Stimulation from any one of these receptors may directly stimulate the proliferation and cytokine production of individual T cell subsets.

In embodiments, the extracellular domain or antibody binding domain (e.g. CDR3, Fab, scFv domain, etc.) may be used to produce a soluble protein to competitively inhibit signaling by that receptor's ligand. For instance, without limitation, competitive inhibition of PD-L1 or PD-L2 could be achieved using PD-1, or competitive inhibition of PVR could be achieved using TIGIT. In embodiments, the extracellular domain or antibody binding domain (e.g. CDR3, Fab, scFv domain, etc.) may be used to provide artificial signaling.

In embodiments, the present heterodimeric proteins deliver or mask an immune inhibitory signal. In embodiments, the present heterodimeric proteins deliver or mask an immune stimulatory signal.

In various embodiments, the present heterodimeric proteins comprise two independent binding domains, each from one subunit of a heterodimeric human protein. Illustrative proteins that may be formed as part of the heterodimeric protein of the invention are provided in Table 1. In various embodiments, the present heterodimeric proteins have one of the illustrative proteins provided in Table 1. In various embodiments, the present heterodimeric proteins have two of the illustrative proteins provided in Table 1.

TABLE 1
Illustrative butyrophilin family protein which may be
incorporated into the present compositions and methods
include the following proteins (as used herein, “Entry”
refers to the protein entry in the Uniprot database and
“Entry name” refers to the protein entry in the Uniprot
database):

| | | | SEQ |
|---|---|---|---|
| Entry/ | Protein names | | ID |
| Name | Gene names | ECD Sequence | NO |
| Q7KYR7 | Butyrophilin | QFIVVGPTDPILATVGENTTLRCHLSPEKNAEDMEVRW | 36 |
| BT2A1_ | subfamily 2 | FRSQFSPAVFVYKGGRERTEEQMEEYRGRTTFVSKDIS | |
| HUMAN | member A1 | RGSVALVIHNITAQENGTYRCYFQEGRSYDEAILHLVV | |
| | BTN2A1 BT2.1, | AGLGSKPLISMRGHEDGGIRLECISRGWYPKPLTVWRD | |
| | BTF1 | PYGGVAPALKEVSMPDADGLFMVTTAVIIRDKSVRNMS | |
| | | CSINNTLLGQKKESVIFIPESFMPSVSPCA | |
| H7BYC3 | Butyrophilin | | |
| H7BYC3_ | subfamily 2 | | |
| HUMAN | member A1 | | |
| | BTN2A1 | | |
| H7C542 | Butyrophilin | | |
| H7C542_ | subfamily 2 | | |
| HUMAN | member A1 | | |
| | BTN2A1 | | |
| C9JNC3 | Butyrophilin | | |
| C9JNC3_ | subfamily 2 | | |
| HUMAN | member A1 | | |
| | BTN2A1 | | |
| O00481 | Butyrophilin | QFSVLGPSGPILAMVGEDADLPCHLFPTMSAETMELKW | 45 |
| BT3A1_ | subfamily 3 | VSSSLRQVVNVYADGKEVEDRQSAPYRGRTSILRDGIT | |
| HUMAN | member A1 | AGKAALRIHNVTASDSGKYLCYFQDGDFYEKALVELKV | |
| | BTN3A1 BTF5 | AALGSDLHVDVKGYKDGGIHLECRSTGWYPQPQIQWSN | |
| | | NKGENIPTVEAPVVADGVGLYAVAASVIMRGSSGEGVS | |
| | | CTIRSSLLGLEKTASISIADPFFRSAQRWIAALAG | |
| E7EPR2 | Butyrophilin | | |
| E7EPR2_ | subfamily 3 | | |
| HUMAN | member A1 | | |
| | BTN3A1 | | |
| E9PFB8 | Butyrophilin | | |
| | subfamily 3 | | |
| | member A1 | | |
| E9PFB8_ | BTN3A1 | | |
| HUMAN | | | |
| A6PVC0 | Butyrophilin | | |
| A6PVC0_ | subfamily 3 | | |
| HUMAN | member A1 | | |
| | BTN3A1 | | |

In various embodiments, the present heterodimeric proteins may be engineered to target one or more molecules that reside on human leukocytes including, without limitation, the extracellular domains (where applicable) of SLAMF4, IL-2Rα, IL-2Rβ, ALCAM, B7-1, IL-4 R, B7-H3, BLAME/SLAMFS, CEACAM1, IL-6 R, IL-7Rα, IL-10Rα, IL-10Rβ, IL-12Rβ1, IL-12Rβ2, CD2, IL-13Rα1, IL-13, CD3, CD4, ILT2/CDS5j, ILT3/CDS5k, ILT4/CDS5d, ILT5/CDS5a, lutegrin α4/CD49d, CDS, Integrin αE/CD103, CD6, Integrin αM/CD 11 b, CDS, Integrin αX/CD11c, Integrin β2/CDIS, KIR/CD15S, KIR2DL1, CD2S, KIR2DL3, KIR2DL4/CD15Sd, CD31/PECAM-1, KIR2DS4, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 Rγ, Osteopontin, CRACC/SLAMF7, PD-1, CRTAM, PSGL-1, CTLA-4, CX3CR1, CX3CL1, L-Selectin, SIRP β1, SLAM, TCCR/WSX-1, DNAM-1, Thymopoietin, EMMPRIN/CD147, TIM-1, EphB6, TIM-2, TIM-3, TIM-4, Fcγ RIII/CD16, TIM-6, Granulysin, ICAM-1/CD54, ICAM-2/CD102, IFN-γR1, IFN-γ R2, TSLP, IL-1 R1 and TSLP R.

In embodiments, the present heterodimeric proteins may be engineered to target one or more molecules involved in immune inhibition, including for example: CTLA-4, PD-L1, PD-L2, PD-1, BTLA, HVEM, TIM3, GAL9, LAG3, VISTANSIG8, KIR, 2B4, TIGIT, CD160 (also referred to as BY55), CHK 1 and CHK2 kinases, A2aR, CEACAM (e.g., CEACAM-1, CEACAM-3 and/or CEACAM-5), and various B-7 family ligands (including, but are not limited to, B7-1, B7-2, B7-DC, B7-H1, B7-H2, B7-H3, B7-H4, B7-H5, B7-H6 and B7-H7).

In embodiments, the present heterodimeric proteins comprise an extracellular domain of an immune inhibitory agent. In embodiments, the present heterodimeric proteins comprise an antibody binding domain (e.g. CDR3, Fab, scFv domain, etc.) directed against an immune inhibitory agent.

In embodiments, the present heterodimeric proteins comprise an extracellular domain of a soluble or membrane protein which has immune inhibitory properties. In embodiments, the present heterodimeric proteins comprise an antibody binding domain (e.g. CDR3, Fab, scFv domain, etc.) which has immune inhibitory properties

In embodiments, the present heterodimeric proteins simulate binding of an inhibitory signal ligand to its cognate receptor but inhibit the inhibitory signal transmission to an immune cell (e.g., a T cell, macrophage or other leukocyte).

In various embodiments, the heterodimeric protein comprises an immune inhibitory receptor extracellular domain or antibody binding domain (e.g. CDR3, Fab, scFv domain, etc.) and an immune stimulatory ligand extracellular domain or antibody binding domain (e.g. CDR3, Fab, scFv domain, etc.) which can, without limitation, deliver an immune stimulation to a T cell while masking a tumor cell's immune inhibitory signals. In various embodiments, the heterodimeric protein delivers a signal that has the net result of T cell activation.

In embodiments, the present heterodimeric proteins comprise an extracellular domain of a soluble or membrane protein which has immune stimulatory properties. In embodiments, the present heterodimeric proteins comprise an antibody binding domain (e.g. CDR3, Fab, scFv domain, etc.) which has immune stimulatory properties.

In various embodiments, the present heterodimeric protein may comprise variants of any of the known cytokines, growth factors, and/or hormones. In various embodiments, the present heterodimeric proteins may comprise variants of any of the known receptors for cytokines, growth factors, and/or hormones. In various embodiments, the present heterodimeric proteins may comprises variants of any of the known extracellular domains, for instance, a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the known amino acid or nucleic acid sequences.

In various embodiments, the present heterodimeric protein may comprise an amino acid sequence having one or more amino acid mutations relative to any of the known protein sequences. In embodiments, the one or more amino acid mutations may be independently selected from substitutions, insertions, deletions, and truncations.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g., selenocysteine, pyrrolysine, N-formylmethionine R-alanine, GABA and 6-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the heterodimeric proteins by reference to the genetic code, including taking into account codon degeneracy.

In various embodiments, the present chimeric protein is or comprises an amino acid sequence having at least 90%, or at least 91%, or at least 92%, or at least 93%, or at least 94%, or at least 95%, or at least 96%, or at least 97%, or at least 98%, or at least 98%, or at least 99% (e.g. about 90%, or about 91%, or about 92%, or about 93%, or about 94%, or about 95%, or about 96%, or about 97%, or about 98%, or about 98%, or about 99%) sequence identity to one or more of SEQ ID NOs:33, 34, and 37 to 42, each optionally with the leader sequence (as indicated with double underlining elsewhere herein, or, in embodiments: MEFGLSWVFLVAIIKGVQC (SEQ ID NO: 18) omitted. In various embodiments, the present chimeric protein is or comprises an amino acid sequence having at least 90%, or at least 91%, or at least 92%, or at least 93%, or at least 94%, or at least 95%, or at least 96%, or at least 97%, or at least 98%, or at least 98%, or at least 99% (e.g. about 90%, or about 91%, or about 92%, or about 93%, or about 94%, or about 95%, or about 96%, or about 97%, or about 98%, or about 98%, or about 99%) sequence identity to one or more of SEQ ID NOs:43, 44 and 56-70, each optionally with the leader sequence (as indicated with double underlining elsewhere herein, or, in embodiments: MEFGLSWVFLVAIIKGVQC (SEQ ID NO: 18) omitted

In any of these sequence, the core domain having the following amino acid sequence is or comprises an amino acid sequence having at least 90%, or at least 91%, or at least 92%, or at least 93%, or at least 94%, or at least 95%, or at least 96%, or at least 97%, or at least 98%, or at least 98%, or at least 99% (e.g. about 90%, or about 91%, or about 92%, or about 93%, or about 94%, or about 95%, or about 96%, or about 97%, or about 98%, or about 98%, or about 99%) sequence identity to SEQ ID NO: 16.

In various embodiments, the present heterodimeric proteins are capable of, and can be used in methods comprising, promoting immune activation (e.g., against tumors). In various embodiments, the present heterodimeric proteins are capable of, and can be used in methods comprising, suppressing immune inhibition (e.g., that allows tumors to survive). In various embodiments, the present heterodimeric protein provides improved immune activation and/or improved suppression of immune inhibition.

In various embodiments, the present heterodimeric proteins are capable of, or can be used in methods comprising, modulating the amplitude of an immune response, e.g., modulating the level of effector output. In embodiments, e.g., when used for the treatment of cancer, the present heterodimeric protein alters the extent of immune stimulation as compared to immune inhibition to increase the amplitude of a T cell response, including, without limitation, stimulating increased levels of cytokine production, proliferation or target killing potential.

In embodiments, a subject is further administered autologous or allogeneic gamma delta T cells that were expanded ex vivo.

In embodiments, a subject is further administered autologous or allogeneic T cells that express a Chimeric Antigen Receptor (i.e., CAR-T cells). CAR-T cells are described in, as examples, Eshhar, et al., PNAS USA. 90(2):720-724, 1993; Geiger, et al., J Immunol. 162(10):5931-5939, 1999; Brentjens, et al., Nat Med. 9(3):279-286, 2003; Cooper, et al., Blood 101(4):1637-1644, 2003; Imai, et al., Leukemia. 18:676-684, 2004, Pang, et al., Mol Cancer. 2018; 17:91, and Schmidts, et al., Front. Immunol 2018; 9:2593; the entire contents of which are hereby incorporated by reference.

In embodiments, the heterodimeric proteins act synergistically when used in combination with Chimeric Antigen Receptor (CAR) T-cell therapy. In an illustrative embodiment, the heterodimeric proteins act synergistically when used in combination with CAR T-cell therapy in treating a tumor or cancer. In an embodiment, the heterodimeric proteins act synergistically when used in combination with CAR T-cell therapy in treating blood-based tumors. In an embodiment, the heterodimeric proteins act synergistically when used in combination with CAR T-cell therapy in treating solid tumors. For example, use of heterodimeric proteins and CAR T-cells may act synergistically to reduce or eliminate the tumor or cancer, or slow the growth and/or progression and/or metastasis of the tumor or cancer. In various embodiments, the heterodimeric proteins of the invention induce CAR T-cell division. In various embodiments, the heterodimeric proteins of the invention induce CAR T-cell proliferation. In various embodiments, the heterodimeric proteins of the invention prevents anergy of the CAR T cells.

In various embodiments, the CAR T-cell therapy comprises CAR T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MET, MUC1, MUC16, ROR-1, IL13Rα2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, GPC3, human epidermal growth factor receptor 2 (HER2), κ light chain, mesothelin, EGFR, EGFRvIII, ErbB, fibroblast activation protein (FAP), carcinoembryonic antigen (CEA), PMSA, Receptor Tyrosine Kinase Like Orphan Receptor 1 (ROR1), TAG72, and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

Exemplary CAR T-cell therapy include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio) and CAR T-cells developed by Innovative Cellular Therapeutics.

In embodiments, the CAR-T cells are autologous or allogeneic gamma delta T cells.

In various embodiments the present heterodimeric proteins, in some embodiments are capable of, or find use in methods involving, masking an inhibitory ligand on the surface of a tumor cell and replacing that immune inhibitory ligand with an immune stimulatory ligand. Accordingly, the present heterodimeric proteins, in some embodiments are capable of, or find use in methods involving, reducing or eliminating an inhibitory immune signal and/or increasing or activating an immune stimulatory signal. For example, a tumor cell bearing an inhibitory signal (and thus evading an immune response) may be substituted for a positive signal binding on a T cell that can then attack a tumor cell. Accordingly, In embodiments, an inhibitory immune signal is masked by the present heterodimeric proteins and a stimulatory immune signal is activated. Such beneficial properties are enhanced by the single construct approach of the present heterodimeric proteins. For instance, the signal replacement can be effected nearly simultaneously and the signal replacement is tailored to be local at a site of clinical importance (e.g., the tumor microenvironment).

In various embodiments, the present heterodimeric proteins are capable of, or find use in methods comprising, stimulating or enhancing the binding of immune stimulatory receptor/ligand pairs.

In other embodiments, the present heterodimeric proteins are capable of, or find use in methods involving, enhancing, restoring, promoting and/or stimulating immune modulation. In embodiments, the present heterodimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In embodiments, the present heterodimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: proinflammatory cytokine production or T cell migration or T cell tumor infiltration.

In embodiments, the present heterodimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the present heterodimeric proteins are capable of, or find use in methods involving, inhibiting and/or causing a decrease in recruitment of immunosuppressive cells (e.g., myeloid-derived suppressor cells (MDSCs), regulatory T cells (Tregs), tumor associated neutrophils (TANs), M2 macrophages, and tumor associated macrophages (TAMs) to the tumor and/or tumor microenvironment (TME). In embodiments, the present therapies may alter the ratio of M1 versus M2 macrophages in the tumor site and/or TME to favor M1 macrophages.

In embodiments, the heterotrimeric protein modulates the function of gamma delta T cells.

In various embodiments, the present heterodimeric proteins are capable of, and can be used in methods comprising, inhibiting and/or reducing T cell inactivation and/or immune tolerance to a tumor, comprising administering an effective amount of a heterodimeric protein described herein to a subject. In embodiments, the present heterodimeric proteins are able to increase the serum levels of various cytokines including, but not limited to, one or more of IFNγ, IL-2, IL-4, IL-5, IL-6, IL-9, IL-10, IL-13, IL-17A, IL-17F, and IL-22. In embodiments, the present heterodimeric proteins are capable of enhancing IL-2, IL-4, IL-5, IL-10, IL-13, IL-17A, IL-22, or IFNγ in the serum of a treated subject.

In various embodiments, the present heterodimeric proteins inhibit, block and/or reduce cell death of an anti-tumor CD8+ and/or CD4+T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections and cancer. This dysfunction is defined by poor proliferative and/or effector functions, sustained expression of inhibitory receptors and a transcriptional state distinct from that of functional effector or memory T cells. Exhaustion prevents optimal control of infection and tumors. In addition, an anti-tumor CD8+ and/or CD4+T cell refers to T cells that can mount an immune response to a tumor. Illustrative pro-tumor T cells include, but are not limited to, Tregs, CD4+ and/or CD8+T cells expressing one or more checkpoint inhibitory receptors, Th2 cells and Th17 cells. Checkpoint inhibitory receptors refers to receptors (e.g., CTLA-4, B7-H3, B7-H4, TIM-3) expressed on immune cells that prevent or inhibit uncontrolled immune responses.

In various embodiments, the present heterodimeric proteins are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL-7R/CD127⁺); CD8⁺ effector T cells (e.g., αβ TCR, CD3⁺, CD8⁺, CCR7⁺, CD62Lhi, IL-7R/CD127⁺); effector memory T cells (e.g., CD62Llow, CD44⁺, TCR, CD3⁺, IL-7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺CD62L⁻) and late effector memory T cells (CD27⁻CD62L⁻) (TemE and TemL, respectively); CD127(⁺)CD25(low/−) effector T cells; CD127(−)CD25(−) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(−) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25-regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11+ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-β, TNF-α, Galectin-1, IFN-γ and/or MCP1.

In various embodiments, the present heterodimeric proteins are capable of, and can be used in methods comprising, transiently stimulating effector T cells for no longer than about 12 hours, about 24 hours, about 48 hours, about 72 hours or about 96 hours or about 1 week or about 2 weeks. In various embodiments, the present heterodimeric proteins are capable of, and can be used in methods comprising, transiently depleting or inhibiting regulatory T cells for no longer than about 12 hours, about 24 hours, about 48 hours, about 72 hours or about 96 hours or about 1 week or about 2 weeks. In various embodiments, the transient stimulation of effector T cells and/or transient depletion or inhibition of regulatory T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In various embodiments, the present heterodimeric proteins provide advantages including, without limitation, ease of use and ease of production. This is because two distinct immunotherapy agents are combined into a single product which allows for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present heterodimeric proteins are easier and more cost effective to manufacture.

In various embodiments, the present heterodimeric proteins provide synergistic therapeutic effects as it allows for improved site-specific interplay of two immunotherapy agents. In embodiments, the present heterodimeric proteins provide the potential for reducing off-site and/or systemic toxicity.

In embodiments, the first domain and/or the heterodimeric protein modulates or is capable of modulating a γδ (gamma delta) T cell. In embodiments, the gamma delta T cell is Vγ9δ2 T cell. In embodiments, the modulation of a gamma delta T cell is activation of a gamma delta T cell. In embodiments, the heterodimeric protein is capable of forming a synapse between a gamma delta T cell and a tumor cell and/or the heterodimeric protein is capable of contemporaneous activation and targeting of gamma delta T cells to tumor cells.

The Chimeric Proteins of the Current Disclosure

In one aspect, the current disclosure relates to a chimeric protein of a general structure of: N terminus-(a)-(b)-(c)-C terminus, wherein: (a) is the first domain comprising the general structure of (a1)-SL-(a2), wherein (a1) is an extracellular domain (ECD) of a butyrophilin family protein, or a fragment thereof, (a2) is an extracellular domain (ECD) of a butyrophilin family protein, or a fragment thereof, and SL is a second linker adjoins (a1) and (a2) comprising a flexible amino acid sequence of about 4 to about 50 amino acids length, and (c) is a second domain comprising a targeting domain, the targeting domain being selected from (i) an antibody, antibody-like molecule, or antigen binding fragment thereof, and (ii) an extracellular domain of a membrane protein. (b) is linker that adjoins the first and second domains, wherein the a linker comprises at least one cysteine residue capable of forming a disulfide bond.

In embodiments, the chimeric protein is as depicted in FIG. 11, optionally comprising a polypeptide having an amino acid sequence that has at least about 95% identity with an amino acid sequence selected from SEQ ID NOs: 43, 44 and 56-70. In embodiments, the tetrameric chimeric protein is as depicted in FIG. 11, optionally comprising a polypeptide having an amino acid sequence that has an amino acid sequence selected from SEQ ID NOs: 43, 44 and 56-70.

The First Domain

In embodiments, the first domain comprises is a general structure of:

(a1)-SL-(a2), wherein

(a1) is an extracellular domain (ECD) of a butyrophilin family protein, or a fragment thereof, (a2) is an extracellular domain (ECD) of a butyrophilin family protein, or a fragment thereof, and SL is a second linker adjoins (a1) and (a2) comprising a flexible amino acid sequence of about 4 to about 50 amino acids length.

In embodiments, the first domain comprises two of the same butyrophilin family proteins. In embodiments, wherein the first domain comprises two different butyrophilin family proteins. In embodiments, the butyrophilin family proteins comprise a V-type domain.

In embodiments, the (a1) and (a2) are two of the same butyrophilin family proteins. In embodiments, the (a1) and (a2) are different butyrophilin family proteins. In embodiments, the (a1) and/or (a2) is a fragment of the butyrophilin family protein comprising a variable domain. In embodiments, the (a1) and (a2) comprise butyrophilin family proteins independently selected from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the butyrophilin family proteins are independently selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

In some embodiments, the first domain comprises a fragment of butyrophilin family proteins, wherein the fragment is capable of binding a gamma delta T cell receptor and is optionally an extracellular domain, optionally comprising a variable domain. In some embodiments, the first domain comprises a fragment of butyrophilin family proteins, wherein the fragment is capable of binding a gamma delta T cell receptor optionally selected from a Vγ4 and Vγ9δ2 TCR.

In some embodiments, the first domain comprises two of the same butyrophilin family proteins. In some embodiments, wherein the first domain comprises two different butyrophilin family proteins. In some embodiments, the butyrophilin family proteins comprise a V-type domain. Suitable butyrophilin family proteins or fragments thereof are derived from the native butyrophilin family proteins that comprise a B30.2 domain in the cytosolic tail of the full length protein.

An illustrative amino acid sequence of human BTNL3 suitable in the present technology is the following:

(SEQ ID NO: 80)
QWQVTGPGKFVQALVGEDAVFSCSLFPETSAEAMEVRFFRNQFHAVVHLY
RDGEDWESKQMPQYRGRTEFVKDSIAGGRVSLRLKNITPSDIGLYGCWFS
SQIYDEEATWELRVAALGSLPLISIVGYVDGGIQLLCLSSGWFPQPTAKW
KGPQGQDLSSDSRANADGYSLYDVEISIIVQENAGSILCSIHLAEQSHEV
ESKVLIGETFFQPSPWRLAS

The amino acid sequence of extracellular domain of human BTN2A1, which is an illustrative amino acid sequence of human BTN2A1 suitable in the current disclosure is the following:

(SEQ ID NO: 35)
QFIVVGPTDPILATVGENTTLRCHLSPEKNAEDMEVRWFR
SQFSPAVFVYKGGRERTEEQMEEYRGRTTFVSKDISRGSV
ALVIHNITAQENGTYRCYFQEGRSYDEAILHLVVAGLGSK
PLISMRGHEDGGIRLECISRGWYPKPLTVWRDPYGGVAPA
LKEVSMPDADGLFMVTTAVIIRDKSVRNMSCSINNTLLGQ
KKESVIFIPESFMPSVSPCA

In some embodiments, the fragment of extracellular domain of human BTN2A1, which is a variable domain of human BTN2A1 suitable in the current disclosure is the following:

(SEQ ID NO: 71)
QFIVVGPTDPILATVGENTTLRCHLSPEKNAEDMEVRWFR
SQFSPAVFVYKGGRERTEEQMEEYRGRTTFVSKDISRGSV
ALVIHNITAQENGTYRCYFQEGRSYDEAILHLV

The amino acid sequence of extracellular domain of human BTN3A1, which is an illustrative amino acid sequence of human BTN3A1 suitable in the current disclosure is the following:

(SEQ ID NO: 19)
QFSVLGPSGPILAMVGEDADLPCHLFPTMSAETMELKWVS
SSLRQVVNVYADGKEVEDRQSAPYRGRTSILRDGITAGKA
ALRIHNVTASDSGKYLCYFQDGDFYEKALVELKVAALGSD
LHVDVKGYKDGGIHLECRSTGWYPQPQIQWSNNKGENIPT
VEAPVVADGVGLYAVAASVIMRGSSGEGVSCTIRSSLLGL
EKTASISIADPFFRSAQRWIAALAG

In some embodiments, the fragment of extracellular domain of human BTN3A1, which is a variable of human BTN2A1 suitable in the current disclosure is the following:

(SEQ ID NO: 72)
AQFSVLGPSGPILAMVGEDADLPCHLFPTMSAETMELKWV
SSSLRQVVNVYADGKEVEDRQSAPYRGRTSILRDGITAGK
AALRIHNVTASDSGKYLCYFQDGDFYEKALVELKVA

An illustrative amino acid sequence of human BTN3A2 suitable in the present technology

(SEQ ID NO: 81)
QFSVLGPSGPILAMVGEDADLPCHLFPTMSAETMELKWVS
SSLRQVVNVYADGKEVEDRQSAPYRGRTSILRDGITAGKA
ALRIHNVTASDSGKYLCYFQDGDFYEKALVELKVAALGSN
LHVEVKGYEDGGIHLECRSTGWYPQPQIQWSNAKGENIPA
VEAPVVADGVGLYEVAASVIMRGGSGEGVSCIIRNSLLGL
EKTASISIADPFFRSAQPW

An illustrative amino acid sequence of human BTNL8 suitable in the present technology is as follows:

(SEQ ID NO: 82)
QWQVFGPDKPVQALVGEDAAFSCFLSPKTNAEAMEVRFFR
GQFSSVVHLYRDGKDQPFMQMPQYQGRTKLVKDSIAEGRI
SLRLENITVLDAGLYGCRISSQSYYQKAIWELQVSALGSV
PLISITGYVDRDIQLLCQSSGWFPRPTAKWKGPQGQDLST
DSRTNRDMHGLFDVEISLTVQENAGSISCSMRHAHLSREV
ESRVQIGDTFFEPISWHLATK

In various embodiments, the present chimeric proteins comprise two independent binding domains, each from one subunit of a heterodimeric human protein. Illustrative proteins that may be formed as part of the heterodimeric protein of the invention are provided in Table 2. In various embodiments, the present heterodimeric proteins have one of the illustrative proteins provided in Table 2. In various embodiments, the present heterodimeric proteins have two of the illustrative proteins provided in Table 2.

TABLE 2
Illustrative butyrophilin-like (BTNL) family
protein which may be incorporated into the
present compositions and methods include the
following proteins (as used herein, “Entry”
refers to the protein entry in the Uniprot
database and “Entry name” refers to the
protein entry in the Uniprot database):

| | | | SEQ |
|---|---|---|---|
| Entry/ | Protein names | | ID |
| Name | Gene names | ECD Sequence | NO |
| Q13410 | Butyrophilin | APFDVIGPPEPILAVVGEDA | 83 |
| BT1A1_ | subfamily 1 | ELPCRLSPNASAEHLELRWF | |
| HUMAN | member A1 | RKKVSPAVLVHRDGREQEAE | |
| | Butyrophilin | QMPEYRGRATLVQDGIAKGR | |
| | subfamily 1 | VALRIRGVRVSDDGEYTCFF | |
| | member A1; | REDGSYEEALVHLKVAALGS | |
| | BTN1A1 BTN | DPHISMQVQENGEICLECTS | |
| | | VGWYPEPQVQWRTSKGEKFP | |
| | | STSESRNPDEEGLFTVAASV | |
| | | IIRDTSAKNVSCYIQNLLLG | |
| | | QEKKVEISIPASSLPR | |
| Q13410 | Butyrophilin | APFDVIGPPEPILAVVGEDA | 84 |
| BT1A1_ | subfamily 1 | ELPCRLSPNASAEHLELRWF | |
| HUMAN | member A1 | RKKVSPAVLVHRDGREQEAE | |
| | BTN1A1 BTN | QMPEYRGRATLVQDGIAKGR | |
| | | VALRIRGVRVSDDGEYTCFF | |
| | | REDGSYEEALVHLKVAALGS | |
| | | DPHISMQVQENGEICLECTS | |
| | | VGWYPEPQVQWRTSKGEKFP | |
| | | STSESRNPDEEGLFTVAASV | |
| | | IIRDTSAKNVSCYIQNLLLG | |
| | | QEKKVEISIPASSLP | |
| Q4VAN1Q4 | BTN1A1 protein | | |
| VAN1_ | BTN1A1 | | |
| HUMAN | | | |
| Q4VAN2 | Butyrophilin, | | |
| Q4VAN2_ | subfamily 1, | | |
| HUMAN | member A. | | |
| | BTN1A1 | | |
| Q9UIRO | Butyrophilin- | KQSEDFRVIGPAHPILAGVG | 85 |
| BTNL2_ | like | EDALLTCQLLPKRTTMHVEV | |
| HUMAN | protein 2 | RWYRSEPSTPVFVHRDGVEV | |
| | BTNL2 | TEMQMEEYRGWVEWIENGIA | |
| | | KGNVALKIHNIQPSDNGQYW | |
| | | CHFQDGNYCGETSLLLKVAG | |
| | | LGSAPSIHMEGPGESGVQLV | |
| | | CTARGWFPEPQVYWEDIRGE | |
| | | KLLAVSEHRIQDKDGLFYAE | |
| | | ATLVVRNASAESVSCLVHNP | |
| | | VLTEEKGSVISLPEKLQTEL | |
| | | ASLKVNGPSQPILVRVGEDI | |
| | | QLTCYLSPKANAQSMEVRWD | |
| | | RSHRYPAVHVYMDGDHVAGE | |
| | | QMAEYRGRTVLVSDAIDEGR | |
| | | LTLQILSARPSDDGQYRCLF | |
| | | EKDDVYQEASLDLKVVSLGS | |
| | | SPLITVEGQEDGEMQPMCSS | |
| | | DGWFPQPHVPWRDMEGKTIP | |
| | | SSSQALTQGSHGLFHVQTLL | |
| | | RVTNISAVDVTCSISIPFLG | |
| | | EEKIATFSLSGW | |
| F8WBA1 | Butyrophilin- | | |
| F8WBA1_ | like | | |
| HUMAN | protein 2 | | |
| | BTNL2 | | |
| F6UPS5 | Butyrophilin- | | |
| F6UPS5_ | like | | |
| HUMAN | protein 2 | | |
| | BTNL2 | | |
| F8WDK6 | Butyrophilin- | | |
| F8WDK6 | like | | |
| HUMAN | protein 2 | | |
| | BTNL2 | | |
| A0A0G2JJ | Butyrophilin- | | |
| 84 | like | | |
| A0A0G2JJ | protein 2 | | |
| 84_HUMAN | BTNL2 | | |
| X5D146 | BTNL2 | | |
| X5D146_ | BTNL2 | | |
| HUMAN | | | |
| A0A0G2JP | BTNL2 | | |
| B7 | | | |
| A0A0G2JP | | | |
| B7_ | | | |
| HUMAN | | | |
| AOPJV4 | BTNL2 protein | | |
| AOPJV4_ | BTNL2 | | |
| HUMAN | | | |
| 17HPB5 | Butyrophilin- | | |
| 17HPB5_ | like | | |
| HUMAN | 2 (MHC class II | | |
| | a . . . | | |
| | BTNL2 RP5- | | |
| | 107715.2-002 | | |
| A0A1U9X7 | BTNL2 | | |
| B7 | | | |
| A0A1U9X7 | | | |
| B7_ | | | |
| HUMAN | | | |
| X5CF33 | BTNL2 | | |
| X5CF33_ | BTNL2 hCG_ | | |
| HUMAN | 43715 | | |
| A0A1U9X7 | | | |
| C0 | | | |
| A0A1U9X7 | BTNL2 | | |
| CO_HUMAN | | | |
| A0A1U9X7 | Truncated | | |
| C3 | BTNL2 | | |
| A0A1U9X7 | | | |
| C3_ | | | |
| HUMAN | | | |
| A0A1U9X7 | Truncated | | |
| C4 | BTNL2 | | |
| A0A1U9X7 | | | |
| C4_ | | | |
| HUMAN | | | |
| Q7KYR7 | Butyrophilin | QFIVVGPTDPILATVGENTT | 36 |
| BT2A1_ | subfamily 2 | LRCHLSPEKNAEDMEVRWFR | |
| HUMAN | member A1 | SQFSPAVFVYKGGRERTEEQ | |
| | BTN2A1 BT2.1, | MEEYRGRTTFVSKDISRGSV | |
| | BTF1 | ALVIHNITAQENGTYRCYFQ | |
| | | EGRSYDEAILHLVVAGLGSK | |
| | | PLISMRGHEDGGIRLECISR | |
| | | GWYPKPLTVWRDPYGGVAPA | |
| | | LKEVSMPDADGLFMVTTAVI | |
| | | IRDKSVRNMSCSINNTLLGQ | |
| | | KKESVIFIPESFMPSVSPCA | |
| H7BYC3 | Butyrophilin | | |
| H7BYC3_ | subfamily 2 | | |
| HUMAN | member A1 | | |
| | BTN2A1 | | |
| H7C542 | Butyrophilin | | |
| H7C542_ | subfamily 2 | | |
| HUMAN | member A1 | | |
| | BTN2A1 | | |
| C9JNC3 | Butyrophilin | | |
| C9JNC3_ | subfamily 2 | | |
| HUMAN | member A1 | | |
| | BTN2A1 | | |
| Q8WVV5 | Butyrophilin | QFTVVGPANPILAMVGENTT | 86 |
| BT2A2_ | subfamily 2 | LRCHLSPEKNAEDMEVRWFR | |
| HUMAN | member A2 | SQFSPAVFVYKGGRERTEEQ | |
| | BTN2A2 BT2.2, | MEEYRGRITFVSKDINRGSV | |
| | BTF2 | ALVIHNVTAQENGIYRCYFQ | |
| | | EGRSYDEAILRLVVAGLGSK | |
| | | PLIEIKAQEDGSIWLECISG | |
| | | GWYPEPLTVWRDPYGEVVPA | |
| | | LKEVSIADADGLFMVTTAVI | |
| | | IRDKYVRNVSCSVNNTLLGQ | |
| | | EKETVIFIPESFMPSASPWM | |
| | | VALAVILTASPWM | |
| A0A024R0 | Butyrophilin, | | |
| 38 | subfamily 2, | | |
| A0A024R0 | member A . . . | | |
| 38_ | BTN2A2 hCG_ | | |
| HUMAN | 1980289 | | |
| C9J8J5 | Butyrophilin | | |
| C9J8J5_ | subfamily 2 | | |
| HUMAN | member A2 | | |
| | BTN2A2 | | |
| C9IZY2 | Butyrophilin | | |
| C9IZY2_ | subfamily 2 | | |
| HUMAN | member A2 | | |
| | BTN2A2 | | |
| B4E3J1 | cDNA | | |
| B4E3J1_ | FLJ52852, | | |
| HUMAN | highly similar | | |
| | to | | |
| | Ho . . . | | |
| C91Y66 | Butyrophilin | | |
| C91Y66_ | subfamily 2 | | |
| HUMAN | member A2 | | |
| | BTN2A2 | | |
| C9J8R3 | Butyrophilin | | |
| C9J8R3_ | subfamily 2 | | |
| HUMAN | member A2 | | |
| | BTN2A2 | | |
| C9JAJ6 | Butyrophilin | | |
| C9JAJ6_ | subfamily 2 | | |
| HUMAN | member A2 | | |
| | BTN2A2 | | |
| C9JWH2 | Butyrophilin | | |
| C9JWH2_ | subfamily 2 | | |
| HUMAN | member A2 | | |
| | BTN2A2 | | |
| H7C4E8 | Butyrophilin | | |
| H7C4E8_ | subfamily 2 | | |
| HUMAN | member A2 | | |
| | BTN2A2 | | |
| F8WC65 | Butyrophilin | | |
| F8WC65_ | subfamily 2 | | |
| HUMAN | member A2 | | |
| | BTN2A2 | | |
| Q96KV6 | Putative | QVTVVGPTDPILAMVGENTT | 87 |
| BT2A3_ | butyrophilin | LRCCLSPEENAEDMEVRWFQ | |
| HUMAN | subfamily | SQFSPAVFVYKGGRERTEEQ | |
| | 2 m . . . | KEEYRGRTTFVSKDSRGSVA | |
| | BTN2A3P BTN | LIIHNVTAEDNGIYQCYFQE | |
| | 2A3 | GRSCNEAILHLVVAGLDSEP | |
| | | VIEMRDHEDGGIQLECISGG | |
| | | WYPKPLTVWRDPYGEVVPAL | |
| | | KEVSTPDADSLFMVTTAVII | |
| | | RDKSVRNVSCSINDTLLGQK | |
| | | KESVIFIPESFMPSRSPCV | |
| Q6UXE8 | Butyrophilin- | QWQVTGPGKFVQALVGEDAV | 88 |
| BTNL3_ | like | FSCSLFPETSAEAMEVRFFR | |
| HUMAN | protein 3 | NQFHAVVHLYRDGEDWESKQ | |
| | BTNL3 BTNLR, | MPQYRGRTEFVKDSIAGGRV | |
| | COLF4100, | SLRLKNI | |
| | UNQ744/ | TPSDIGLYGCWFSSQIYDEE | |
| | PRO1472 | ATWELRVAALGSLPLISIVG | |
| | | YVDGGIQLLCLSSGWFPQPT | |
| | | AKWKGPQGQDLSSDSRANAD | |
| | | GYSLYDVEISIIVQENAGSI | |
| | | LCSIHLAEQSHEVESKVLIG | |
| | | ETFFQPSPWRLAS | |
| L8EAU7 | Alternative | | |
| L8EAU7_ | protein BTNL3 | | |
| HUMAN | BTNL3 | | |
| O00481 | Butyrophilin | QFSVLGPSGPILAMVGEDAD | 45 |
| BT3A1_ | subfamily 3 | LPCHLFPTMSAETMELKWVS | |
| HUMAN | member A1 | SSLRQVVNVYADGKEVEDRQ | |
| | BTN3A1 BTF5 | SAPYRGRTSILRDGITAGKA | |
| | | ALRIHNVTASDSGKYLCYFQ | |
| | | DGDFYEKALVELKVAALGSD | |
| | | LHVDVKGYKDGGIHLECRST | |
| | | GWYPQPQIQWSNNKGENIPT | |
| | | VEAPVVADGVGLYAVAASVI | |
| | | MRGSSGEGVSCTIRSSLLGL | |
| | | EKTASISIADPFFRSAQRWI | |
| | | AALAG | |
| E7EPR2 | Butyrophilin | | |
| E7EPR2_ | subfamily 3 | | |
| HUMAN | member A1 | | |
| | BTN3A1 | | |
| E9PFB8 | Butyrophilin | | |
| E9PFB8_ | subfamily 3 | | |
| HUMAN | member A1 | | |
| | BTN3A1 | | |
| A6PVC0 | Butyrophilin | | |
| A6PVC0_ | subfamily 3 | | |
| HUMAN | member A1 | | |
| | BTN3A1 | | |
| P78410 | Butyrophilin | QFSVLGPSGPILAMVGEDAD | 89 |
| BT3A2_ | subfamily 3 | LPCHLFPTMSAETMELKWVS | |
| HUMAN | member A2 | SSLRQVVNVYADGKEVEDRQ | |
| | BTN3A2 BT3.2, | SAPYRGRTSILRDGITAGKA | |
| | BTF3, BTF4 | ALRIHNVTASDSGKYLCYFQ | |
| | | DGDFYEKALVELKVAALGSN | |
| | | LHVEVKGYEDGGIHLECRST | |
| | | GWYPQPQIQWSNAKGENIPA | |
| | | VEAPVVADGVGLYEVAASVI | |
| | | MRGGSGEGVSCIIRNSLLGL | |
| | | EKTASISIADPFFRSAQPW | |
| A0A024QZ | Butyrophilin, | | |
| Z1 | subfamily 3, | | |
| A0A024QZ | member A . . . | | |
| Z1_ | BTN3A2 hCG_ | | |
| HUMAN | 17993 | | |
| S4R3NO | Butyrophilin | | |
| S4R3NO_ | subfamily 3 | | |
| HUMAN | member A2 | | |
| | BTN3A2 | | |
| E9PJE9 | Butyrophilin | | |
| E9PJE9_ | subfamily 3 | | |
| HUMAN | member A2 | | |
| | BTN3A2 | | |
| E9PIU5 | Butyrophilin | | |
| E9PIU5_ | subfamily 3 | | |
| HUMAN | member A2 | | |
| | BTN3A2 | | |
| E9PRR1 | Butyrophilin | | |
| E9PRR1_ | subfamily 3 | | |
| HUMAN | member A2 | | |
| | BTN3A2 | | |
| E9PRX1 | Butyrophilin | | |
| E9PRX1_ | subfamily 3 | | |
| HUMAN | member A2 | | |
| | BTN3A2 | | |
| O00478 | Butyrophilin | QFSVLGPSGPILAMVGEDAD | 90 |
| BT3A3_ | subfamily 3 | LPCHLFPTMSAETMELRWVS | |
| HUMAN | member A3 | SSLRQVVNVYADGKEVEDRQ | |
| | BTN3A3 BTF3 | SAPYRGRTSILRDGITAGKA | |
| | | ALRIHNVTASDSGKYLCYFQ | |
| | | DGDFYEKALVELKVAALGSD | |
| | | LHIEVKGYEDGGIHLECRST | |
| | | GWYPQPQIKWSDTKGENIPA | |
| | | VEAPVVADGVGLYAVAASVI | |
| | | MRGSSGGGVSCIIRNSLLGL | |
| | | EKTASISIADPFFRSAQPW | |
| A0A024R0 | Butyrophilin, | | |
| 42 | subfamily 3, | | |
| A0A024R0 | member A . . . | | |
| 42_ | BTN3A3 hCG_ | | |
| HUMAN | 17992 | | |
| A0A089GIA | Butyrophilin | | |
| 6 | subfamily 3 | | |
| A0A089GIA | member | | |
| 6_HUMAN | A3 . . . | | |
| | BTN3A3 | | |
| C9JUV8 | Butyrophilin | | |
| C9JUV8_ | subfamily 3 | | |
| HUMAN | member A3 | | |
| | BTN3A3 | | |
| C9JQT8 | Butyrophilin | | |
| C9JQT8_ | subfamily 3 | | |
| HUMAN | member A3 | | |
| | BTN3A3 | | |
| C9JVU4 | Butyrophilin | | |
| C9JVU4_ | subfamily 3 | | |
| HUMAN | member A3 | | |
| | BTN3A3 | | |
| C9J3Q8 | Butyrophilin | | |
| C9J3Q8_ | subfamily 3 | | |
| HUMAN | member A3 | | |
| | BTN3A3 | | |
| C9JZT5 | Butyrophilin | | |
| C9JZT5_ | subfamily 3 | | |
| HUMAN | member A3 | | |
| | BTN3A3 | | |
| C9J877 | Butyrophilin | | |
| C9J877_ | subfamily 3 | | |
| HUMAN | member A3 | | |
| | BTN3A3 | | |
| C9JNZ3 | Butyrophilin | | |
| C9JNZ3_ | subfamily 3 | | |
| HUMAN | member A3 | | |
| | BTN3A3 | | |
| Q6UX41 | Butyrophilin- | QWQVFGPDKPVQALVGEDAA | 91 |
| BTNL8_ | like | FSCFLSPKTNAEAMEVRFFR | |
| HUMAN | protein 8 | GQFSSVVHLYRDGKDQPFMQ | |
| | BTNL8 UNQ702/ | MPQYQGRTKLVKDSIAEGRI | |
| | PRO1347 | SLRLENITVLDAGLYGCRIS | |
| | | SQSYYQKAIWELQVSALGSV | |
| | | PLISITGYVDRDIQLLCQSS | |
| | | GWFPRPTAKWKGPQGQDLST | |
| | | DSRTNRDMHGLFDVEISLTV | |
| | | QENAGSISCSMRHAHLSREV | |
| | | ESRVQIGDTFFEPISWHLAT | |
| | | K | |
| D6RIR7 | Butyrophilin- | | |
| D6RIR7_ | like protein 8 | | |

| | | | |
|---|---|---|---|
| HUMAN | BTNL8 | | |
| D6R918 | Butyrophilin- | | |
| D6R918_ | like | | |
| HUMAN | protein 8 | | |
| | BTNL8 | | |
| Q6UXG8 | Butyrophilin- | SSEVKVLGPEYPILALVGEE | 92 |
| BTNL9_ | like | VEFPCHLWPQLDAQQMEIRW | |
| HUMAN | protein 9 | FRSQTFNVVHLYQEQQELPG | |
| | BTNL9 UNQ19 | RQMPAFRNRTKLVKDDIAYG | |
| | 00/PRO4346 | SVVLQLHSIIPSDKGTYGCR | |
| | | FHSDNFSGEALWELEVAGLG | |
| | | SDPHLSLEGFKEGGIQLRLR | |
| | | SSGWYPKPKVQWRDHQGQCL | |
| | | PPEFEAIVWDAQDLFSLETS | |
| | | VVVRAGALSNVSVSIQNLLL | |
| | | SQKKELVVQIADVFVPGASA | |
| | | WK | |
| A0A1S5UZ | Butyrophilin- | | |
| 21 | like | | |
| A0A1S5UZ | protein 9 | | |
| 21_ | BTNL9 | | |
| HUMAN | | | |
| B7Z4Y8 | Butyrophilin- | | |
| B7Z4Y8_ | like | | |
| HUMAN | protein 9 | | |
| | BTNL9 | | |
| Q8N324 | BTNL9 protein | | |
| Q8N324_ | BTNL9 | | |
| HUMAN | | | |
| A8MVZ5 | Butyrophilin- | SIWKADFDVTGPHAPILAMA | 93 |
| BTNLA_ | like | GGHVELQCQLFPNISAEDME | |
| HUMAN | protein 10 | LRWYRCQPSLAVHMHERGMD | |
| | BTNL10 | MDGEQKWQYRGRTTFMSDHV | |
| | | ARGKAMVRSHRVTTFDNRTY | |
| | | CCRFKDGVKFGEATVQVQVA | |
| | | GLGREPRIQVTDQQDGVRAE | |
| | | CTSAGCFPKSWVERRDFRGQ | |
| | | ARPAVTNLSASATTRLWAVA | |
| | | SSLTLWDRAVEGLSCSISSP | |
| | | LLPERRKVAESHLPATFSRS | |
| | | SQFTAWKA | |

In embodiments, the first domain comprises a polypeptide having (a1) an amino acid sequence having at least 90%, or 95%, or 97%, or 98%, or 99% identity with an amino acid sequence selected from SEQ ID NOs: 19, 35-36, 45, 71-72, 80-93, and (a2) an amino acid sequence having at least 90%, or 95%, or 97%, or 98%, or 99% identity with an amino acid sequence selected from SEQ ID NOs: 19, 35-36, 45, 71-72, 80-93. In embodiments, the first domain comprises a polypeptide having an amino acid sequence of: (a1) any one of SEQ ID NOs: 19, 35-36, 45, 71-72, 80-93; and (a2) any one of SEQ ID NOs: 19, 35-36, 45, 71-72, 80-93. In embodiments, the first domain comprises extracellular domains of: (i) BTNL3 and BTNL8; (ii) BTN2A1 and BTN3A1; (iii) BTN3A1 and BTN3A2; or (iv) BTN3A1 and BTN3A3. In embodiments, the first domain comprises variable domains of: (i) BTNL3 and BTNL8; (ii) BTN2A1 and BTN3A1; (iii) BTN3A1 and BTN3A2; or (iv) BTN3A1 and BTN3A3.

In embodiments, the present chimeric protein comprises the extracellular domains of two butyrophilin family of proteins independently selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL. In embodiments, the present chimeric protein comprises the variable domains of two butyrophilin family of protein independently selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL. For instance, the chimeric protein may comprise two butyrophilin family of proteins, or variants, variable domains or functional fragments thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with two amino acid sequences independently selected from SEQ ID NOs: 19, 35-36, 45, 71-72, 80-93.

In embodiments, the second linker comprises an amino acid sequence of general formula G(G₃S)ₘ or GGGSₙ wherein m and n are integers in the range 1 to 16. In embodiments, the second linker is a flexible amino acid sequence. Exemplary second linkers are G(G₃S)ₘ, or GGGSₙ where m or n is 2-6, for example, GGGGSGGGS (SEQ ID NO: 73), GGGGSGGGGSGGGGS (SEQ ID NO: 74), GGGGSGGGSGGGS (SEQ ID NO: 75), GGGSGGGSGGGSGGGS (SEQ ID NO: 76), GGGGSGGGSGGGSGGGS (SEQ ID NO: 77), GGGGSGGGGS (SEQ ID NO: 78), and GGGGSGGGGSGGGGSGGGGS (SEQ ID NO: 79).

The Second Domain Comprising a Targeting Domain

The heterodimeris proteins of any of the embodiments disclosed herein comprise a second domain comprising a targeting domain. In some embodiments, the targeting domain is an antibody-like molecule, or antigen binding fragment thereof. In some embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In some embodiments, the antibody-like molecule is an scFv. In embodiments, the targeting domain specifically binds one of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, and GD3. In embodiments, the targeting domain specifically binds CD19. In embodiments, the targeting domain specifically binds PSMA.

In embodiments, the targeting domain specifically binds CD33. In embodiments, the targeting domain specifically binds CLL-1.

Illustrative sequences of second domain comprising a targeting domain are provided below:

In one aspect, the current disclosure relates to a heterodimeric protein a second domain comprising a targeting domain that specifically binds to CD19.

The heterodimeric proteins of any of the embodiments disclosed herein comprise a second domain comprising a targeting domain. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv. In embodiments, the targeting domain is an extracellular domain. In embodiments, the targeting domain is capable of binding an antigen on the surface of a cancer cell. In embodiments, the targeting domain specifically binds one of CD19, PSMA, GD2, PSCA, BCMA, CD123, B7-H3, CD20, CD30, CD33, CD38, CEA, CLEC12A, DLL3, EGFRvIII, EpCAM, CD307, FLT3, GPC3, gpA33, HER2, MUC16, P-cadherin, SSTR2, and mesothelin. In embodiments, the targeting domain comprises a portion of the extracellular domain of LAG-3, PD-1, TIGIT, CD19, or PSMA. In embodiments, the targeting domain specifically binds PSMA. In embodiments, the targeting domain specifically binds CD19.

In embodiments, the targeting domain is an antibody, or an antigen binding fragment thereof. In embodiments, the binding fragment comprises an Fv domain. In embodiments, the targeting domain is an antibody-like molecule, or antigen binding fragment thereof. In embodiments, the binding fragment comprises an scFv domain.

Illustrative sequences of second domain comprising a targeting domain are provided below:

An illustrative targeting domain is scFVh19, which is the heavy chain variable domain of an scFV specific to human CD19, and has the following sequence:

(SEQ ID NO: 20)
DIQLTQSPASLAVSLGQRATISCKASQSVDYDGDSYLNWY
QQIPGQPPKLLIYDASNLVSGIPPRFSGSGSGTDFTLNIH
PVEKVDAATYHCQQSTEDPWTFGGGTKLEIK

An illustrative targeting domain is scFVlh19, which is light chain variable domain of an scFV specific to human CD19, and has the following sequence:

(SEQ ID NO: 21)
EVQLVESGGGLVQPGGSLTLSCAASRFMISEYHMHWVRQA
PGKGLEWVSTINPAGTTDYAESVKGRFTISRDNAKNTLYL
QMNSLKPEDTAVYYCDSYGYRGQGTQVTV

An illustrative targeting domain is scFvCD19, which an scFV specific to human CD19, and has the following sequence:

(SEQ ID NO: 22)
QVQLQQSGAELVRPGSSVKISCKASGYAFSSYWMNWVKQR
PGQGLEWIGQIWPGDGDTNYNGKFKGKATLTADESSSTAY
MQLSSLASEDSAVYFCARRETTTVGRYYYAMDYWGQGTTV
TVSSGGGGSGGGGSGGGGSDIQLTQSPASLAVSLGQRATI
SCKASQSVDYDGDSYLNWYQQIPGQPPKLLIYDASNLVSG
IPPRFSGSGSGTDFTLNIHPVEKVDAATYHCQQSTEDPWT
FGGGTKLEIK

An illustrative targeting domain is 19scFv3, which an scFV specific to human CD19, and has the following sequence:

(SEQ ID NO: 23)
DIQMTQTTSSLSASLGDRVTISCRASQDISKYLNWYQQKP
DGTVKLLIYHTSRLHSGVPSRFSGSGSGTDYSLTISNLEQ
EDIATYFCQQGNTLPYTFGGGTKLEITGGGSGGGSGGGSE
VKLQESGPGLVAPSQSLSVTCTVSGVSLPDYGVSWIRQPP
RKGLEWLGVIWGSETTYYNSALKSRLTIIKDNSKSQVFLK
MNSLQTDDTAIYYCAKHYYYGGSYAMDYWGQGTSVTVSS

An illustrative targeting domain is scFvCD19VHVL, which an scFV specific to mouse CD19, and has the following sequence:

(SEQ ID NO: 24)
EVQLQQSGAELVRPGTSVKLSCKVSGDTITFYYMHFVKQR
PGQGLEWIGRIDPEDESTKYSEKFKNKATLTADTSSNTAY
LKLSSLTSEDTATYFCIYGGYYFDYWGQGVMVTVSSGGGG
SGGGGSGGGGSDIQMTQSPASLSTSLGETVTIQCQASEDI
YSGLAWYQQKPGKSPQLLIYGASDLQDGVPSRFSGSGSGT
QYSLKITSMQTEDEGVYFCQQGLTYPRTFGGGTKLELK

An illustrative targeting domain is scFvCD19VLVH, which an scFV specific to mouse CD19, and has the following sequence:

(SEQ ID NO: 25)
DIQMTQSPASLSTSLGETVTIQCQASEDIYSGLAWYQQKP
GKSPQLLIYGASDLQDGVPSRFSGSGSGTQYSLKITSMQT
EDEGVYFCQQGLTYPRTFGGGTKLELKGGGGSGGGGSGGG
GSEVQLQQSGAELVRPGTSVKLSCKVSGDTITFYYMHFVK
QRPGQGLEWIGRIDPEDESTKYSEKFKNKATLTADTSSNT
AYLKLSSLTSEDTATYFCIYGGYYFDYWGQGVMVTVSS

An illustrative targeting domain is scFVIPSMA, which is light chain variable domain of an scFV specific to human PSMA, and has the following sequence:

(SEQ ID NO: 26)
RKGGKRGSGSGQTVVTQEPSLTVSPGGTVTLTCASSTGAV
TSGNYPNWVQQKPGQAPRGLIGGTKFLVPGTPARFSGSLL
GGKAALTLSGVQPEDEAEYYCTLWYSNRWVFGGGTKLTVL

An illustrative targeting domain is GD2scFv3, which an scFV specific to human GD2, and has the following sequence

(SEQ ID NO: 27)
GTDFTLKISRVEAEDLGVYFCSQSTHVPPLTFGAGTKLEL
KGGGSGGGSGGGSEVQLLQSGPELEKPGASVMISCKASGS
SFTGYNMNWVRQNIGKSLEWIGAIDPYYGGTSYNQKFKGR
ATLTVDKSSSTAYMHLKSLTSEDSAVYYCVSGMKYWGQGT
SVTVSS

An illustrative targeting domain is CD33scFv-3, which an scFV specific to human CD33, and has the following sequence (the linker joining the variable regions of the heavy (V_{H}) and light chains (V_{L}) is shown by an underline):

(SEQ ID NO: 94)
QVQLVQSGGGLVQPGGSLRLSCAASGFTFSSYAMSWVRQAPGKGLEWVSA
ISGSGGSTYYADSVKGRFTISRDNSKNTLYLQMNSLRAEDTAVYYCAKED
TIRGPNYYYYGMDVWGQGTTVTVSSASGGGGSGGGGSGGGGSETTLTQSP
SSVSASVGDRVSITCRASQDIDTWLAWYQLKPGKAPKLLMYAASNLQGGV
PSRFSGSGSGTDFILTISSLQPEDFATYYCQQASIFPPTFGGGTKVDIK

An illustrative targeting domain is CD33scFv-4, which an scFV specific to human CD33, and has the following sequence (the linker joining the variable regions of the heavy (V_{H}) and light chains (V_{L}) is shown by an underline):

(SEQ ID NO: 95)
QVQLVQSGAEVKKPGESLKISCKGSGYSFTSYWIGWVRQMPGKGLEWMGI
IYPGDSDTRYSPSFQGQVTISADKSITTAYLQWSSLRASDSAMYYCARGG
YSDYDYYFDFWGQGTLVTVSSASGGGGSGGGGSGGGGSEIVMTQSPLSLP
VTPGEPASISCRSSQSLLHSNGYNYLDWYLQKPGQSPQLLIYLGSNRASG
VPDRFSGSGSGTDFTLKISRVEAEDVGVYYCMQALQTPFTFGGGTKVEIK

An illustrative targeting domain is CD33scFv-5, which an scFV specific to human CD33, and has the following sequence (the linker joining the variable regions of the heavy (V_{H}) and light chains (V_{L}) is shown by an underline):

(SEQ ID NO: 96)
QVQLVQSGGDLAQPGRSLRLSCAASGFTFDDYAMHWVRQAPGKGLEWVAV
IWPDGGQKYYGDSVKGRFTVSRDNPKNTLYLQMNSLRAEDTAIYYCVRHF
NAWDYWGQGTLVTVSSASGGGGSGGGGSGGGGSDIQLTQSPSSLSAYVGG
RVTITCQASQGISQFLNWFQQKPGKAPKLLISDASNLEPGVPSRFSGSGS
GTDFTFTITNLQPEDIATYYCQQYDDLPLTFGGGTKVEIK

An illustrative targeting domain is CD33scFv-6, which an scFV specific to human CD33, and has the following sequence (the linker joining the variable regions of the heavy (V_{H}) and light chains (V_{L}) is shown by an underline):

(SEQ ID NO: 97)
QVQLVQSGGGVVQPGKSLRLSCAASGFTFSIFAMHWVRQAPGKGLEWVAT
ISYDGSNAFYADSVEGRFTISRDNSKDSLYLQMDSLRPEDTAVYYCVKAG
DGGYDVFDSWGQGTLVTVSSASGGGGSGGGGSGGGGSEIVMTQSPLSLPV
TPGEPASISCRSSQSLLHSNGYNYLDWYLQKPGQSPQLLIYLGSNRASGV
PDRFSGSGSGTDFTLKISRVEAEDVGVYYCMQALQTPTFGPGTKVDIK

An illustrative targeting domain is CD33scFv-7, which an scFV specific to human CD33, and has the following sequence (the linker joining the variable regions of the heavy (V_{H}) and light chains (V_{L}) is shown by an underline):

(SEQ ID NO: 98)
EVQLVESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKET
DYYGSGTFDYWGQGTLVTVSSASGGGGSGGGGSGGGGSDIQMTQSPSSLS
ASVGDRVTISCRASQGIGIYLAWYQQRSGKPPQLLIHGASTLQSGVPSRF
SGSGSGTDFTLTISSLQPEDFASYWCQQSNNFPPTFGQGTKVEIK

An illustrative targeting domain is CD33scFv-9, which an scFV specific to human CD33, and has the following sequence (the linker joining the variable regions of the heavy (V_{H}) and light chains (V_{L}) is shown by an underline):

(SEQ ID NO: 99)
QVQLVQSGAEVKKPGESLKISCKGSGYSFTNYWIGWVRQMPGKGLEWMGI
IYPGDSDTRYSPSFQGQVTISADKSISTAYLQWSSLKASDTAMYYCARHG
PSSWGEFDYWGQGTLVTVSSASGGGGSGGGGSGGGGSDIRLTQSPSSLSA
SVGDRVTITCRASQSISSYLNWYQQKPGKAPKLLIYAASSLQSGVPSRFS
GSGSGTDFTLTISSLQPEDFATYYCQQSYSTPLTFGGGTKVDIK

An illustrative targeting domain is CD33scFv-10, which an scFV specific to human CD33, and has the following sequence (the linker joining the variable regions of the heavy (V_{H}) and light chains (V_{L}) is shown by an underline):

(SEQ ID NO: 100)
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSASGGGGSGGGGSGGGGSDIQLTQSPSTLSASVGD
RVTITCRASESLDNYGIRFLTWFQQKPGKAPKLLMYAASNQGSGVPSRFS
GSGSGTEFTLTISSLQPDDFATYYCQQTKEVPWSFGQGTKVEVK

An illustrative targeting domain is CD20scFv-1, which an scFV specific to human CD20, and has the following sequence (the variable regions of the heavy clain (V_{H}) is shown in a boldface font, the variable regions of the light chain (V_{L}) is indicated in an italics font, and the linker joining V_{H} and V_{L} is shown by an underline):

(SEQ ID NO: 101)
EVQLVESGGGLVQPGRSLRLSCVASGFTFNDYAMHWVRQAPGKGLEWVSV
ISWNSDSIGYADSVKGRFTISRDNAKNSLYLQMHSLRAEDTALYYCAKDN
HYGSGSYYYYQYGMDVWGQGTTVTVSSGGGGSGGGGSGGGGSGGGGSAEI
VMTQSPATLSVSPGERATLSCRASQSVSSNLAWYQQKPGQAPRLLIYGAS
TRATGIPARFSGSGSGTEFTLTISSLQSEDFAVYYCQHYINWPLTFGGGT
KVEIK

An illustrative targeting domain is CD20scFv-2, which an scFV specific to human CD20, and has the following sequence (the variable regions of the heavy clain (V_{H}) is shown in a boldface font, the variable regions of the light chain (V_{L}) is indicated in an italics font, and the linker joining V_{H} and V_{L} is shown by an underline):

(SEQ ID NO: 102)
EVQLVQSGAEVKKPGESLKISCKGSGRTFTSYNMHWVRQMPGKGLEWMGA
IYPLTGDTSYNQKSKLQVTISADKSISTAYLQWSSLKASDTAMYYCARST
YVGGDWQFDVWGKGTTVTVSSGGGGSGGGGSGGGGSGGGGSEIVLTQSPG
TLSLSPGERATLSCRASSSVPYIHWYQQKPGQAPRLLIYATSALASGIPD
RESGSGSGTDFTLTISRLEPEDFAVYYCQQWLSHPPTFGQGTKLEIK

An illustrative targeting domain is CD20scFv-3, which an scFV specific to human CD20, and has the following sequence (the linker joining the variable regions of the heavy clain (V_{H}) and the variable regions of the light chain (V_{L}) is shown by an underline):

(SEQ ID NO: 103)
QVQLVQSGAEVKKPGASVKVSCKASGYTFTSYNMHWVRQAPGQGLEWMGA
IYPGNGDTSYNQKFQGRVTITADKSISTAYMELSSLRSEDTAVYYCARST
YYGGDWYFNVWGAGTLVTVSSGGGGSGGGGSGGGGSGGGGSQIVLTQSPS
SLSASVGDRVTITCRASSSVSYIHWFQQKPGKSPKPLIYATSNLASGVPV
RFSGSGSGTDYTLTISSLQPEDFATYYCQQWTSNPPTFGGGTKVEIK

An illustrative targeting domain is CD20scFv-4, which an scFV specific to human CD20, and has the following sequence (the linker joining the variable regions of the heavy clain (V_{H}) and the variable regions of the light chain (V_{L}) is shown by an underline):

(SEQ ID NO: 104)
EVQLVESGGGLVQPDRSLRLSCAASGFTFHDYAMHWVRQAPGKGLEWVST
ISWNSGTIGYADSVKGRFTISRDNAKNSLYLQMNSLRAEDTALYYCAKDI
QYGNYYYGMDVWGQGTTVTVSSGGGGSGGGGSGGGGSGGGGSEIVLTQSP
ATLSLSPGERATLSCRASQSVSSYLAWYQQKPGQAPRLLIYDASNRATGI
PARFSGSGSGTDFTLTISSLEPEDFAVYYCQQRSNWPITFGQGTRLEIK

An illustrative targeting domain is GPRC5DscFv-1, which an scFV specific to human GPRC5D, and has the following sequence (the linker joining the variable regions of the heavy clain (V_{H}) and the variable regions of the light chain (V_{L}) is shown by an underline):

(SEQ ID NO: 105)
SSELTQDPAVSVALGQTVRITCQGDSLRSYYASWYQQKPGQAPVLVIYGK
NNRPSGIPDRFSGSSSGNTASLTITGAQAEDEADYYCNSRDSSGNPPVVF
GGGTKLTVLGSRGGGGSGGGGSGGGGSLEMAQVQLVESGGGLVHPGGSLR
LSCAASGFTFRSHSMNWVRQAPGKGLEWVSSISSDSTYTYYADSVKGRFT
ISRDNAKNSLYLQMNSLRAEDTAVYYCARSGGQWKYYDYWGQGTLVTVSS

An illustrative targeting domain is GPRC5DscFv-2, which an scFV specific to human GPRC5D, and has the following sequence (the linker joining the variable regions of the heavy clain (V_{H}) and the variable regions of the light chain (V_{L}) is shown by an underline):

(SEQ ID NO: 106)
QSVVTQPPSMSAAPGQQVTISCSGGNSNIERNYVSWYLQLPGTAPKLVIF
DNDRRPSGIPDRFSGSKSGTSATLGITGLQTGDEADYYCGTWDSSLRGWV
FGGGTKLTVLGSRGGGGSGGGGSGGGGSLEMAEVQLVESGGGLIQPGGSL
RLSCAASGFTFSNYAMNWVRQAPGKGLEWVSTINGRGSSTIYADSVKGRF
TISRDNSKNTLYLQMNSLRAEDTATYYCARYISRGLGDSWGQGTLVTV

An illustrative targeting domain is Trop2-1_vHvL, which an scFV specific to human Trop2, and has the following sequence (the linker joining the variable regions of the heavy clain (V_{H}) and the variable regions of the light chain (V_{L}) is shown by an underline):

(SEQ ID NO: 107)
QVQLQQSGSELKKPGASVKVSCKASGYTFTNYGMNWVKQAPGQGLKWMGW
INTYTGEPTYTDDFKGRFAFSLDTSVSTAYLQISSLKADDTAVYFCARGG
FGSSYWYFDVWGQGSLVTVSSGGGGGGGGSGGGGSDIQLTQSPSSLSASV
GDRVSITCKASQDVSIAVAWYQQKPGKAPKLLIYSASYRYTGVPDRFSGS
GSGTDFTLTISSLQPEDFAVYYCQQHYITPLTFGAGTKVEIKR

An illustrative targeting domain is Trop2-1_vLvH, which an scFV specific to human Trop2, and has the following sequence (the linker joining the variable regions of the heavy clain (V_{H}) and the variable regions of the light chain (V_{L}) is shown by an underline):

(SEQ ID NO: 108)
DIQLTQSPSSLSASVGDRVSITCKASQDVSIAVAWYQQKPGKAPKLLIYS
ASYRYTGVPDRFSGSGSGTDFTLTISSLQPEDFAVYYCQQHYITPLTFGA
GTKVEIKRGGGGSGGGGSGGGGSQVQLQQSGSELKKPGASVKVSCKASGY
TFTNYGMNWVKQAPGQGLKWMGWINTYTGEPTYTDDFKGRFAFSLDTSVS
TAYLQISSLKADDTAVYFCARGGFGSSYWYFDVWGQGSLVTVSS

An illustrative targeting domain is Trop2-2_vHvL, which an scFV specific to human Trop2, and has the following sequence (the linker joining the variable regions of the heavy clain (V_{H}) and the variable regions of the light chain (V_{L}) is shown by an underline):

(SEQ ID NO: 109)
QIQLVQSGPELKKPGETVKISCKASGYTFTNYGMNWVKQAPGKGLKWMG
WINTKTGEPTYAEEFKGRFAFSLETSASTAYLQINNLKKEDTATYFCGR
GGYGSSYWYFDVWGAGTTVTVSSGGGGSGGGGGGGGSDIVMTQSHKFMS
TSVGDRVSITCKASQDVSIAVAWYQQKPGQSPKVLIYSASYRYTGVPDR
FTGSGSGTDFTFTISRVQAEDLAVYYCQQHYITPLTFGAGTKLELK

An illustrative targeting domain is Trop2-2_vLvH, which an scFV specific to human Trop2, and has the following sequence (the linker joining the variable regions of the heavy clain (V_{H}) and the variable regions of the light chain (V_{L}) is shown by an underline):

(SEQ ID NO: 110)
DIVMTQSHKFMSTSVGDRVSITCKASQDVSIAVAWYQQKPGQSPKVLIY
SASYRYTGVPDRFTGSGSGTDFTFTISRVQAEDLAVYYCQQHYITPLTF
GAGTKLELKGGGGSGGGGSGGGGSQIQLVQSGPELKKPGETVKISCKAS
GYTFTNYGMNWVKQAPGKGLKWMGWINTKTGEPTYAEEFKGRFAFSLET
SASTAYLQINNLKKEDTATYFCGRGGYGSSYWYFDVWGAGTTVTVSS

An illustrative targeting domain is CEACAM5-1_vHvL, which an scFV specific to human CEACAM5, and has the following sequence (the linker joining the variable regions of the heavy clain (V_{H}) and the variable regions of the light chain (V_{L}) is shown by an underline):

(SEQ ID NO: 111)
EVQLVESGGGVVQPGRSLRLSCSASGFDFTTYWMSWVRQAPGKGLEWIG
EIHPDSSTINYAPSLKDRFTISRDNAKNTLFLQMDSLRPEDTGVYFCAS
LYFGFPWFAYWGQGTPVTVSSGGGGSGGGGSGGGGSDIQLTQSPSSLSA
SVGDRVTITCKASQDVGTSVAWYQQKPGKAPKLLIYWTSTRHTGVPSRF
SGSGSGTDFTFTISSLQPEDIATYYCQQYSLYRSFGQGTKVEIKR

An illustrative targeting domain is CEACAM5-1_vLvH, which an scFV specific to human CEACAM5, and has the following sequence (the linker joining the variable regions of the heavy clain (V_{H}) and the variable regions of the light chain (V_{L}) is shown by an underline):

(SEQ ID NO: 112)
DIQLTQSPSSLSASVGDRVTITCKASQDVGTSVAWYQQKPGKAPKLLIY
WTSTRHTGVPSRFSGSGSGTDFTFTISSLQPEDIATYYCQQYSLYRSFG
QGTKVEIKRGGGGSGGGGSGGGGSEVQLVESGGGVVQPGRSLRLSCSAS
GFDFTTYWMSWVRQAPGKGLEWIGEIHPDSSTINYAPSLKDRFTISRDN
AKNTLFLQMDSLRPEDTGVYFCASLYFGFPWFAYWGQGTPVTVSS

An illustrative targeting domain is CEACAM5-2_vHvL, which an scFV specific to human CEACAM5, and has the following sequence (the linker joining the variable regions of the heavy clain (V_{H}) and the variable regions of the light chain (V_{L}) is shown by an underline):

(SEQ ID NO: 113)
EVQLQESGPGLVKPGGSLSLSCAASGFVFSSYDMSWVRQTPERRLEWVA
YISSGGGITYFPSTVKGRFTVSRDNAKNTLYLQMNSLTSEDTAIYYCAA
HYFGSSGPFAYWGQGTLVTVSAGGGGSGGGGSGGGGSDIQMTQSPASLS
ASVGDTVTITCRASENIFSYLAWYQQKPGKSPKLLVYNTKTLAEGVPSR
FSGSGSGTQFSLTISSLQPEDFGSYYCQHHYGTPFTFGSGTKLEIK

An illustrative targeting domain is CEACAM5-2_vLvH, which an scFV specific to human CEACAM5, and has the following sequence (the linker joining the variable regions of the heavy clain (V_{H}) and the variable regions of the light chain (V_{L}) is shown by an underline):

(SEQ ID NO: 114)
DIQMTQSPASLSASVGDTVTITCRASENIFSYLAWYQQKPGKSPKLLVY
NTKTLAEGVPSRFSGSGSGTQFSLTISSLQPEDFGSYYCQHHYGTPFTF
GSGTKLEIKGGGGSGGGGSGGGGSVQLQESGPGLVKPGGSLSLSCAASG
FVFSSYDMSWVRQTPERRLEWVAYISSGGGITYFPSTVKGRFTVSRDNA
KNTLYLQMNSLTSEDTAIYYCAAHYFGSSGPFAYWGQGTLVTVSA

An illustrative targeting domain is CEACAM5-3_vHvL, which an scFV specific to human CEACAM5, and has the following sequence (the linker joining the variable regions of the heavy clain (V_{H}) and the variable regions of the light chain (V_{L}) is shown by an underline):

(SEQ ID NO: 115)
EVOLVESGGGLVQPGRSLRLSCAASGFTVSSYWMHWVRQAPGKGLEWVG
FIRNKANGGTTEYAASVKGRFTISRDDSKNTLYLQMNSLRAEDTAVYYC
ARDRGLRFYFDYWGQGTTVTVSSGGGGSGGGGSGGGGSQAVLTQPASLS
ASPGASASLTCTLRRGINVGAYSIYWYQQKPGSPPQYLLRYKSDSDKQQ
GSGVSSRFSASKDASANAGILLISGLQSEDEADYYCMIWHSGASAVFGG
GTKLTVL

An illustrative targeting domain is CEACAM5-3_vLvH, which an scFV specific to human CEACAM5, and has the following sequence (the linker joining the variable regions of the heavy clain (V_{H}) and the variable regions of the light chain (V_{L}) is shown by an underline):

(SEQ ID NO: 116)
QAVLTQPASLSASPGASASLTCTLRRGINVGAYSIYWYQQKPGSPPQYL
LRYKSDSDKQQGSGVSSRFSASKDASANAGILLISGLQSEDEADYYCMI
WHSGASAVFGGGTKLTVLGGGGSGGGGSGGGGSEVQLVESGGGLVQPGR
SLRLSCAASGFTVSSYWMHWVRQAPGKGLEWVGFIRNKANGGTTEYAAS
VKGRFTISRDDSKNTLYLQMNSLRAEDTAVYYCARDRGLRFYFDYWGQG
TTVTVSS

An illustrative targeting domain is CLL1-1_vHvL, which an scFV specific to human CLL1, and has the following sequence (the linker joining the variable regions of the heavy clain (V_{H}) and the variable regions of the light chain (V_{L}) is shown by an underline):

(SEQ ID NO: 117)
QVQLVQSGGGVVQPGRSLRLSCVASGFTFSSYGMHWVRQAPGKGLEWVA
AIWYNGRKQDYADSVKGRFTISRDNSKNTLYLQMNSLRAEDTAVYYCTR
GTGYNWFDPWGQGTLVTVSSGGGGSGGGGSGGGGSDIQMTQSPSSLSAS
VGDRVTITCRASQSISSYLNWYQQKPGKAPKLLIYAASSLQSGVPSRFS
GSGSGTDFTLTISSLQPEDFATYYCQQSYSTPPTFGQGTKVEIK

An illustrative targeting domain is CLL1-1_vLvH, which an scFV specific to human CLL1, and has the following sequence (the linker joining the variable regions of the heavy clain (V_{H}) and the variable regions of the light chain (V_{L}) is shown by an underline):

(SEQ ID NO: 118)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIKGGGGSGGGGSGGGGSQVQLVQSGGGVVQPGRSLRLSCVAS
GFTFSSYGMHWVRQAPGKGLEWVAAIWYNGRKQDYADSVKGRFTISRDN
SKNTLYLQMNSLRAEDTAVYYCTRGTGYNWFDPWGQGTLVTVSS

An illustrative targeting domain is CLL1-2_vHvL, which an scFV specific to human CLL1, and has the following sequence (the linker joining the variable regions of the heavy clain (V_{H}) and the variable regions of the light chain (V_{L}) is shown by an underline):

(SEQ ID NO: 119)
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSGGGGSGGGGSGGGGSDIQLTQSPSS
LSASVGDRVSFTCQASQDINNFLNWYQQKPGKAPKLLIYDASNLETGVP
SRFSGSGSGTDFTFTISSLQPEDIATYYCQQYGNLPFTFGGGTKVEIKR

An illustrative targeting domain is CLL1-2_vLvH, which an scFV specific to human CLL1, and has the following sequence (the linker joining the variable regions of the heavy clain (V_{H}) and the variable regions of the light chain (V_{L}) is shown by an underline):

(SEQ ID NO: 120)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTF
GGGTKVEIKRGGGGSGGGGSGGGGSQVQLQESGPGLVKPSETLSLTCTV
SGGSISSYYWSWIRQPPGKGLEWIGYIYYSGSTNYNPSLKSRVTISVDT
SKNQFSLKLSSVTAADTAVYYCVSLVYCGGDCYSGFDYWGQGTLVTVSS

An illustrative targeting domain is ROR1-vHvL-1, which an scFV specific to human ROR1, and has the following sequence (the linker joining the variable regions of the heavy clain (V_{H}) and the variable regions of the light chain (V_{L}) is shown by an underline):

(SEQ ID NO: 121)
QVQLVQSGAEVKKPGASVKVSCKASGYTFTSYYMHWVRQAPGQGLEWMG
IINPNGGSTSYAQKFQGRVTMTRDTSTSTVYMELSSLRSEDTAVYYCAR
DSSYDAFDIWGQGTMVTVSSGGGGSGGGGSGGGGSAIQLTQSPSTLSAS
VGDRVTItCQASQDISNYLNWYQQKPGKAPKLLINDASYLETGVPSRFS
GSGSGTDFTLTISSLQPEDIATYYCQQYESLPYTFGQGTKLEIK

An illustrative targeting domain is ROR1-vLvH-1, which an scFV specific to human ROR1, and has the following sequence (the linker joining the variable regions of the heavy clain (V_{H}) and the variable regions of the light chain (V_{L}) is shown by an underline):

(SEQ ID NO: 122)
AIQLTQSPSTLSASVGDRVTItCQASQDISNYLNWYQQKPGKAPKLLIN
DASYLETGVPSRFSGSGSGTDFTLTISSLQPEDIATYYCQQYESLPYTF
GQGTKLEIKGGGGSGGGGSGGGGSQVQLVQSGAEVKKPGASVKVSCKAS
GYTFTSYYMHWVRQAPGQGLEWMGIINPNGGSTSYAQKFQGRVTMTRDT
STSTVYMELSSLRSEDTAVYYCARDSSYDAFDIWGQGTMVTVSS

An illustrative targeting domain is ROR1-vLvH-2, which an scFV specific to human ROR1, and has the following sequence (the linker joining the variable regions of the heavy clain (V_{H}) and the variable regions of the light chain (V_{L}) is shown by an underline):

(SEQ ID NO: 123)
QVTLKESGGGLVKPGGSLRLSCAASGFTFSNAWMSWVRQAPGKGLEWVG
RIKSKTDGGTTDYAAPVKGRFTISRDDSKNTLYLQMNSLKTEDTAVYYC
ARDFGRWSYYFDYWSQGTLVTVSSGGGGSGGGGSGGGGSQSVLTQPSSV
SGTPGQRVTISCSGSSSNIGSNYVYWYQQLPGTAPKWYRNNQRPSGVPD
RFSGSKSGTSASLAISGLRSEDEADYYCAAWDDSLSGVVFGGGTKLTVL

An illustrative targeting domain is ROR1-vHvL-2, which an scFV specific to human ROR1, and has the following sequence (the linker joining the variable regions of the heavy clain (V_{H}) and the variable regions of the light chain (V_{L}) is shown by an underline):

(SEQ ID NO: 124)
QSVLTQPSSVSGTPGQRVTISCSGSSSNIGSNYVYWYQQLPGTAPKWYR
NNQRPSGVPDRFSGSKSGTSASLAISGLRSEDEADYYCAAWDDSLSGVV
FGGGTKLTVLGGGGSGGGGSGGGGSQVTLKESGGGLVKPGGSLRLSCAA
SGFTFSNAWMSWVRQAPGKGLEWVGRIKSKTDGGTTDYAAPVKGRFTIS
RDDSKNTLYLQMNSLKTEDTAVYYCARDFGRWSYYFDYWSQGTLVTVSS

An illustrative targeting domain is ROR1-vHvL-3, which an scFV specific to human ROR1, and has the following sequence (the linker joining the variable regions of the heavy clain (V_{H}) and the variable regions of the light chain (V_{L}) is shown by an underline):

(SEQ ID NO: 125)
EVQLVESGGGLVQPGRSLRLSCTASGFTFGDYAMSWVRQAPGKGLEWVS
SISGSGRSTDHADYVKGRFTISRDNSKNTVYLQMNRLRAEDTAVYYCAK
VSNYEYYFDYWAQGTLTVSSGGGGSGGGGSGGGGSEIVLTQSPSVSVAP
GQTARITCGGSNIGSESVNWYQWKSGQVPVLVVSDTTDPRSGIPGRFTG
TRSGTTATLTISGVEAGDEADYHCQVWDDTGDHPVFGGGTKLTVL

An illustrative targeting domain is ROR1-vLvH-3, which an scFV specific to human ROR1, and has the following sequence (the linker joining the variable regions of the heavy clain (V_{H}) and the variable regions of the light chain (V_{L}) is shown by an underline):

(SEQ ID NO: 126)
EIVLTQSPSVSVAPGQTARITCGGSNIGSESVNWYQWKSGQVPVLVVSD
TTDPRSGIPGRFTGTRSGTTATLTISGVEAGDEADYHCQVWDDTGDHPV
FGGGTKLTVLGGGGSGGGGSGGGGSEVQLVESGGGLVQPGRSLRLSCTA
SGFTFGDYAMSWVRQAPGKGLEWVSSISGSGRSTDHADYVKGRFTISRD
NSKNTVYLQMNRLRAEDTAVYYCAKVSNYEYYFDYWAQGTLTVSS

In embodiments, the second domain of the chimeric protein comprises a polypeptide having an amino acid sequence selected from SEQ ID NOs: 20-27 and 94-126. In embodiments, the second domain of the chimeric protein comprises a polypeptide having an amino acid sequence that has at least about 95% identity with an amino acid sequence selected from SEQ ID NOs: 20-23 and 94-126. In embodiments, the second domain of the alpha chain and/or beta chain comprises a polypeptide having an amino acid sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence an amino acid sequence selected from SEQ ID NOs: 20-23 and 94-126.

In embodiments, in addition to the butyrophilin family protein, the chimeric proteins further comprise a portion of the extracellular domain of LAG-3, PD-1, or TIGIT and which is capable of binding its receptor/ligand on the surface of a cancer cell. In embodiments, in addition to the butyrophilin family protein, the chimeric proteins further comprise an antibody or fragment thereof (e.g., comprising a portion of the antigen-binding domain of an antibody and/or a CDR3 that binds a tumor epitope) and which is capable of binding an antigen on the surface of a cancer cell.

In embodiments, in addition to the butyrophilin family protein, the chimeric proteins further comprise a portion of the extracellular domain of LAG-3, PD-1, TIGIT, CD19, PSMA, or antibody-derived binding domain (e.g. CDR3, Fab, scFv domain, etc.) targeting a tumor antigen (such as CD19 or PSMA) and which is capable of binding its receptor/ligand on the surface of a cancer cell. In embodiments, in addition to the BTNL family protein, the chimeric proteins further comprise an antibody or fragment thereof (e.g., comprising a portion of the antigen-binding domain of an antibody) and which is capable of binding an antigen on the surface of a cancer cell.

In an illustrative embodiment, the second domain is a receptor for EGP such as EGFR (ErbB1), ErbB2, ErbB3 and ErbB4.

In an illustrative embodiment, the second domain is a receptor for insulin or an insulin analog such as the insulin receptor and/or IGF1 or IGF2 receptor.

In an illustrative embodiment, the second domain is a receptor for EPO such as the EPO receptor (EPOR) receptor and/or the ephrin receptor (EphR)

In various embodiments, the chimeric protein may comprise a domain of a soluble (e.g., non-membrane associated) protein. In various embodiments, the chimeric protein may comprise a fragment of the soluble protein which is involved in signaling (e.g., a portion of the soluble protein which interacts with a receptor).

In various embodiments, the chimeric protein may comprise the extracellular domain of a transmembrane protein. In various embodiments, one of the extracellular domains transduces an immune inhibitory signal and one of the extracellular domains transduces an immune stimulatory signal.

In some embodiments, an extracellular domain refers to a portion of a transmembrane protein which is capable of interacting with the extracellular environment. In various embodiments, an extracellular domain refers to a portion of a transmembrane protein which is sufficient to bind to a ligand or receptor and effective transmit a signal to a cell. In various embodiments, an extracellular domain is the entire amino acid sequence of a transmembrane protein which is external of a cell or the cell membrane. In various embodiments, an extracellular domain is the that portion of an amino acid sequence of a transmembrane protein which is external of a cell or the cell membrane and is needed for signal transduction and/or ligand binding as may be assayed using methods know in the art (e.g., in vitro ligand binding and/or cellular activation assays).

In various embodiments, the chimeric protein may comprise an antibody binding domain (e.g. CDR3, Fab, scFv domain, etc.). In various embodiments, one of the antibody binding domains transduces an immune inhibitory signal and one of the antibody binding domains transduces an immune stimulatory signal.

In some embodiments, an immune inhibitory signal refers to a signal that diminishes or eliminates an immune response. For example, in the context of oncology, such signals may diminish or eliminate antitumor immunity. Under normal physiological conditions, inhibitory signals are useful in the maintenance of self-tolerance (e.g., prevention of autoimmunity) and also to protect tissues from damage when the immune system is responding to pathogenic infection. For instance, without limitation, immune inhibitory signal may be identified by detecting an increase in cellular proliferation, cytokine production, cell killing activity or phagocytic activity when such an inhibitory signal is blocked.

In some embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes. Specific examples include direct stimulation of cytokine receptors such as IL-2R, IL-7R, IL-15R, IL-17R or IL-21R using fusion proteins encoding the ligands for such receptors (IL-2, IL-7, IL-15, IL-17 or IL-21, respectively). Stimulation from any one of these receptors may directly stimulate the proliferation and cytokine production of individual T cell subsets.

In some embodiments, the extracellular domain or antibody binding domain (e.g. CDR3, Fab, scFv domain, etc.) may be used to produce a soluble protein to competitively inhibit signaling by that receptor's ligand. For instance, without limitation, competitive inhibition of PD-L1 or PD-L2 could be achieved using PD-1, or competitive inhibition of PVR could be achieved using TIGIT. In some embodiments, the extracellular domain or antibody binding domain (e.g. CDR3, Fab, scFv domain, etc.) may be used to provide artificial signaling.

In some embodiments, the present chimeric proteins deliver or mask an immune inhibitory signal. In some embodiments, the present chimeric proteins deliver or mask an immune stimulatory signal.

In embodiments, the targeting domain is capable of binding an antigen on the surface of a cancer cell. In embodiments, the targeting domain comprises an extracellular domain of a membrane protein selected from LAG-3, PD-1, TIGIT, CD19, or PSMA.

In embodiments, the second domain comprises an extracellular domain of a LAG-3 protein.

In embodiments, the second domain comprises an extracellular domain of a PD-1 protein.

In embodiments, the second domain comprises an extracellular domain of a TIGIT protein.

The Linker Domain that Adjoins the First and the Second Domain

The linker of any of the embodiments disclosed herein are suitable.

In various embodiments, each of the first and/or second charge polarized core domains further comprise a linker (e.g., a stabilizing domain) which adjoins the proteins having positively or negatively charged amino acids. In embodiments, the linker (e.g., a stabilizing domain) is optionally selected from a flexible amino acid sequence, IgG hinge region, or antibody sequence. In an embodiment, the linker (e.g., a stabilizing domain) comprises the hinge-CH2-CH3 Fc domain derived from IgG1, optionally human IgG1. In another embodiment, the linker (e.g., a stabilizing domain) comprises the hinge-CH2-CH3 Fc domain derived from IgG4, optionally human IgG4.

Illustrative sequences of linkers that adjoins the first and second domains, also referred to herein as a core domain are provided below:

In embodiments, the core domain has the following sequence:

(SEQ ID NO: 15)
SKYGPPCPPCPAPEFLGGPSVFLFPPKPKDQLMISRTPEVTCVVVDVSQ
EDPEVQFNWYVDGVEVHNAKTKPREEQFNSTYRVVSVLTVLHQDWLSGK
EYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHNHYTQKSLSLSLGKIEGRMD.

In embodiments, the core domain has the following sequence:

(SEQ ID NO: 28)
CPPCPAPEFLGGPSVFLFPPKPKDQLMISRTPEVTCVVVDVSQEDPEVQ
FNWYVDGVEVHNAKTKPREEQFNSTYRVVSVLTVLHQDWLSGKEYKCKV
SSKGLPSSIEKTISNATGQPREPQVYTLPPSQEEMTKNQVSLTCLVKGF
YPSDIAVEWESNGQPENNYKTTPPVLDSDGSFFLYSRLTVDKSRWQEGN
VFSCSVLHEALHNHYTQKSLSLSLGK.

In embodiments, the core domain is a KIHT22Y protein having the following sequence:

(SEQ ID NO: 29)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLYCLVKGFYPSDIAVEWESNGQPENNYKTTPPVLDSDGSFFLYSKLT
VDKSRWQQGNVFSCSVMHEALHNHYTQKSLSLSPGK.

In embodiments, the core domain is a KIHY86T protein having the following sequence:

(SEQ ID NO: 30)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLTSKLT
VDKSRWQQGNVFSCSVMHEALHNHYTQKSLSLSPGK.

In embodiments, the core domain is a KIHY86T protein having the following sequence:

(SEQ ID NO: 31)
VPRDCGCKPCICTVPEVSSVFIFPPKPKDVLTITLTPKVTCVVVDISKD
DPEVQFSWFVDDVEVHTAQTQPREEQFNSTFRSVSELPIMHQQWLNGKE
FKCRVNSAAFPAPIEKTISKTKGRPKAPQVYTIPPPKEQMAKDKVSLTC
MITDFFPEDITVEWQWNGQPAENYKNTQPIMDTDGSYFVYSKLNVQKSN
WEAGNTFTCSVLHEGLHNHHTEKSLSHSPGI.

The sequence of an illustrative charge polarized core domain (positive-negative) is provided below:

(SEQ ID NO: 16)
GSGSRKGGKRGSKYGPP

[symbol]

[symbol]

[symbol]

[symbol]

[symbol]

[symbol]

[symbol]

DEGGEDGSGS.

The sequence of an illustrative charge polarized core domain (negative-positive) is provided below:

(SEQ ID NO: 17)
GSGSDEGGEDGSKYGPP

[symbol]

[symbol]

[symbol]

[symbol]

[symbol]

[symbol]

[symbol]

RKGGKRGSGS

The sequence of an illustrative charge polarized core domain (negative-positive) is provided below:

(SEQ ID NO: 32)

[symbol]

[symbol]

[symbol]

[symbol]

[symbol]

[symbol]

[symbol]

.

The sequence of an illustrative Fc domains containing knob-in-hole (KIH) mutations are provided below:

(SEQ ID NO: 52)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPCRDELTKNQ
VSLWCLVKGFYPSDIAVEWESNGQPENNYKTTPPVLDSDGSFFLYSKLT
VDKSRWQQGNVFSCSVMHEALHNHYTQKSLSLSPGKIEGRMD.

The sequence of an illustrative Fc domains containing knob-in-hole (KIH) mutations are provided below:

(SEQ ID NO: 53)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVCTLPPSRDELTKNQ
VSLSCAVKGFYPSDIAVEWESNGQPENNYKTTPPVLDSDGSFFLVSKLT
VDKSRWQQGNVFSCSVMHEALHNHYTQKSLSLSPGKIEGRMD.

The sequence of an illustrative Fc domains containing knob-in-hole (KIH) mutations and FcRn mutations are provided below:

(SEQ ID NO: 54)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPCRDELTKNQ
VSLWCLVKGFYPSDIAVEWESNGQPENNYKTTPPVLDSDGSFFLYSKLT
VDKSRWQQGNVFSCSVLHEALHSHYTQKSLSLSPGKIEGRMD.

The sequence of an illustrative Fc domains containing knob-in-hole (KIH) mutations and FcRn mutations are provided below:

(SEQ ID NO: 55)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVCTLPPSRDELTKNQ
VSLSCAVKGFYPSDIAVEWESNGQPENNYKTTPPVLDSDGSFFLVSKLT
VDKSRWQQGNVFSCSVLHEALHSHYTQKSLSLSPGKIEGRMD.

In embodiments, the linker comprises the hinge-CH2-CH3 Fc domain. In embodiments, he hinge-CH2-CH3 Fc domain is derived from IgG1, optionally human IgG1. In embodiments, the hinge-CH2-CH3 Fc domain is derived from IgG4, optionally human IgG4. In embodiments, the hinge-CH2-CH3 Fc domain comprises a polypeptide having an amino acid sequence with at least 90%, or 95%, or 97%, or 98%, or 99% identity with a polypeptide selected from SEQ ID NOs: 16-17, 28-32, and 52-55.

In embodiments, the first domain and/or the chimeric protein modulates or is capable of modulating a γδ (gamma delta) T cell. In embodiments, the gamma delta T cell expresses Vγ4 or Vγ9δ2. In embodiments, the first domain comprises BTNL3 and BTNL8 and it modulates a Vγ4-expressing T cell. In embodiments, the first domain modulates a Vγ9δ2-expressing T cell. In embodiments, the first domain comprises: (a) BTN2A1 and BTN3A1, (b) BTN3A1 and BTN3A2, or (c) BTN3A1 and BTN3A3. In embodiments, the modulation of a gamma delta T cell is activation of a gamma delta T cell. In embodiments, the chimeric protein is capable of forming a synapse between a gamma delta T cell and a tumor cell and/or the chimeric protein is capable of contemporaneous activation and targeting of gamma delta T cells to tumor cells.

In embodiments, the chimeric protein is a homodimer.

In one aspect, the current disclosure relates to a pharmaceutical composition, comprising the chimeric protein of any of the embodiments disclosed herein.

In one aspect, the current disclosure relates to an expression vector, comprising a nucleic acid encoding the first and/or second polypeptide chains of the chimeric protein of any of the embodiments disclosed herein. In embodiments, the expression vector is a mammalian expression vector. In embodiments, the expression vector comprises DNA or RNA.

In one aspect, the current disclosure relates to a host cell, comprising the expression vector of any of the embodiments disclosed herein.

Diseases; Methods of Treatment, and Patient Selections

In one aspect, the current disclosure provides a method of treating cancer, comprising administering to a subject in need thereof an effective amount of a pharmaceutical composition of any of the embodiments disclosed herein to a subject in need thereof. In embodiments, the cancer is a lymphoma. In embodiments, the cancer is a leukemia. In embodiments, the cancer is a Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In embodiments, the cancer is prostate cancer. In embodiments, the cancer is an epithelial-derived carcinoma. In embodiments, the cancer is known to express the antigenic target of the second domain of the heterodimeric protein. In embodiments, the cancer is known to contain mutations which limit recognition by alpha beta T cells, including but not limited to mutations in MHC I, beta 2 microglobulin, TAP, etc.

In embodiments, the subject is further administered autologous or allogeneic gamma delta T cells that were expanded ex vivo. In embodiments, the autologous or allogeneic gamma delta T cells express a Chimeric Antigen Receptor. In embodiments, the subject is further administered autologous or allogeneic T cells that express a Chimeric Antigen Receptor.

In one aspect, the current disclosure provides a method of treating an autoimmune disease or disorder, comprising administering an effective amount of a pharmaceutical composition of any of the embodiments disclosed herein to a subject in need thereof, wherein the autoimmune disease or disorder is optionally selected from rheumatoid arthritis, systemic lupus erythematosus, diabetes mellitus, ankylosing spondylitis, Sjögren's syndrome, inflammatory bowel diseases (e.g., colitis ulcerosa, Crohn's disease), multiple sclerosis, sarcoidosis, psoriasis, Grave's disease, Hashimoto's thyroiditis, psoriasis, hypersensitivity reactions (e.g., allergies, hay fever, asthma, and acute edema cause type I hypersensitivity reactions), and vasculitis.

In various embodiments, the current disclosure pertains to the use of the heterodimeric proteins for the treatment of one or more autoimmune diseases or disorders. In various embodiments, the treatment of an autoimmune disease or disorder may involve modulating the immune system with the present heterodimeric proteins to favor immune inhibition over immune stimulation. Illustrative autoimmune diseases or disorders treatable with the present heterodimeric proteins include those in which the body's own antigens become targets for an immune response, such as, for example, rheumatoid arthritis, systemic lupus erythematosus, diabetes mellitus, ankylosing spondylitis, Sjögren's syndrome, inflammatory bowel diseases (e.g., colitis ulcerosa, Crohn's disease), multiple sclerosis, sarcoidosis, psoriasis, Grave's disease, Hashimoto's thyroiditis, psoriasis, hypersensitivity reactions (e.g., allergies, hay fever, asthma, and acute edema cause type I hypersensitivity reactions), and vasculitis.

Illustrative autoimmune diseases or conditions that may be treated or prevented using the heterodimeric protein of the invention include, but are not limited to, multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection), pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In various embodiments, the current disclosure pertains to cancers and/or tumors; for example, the treatment or prevention of cancers and/or tumors. As described elsewhere herein, the treatment of cancer may involve in various embodiments, modulating the immune system with the present heterodimeric proteins to favor immune stimulation over immune inhibition.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the current disclosure include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the cancer is an epithelial-derived carcinoma.

In embodiments, the heterodimeric protein is used to treat a subject that has a treatment-refractory cancer. In embodiments, the heterodimeric protein is used to treat a subject that is refractory to one or more immune-modulating agents. For example, In embodiments, the heterodimeric protein is used to treat a subject that presents no response to treatment, or even progress, after 12 weeks or so of treatment. For instance, In embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, In embodiments, the subject is refractory to an anti-CTLA-4 agent, e.g., ipilimumab (YERVOY)-refractory patients (e.g., melanoma patients). Accordingly, in various embodiments the current disclosure provides methods of cancer treatment that rescue patients that are non-responsive to various therapies, including monotherapy of one or more immune-modulating agents.

In various embodiments, the current disclosure provides heterodimeric proteins which target a cell or tissue within the tumor microenvironment. In embodiments, the cell or tissue within the tumor microenvironment expresses one or more targets or binding partners of the heterodimeric protein. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In various embodiments, the present heterodimeric protein targets a cancer cell. In embodiments, the cancer cell expresses one or more of targets or binding partners of the heterodimeric protein.

In various embodiments, the heterodimeric protein of the invention may target a cell (e.g., cancer cell or immune cell) that expresses any of the receptors as described herein. For example, the heterodimeric protein of the invention may target a cell that expresses any of the receptors for a cytokine, growth factor, and/or hormone as described herein.

In embodiments, the present methods provide treatment with the heterodimeric protein in a patient who is refractory to an additional agent, such “additional agents” being described elsewhere herein, inclusive, without limitation, of the various chemotherapeutic agents described herein.

In some aspects, the present chimeric agents are used to eliminate intracellular pathogens. In some aspects, the present chimeric agents are used to treat one or more infections. In embodiments, the present heterodimeric proteins are used in methods of treating viral infections (including, for example, HIV and HCV), parasitic infections (including, for example, malaria), and bacterial infections. In various embodiments, the infections induce immunosuppression. For example, HIV infections often result in immunosuppression in the infected subjects. Accordingly, as described elsewhere herein, the treatment of such infections may involve, in various embodiments, modulating the immune system with the present heterodimeric proteins to favor immune stimulation over immune inhibition. Alternatively, the current disclosure provides methods for treating infections that induce immunoactivation. For example, intestinal helminth infections have been associated with chronic immune activation. In these embodiments, the treatment of such infections may involve modulating the immune system with the present heterodimeric proteins to favor immune inhibition over immune stimulation.

In various embodiments, the current disclosure provides methods of treating viral infections including, without limitation, acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In embodiments, the viral infection is caused by a virus of family Flaviviridae. In embodiments, the virus of family Flaviviridae is selected from Yellow Fever Virus, West Nile virus, Dengue virus, Japanese Encephalitis Virus, St. Louis Encephalitis Virus, and Hepatitis C Virus. In other embodiments, the viral infection is caused by a virus of family Picornaviridae, e.g., poliovirus, rhinovirus, coxsackievirus. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus.

In various embodiments, the current disclosure provides methods of treating parasitic infections such as protozoan or helminths infections. In embodiments, the parasitic infection is by a protozoan parasite. In embodiments, the oritiziab parasite is selected from intestinal protozoa, tissue protozoa, or blood protozoa. Illustrative protozoan parasites include, but are not limited to, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falcipanum, Trichomonas vaginalis, and Histomonas meleagridis. In embodiments, the parasitic infection is by a helminthic parasite such as nematodes (e.g., Adenophorea). In embodiments, the parasite is selected from Secementea (e.g., Trichuris trichiura, Ascaris lumbricoides, Enterobius vermicularis, Ancylostoma duodenale, Necator americanus, Strongyloides stercoralis, Wuchereria bancrofti, Dracunculus medinensis). In embodiments, the parasite is selected from trematodes (e.g., blood flukes, liver flukes, intestinal flukes, and lung flukes). In embodiments, the parasite is selected from: Schistosoma mansoni, Schistosoma haematobium, Schistosoma japonicum, Fasciola hepatica, Fasciola gigantica, Heterophyes, Paragonimus westermani. In embodiments, the parasite is selected from cestodes (e.g., Taenia solium, Taenia saginata, Hymenolepis nana, Echinococcus granulosus).

In various embodiments, the current disclosure provides methods of treating bacterial infections. In various embodiments, the bacterial infection is by gram-positive bacteria, gram-negative bacteria, aerobic and/or anaerobic bacteria. In various embodiments, the bacteria are selected from, but not limited to, Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms. In embodiments, the bacteria is selected from, but not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In still another other aspect, the current disclosure is directed toward methods of treating and preventing T cell-mediated diseases and disorders, such as, but not limited to diseases or disorders described elsewhere herein and inflammatory disease or disorder, graft-versus-host disease (GVHD), transplant rejection, and T cell proliferative disorder.

In some aspects, the present chimeric agents are used in methods of activating a T cell, e.g., via the extracellular domain having an immune stimulatory signal or antibody binding domain (e.g. CDR3, Fab, scFv domain, etc.) having an immune stimulatory signal.

In some aspects, the present chimeric agents are used in methods of preventing the cellular transmission of an immunosuppressive signal.

Combination Therapies and Conjugation

In embodiments, the invention provides for heterodimeric proteins and methods that further comprise administering an additional agent to a subject. In embodiments, the invention pertains to co-administration and/or co-formulation. Any of the compositions described herein may be co-formulated and/or co-administered.

In embodiments, any heterodimeric protein described herein acts synergistically when co-administered with another agent and is administered at doses that are lower than the doses commonly employed when such agents are used as monotherapy. In various embodiments, any agent referenced herein may be used in combination with any of the heterodimeric proteins described herein.

In various embodiments, any of the heterodimeric proteins disclosed herein may be co-administered with another heterodimeric protein disclosed herein. Without wishing to be bound by theory, it is believed that a combined regimen involving the administration of one or more heterodimeric proteins which induce an innate immune response and one or more heterodimeric proteins which induce an adaptive immune response may provide synergistic effects (e.g., synergistic anti-tumor effects).

In various embodiments, any heterodimeric protein which induces an innate immune response may be utilized in the current disclosure. In various embodiments, any heterodimeric protein which induces an adaptive immune response may be utilized in the current disclosure.

In embodiments, inclusive of, without limitation, cancer applications, the current disclosure pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In various embodiments, inclusive of, without limitation, cancer applications, the present additional agent is one or more immune-modulating agents selected from an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), MK-3475 (MERCK), BMS 936559 (BRISTOL MYERS SQUIBB), atezolizumab (TECENTRIQ, GENENTECH), MPDL3280A (ROCHE), an agent that increases and/or stimulates CD137 (4-1BB) and/or the binding of CD137 (4-1BB) with one or more of 4-1BB ligand (byway of non-limiting example, urelumab (BMS-663513 and anti-4-1BB antibody), and an agent that blocks, reduces and/or inhibits the activity of CTLA-4 and/or the binding of CTLA-4 with one or more of AP2M1, CD80, CD86, SHP-2, and PPP2R5A and/or the binding of OX40 with OX40L (by way of non-limiting example GBR 830 (GLENMARK), MEDI6469 (MEDIMMUNE).

In embodiments, inclusive of, without limitation, infectious disease applications, the current disclosure pertains to anti-infectives as additional agents. In embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In embodiments, inclusive, without limitation, of autoimmune applications, the additional agent is an immunosuppressive agent. In embodiments, the immunosuppressive agent is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the current disclosure include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the current disclosure, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin).

In embodiments, the heterodimeric proteins (and/or additional agents) described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids. In still other embodiments, the heterodimeric proteins (and/or additional agents) described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

The heterodimeric proteins (and/or additional agents) described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Formulations

In one aspect, the current disclosure provides a pharmaceutical composition, comprising the heterodimeric protein of any of the embodiments disclosed herein.

The heterodimeric proteins (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions described herein are in the form of a pharmaceutically acceptable salt.

Further, any heterodimeric protein (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are resuspended in a saline buffer (including, without limitation TBS, PBS, and the like).

In various embodiments, the heterodimeric proteins may by conjugated and/or fused with another agent to extend half-life or otherwise improve pharmacodynamic and pharmacokinetic properties. In embodiments, the heterodimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In various embodiments, each of the individual heterodimeric proteins is fused to one or more of the agents described in BioDrugs (2015) 29:215-239, the entire contents of which are hereby incorporated by reference.

Administration, Dosing, and Treatment Regimens

The current disclosure includes the described heterodimeric protein (and/or additional agents) in various formulations. Any heterodimeric protein (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the heterodimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the heterodimeric protein (and/or additional agents) of the current disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any heterodimeric protein (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In most instances, administration results in the release of any agent described herein into the bloodstream.

Any heterodimeric protein (and/or additional agents) described herein can be administered orally. Such heterodimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In one embodiment, for instance in the treatment of cancer, the heterodimeric protein (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In various embodiments, for instance in the treatment of cancer, the heterodimeric protein (and/or additional agents) are administered intratumorally.

In the various embodiments, the present heterodimeric protein allows for a dual effect that provides less side effects than are seen in conventional immunotherapy (e.g., treatments with one or more of OPDIVO, KEYTRUDA, YERVOY, and TECENTRIQ). For example, the present heterodimeric proteins reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any heterodimeric protein (and/or additional agents) described herein as well as the dosing schedule can depend on various parameters, including, but not limited to, the disease being treated, the subject's general health, and the administering physician's discretion. Any heterodimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In various embodiments any heterodimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In various embodiments, the current disclosure relates to the co-administration of a heterodimeric protein which induces an innate immune response and another heterodimeric protein which induces an adaptive immune response. In such embodiments, the heterodimeric protein which induces an innate immune response may be administered before, concurrently with, or subsequent to administration of the heterodimeric protein which induces an adaptive immune response. For example, the heterodimeric proteins may be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart. In an illustrative embodiment, the heterodimeric protein which induces an innate immune response and the heterodimeric protein which induces an adaptive response are administered 1 week apart, or administered on alternate weeks (i.e., administration of the heterodimeric protein inducing an innate immune response is followed 1 week later with administration of the heterodimeric protein which induces an adaptive immune response and so forth).

The dosage of any heterodimeric protein (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of any heterodimeric protein (and/or additional agents) described herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent described herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the heterodimeric protein (and/or additional agents) described herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the heterodimeric protein (and/or additional agents) is in a range of 10 about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any heterodimeric protein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any heterodimeric protein (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any heterodimeric protein (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any heterodimeric protein (and/or additional agents) described herein can be administered in a single daily dose, or the total daily dosage can be administered in divided doses of two, three or four times daily. Furthermore, any heterodimeric protein (and/or additional agents) described herein can be administered continuously rather than intermittently throughout the dosage regimen.

Cells and Nucleic Acids

In one aspect, the current disclosure provides an expression vector, comprising a nucleic acid encoding the first and/or second polypeptide chains of the heterodimeric protein of any of any of the embodiments disclosed herein. In embodiments, the expression vector is a mammalian expression vector. In embodiments, the expression vector comprises DNA or RNA. In embodiments, In one aspect, the current disclosure provides a host cell comprising the expression vector of any one of the embodiments disclosed herein.

In various embodiments, the current disclosure provides an expression vector, comprising a nucleic acid encoding the heterodimeric protein (e.g., a heterodimeric protein comprising a first and second polypeptide chains) described herein. In various embodiments, the expression vector comprises DNA or RNA. In various embodiments, the expression vector is a mammalian expression vector.

Both prokaryotic and eukaryotic vectors can be used for expression of the heterodimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the heterodimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the fusion proteins in recombinant host cells.

In embodiments, expression vectors of the invention comprise a nucleic acid encoding at least the first and/or second polypeptide chains of the heterodimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In an embodiment, the cell is a tumor cell. In another embodiment, the cell is a non-tumor cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In an embodiment, the current disclosure contemplates the use of inducible promoters capable of effecting high level of expression transiently in response to a cue. For example, when in the proximity of a tumor cell, a cell transformed with an expression vector for the heterodimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In one aspect, the invention provides expression vectors for the expression of the heterodimeric proteins (and/or additional agents) that are viral vectors. Many viral vectors useful for gene therapy are known (see, e.g., Lundstrom, Trends Biotechnol., 21: 117, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In one embodiment, the invention provides methods of transducing a human cell in vivo, comprising contacting a solid tumor in vivo with a viral vector of the invention.

In various embodiments, the current disclosure provides a host cell, comprising the expression vector comprising the heterodimeric protein described herein.

Expression vectors can be introduced into host cells for producing the present heterodimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E. G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection, or from commercial suppliers.

Cells that can be used for production of the present heterodimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor or infectious disease being treated or prevented, and can be determined by one of skill in the art.

Production and purification of Fc-containing macromolecules (such as Fc fusion proteins) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In various embodiments, production of the heterodimeric proteins contemplated herein may have unique characteristics as compared to traditional Fc containing macromolecules. In certain examples, the heterodimeric proteins may be purified using specific chromatography resins, or using chromatography methods that do not depend upon Protein A capture. In other embodiments, the heterodimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The heterodimeric proteins obtained herein may be additionally ‘polished’ using methods that are specified in the art. In embodiments, the heterodimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In other embodiments, the heterodimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

Methods of Making the Heterodimeric Proteins of the Current Disclosure

Also disclosed herein are methods for making a heterodimeric protein comprising (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domain and which facilitates heterodimerization. In some embodiments, the heterodimeric protein comprises two of the same butyrophilin family proteins or two different butyrophilin family proteins. In some embodiments, the heterodimeric protein comprises two individual polypeptide chains which self-associate. Such heterodimeric proteins are disclosed in WO 2020/146393, the entire contents of which is incorporated herein by reference.

In embodiments, the first domain comprises a butyrophilin family protein is from BTN1A1, BTN2A1, BTN2A2, BTN2A3, BTN3A1, BTN3A2, BTN3A3, BTNL2, BTNL3, BTNL8, BTNL9, BTNL10, and SKINTL. In embodiments, the butyrophilin family protein is selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL. The targeting domain may be of any of the embodiments disclosed herein. The linker may be of any of the embodiments disclosed herein.

Ordinarily, an heterodimeric protein is prepared by at least one purification step. Exemplary purification steps include chromatography (without limitation, e.g. affinity chromatography). The methods of purification are well known in the art of protein purification and antibody purification. The steps in purification process are disclosed in U.S. Pat. Nos. 5,429,746; 9,708,365; 10,570,434; 10,533,045; 9,631,007; 7,691,980; 9,938,317, the entire contents of each of which is incorporated herein by reference.

In exemplary embodiments, the heterodimeric proteins provided herein include two variant Fc domain sequences. Such variant Fc domains include amino acid modifications to facilitate the self-assembly and/or purification of the heterodimeric proteins. Exemplary amino acid modifications that facilitate the production and purification of heterodimeric proteins include “skew” variants (e.g., the “knobs and holes” and the “charge pairs” variants described herein) as well as “pI variants,” which allow purification of heterodimeric proteins. As is generally described in U.S. Pat. No. 9,605,084, which is hereby incorporated by reference in its entirety, useful mechanisms for heterodimerization include “knobs and holes” (“KIH”) as described in U.S. Pat. No. 9,605,084, “electrostatic steering” or “charge pairs” as described in U.S. Pat. No. 9,605,084, which is hereby incorporated by reference in its entirety, pI variants as described in U.S. Pat. No. 9,605,084, which is hereby incorporated by reference in its entirety, and general additional Fc variants as outlined in U.S. Pat. No. 9,605,084, which is hereby incorporated by reference in its entirety.

Methods that Use Single Gene Vectors

In one aspect, the current disclosure provides a method of making a heterodimeric protein, the method comprising (i) providing a cell comprising a single gene vector encoding an alpha chain and/or a single gene vector encoding a beta chain; (ii) cultivating the cell, and (ii) and making the heterodimeric protein from culture supernatant, and/or lysate of the cell.

In one aspect, the current disclosure provides a method for manufacturing a heterodimeric protein, the method comprising: a) providing a population of cells (without limitations, e.g., ExpiCHO and Expi293 cells); b) transducing the population of cells with two single gene vectors (SGV) expressing an alpha chain (without limitation, e.g., BTN2A1-Fc-CD19scFv) and a beta chain (without limitation, e.g., BTN3A1-Fc-CD19scFv); c) culturing the transduced population of cells to proliferate; and d) extracting and/or purifying the heterodimeric protein from culture supernatant, and/or lysate of the transduced population of cells.

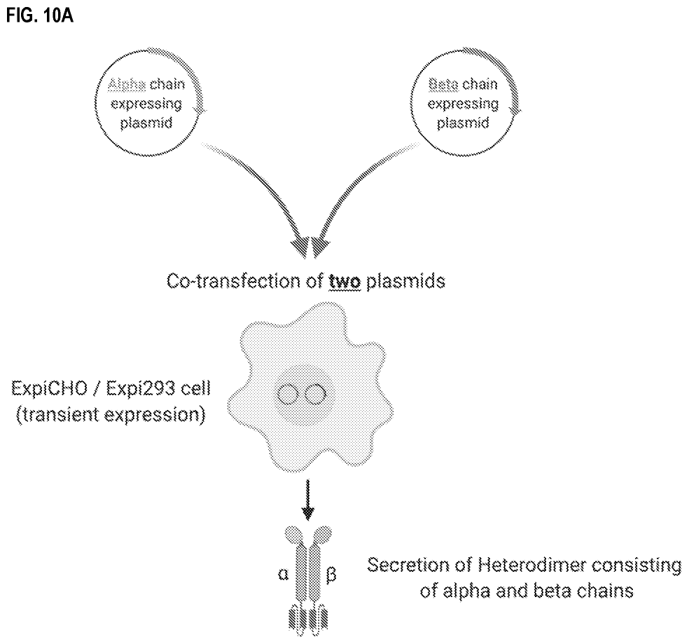

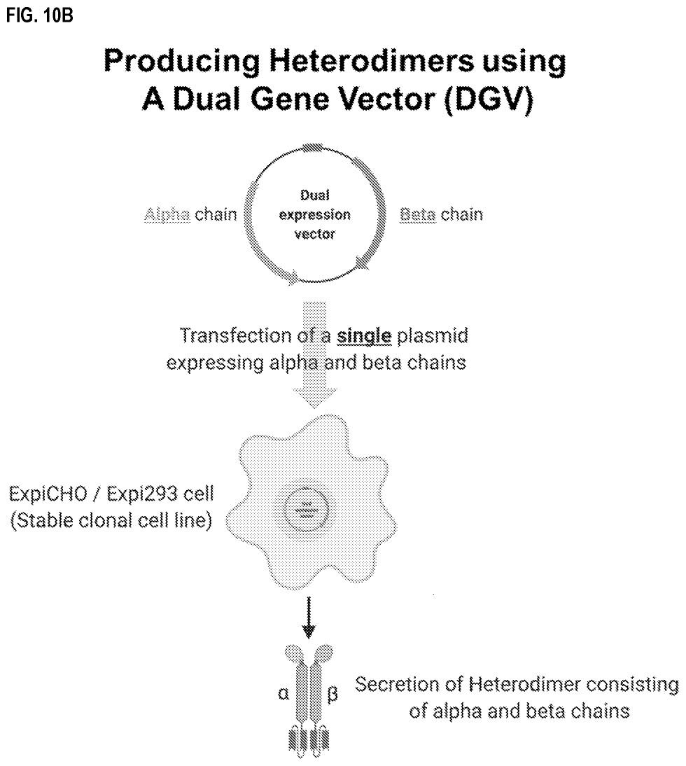

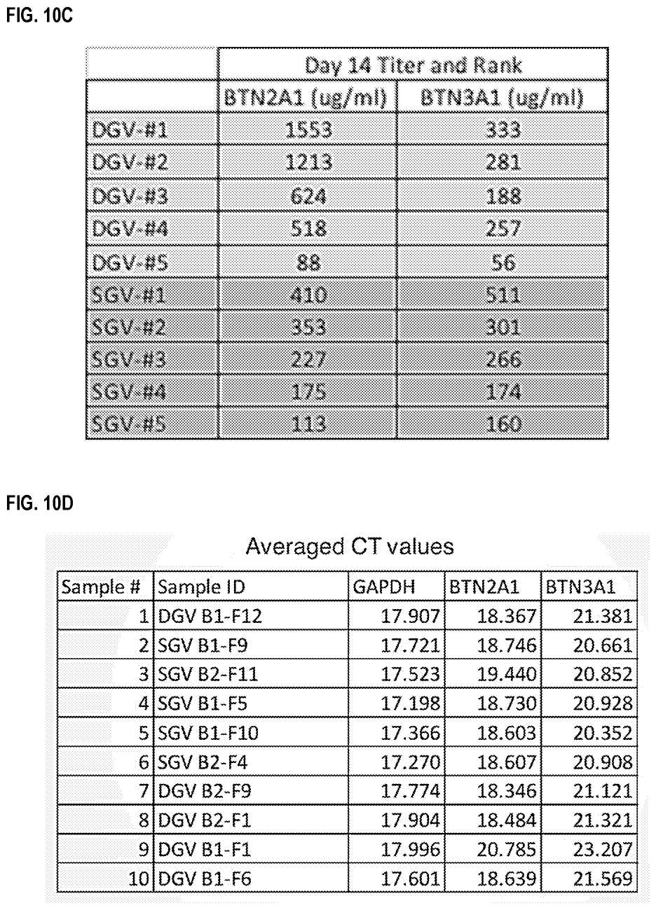

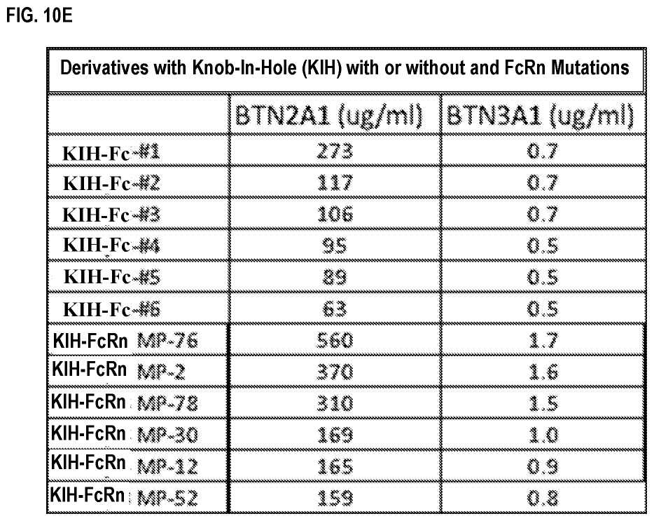

In embodiments, such method is as depicted in FIG. 10A. In embodiments, a cell (without limitations, e.g., an ExpiCHO and a Expi293 cell) is co-transfected with two single gene vectors (SGV) expressing an alpha chain (without limitation, e.g., BTN2A1-Fc-CD19scFv) and a beta chain (without limitation, e.g., BTN3A1-Fc-CD19scFv). In embodiments, the cell is substantially simultaneously transfected with the two single gene vectors (SGV) expressing the alpha chain (without limitation, e.g., BTN2A1-Fc-CD19scFv) and the beta chain (without limitation, e.g., BTN3A1-Fc-CD19scFv). In embodiments, the cell is sequentially transfected with the two single gene vectors (SGV) expressing the alpha chain (without limitation, e.g., BTN2A1-Fc-CD19scFv) and the beta chain (without limitation, e.g., BTN3A1-Fc-CD19scFv). In embodiments, the cell is transfected first with the single gene vector (SGV) expressing the alpha chain (without limitation, e.g., BTN2A1-Fc-CD19scFv) before transfecting with the single gene vector (SGV) expressing the beta chain (without limitation, e.g., BTN3A1-Fc-CD19scFv). In embodiments, the cell is transfected with the single gene vector (SGV) expressing the alpha chain (without limitation, e.g., BTN2A1-Fc-CD19scFv) after first transfecting with the single gene vector (SGV) expressing the beta chain (without limitation, e.g., BTN3A1-Fc-CD19scFv). In embodiments, the cell that is cotransfected with the two single gene vectors (SGV) expressing the alpha chain (without limitation, e.g., BTN2A1-Fc-CD19scFv) and the beta chain (without limitation, e.g., BTN3A1-Fc-CD19scFv) is isolated, enriched or purified. In embodiments, the cell that is cotransfected with the two single gene vectors (SGV) expressing the alpha chain (without limitation, e.g., BTN2A1-Fc-CD19scFv) and the beta chain (without limitation, e.g., BTN3A1-Fc-CD19scFv) is not isolated, enriched, or purified. In embodiments, the cell that is cotransfected with the two single gene vectors (SGV) expressing the alpha chain (without limitation, e.g., BTN2A1-Fc-CD19scFv) and the beta chain (without limitation, e.g., BTN3A1-Fc-CD19scFv), which is optionally isolated, enriched, or purified, is cultured in vitro. In embodiments, the cell that is cotransfected with the two single gene vectors (SGV) expressing the alpha chain (without limitation, e.g., BTN2A1-Fc-CD19scFv) and the beta chain (without limitation, e.g., BTN3A1-Fc-CD19scFv) is expanded in culture. In embodiments, the heterodimeric protein is extracted and/or purified from culture supernatant, and/or lysate of the cell that is cotransfected with the two single gene vectors (SGV) expressing the alpha chain (without limitation, e.g., BTN2A1-Fc-CD19scFv) and the beta chain (without limitation, e.g., BTN3A1-Fc-CD19scFv).

In embodiments, the heterodimeric protein comprises the alpha chain and the beta chain, wherein the alpha chain and the beta chain comprise (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the first domain comprising one or more butyrophilin family proteins, or a fragment thereof of the first and the second polypeptide chain are the same. In embodiments, the second domain comprising a targeting domain of the first and the second polypeptide chain are the same. In embodiments, the linker that adjoins the first and second domains are the same.

In embodiments, the heterodimeric protein comprises the alpha chain and the beta chain, wherein the alpha chain comprises: (a) a first domain comprising butyrophilin family protein is selected from BTN1A1, BTN2A1, BTN2A2, BTN2A3, BTN3A1, BTN3A2, BTN3A3, BTNL2, BTNL3, BTNL8, BTNL9, BTNL10, and SKINTL, or a fragment thereof; (b) a second domain comprising a targeting domain; and (c) a linker that adjoins the first and second domains; and wherein the beta chain comprises: (a) a first domain comprising butyrophilin family protein is selected from BTN1A1, BTN2A1, BTN2A2, BTN2A3, BTN3A1, BTN3A2, BTN3A3, BTNL2, BTNL3, BTNL8, BTNL9, BTNL10, and SKINTL, or a fragment thereof; (b) a second domain comprising a targeting domain that specifically binds to CD19; and (c) a linker that adjoins the first and second domains. In embodiments, the butyrophilin family protein is selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL. In embodiments, the targeting domain is the targeting domain of any embodiment disclosed herein. In embodiments, the linker is the linker of any embodiment disclosed herein.

In embodiments, the heterodimeric protein comprises the alpha chain and the beta chain, wherein the alpha chain comprises: (a) a first domain comprising BTN2A1, or a fragment thereof (without limitation, e.g. a variable domain); (b) a second domain comprising a targeting domain, the targeting domain being selected from an (i) antibody, antibody-like molecule, or antigen binding fragment thereof, and (ii) a extracellular domain; and (c) a linker that adjoins the first and second domains; and wherein the beta chain comprises: (a) a first domain comprising BTN3A1, or a fragment thereof (without limitation, e.g. a variable domain); (b) a second domain comprising a targeting domain, the targeting domain being selected from an (i) antibody, antibody-like molecule, or antigen binding fragment thereof, and (ii) a extracellular domain; and (c) a linker that adjoins the first and second domains.

Methods that Use Dual Gene Vectors

In one aspect, the current disclosure provides a method of making a heterodimeric protein, the method comprising (i) providing a cell comprising a dual gene vector encoding an alpha chain and a beta chain; (ii) cultivating the cell, and (ii) and making the heterodimeric protein from culture supernatant, and/or lysate of the cell.

In one aspect, the current disclosure provides a method for manufacturing a heterodimeric protein, the method comprising: a) providing a population of cells (without limitations, e.g., ExpiCHO and Expi293 cells); b) transducing the population of cells with a dual gene vector (DGV) expressing an alpha chain (without limitation, e.g., BTN2A1-Fc-CD19scFv) and a beta chain (without limitation, e.g., BTN3A1-Fc-CD19scFv); c) culturing the transduced population of cells to proliferate; and d) extracting and/or purifying the heterodimeric protein from culture supernatant, and/or lysate of the transduced population of cells.

In embodiments, such method is as depicted in FIG. 10B. In embodiments, a cell (without limitations, e.g., an ExpiCHO and an Expi293 cell) is transfected with a dual gene vector (DGV) expressing an alpha chain (without limitation, e.g., BTN2A1-Fc-CD19scFv) and a beta chain (without limitation, e.g., BTN3A1-Fc-CD19scFv). In embodiments, the cell that is transfected with the dual gene vector (DGV) expressing the alpha chain (without limitation, e.g., BTN2A1-Fc-CD19scFv) and the beta chain (without limitation, e.g., BTN3A1-Fc-CD19scFv) is isolated, enriched or purified. In embodiments, the cell that is transfected with the dual gene vector (DGV) expressing the alpha chain (without limitation, e.g., BTN2A1-Fc-CD19scFv) and the beta chain (without limitation, e.g., BTN3A1-Fc-CD19scFv), which is optionally isolated, enriched, or purified, is cultured in vitro. In embodiments, the cell that is transfected with the dual gene vector (DGV) expressing the alpha chain (without limitation, e.g., BTN2A1-Fc-CD19scFv) and the beta chain (without limitation, e.g., BTN3A1-Fc-CD19scFv) is expanded in culture. In embodiments, the heterodimeric protein is extracted and/or purified from culture supernatant, and/or lysate of the cell that is transfected with the dual gene vector (DGV) expressing the alpha chain (without limitation, e.g., BTN2A1-Fc-CD19scFv) and the beta chain (without limitation, e.g., BTN3A1-Fc-CD19scFv).

In embodiments, the heterodimeric protein comprises the alpha chain and the beta chain, wherein the alpha chain and the beta chain comprise (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the first domain comprising one or more butyrophilin family proteins, or a fragment thereof of the first and the second polypeptide chain are the same. In embodiments, the second domain comprising a targeting domain of the first and the second polypeptide chain are the same. In embodiments, the linker that adjoins the first and second domains are the same.

In embodiments, the heterodimeric protein comprises the alpha chain and the beta chain, wherein the alpha chain comprises: (a) a first domain comprising butyrophilin family protein is selected from BTN1A1, BTN2A1, BTN2A2, BTN2A3, BTN3A1, BTN3A2, BTN3A3, BTNL2, BTNL3, BTNL8, BTNL9, BTNL10, and SKINTL, or a fragment thereof; (b) a second domain comprising a targeting domain; and (c) a linker that adjoins the first and second domains; and wherein the beta chain comprises: (a) a first domain comprising butyrophilin family protein is selected from BTN1A1, BTN2A1, BTN2A2, BTN2A3, BTN3A1, BTN3A2, BTN3A3, BTNL2, BTNL3, BTNL8, BTNL9, BTNL10, and SKINTL, or a fragment thereof; (b) a second domain comprising a targeting domain that specifically binds to CD19; and (c) a linker that adjoins the first and second domains. In embodiments, the butyrophilin family protein is selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL. In embodiments, the targeting domain is the targeting domain of any embodiment disclosed herein. In embodiments, the linker is the linker of any embodiment disclosed herein.

In embodiments, the heterodimeric protein comprises the alpha chain and the beta chain, wherein the alpha chain comprises: (a) a first domain comprising BTN2A1, or a fragment thereof (without limitation, e.g. a variable domain); (b) a second domain comprising a targeting domain, the targeting domain being selected from an (i) antibody, antibody-like molecule, or antigen binding fragment thereof, and (ii) a extracellular domain; and (c) a linker that adjoins the first and second domains; and wherein the beta chain comprises: (a) a first domain comprising BTN3A1, or a fragment thereof (without limitation, e.g. a variable domain); (b) a second domain comprising a targeting domain, the targeting domain being selected from an (i) antibody, antibody-like molecule, or antigen binding fragment thereof, and (ii) a extracellular domain; and (c) a linker that adjoins the first and second domains.

Kits

The invention provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1: Construction and Characterization of an Illustrative BTN2A1/3A1-Fc-CD19scFv Heterodimeric Protein

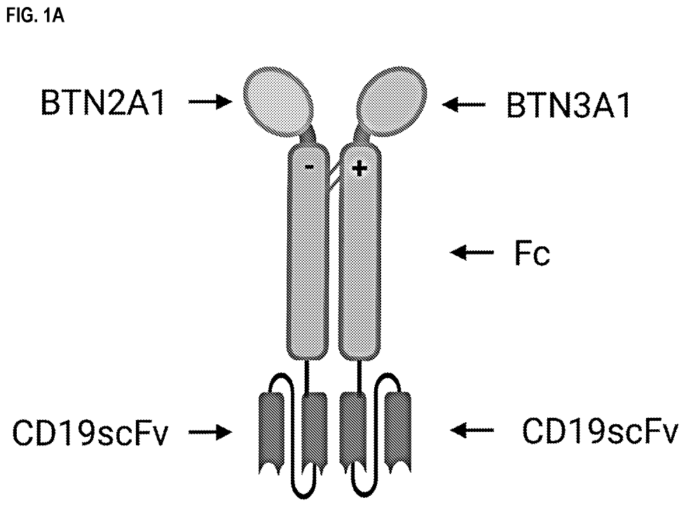

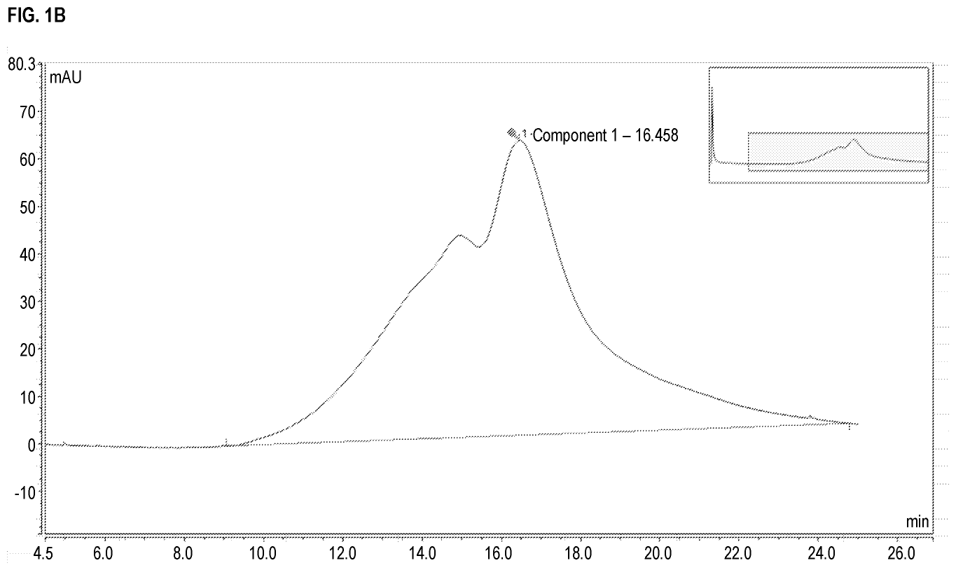

The heterodimeric proteins of the present technology comprise a dimer of two chimeric proteins, each comprising a butyrophilin family member, a core domain, and an antigen-targeting domain. The “BTN2A1/3A1-Fc-CD19scFv” construct included an alpha chain comprising an extracellular domain (ECD) of human BTN2A1 fused to a CD19scFv via a hinge-CH2-CH3 Fc domain, and a beta chain comprising an extracellular domain (ECD) of human BTN3A1 fused to a CD19scFv via a hinge-CH2-CH3 Fc domain. See, FIG. 1A. Constructs encoding BTN2A1-Fc-CD19scFv protein (alpha chain) and BTN3A1-Fc-CD19scFv protein (beta chain) were generated. This GAmma DELta T cell ENgager construct also is referred to herein as the BTN2A1/3A1-Fc-CD19scFv ‘GADLEN’ protein.

The BTN2A1/3A1-Fc-CD19scFv heterodimer protein that was produced via a transient co-transfection in Expi293 cells of two plasmids encoding 1) the BTN2A1-alpha-CD19scFv protein and 2) the BTN3A1-beta-CD19scFv protein. The alpha and beta constructs encoded a BTN2A1-Fc-CD19scFv (‘alpha’ chain) and a BTN3A1-Fc-CD19scFv (‘beta’ chain). The alpha and beta chains contained charged polarized linker domains which facilitated heterodimerization of the desired the BTN2A1/3A1-Fc-CD19scFv GADLEN protein. The cell culture supernatant from the transient transfection was harvested 6 days following transfection and purified over a FcXL chromatography resin. As shown in FIG. 1B, the FcXL chromatography revealed the resultant protein was substantially pure.

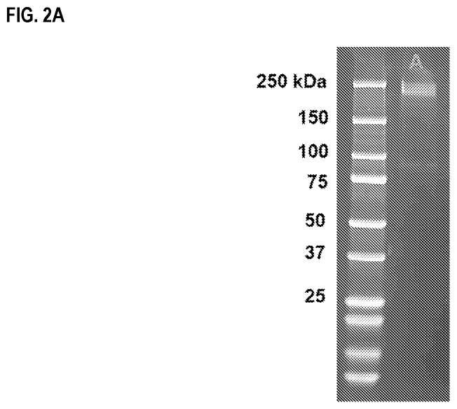

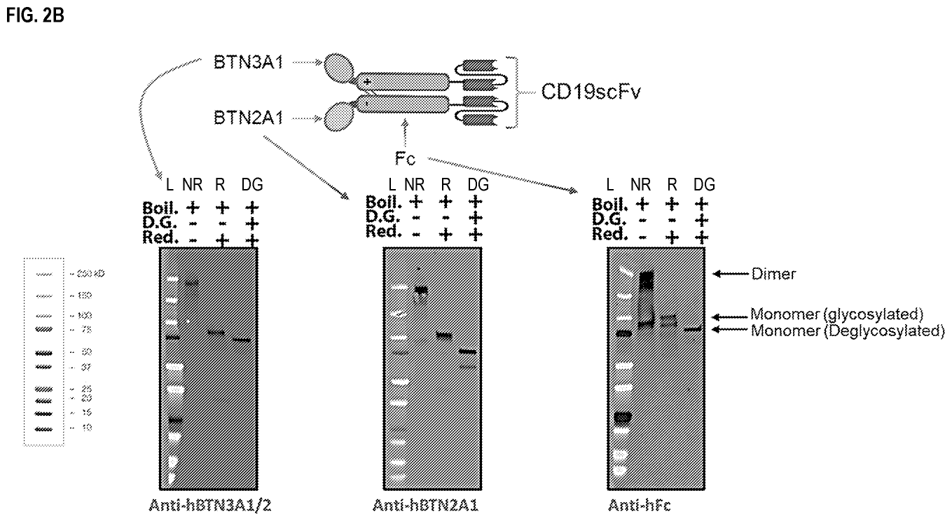

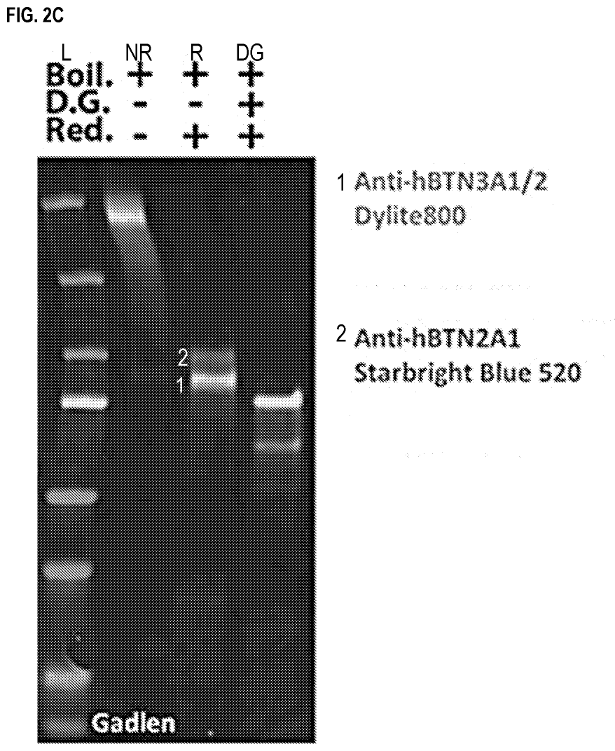

Purity of the protein was further assessed using non-reducing sodium dodecyl sulfate polyacrylamide gel electrophoresis (SDS-PAGE). As shown in FIG. 2A, Coomassie blue-stained gels revealed that the BTN2A1/3A1-Fc-CD19scFv GADLEN protein had >90% purity (see also FIGS. 2B and 2C).

The purified protein was further analyzed by western blot using non-reducing, reducing, and both reducing and deglycosylating conditions, following detection with an anti-human BTN2A1 antibody, an anti-human BTN3A1 antibody, or an anti-mouse Fc antibody. Non-reduced BTN2A1/3A1-Fc-CD19scFv GADLEN protein ran as a single band (See lanes “L” in FIG. 2B) indicative of covalent complex formation between the BTN2A1-alpha-CD19scFv and BTN3A1-beta-CD19scFv chains. As shown in FIG. 2B, the blots probed with the anti-Fc antibody revealed two bands with the protein prepared under reducing but non-deglycosylated condition (See lane “R” in FIG. 2B). Gels probed with the anti-human BTN2A1 and the anti-human BTN3A1 antibodies indicated bands with mobility corresponding to the two bands revealed in the anti Fc-probed blot.

Interestingly, protein prepared under both reduced and deglycosylated (lane “DG”) conditions resulted in a single band, which could be detected with any of the anti-human BTN2A1, anti-human BTN3A1, or anti-mouse Fc antibodies.

to facilitate contemporaneous detection of BTN2A1-alpha-CD19scFv and BTN3A1-beta-CD19scFv monomers, the purified BTN2A1/3A1-Fc-CD19scFv GADLEN protein was analyzed by Western blot using non-reduced (lane “NR”), reduced (lane “R”) and both reduced and deglycosylated (lane “DG”) conditions, following detection with an anti-human BTN2A1 antibody conjugated with Starbright Blue 520 and anti-human BTN3A1 antibody conjugated with Dylite800. As shown in FIG. 2C, the dual color western blot analysis of indicated the presence of BTN2A1-alpha and BTN3A1-beta chains in reduced but non-deglycosylated condition. Specifically, the blue BTN2A1-alpha-CD19scFv band migrated slower than the green BTN3A1-beta-CD19scFv monomer (See lane “R” in FIG. 2C). BTN2A1/3A1-Fc-CD19scFv GADLEN protein prepared under non-reduced condition (lane “NR” in FIG. 2C) and both reduced and deglycosylated condition (lane “DG” in FIG. 2C) ran as a single blue-green band.

These results indicate the presence of a disulfide-linked dimeric protein that reduces to two individual proteins (following disruption of the interchain disulfide bonds with R-mercaptoethanol). These data further suggested based on the similarity between the reduced and both reduced and deglycosylated lanes that the BTN2A1/3A1-Fc-CD19scFv GADLEN is glycosylated. These data further suggested that BTN2A1-Fc-CD19scFv was glycosylated more than BTN3A1-Fc-CD19scFv.

### Example 2: Binding of BTN2A1/3A1-Fc-CD19scFv GADLEN Protein to CD19

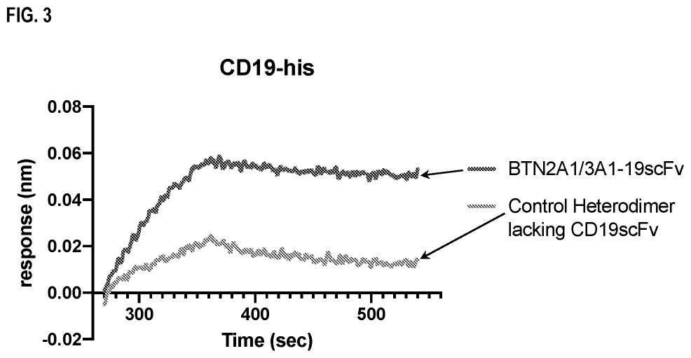

To study the binding kinetics of binding of the CD19scFv present in the BTN2A1/3A1-Fc-CD19scFv GADLEN protein, binding assays were performed using the Octet system (ForteBio). Briefly, recombinant CD19-His protein was immobilized on a biosensor and the BTN2A1/3A1-Fc-CD19scFv GADLEN protein or a control heterodimer lacking CD19scFv was added. The binding response of the BTN2A1/3A1-Fc-CD19scFv GADLEN protein to CD19-His protein was plotted in real time on a sensorgram trace. As shown in FIG. 3, the BTN2A1/3A1-Fc-CD19scFv GADLEN protein bound to recombinant CD19-His protein with a time-dependent and saturable kinetics. In comparison, the control heterodimer lacking CD19scFv showed only background signal. These experiemnts revealed the following binding parameters for the binding of the BTN2A1/3A1-Fc-CD19scFv GADLEN protein to human CD19:

| Heterodimer | | Kₒₙ | K_{dis} | K_{D} | Full |
|---|---|---|---|---|---|
| Construct | Ligand | (1/Ms) | (1/s) | (nM) | R² |
| BTN2A1/3A1-Fc- | Human | 4.86E+04 | 6.72E−04 | 13.8 | 0.992 |
| CD19scFv | CD19 | | | | |

These results demonstrate that the CD19scFv located at C-terminus of the BTN2A1/3A1-Fc-CD19scFv GADLEN protein disclosed herein specifically binds to CD19.

### Example 3: Contemporaneous Binding of BTN2A1/3A1-Fc-CD19scFv GADLEN Protein to CD19 and a BTN2A1/BTN3A1 Ligand

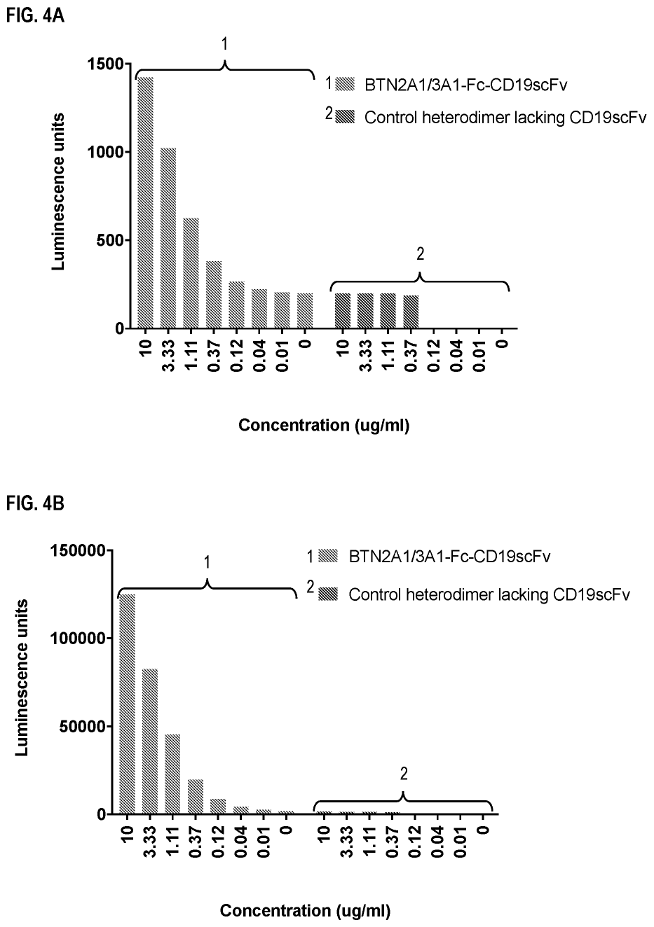

The BTN2A1/3A1-Fc-CD19scFv GADLEN protein harbors an extracellular domains (ECDs) of BTN2A1 and BTN3A1. Whether the ECDs of BTN2A1 and BTN3A1 protein present and the CD19scFv present in the native BTN2A1/3A1-Fc-CD19scFv GADLEN protein can contemporaneously to their ligand was explored next using a Meso Scale Discovery (MSD) ELISA-based assay. Recombinant CD19 protein was coated on plates and increasing amounts of the BTN2A1/3A1-Fc-CD19scFv GADLEN protein or a heterodimer lacking CD19scFv were added to the plates for capture by the plate-bound recombinant CD19 protein. The binding was detected using an anti-BTN2A1 antibody. As shown in FIG. 4A, the BTN2A1/3A1-Fc-CD19scFv GADLEN protein but not the heterodimer lacking CD19scFv exhibited a dose-dependent binding. Since generation of signal in this assay requires contemporaneous binding to recombinant CD19 protein and the anti-BTN2A1 antibody, these data demonstrate that the BTN2A1/3A1-Fc-CD19scFv GADLEN protein can contemporaneously bind to CD19 protein and a BTN2A1 ligand.

In another experiment, recombinant CD19 protein was coated on plates and increasing amounts of the BTN2A1/3A1-Fc-CD19scFv GADLEN protein or a heterodimer lacking CD19scFv were added to the plates for capture by the plate-bound recombinant CD19 protein. The binding was detected using an anti-BTN3A1 antibody. As shown in FIG. 4B, the BTN2A1/3A1-Fc-CD19scFv GADLEN protein but not the heterodimer lacking CD19scFv exhibited a dose-dependent binding. Since generation of signal in this assay requires contemporaneous binding to recombinant CD19 protein and the anti-BTN3A1 antibody, these data demonstrate that the BTN2A1/3A1-Fc-CD19scFv GADLEN protein can contemporaneously bind to CD19 protein and a BTN3A1 ligand.

These data indicate that the BTN2A1/3A1-Fc-CD19scFv GADLEN protein can contemporaneously bind CD19 and a buryrophilin BTN2A1/3A1 ligand. Collectively, these data demonstrate that the BTN2A1/3A1-Fc-CD19scFv GADLEN protein can both CD19scFv and buryrophilin BTN2A1/3A1 ends can contemporaneously bind their ligands.

### Example 4: Contemporaneous Binding of BTN2A1/3A1-Fc-CD19scFv GADLEN Protein to BTN2A1 and BTN3A1 Ligands

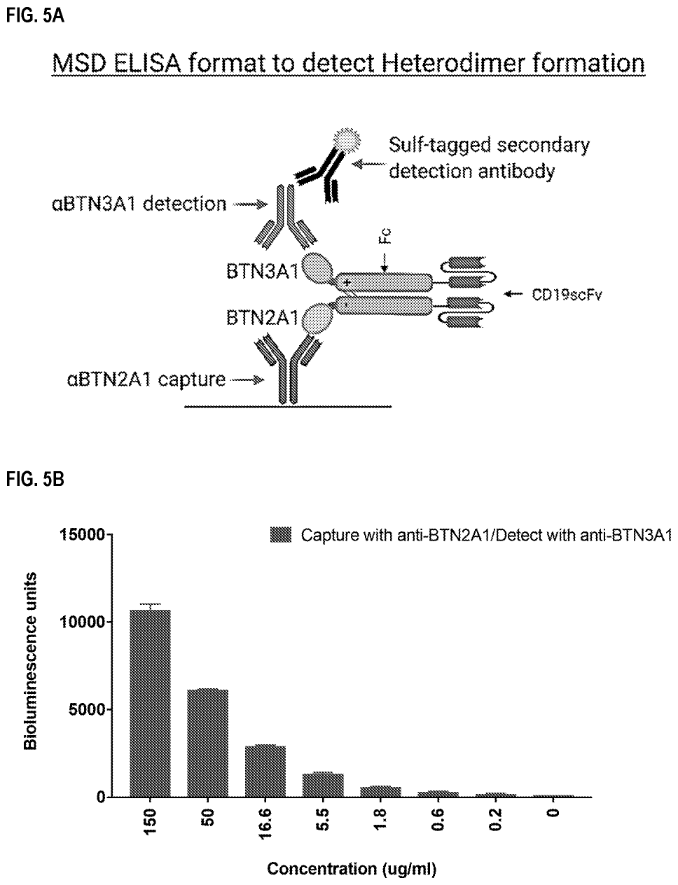

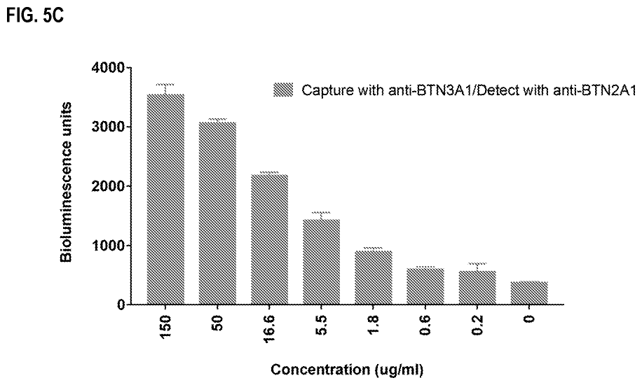

Whether the ECDs of BTN2A1 and BTN3A1 protein present and the CD19scFv present in the native BTN2A1/3A1-Fc-CD19scFv GADLEN protein can contemporaneously to their ligands was explored next using an MSD ELISA-based assay. FIG. 5A shows a schematic representation of the MSD ELISA assay. An anti-BTN2A1 antibody was coated on plates and increasing amounts of the BTN2A1/3A1-Fc-CD19scFv GADLEN protein were added to the plates for capture by the plate-bound anti-BTN2A1 antibody. The binding was detected using an anti-BTN3A1 antibody. As shown in FIG. 5B, the BTN2A1/3A1-Fc-CD19scFv GADLEN protein exhibited a dose-dependent binding. Since generation of signal in this assay requires contemporaneous binding to the plate-bound anti-BTN2A1 antibody and the anti-BTN3A1 antibody, these data demonstrate that the BTN2A1/3A1-Fc-CD19scFv GADLEN protein could bridge the plate-bound anti-BTN2A1 antibody and the anti-BTN3A1 antibody.

In another experiment, an anti-BTN3A1 antibody was coated on plates and increasing amounts of the BTN2A1/3A1-Fc-CD19scFv GADLEN protein were added to the plates for capture by the plate-bound anti-BTN3A1 antibody. The binding was detected using an anti-BTN2A1 antibody. As shown in FIG. 5C, the BTN2A1/3A1-Fc-CD19scFv GADLEN protein exhibited a dose-dependent binding. Since generation of signal in this assay requires contemporaneous binding to the plate-bound anti-BTN3A1 antibody and the anti-BTN2A1 antibody, these data demonstrate that the BTN2A1/3A1-Fc-CD19scFv GADLEN protein could bridge the plate-bound anti-BTN3A1 antibody and the anti-BTN2A1 antibody.

These data indicate that the BTN2A1/3A1-Fc-CD19scFv GADLEN protein can contemporaneously bind the anti-BTN3A1 antibody and the anti-BTN2A1 antibody. Collectively, these data demonstrate that the BTN2A1/3A1-Fc-CD19scFv GADLEN protein can contemporaneously bind to both a buryrophilin BTN2A1 ligand/receptor and a BTN3A1 ligand/receptor.

### Example 5: CD19-Dependent Cell-Surface Binding by BTN2A1/3A1-Fc-CD19scFv GADLEN Protein

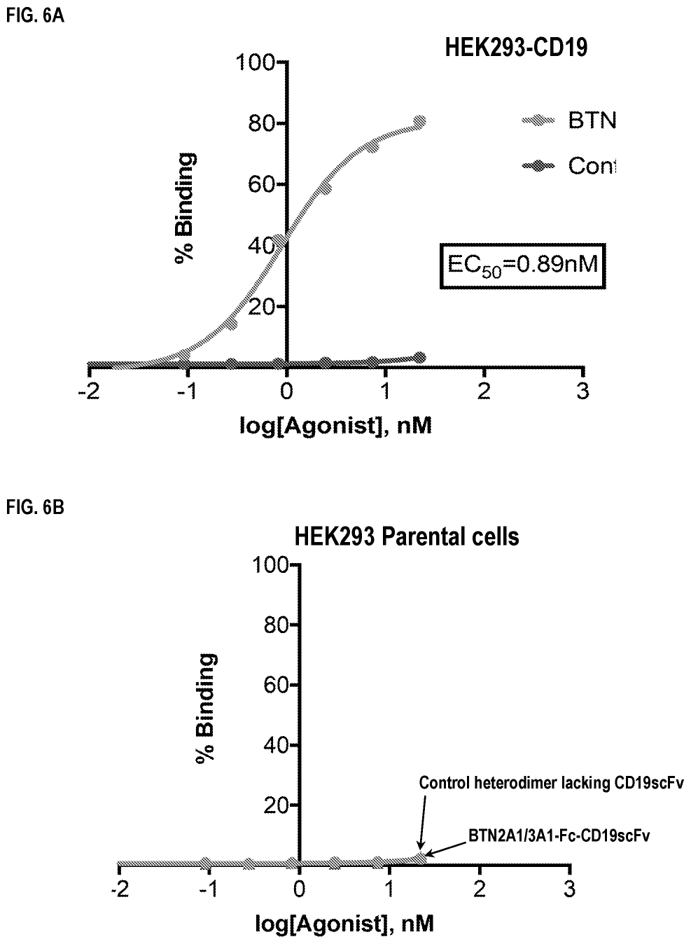

To study binding of the BTN2A1/3A1-Fc-CD19scFv GADLEN protein to CD19+ cells, HEK293 cells expressing CD19 on surface (HEK293-CD19 cells) and HEK293 parental cells were used. Increasing amounts of the BTN2A1/3A1-Fc-CD19scFv GADLEN protein or a control heterodimer that lacks CD19scFv, which was used as a negative control for binding, were added to HEK293-CD19 cells. The HEK293-CD19 cell-bound BTN2A1/3A1-Fc-CD19scFv GADLEN protein was detected using anti-Fc antibody, and assayed using flow cytometry. As shown in FIG. 6A, the BTN2A1/3A1-Fc-CD19scFv GADLEN protein exhibited a dose-dependent and saturable binding to the HEK293-CD19 cells. In contrast, the heterodimer lacking CD19scFv showed only background level of binding. The data showed that the BTN2A1/3A1-Fc-CD19scFv GADLEN protein bound the HEK293-CD19 cells with an EC₅₀ of 0.89 nM.

In another experiment, increasing amounts of the BTN2A1/3A1-Fc-CD19scFv GADLEN protein or a control heterodimer that lacks CD19scFv were added to the parental HEK293 cells. Binding BTN2A1/3A1-Fc-CD19scFv GADLEN protein was assayed using flow cytometry as in FIG. 6A. As shown in FIG. 6B, both the BTN2A1/3A1-Fc-CD19scFv GADLEN protein and the heterodimer lacking CD19scFv exhibited only background level of binding.

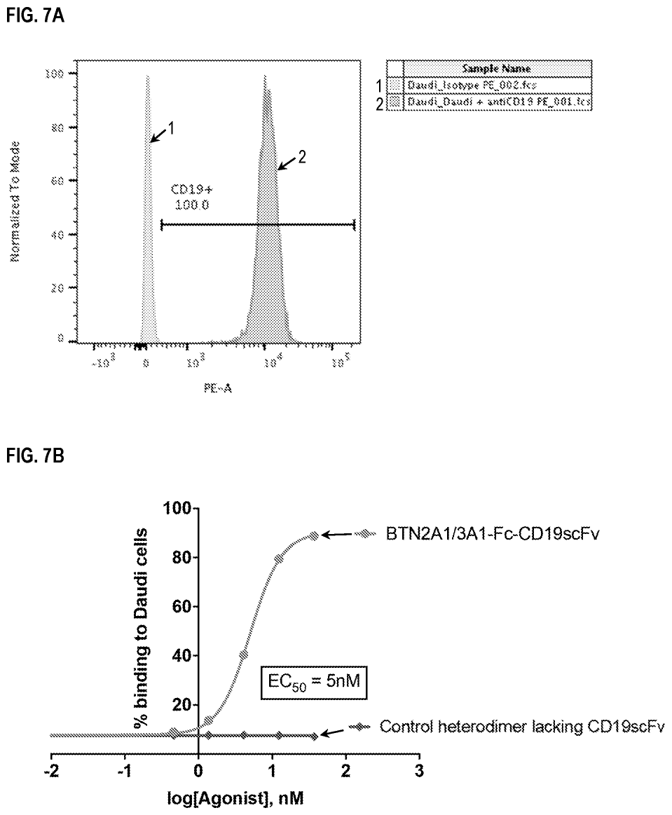

Binding of the BTN2A1/3A1-Fc-CD19scFv GADLEN protein to CD19+ cells, was further explored using Daudi cells, which express CD19 on surface. The expression of CD19 on the surface of Daudi cells was confirmed using flow cytometry. As shown in FIG. 7A, an anti-CD19 antibody but not an isotype control was able to stain Daudi cells confirming that Daudi cells are CD19+.

Increasing amounts of the BTN2A1/3A1-Fc-CD19scFv GADLEN protein or a control heterodimer lacking CD19scFv were added to Daudi cells and binding was detected using flow cytometry. As shown in FIG. 7B, the BTN2A1/3A1-Fc-CD19scFv GADLEN protein but not the control heterodimer lacking CD19scFv bound Daudi cells in a dose-dependent and saturable manner. These data again showed that the BTN2A1/3A1-Fc-CD19scFv GADLEN protein bound the Daudi cells with an EC₅₀ of 5 nM.

These results demonstrate that the BTN2A1/3A1-Fc-CD19scFv GADLEN protein specifically binds CD19+ cells in a dose-dependent and saturable manner.

### Example 6: Binding by BTN2A1/3A1-Fc-CD19scFv GADLEN Protein to γδ Cells

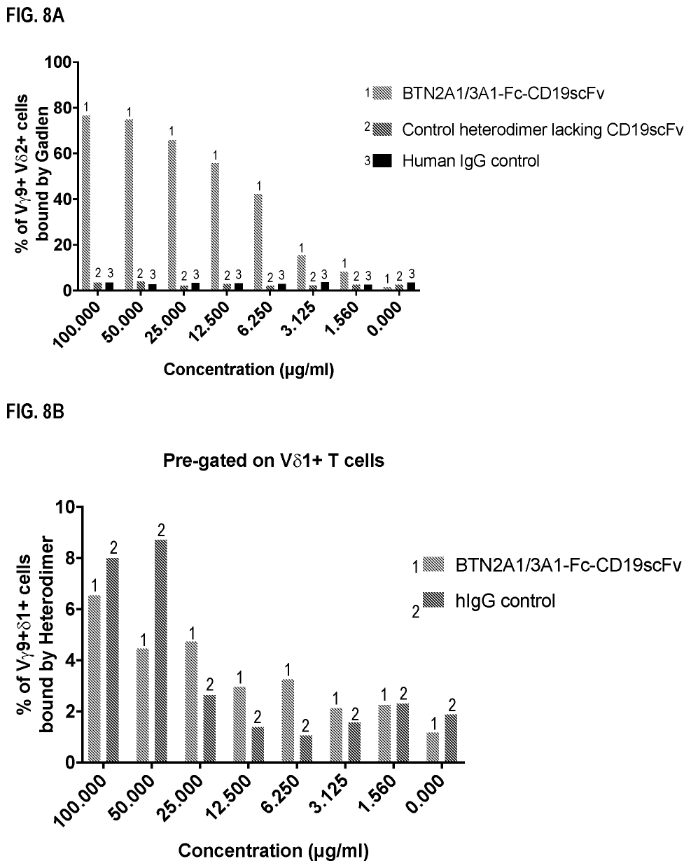

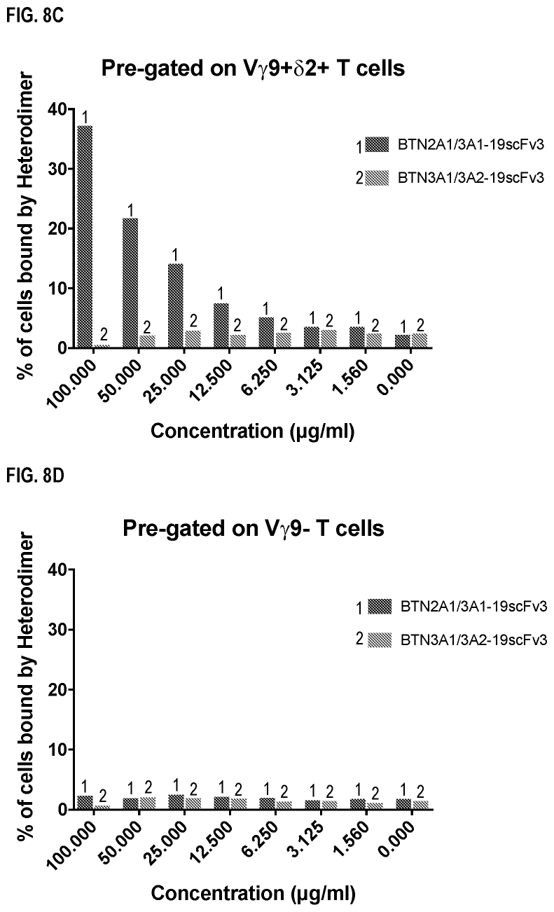

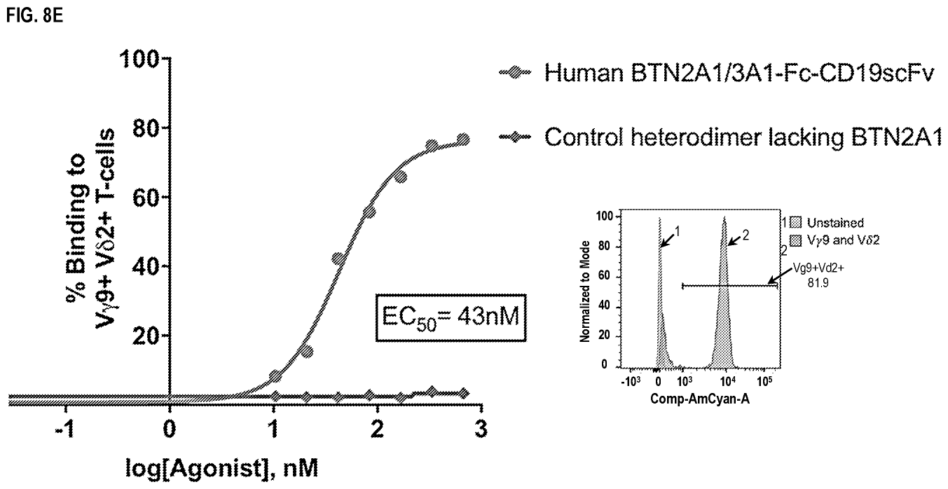

Next, binding of the BTN2A1/3A1-Fc-CD19scFv GADLEN protein to γδ cells was studied. Vγ9+V62+T-cells were isolated and expanded from peripheral blood mononuclear cells (PBMCs) from a healthy donor. The isolated Vγ9+Vδ2+T-cells were incubated with the human BTN2A1/3A1-Fc-CD19scFv GADLEN protein, a control heterodimer protein lacking BTN2A1, or human IgG control. Binding was detected by flow cytometry using an APC conjugated anti-hFc antibody that binds to the Fc-domain of the Heterodimer protein. As shown in FIG. 8A, the human BTN2A1/3A1-Fc-CD19scFv GADLEN protein specifically bound to Vγ9+Vδ2+T-cells. In contrast, a control heterodimer protein lacking CD19scFv, or human IgG control did not bind the Vγ9+Vδ2+ T-cells.

In another experiment, Vγ9+Vδ1+T-cells were isolated and expanded from PBMCs from a healthy donor. The isolated Vγ9+Vδ1+T-cells were incubated with the human BTN2A1/3A1-Fc-CD19scFv GADLEN protein, or human IgG control. Binding was detected by flow cytometry using an APC conjugated anti-hFc antibody that binds to the Fc-domain of the Heterodimer protein. As shown in FIG. 8B, neither the human BTN2A1/3A1-Fc-CD19scFv GADLEN protein not the human IgG control bound the Vγ9+Vδ1+T-cells.

In a further experiment, Vγ9+Vδ2+T-cells were isolated and expanded from peripheral blood mononuclear cells (PBMCs) from a healthy donor. The isolated Vγ9+Vδ2+T-cells were incubated with the human BTN2A1/3A1-Fc-CD19scFv GADLEN or BTN3A1/3A2-Fc-CD19scFv GADLEN proteins. Binding was detected by flow cytometry. As shown in FIG. 8C, the human BTN2A1/3A1-Fc-CD19scFv GADLEN protein but not the BTN3A1/3A2-Fc-CD19scFv GADLEN protein bound to the isolated human Vγ9+Vδ2+T-cells.

In a further experiment, Vγ9+Vδ2+T-cells were isolated and expanded from peripheral blood mononuclear cells (PBMCs) from a healthy donor. As shown in FIG. 8E (inset), the human BTN2A1/3A1-Fc-CD19scFv GADLEN protein bound to human γδ T cells expressing the Vγ9δ2 TCR compared to unstained cells as shown by flow cytometry. Increasing amounts of the human BTN2A1/3A1-Fc-CD19scFv GADLEN protein or a heterodimer lacking BTN2A1 were incubated with the isolated Vγ9+Vδ2+T-cells and binding was detected using flow cytometry. As shown in FIG. 8E, the human BTN2A1/3A1-Fc-CD19scFv GADLEN protein exhibited a dose-dependent binding to human γδ T cells expressing the Vγ9δ2 TCR with an EC₅₀ of 43 nM. In contrast, the heterodimer lacking BTN2A1 did not bind to T cells expressing the Vγ9δ2 TCR.

In yet another experiment, Vγ9− T-cells were isolated and expanded from PBMCs from a healthy donor. The isolated Vγ9− T-cells were incubated with the human BTN2A1/3A1-Fc-CD19scFv GADLEN or BTN3A1/3A2-Fc-CD19scFv GADLEN proteins. Binding was detected by flow cytometry using an APC conjugated anti-hFc antibody that binds to the Fc-domain of the heterodimer protein. As shown in FIG. 8D, neither of the human BTN2A1/3A1-Fc-CD19scFv GADLEN or BTN3A1/3A2-Fc-CD19scFv GADLEN proteins bound the isolated Vγ9− T-cells.

These results demonstrate that the BTN2A1/3A1-Fc-CD19scFv GADLEN protein specifically binds CD19+ cells in a dose-dependent and saturable manner.

### Example 7: Binding by BTN2A1-Fc-CD19scFv GADLEN Protein to γδ Cells Requires Dimerization

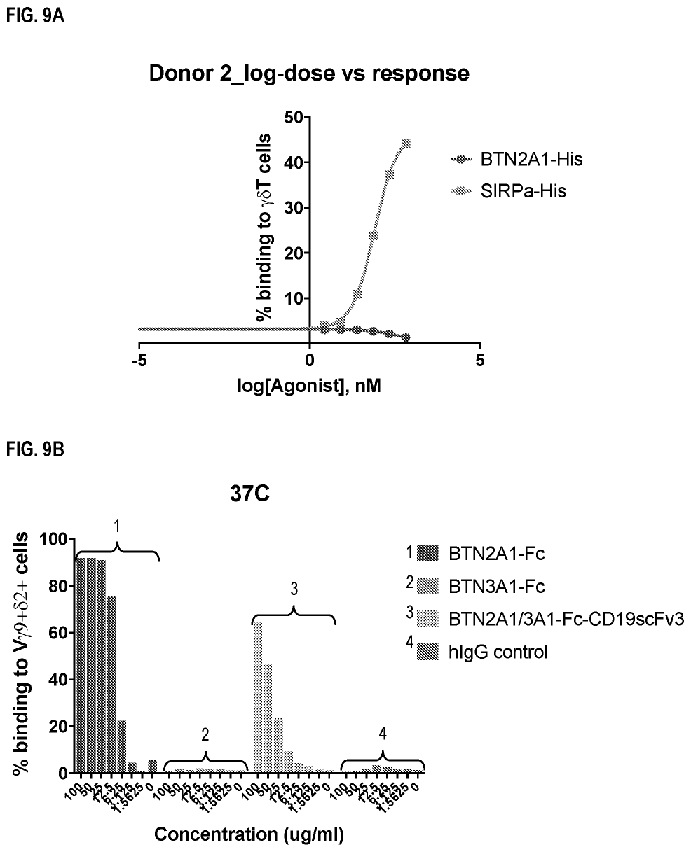

Next, binding to Vγ9+Vδ2+T-cells by BTN2A1-His and SIRPα-His proteins, which exist as monomers in solution was studied. Increasing amounts of BTN2A1-His and SIRPα-His proteins were added to Vγ9+Vδ2+ T-cells. Binding was detected using flow cytometry-based on detection of the His tag. As shown in FIG. 9A, the BTN2A1-His protein did not bind to Vγ9+Vδ2+T-cells. In contrast, SIRPα-His protein, which binds to CD47 on cells bound in a dose-dependent and saturable manner.

To explore this observation further, BTN2A1-Fc, BTN3A1-Fc, the human BTN2A1/3A1-Fc-CD19scFv GADLEN proteins, and human IgG control were used. The BTN2A1-Fc and BTN3A1-Fc proteins exists as a dimer in solution. Vγ9+Vδ2+T-cells were incubated with increasing amounts of BTN2A1-Fc, BTN3A1-Fc, the human BTN2A1/3A1-Fc-CD19scFv GADLEN proteins, and human IgG control. Binding was detected using flow cytometry. As shown in FIG. 9B, BTN2A1-Fc and the human BTN2A1/3A1-Fc-CD19scFv GADLEN protein bound to Vγ9+Vδ2+T-cells in a dose-dependent and saturable manner. In contrast, BTN3A1-Fc protein and human IgG control did not bind to Vγ9+Vδ2+T-cells. These data suggest that BTN2A1 needs to homodimerize in order to interact with the Vγ9+Vδ2 T cell receptor.

Since BTN2A1-Fc protein, which bound to Vγ9+Vδ2+T-cells, exists as a dimer in solution, and BTN2A1-His protein, which did not bind to Vγ9+Vδ2+T-cells, exists as a monomer in solution, these data demonstrate that dimerization of BTN2A1, either homodimerization with BTN2A1, or heterodimerization, with e.g. BTN3A1, is required for binding to Vγ9+Vδ2+T-cells.

### Example 8: Cell Line Development and Production of BTN2A1-Fc-CD19scFv GADLEN

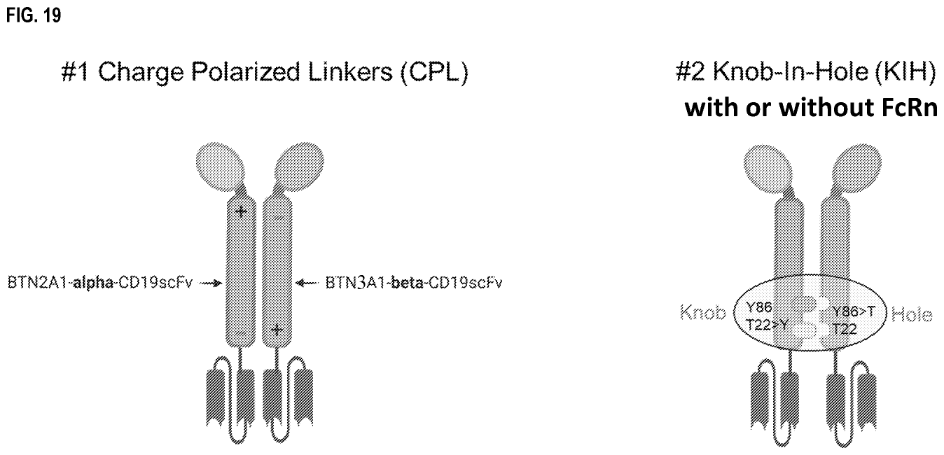

Three versions of the BTN2A1/3A1-Fc-CD19scFv GADLEN protein were generated to compare the charged polarized linker strategy to facilitate heterodimerization versus the knob-in-hole (KIH) mutations: charged polarized linkers, KIH mutations in Fc domain, KIH mutations and FcRn mutations (see FIG. 19). Without being bound by theory is thought that the BTN2A1/3A1-Fc-CD19scFv GADLEN protein having KIH mutations and FcRn mutations increase binding to neonatal Fc receptor.

Cell line development (CLD) for the BTN2A1/3A1-Fc-CD19scFv heterodimeric construct was performed to assess manufacturability using two approaches: co-transfection of two single gene vectors (SGV) expressing the alpha chain and beta chain separately (FIG. 10A), and transfection using a dual gene vector (DGV) that expresses the alpha and beta chain under two separate promoters in a single vector (FIG. 10B).

During the CLD process for all the BTN2A1/3A1-Fc-CD19scFv GADLEN protein molecules, the expression of the alpha and beta chains was evaluated in the mini pools via MSD ELISA and ranked the mini pools in order to down select and enable the selection of the top mini pool that would potentially move to the single cell cloning stage of the process. Mini-pools of BTN2A1-alpha chain (BTN2A1-Fc-CD19scFv) and BTN3A1-beta chain (BTN3A1-Fc-CD19scFv) having charged polarized linkers that were produced by either co-transfection of two single gene vectors (SGV) or transfection using a dual gene vector (DGV) were grown in shake flask cultures. The expression of BTN2A1-alpha chain and BTN3A1-beta chain was analyzed using MSD-ELISA based assays on day 14. The comparison of the protein titers is shown in FIG. 10C. The expression of BTN2A1-alpha chain mRNA and BTN3A1-beta chain mRNA was analyzed using quantitative RT-PCR on day 14. FIG. 10D shows the comparison of BTN2A1-alpha chain mRNA and BTN3A1-beta chain mRNA in SGV and DGV mini-pools. Interestingly, the dual gene vector (DGV) derived mini pools expressed more of the BTN2A1-alpha-CD19scFv chain compared to the BTN3A1-beta-CD19scFv chain, while the SGV-derived mini-pools appeared to express relatively equal amounts of the two chains at the protein and RNA level.

These data indicate that both two single gene vectors (SGV) or a single dual gene vector (DGV) may be used to produce the GADLEN proteins, including the BTN2A1/3A1-Fc-CD19scFv GADLEN protein. As shown herein, co-transfection of two single gene vectors (SGV) produced substantially equal amounts of the two chains. A single dual gene vector (DGV) may be used with further optimization of the expression of the BTN3A1-beta-CD19scFv chain, with respect to e.g., promoter strength and/or mRNA stability.

Additionally, the BTN2A1/3A1-Fc-CD19scFv GADLEN protein constructs where the charged polarized linkers were replaced with other dimerization motifs, such as an Fc domain having KIH mutations and another Fc domain having KIH mutations and FcRn mutations were generated only using the dual gene vector approach. The expression of BTN2A1-alpha chain and BTN3A1-beta chain was analyzed for constructs having KIH mutations in Fc domain (KIH-Fc) and KIH mutations with FcRn mutations (KIH-FcRn) using MSD-ELISA based assays on day 14. The comparison of titers of is shown in FIG. 10E. As shown in FIG. 10E and FIG. 10C, a significant reduction in BTN3A1-beta-CD19scFv expression was observed in constructs having KIH mutations in Fc domain (KIH-Fc) and KIH mutations with FcRn mutations (KIH-FcRn) mini pools compared to the constructs having charged polarized linkers.

The top 12 mini pools from constructs having KIH mutations in Fc domain (KIH-Fc) and KIH mutations with FcRn mutations (KIH-FcRn) will be moved up to the shake flask stage to further assess the expression level of the two chains which would whether the charge polarized linker approach is better suited for heterodimerization over the KIH mutations.

### Example 9: Production of GADLEN Proteins Using Cells Transfected with Two Single Gene Vectors or a Dual Gene Vector

The two approaches of manufacture of the BTN2A1/3A1-Fc-CD19scFv heterodimeric GADLEN protein were studied further.

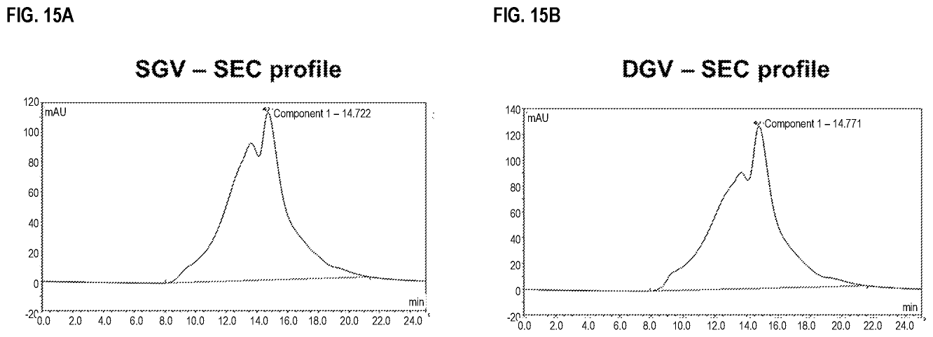

The BTN2A1/3A1-Fc-CD19scFv heterodimeric GADLEN protein was prepared using both the approaches: co-transfection of two single gene vectors (SGV) expressing the alpha chain and beta chain separately (FIG. 10A), and transfection using a dual gene vector (DGV) that expresses the alpha and beta chain under two separate promoters in a single vector (FIG. 10B). The purified proteins were subjected to size exclusion chromatography (SEC) to assess their purity. The size exclusion chromatography (SEC) profile of the BTN2A1/3A1-Fc-CD19scFv heterodimeric GADLEN proteins manufactured using two single gene vectors is shown in FIG. 15A. The size exclusion chromatography (SEC) profile of the BTN2A1/3A1-Fc-CD19scFv heterodimeric GADLEN proteins manufactured using a dual gene vector is shown in FIG. 15B. As shown in FIG. 15A and FIG. 15B, similar SEC profiles were obtained from a BTN2A1/3A1-Fc-CD19scFv construct produced from either the SGV or DGV format. These results demonstrate that the GADLEN heterodimeric can be produced from either one of these production formats.

To further evaluate the a SGV or DGV production formats, the BTN2A1/3A1-Fc-CD19scFv heterodimeric protein produced from either a SGV or DGV production format was analyzed by western blotting to confirm the presence of the BTN2A1-alpha-CD19scFv and BTN3A1-beta-CD19scFv chains in the purified material.

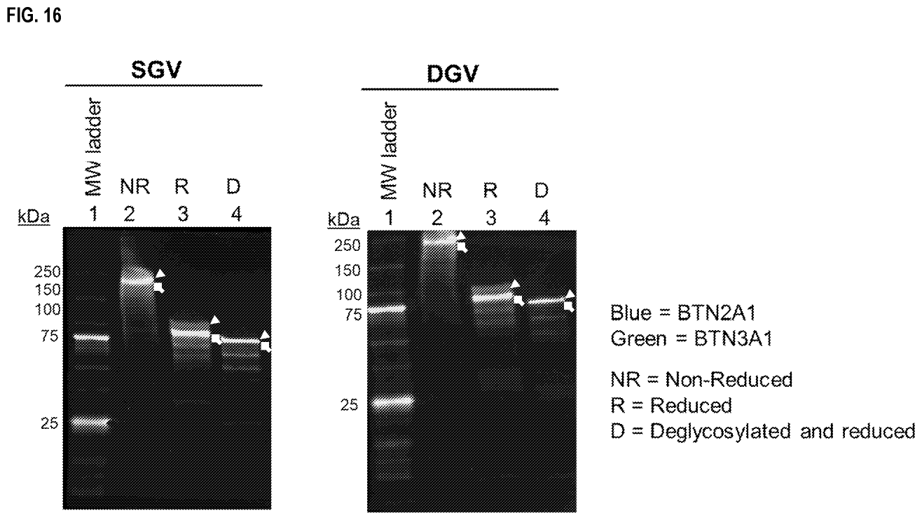

Briefly, the purified proteins were analyzed by western blot following denaturation in the absence of a reducing agent (non-reducing condition), in the presence of beta-mercaptoethanol (reducing condition), or in the presence of both beta-mercaptoethanol and a deglycosylating agent (reducing-deglycosylating condition). The BTN2A1/3A1-Fc-CD19scFv heterodimeric protein was detected with an anti-human BTN2A1 antibody and an anti-human BTN3A1 antibody. To facilitate contemporaneous detection of BTN2A1-alpha-CD19scFv and BTN3A1-beta-CD19scFv monomers, the BTN2A1- and BTN3A1-bound antibodies using infrared (IR) secondary antibodies that were fluorescently conjugated to using two IRDyes were used. As shown in FIG. 16, the protein bands recognized by the anti-BTN2A1 antibody are shown in blue color (triangular arrowheads) and the protein bands recognized by the anti-BTN3A1 antibody are shown in green (square arrowheads). Protein prepared under non reduced conditions (lanes “NR”) resulted in a single band, which could be detected with both the anti-human BTN2A1 and anti-human BTN3A1 antibodies (FIG. 16, left and right panels). Protein prepared under reduced conditions (lanes “R”) resulted in two bands, one each of which could be detected with the anti-human BTN2A1 and anti-human BTN3A1 antibodies (FIG. 16, left and right panels). Interestingly, protein prepared under both reduced and deglycosylated (conditions lane “D”) resulted in a single band, which could be detected with both the anti-human BTN2A1 and anti-human BTN3A1 antibodies (FIG. 16, left and right panels). Based on the similarity between the reduced and both reduced and deglycosylated lanes, the BTN2A1/3A1-Fc-CD19scFv GADLEN construct appears to have few glycosylations. These data further suggested based on the similarity between the reduced and both reduced and deglycosylated lanes that the BTN2A1/3A1-Fc-CD19scFv GADLEN is glycosylated. These data further suggested that BTN2A1-Fc-CD19scFv was glycosylated more than BTN3A1-Fc-CD19scFv.

These results indicate the BTN2A1/3A1-Fc-CD19scFv heterodimeric GADLEN protein manufactured using two single gene vectors (FIG. 16, left panel) or a dual gene vector (FIG. 16, right panel) is a disulfide-linked dimeric protein that reduces to two individual proteins (following disruption of the interchain disulfide bonds with R-mercaptoethanol). These results demonstrate that both production formats produced material that contained both chains as detected using specific antibodies to BTN2A1 and BTN3A1.

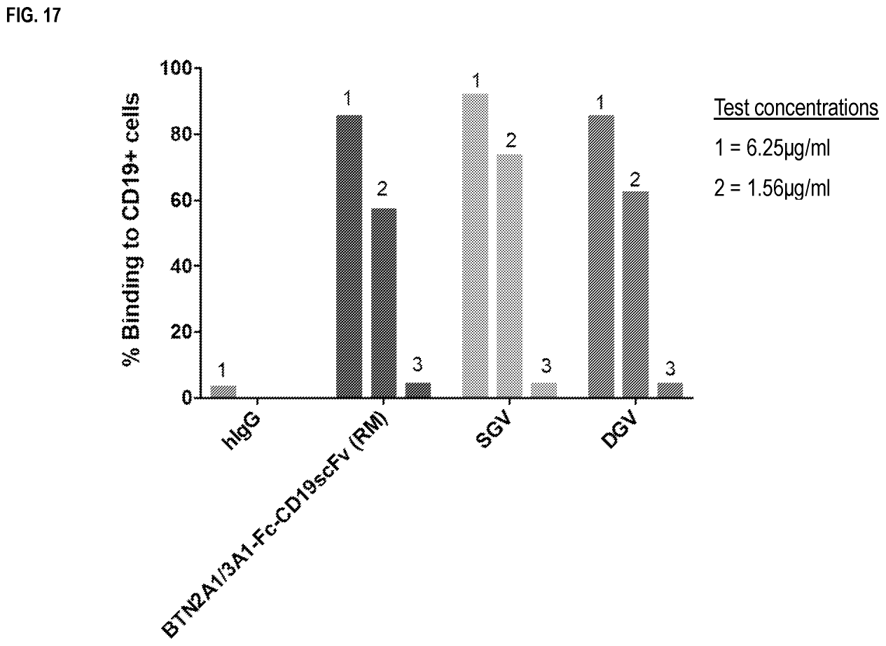

The BTN2A1/3A1-Fc-CD19scFv GADLEN heterodimeric proteins produced from the SGV or DGV production formats were tested for binding to CD19 expressed on a B-cell lymphoma cell line (Daudi). Briefly, Daudi cells were incubated with 6.25 μg, 1.56 μg, or 0 μg of the BTN2A1/3A1-Fc-CD19scFv GADLEN heterodimeric proteins produced from the SGV or DGV production formats or 6.25 μg human IgG, which was used as a negative control. Binding was detected using flow cytometry. As shown in FIG. 17, the BTN2A1/3A1-Fc-CD19scFv heterodimeric GADLEN protein produced from either SGV or DGV was able to bind to CD19 on Daudi cells as well as a BTN2A1/3A1-Fc-CD19scFv reference material.

These results indicate the BTN2A1/3A1-Fc-CD19scFv heterodimeric GADLEN protein manufactured using two single gene vectors or a dual gene vector approach is equally active in binding to CD19.

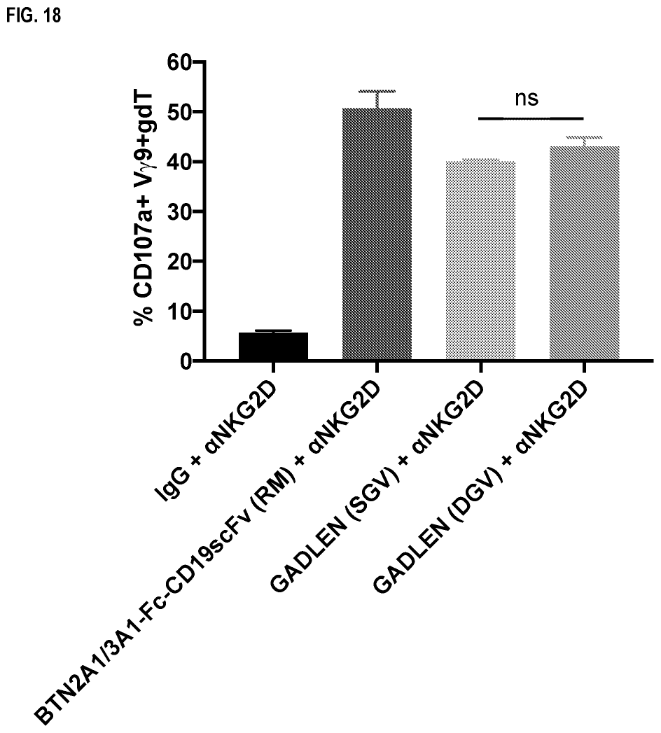

To evaluate the activation of the γδ T cells by the BTN2A1/3A1-Fc-CD19scFv heterodimeric GADLEN protein manufactured using two single gene vectors and a dual gene vector approach, an in vitro assay was used. Briefly, plates were coated with (1) an anti-NKG2D antibody (Clone #149810) and an IgG (a negative control), (2) the anti-NKG2D antibody and the BTN2A1/3A1-Fc-CD19scFv heterodimeric protein (reference material), (3 the anti-NKG2D antibody and the BTN2A1/3A1-Fc-CD19scFv prepared heterodimeric protein using the SGV format, and (4) the anti-NKG2D antibody and the BTN2A1/3A1-Fc-CD19scFv heterodimeric protein prepared using the DGV format. 1×10⁵ human γδ T cells were added to the plates for stimulation by the plate-bound agents and incubated in in 10% FBS+100U/mL recombinant human IL-2 (rhIL-2) for 4 hours at 37° C. in the presence of inhibitors of protein transport to the Golgi complex. After 4 hours, γδ T cells were harvested and stained with anti-CD107a, the degranulation marker of the activated γδ T cells, and analyzed by flow cytometry. The frequency of Vγ9+T cells expressing CD107a was determined by flow cytometry. As shown in FIG. 18, each preparation of the BTN2A1/3A1-Fc-CD19scFv heterodimeric protein, in combination with the anti-NKG2D antibody, was able to stimulate γδ T cells as evidenced by the expression of CD107a. on the other hand, the combination of IgG and the anti-NKG2D antibody produced activation at a background level (FIG. 18).

These results indicate that both the BTN2A1/3A1-Fc-CD19scFv heterodimeric GADLEN protein produced from either a SGV or DGV production format performed similarly in their ability to degranulate γδ T cells.

Collectively, these results indicate that heterodimeric GADLEN proteins may be produced using either co-transfection of two single gene vectors (SGV) expressing the alpha chain and beta chain separately (FIG. 10A), and transfection using a dual gene vector (DGV) that expresses the alpha and beta chain under two separate promoters in a single vector (FIG. 10B).

### Example 10: The BTN2A1-Fc-CD19scFv GADLEN Proteins Having BTN2A1 and BTN3A1 Tandem on Each Chain

Without being bound by theory, it is believed that a tetramer of the two butyrophilin proteins may be involved in the interaction with the Vγ9δ2 TCR. Therefore, the BTN2A1-Fc-CD19scFv GADLEN proteins having BTN2A1 and BTN3A1 tandem on each chain were constructed. As shown in FIG. 11, the new version of the BTN2A1/3A1-Fc-CD19scFv fusion protein where the variable domains of BTN2A1 and BTN3A1 are strung together in tandem and fused to the CD19scFv sequence through the IgG4 Fc sequence were generated. Two such chains would homodimerize to form the functional tetramer unit of BTN2A1 and BTN3A1 for Vγ9δ2 TCR activation (FIG. 11).

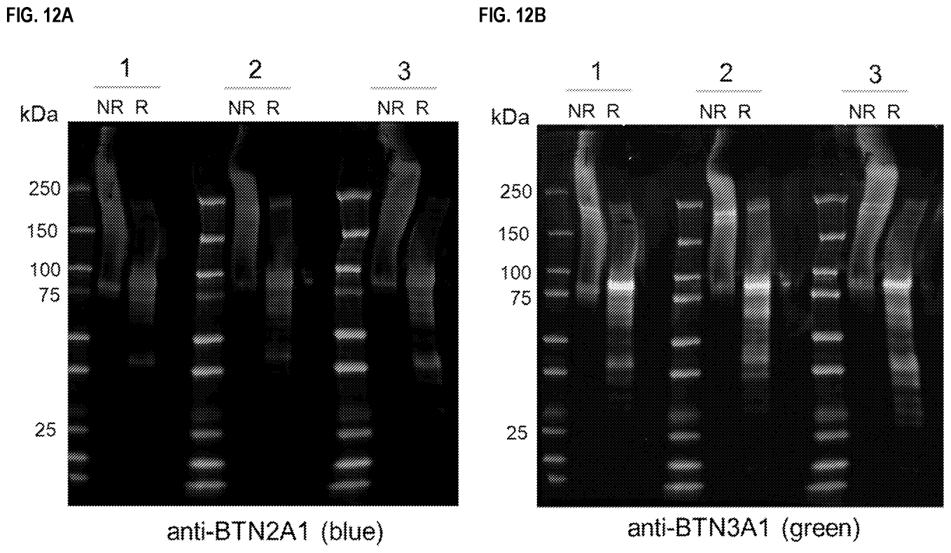

Therefore, the feasibility of using homodimeric GADLEN constructs containing only the variable (V) domains of BTN2A1 and BTN3A1 proteins arranged in tandem in a single polypeptide chain was evaluated. Three different GADLEN constructs containing either an IgG1 or IgG4 derived Fc linkers were generated and purified. The purified protein was analyzed by Western blot under non-reduced (denatured without adding a reducing agent), reduced (denatured in the presence of beta-mercapto ethanol) conditions and resolved by SDS-PAGE. The blots were probed with an anti-human BTN2A1 antibody and an anti-human BTN3A1 antibody. Both BTN2A1 and BTN3A1 were contemporaneously detected by detecting the BTN2A1- and BTN3A1-bound antibodies using infrared (IR) secondary antibodies that were fluorescently conjugated to using two IRDyes that are indicated in a blue (BTN2A1) or green (BTN3A1) color in FIGS. 12A-12B. As shown in FIG. 12A and FIG. 12B, the BTN2A1- and BTN3A1-bound antibodies identified identical bands. Non-reduced condition produced a band consistent with a dimer of monomers seen under reduced conditions.

These results indicate the presence of a disulfide-linked dimeric protein that reduces to a single monomer (following disruption of the interchain disulfide bonds with R-mercaptoethanol). Further each of Fc from IgG1 and the two versions IgG4 showed similar profiles.

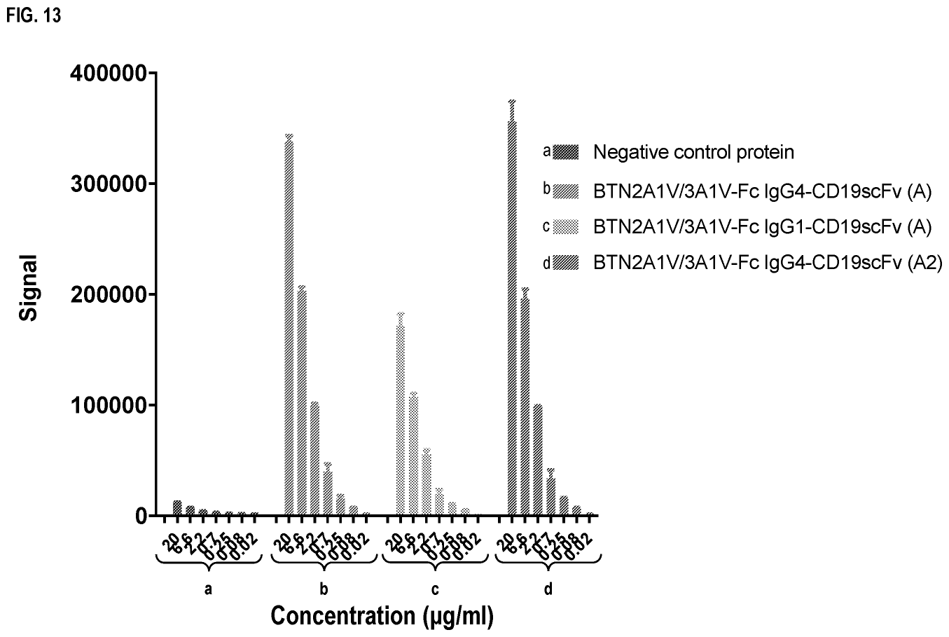

Next, the contemporaneous binding by the BTN2A1V/3A1V-Fc-CD19scFv GADLEN homodimeric proteins to two ligands was examined. An MSD based ELISA method was used to assess whether the homodimeric GADLEN constructs containing the variable domains of BTN2A1 and BTN3A1 were able to bind recombinant CD19. Briefly, recombinant CD19 protein was coated on plates and increasing amounts of the indicated BTN2A1V/3A1V-Fc-CD19scFv GADLEN homodimeric proteins were added to the plates for capture by the plate-bound CD19 protein. The binding was detected using an anti-BTN3A1 antibody followed by a sulfo-tagged anti-rabbit secondary antibody. A protein that is unable to bind both proteins was used as a negative control. As shown in FIG. 13, each of the BTN2A1V/3A1V-Fc-CD19scFv GADLEN homodimeric proteins showed a dose-dependent signal. On the other hand, the negative control showed only a background signal.

These results demonstrate that the GADLEN constructs containing the variable domains of BTN2A1 and BTN3A1 are capable of binding recombinant CD19 protein and contemporaneously bind a BTN3A1 ligand.

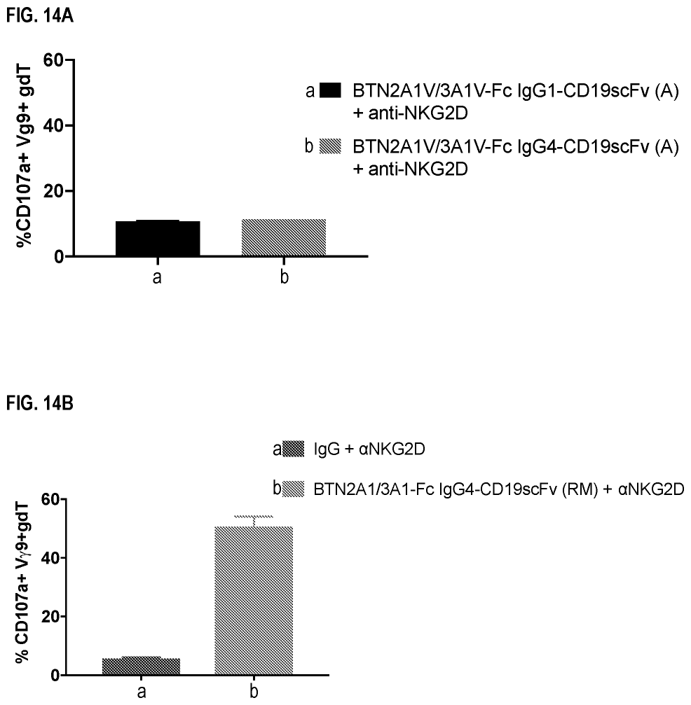

An in vitro assay was used to study the activation of the γδ T cells by the BTN2A1V/3A1V-Fc-CD19scFv homodimeric protein. Briefly, plates were coated with (1) an anti-NKG2D antibody (Clone #149810) and an IgG, (2) the anti-NKG2D antibody and the BTN2A1/3A1-Fc-CD19scFv heterodimeric protein, (3) the anti-NKG2D antibody and the BTN2A1V/3A1V-IgG1-CD19scFv homodimeric protein, and (4) the anti-NKG2D antibody and the BTN2A1V/3A1V-IgG1-CD19scFv homodimeric protein. IgG in combination of the anti-NKG2D antibody was used as a negative control. 1×10⁵ human γδ T cells were added to the plates for stimulation by the plate-bound agents and incubated in in 10% FBS+100U/mL recombinant human IL-2 (rhIL-2) for 4 hours at 37° C. in the presence of inhibitors of protein transport to the Golgi complex. After 4 hours, γδ T cells were harvested and stained with anti-CD107a, the degranulation marker of the activated γδ T cells, and analyzed by flow cytometry. The frequency of Vγ9+T cells expressing CD107a was determined by flow cytometry. As shown in FIG. 14A, about 10% γδ T cells exhibited the expression of CD107a when stimulated with the BTN2A1V/3A1V-IgG1-CD19scFv or BTN2A1V/3A1V-IgG1-CD19scFv homodimeric proteins, in combination with the anti-NKG2D antibody. this level of activation was greater than the background activation produced by the combination of IgG and the anti-NKG2D antibody (FIG. 14B), but lower than the extent of activation produced by the BTN2A1/3A1-IgG1-CD19scFv heterodimeric protein (FIG. 14B) in this in vitro assay.

These results indicate that both the BTN2A1V/3A1V-IgG1-CD19scFv and BTN2A1V/3A1V-IgG1-CD19scFv homodimeric proteins are capable of activating the γδ T cells.

### Example 11: Comparison of the BTN2A1-Fc-CD19scFv Heterodimeric GADLEN Proteins Having Charged Polarized Linkers and Knob-In-Hole (KIH) Mutations for Promoting Heterodimerization and Disfavoring Homodimerization

Three versions of the BTN2A1/3A1-Fc-CD19scFv GADLEN protein were generated to compare the charged polarized linker strategy to facilitate heterodimerization versus the knob-in-hole (KIH) mutations: charged polarized linkers (FIG. 19, left cartoon), KIH mutations in Fc domain, and KIH mutations and FcRn mutations in Fc domains (both FIG. 19, right cartoon). Without being bound by theory is thought that the BTN2A1/3A1-Fc-CD19scFv GADLEN protein having KIH mutations and FcRn mutations increase binding to neonatal Fc receptor. The charged polarized linkers (CPL) and the KIH mutations in Fc domain were designed for favoring heterodimerization and disfavoring homodimerization by promoting association between alpha and beta chains. KIH has been successfully used to generate bi-specific antibodies. See, e.g., Eldesouki et al., Identification and Targeting of Thomsen-Friedenreich and IL1RAP Antigens on Chronic Myeloid Leukemia Stem Cells Using Bi-Specific Antibodies, Onco Targets Ther 14:609-621 (2021).

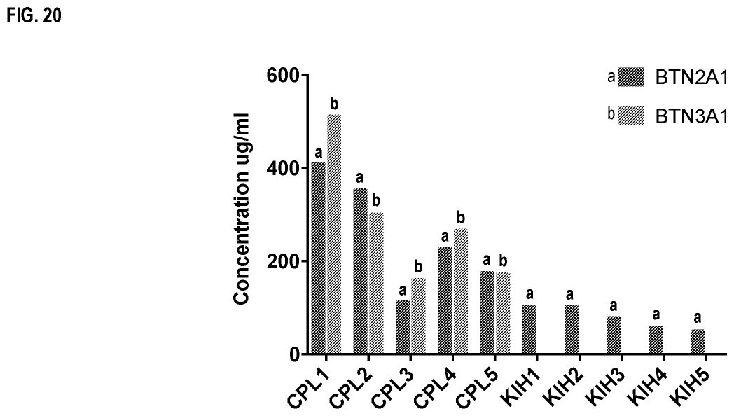

The BTN2A1/3A1-Fc-CD19scFv GADLEN protein having charged polarized linker or the knob-in-hole (KIH) mutations were constructed. Mini pools were generated by transfecting vectors that express the alpha and beta chains of the BTN2A1/3A1-Fc-CD19scFv construct that incorporated either the charged polarized linkers (CPL) and the KIH mutations in the individual alpha and beta chains. The expression levels of each chain (BTN2A1-alpha-CD19scFv and BTN3A1-beta-CD19scFv) in each of the mini pools was quantified by an ELISA method that used a recombinant CD19 protein to capture the heterodimer protein and detect with either a BTN2A1 or BTN3A1 specific antibody. The amounts of the BTN2A1-alpha and BTN3A1-beta chains in the culture supernatants of mini pools were quantitated. As shown in FIG. 20, the mini pools generated using the CPL approach produced equivalent amounts of BTN2A1-alpha and BTN3A1-beta chains in the culture supernatant. On the other hand, surprisingly, the KIH mini pools produced less amounts of BTN2A1-alpha chain and very low amounts of BTN3A1-beta chain in the culture supernatant.

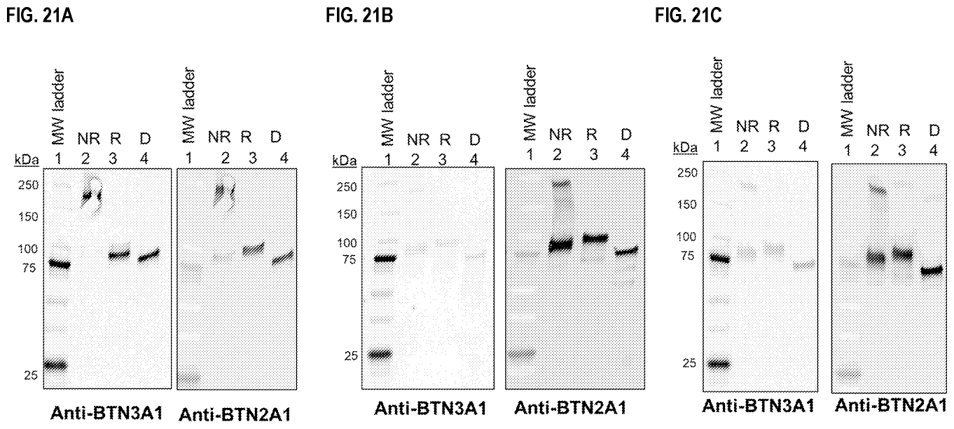

The BTN2A1/3A1-Fc-CD19scFv GADLEN protein having charged polarized linker or the knob-in-hole (KIH) mutations were also analyzed by western blotting. Briefly, the purified proteins were subjected to denaturation in the absence of a reducing agent (non-reducing condition), in the presence of beta-mercaptoethanol (reducing condition), or in the presence of both beta-mercaptoethanol and a deglycosylating agent (reducing-deglycosylating condition) and analyzed by analyzed by western blot. The BTN2A1/3A1-Fc-CD19scFv heterodimeric protein was detected with an anti-human BTN2A1 antibody and an anti-human BTN3A1 antibody. As shown in FIG. 21A, the protein bands recognized by the anti-BTN2A1 and the anti-BTN3A1 antibodies in the BTN2A1/3A1-Fc-CD19scFv GADLEN protein having charged polarized linker strategy showed similar levels of the BTN3A1-containing and BTN2A1-containing chains.

On the other hand, BTN2A1/3A1-Fc-CD19scFv GADLEN protein having produced using the KIH mutations in Fc domain (FIG. 21B), and KIH mutations and FcRn mutations (FIG. 21C) showed lesser expression of BTN3A1-containing chain, with increased BTN2A1-containing chain. These data are consistent with the ELISA data (FIG. 21A) and qPCR data (FIG. 10C, FIG. 10D and FIG. 10E). This suggests the charge polarized linker strategy is superior to KIH in the formation of heterodimeric GADLEN proteins.

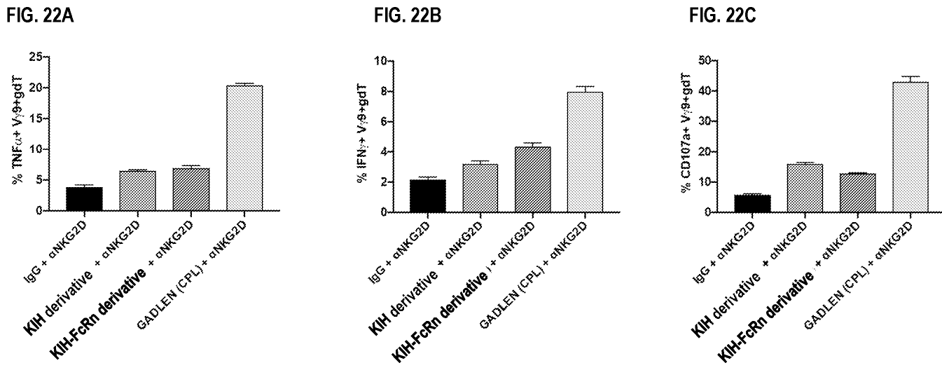

The BTN2A1/3A1-Fc-CD19scFv GADLEN protein having charged polarized linker or the knob-in-hole (KIH) mutations were also analyzed by an in vitro assay for the stimulation of γδ T cells. Briefly, plates were coated with ((1) an anti-NKG2D antibody (Clone #149810) and an IgG (a negative control), (2) the anti-NKG2D antibody and the BTN2A1/3A1-Fc-CD19scFv heterodimeric protein having the knob-in-hole (KIH) mutations, (3 the anti-NKG2D antibody and the BTN2A1/3A1-Fc-CD19scFv heterodimeric protein having the knob-in-hole (KIH) and FcRn mutations, and (4) the anti-NKG2D antibody and the BTN2A1/3A1-Fc-CD19scFv heterodimeric protein having charged polarized linker. 1×10⁵ human γδ T cells were added to the plates for stimulation by the plate-bound agents and incubated in in 10% FBS+100U/mL recombinant human IL-2 (rhIL-2) for 4 hours at 37° C. in the presence of inhibitors of protein transport to the Golgi complex. After 4 hours, γδ T cells were harvested and stained with anti-TNFα, anti-IFNγ or anti-CD107a, the degranulation marker of the activated γδ T cells, and analyzed by flow cytometry. The frequency of Vγ9+T cells expressing cytotoxic cytokines TNFα, IFNγ or the degranulation marker CD107a was determined by flow cytometry. As shown in FIG. 22A, the BTN2A1/3A1-Fc-CD19scFv heterodimeric protein having the knob-in-hole (KIH) mutations with or without FcRn mutations induced lesser γδ T cells to express TNFα. Similarly, the BTN2A1/3A1-Fc-CD19scFv heterodimeric protein having the knob-in-hole (KIH) mutations with or without FcRn mutations induced lesser γδ T cells to express IFNγ (FIG. 22B). Similarly, the BTN2A1/3A1-Fc-CD19scFv heterodimeric protein having the knob-in-hole (KIH) mutations with or without FcRn mutations induced lesser γδ T cells to express CD107a (FIG. 22C).

Collectively, these results show that the use of charge polarized linkers are better suited for heterodimer formation to create GADLENs. These data are suprising, inter alia, because knob-into-hole Fc technology has made huge progress in designing bispecific antibodies with engineered asymmetric CH3 domains, but it does not appear to work for the heterodimeric GADLEN proteins.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A heterodimeric protein comprising an alpha chain and a beta chain,
wherein the alpha chain comprises:
(a) a first domain comprising a BTN2A1 protein, or a fragment thereof;
(b) a second domain comprising a targeting domain that specifically binds to CD19; and
(c) a linker that adjoins the first and second domains;
and
wherein the beta chain comprises:
(a) a first domain comprising a BTN3A1 protein, or a fragment thereof;
(b) a second domain comprising a targeting domain that specifically binds to CD19; and
(c) a linker that adjoins the first and second domains.

2. The heterodimeric chimeric protein of claim 1, wherein the alpha chain and the beta chain self-associate to form the heterodimer.

3. The heterodimeric chimeric protein of claim 1 or claim 2, wherein the first domain of the alpha chain comprises the extracellular domain of BTN2A1 protein.

4. The heterodimeric chimeric protein of any one of claims 1 to 3, wherein the first domain of the alpha chain comprises a polypeptide having an amino acid sequence that has at least about 95% identity with the amino acid sequence of SEQ ID NO: 35 or SEQ ID NO: 71.

5. The heterodimeric chimeric protein of any one of claims 1 to 4, wherein the first domain of the alpha chain comprises a polypeptide having an amino acid sequence that is identical to the amino acid sequence of SEQ ID NO: 35 or SEQ ID NO: 71.

6. The heterodimeric chimeric protein of any one of claims 1 to 5, wherein the first domain of the beta chain comprises the extracellular domain of BTN3A1 protein.

7. The heterodimeric chimeric protein of any one of claims 1 to 6, wherein the first domain of the beta chain comprises a polypeptide having an amino acid sequence that has at least about 95% identity with the amino acid sequence of SEQ ID NO: 19 or SEQ ID NO: 72.

8. The heterodimeric chimeric protein of claim 7, wherein the first domain of the beta chain comprises a polypeptide having an amino acid sequence that is identical to the amino acid sequence of SEQ ID NO: 19 or SEQ ID NO: 72.

9. The heterodimeric protein of any one of claims 1 to 8, wherein the targeting domain is an antibody, or antigen binding fragment thereof.

10. The heterodimeric protein of any one of claims 1 to 8, wherein the targeting domain is an antibody-like molecule, or antigen binding fragment thereof.

11. The heterodimeric protein of claim 10, wherein the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂.

12. The heterodimeric protein of any one of claims 1 to 11, wherein the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus.

13. The heterodimeric protein of claim 12, wherein the linker forms a heterodimer through electrostatic interactions between positively charged amino acid residues and negatively charged amino acid residues on the first and second charge polarized core domains.

14. The heterodimeric protein of claim 12 or claim 13, wherein the first and/or second charge polarized core domain comprises a polypeptide linker, optionally selected from a flexible amino acid sequence, IgG hinge region, or antibody sequence.

15. The heterodimeric protein of any one of claims 12 to 14, wherein the linker is a synthetic linker, optionally PEG.

16. The heterodimeric protein of any one of claims 12 to 15, wherein the linker comprises the hinge-CH2-CH3 Fc domain derived from IgG1, optionally human IgG1.

17. The heterodimeric protein of any one of claims 12 to 16, wherein the linker comprises the hinge-CH2-CH3 Fc domain derived from IgG4, optionally human IgG4.

18. The heterodimeric protein of any one of claims 12 to 17, wherein the first and/or second charge polarized core domain further comprise peptides having positively and/or negatively charged amino acid residues at the amino and/or carboxy terminus of the charge polarized core domain.

19. The heterodimeric protein of claim 13, wherein the positively charged amino acid residues include one or more of amino acids selected from His, Lys, and Arg.

20. The heterodimeric protein of claim 13 or claim 14, wherein the positively charged amino acid residues are present in a peptide comprising positively charged amino acid residues in the first and/or the second charge polarized core domains.

21. The heterodimeric protein of claim 20, wherein the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5).

22. The heterodimeric protein of claim 20 or claim 21, wherein the peptide comprising positively charged amino acid residues comprises the sequence RKGGKR (SEQ ID NO: 11) or GSGSRKGGKRGS (SEQ ID NO: 12).

23. The heterodimeric protein of any one of claims 13 to 22, wherein the negatively charged amino acid residues may include one or more amino acids selected from Asp and Glu.

24. The heterodimeric protein of any one of claims 13 to 23, wherein the negatively charged amino acid residues are present in a peptide comprising negatively charged amino acid residues in the first and/or the second charge polarized core domains.

25. The heterodimeric protein of claim 24, wherein the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where each n is independently an integer 0 to 4) (SEQ ID NO: 6).

26. The heterodimeric chimeric protein of any one of claims 1 to 25, wherein the second domain of the alpha chain and/or beta chain comprises a polypeptide having an amino acid sequence that has at least about 95% identity with an amino acid sequence selected from SEQ ID NOs: 20-23.

27. The heterodimeric chimeric protein of claim 26, wherein the second domain of the alpha chain and/or beta chain comprises a polypeptide having an amino acid sequence that is identical to an amino acid sequence the amino acid sequence selected from SEQ ID NOs: 20-23.

28. The heterodimeric chimeric protein of any one of claims 1 to 17 or 19 to 27, wherein the linker of alpha chain and/or beta chain comprises a polypeptide having an amino acid sequence that has at least about 95% identity with an amino acid sequence selected from SEQ ID NOs: 15-17, 28-32 and 52-55.

29. The heterodimeric chimeric protein of claim 28, wherein the linker of alpha chain and/or beta chain comprises a polypeptide having an amino acid sequence that is identical to an amino acid sequence the amino acid sequence selected from SEQ ID NOs: 15-17, 28-32 and 52-55.

30. The heterodimeric chimeric protein of any one of claims 1 to 29, wherein the alpha chain comprises a polypeptide having an amino acid sequence that has at least about 95% identity with an amino acid sequence selected from SEQ ID NOs: 37-39.

31. The heterodimeric chimeric protein of claim 30, wherein the alpha chain comprises a polypeptide having an amino acid sequence that is identical to an amino acid sequence the amino acid sequence selected from SEQ ID NOs: 37-39.

32. The heterodimeric chimeric protein of any one of claims 1 to 31, wherein the beta chain comprises a polypeptide having an amino acid sequence that has at least about 95% identity with an amino acid sequence selected from SEQ ID NOs: 40-42.

33. The heterodimeric chimeric protein of claim 32, wherein the beta chain comprises a polypeptide having an amino acid sequence that is identical to an amino acid sequence the amino acid sequence selected from SEQ ID NOs: 40-42.

34. The heterodimeric chimeric protein of claim 33, wherein the heterodimeric chimeric protein comprises an amino acid sequence that is identical to an amino acid sequence the amino acid sequence of:
(a) SEQ ID NO: 37 and SEQ ID NO: 40;
(b) SEQ ID NO: 38 and SEQ ID NO: 41; or
(c) SEQ ID NO: 39 and SEQ ID NO: 42.

35. The heterodimeric chimeric protein of any one of claims 1 to 34, wherein the first domain and/or the heterodimeric protein modulates or is capable of modulating a γδ (gamma delta) T cell.

36. The heterodimeric chimeric protein of claim 35, wherein the gamma delta T cell is Vγ9δ2 T cell.

37. The heterodimeric chimeric protein of claim 35, wherein the modulation of a gamma delta T cell is activation of a gamma delta T cell.

38. The heterodimeric chimeric protein of any one of claims 1 to 37, wherein the heterodimeric protein is capable of forming a synapse between a gamma delta T cell and a tumor cell and/or the heterodimeric protein is capable of contemporaneous activation and targeting of gamma delta T cells to tumor cells.

39. A pharmaceutical composition, comprising the heterodimeric protein of any one of claims 1 to 38.

40. An expression vector, comprising a nucleic acid encoding the first and/or second polypeptide chains of the heterodimeric protein of any one of claims 1 to 38.

41. The expression vector of claim 40, wherein the expression vector is a mammalian expression vector.

42. The expression vector of claim 40 or claim 41, wherein the expression vector comprises DNA or RNA.

43. A host cell, comprising the expression vector of any one of claims 40 to 42.

44. A method of contemporaneous activation and targeting of gamma delta T cells to tumor cells comprising administering to a subject in need thereof an effective amount of a pharmaceutical composition of claim 39 to a subject in need thereof.

45. A method of modulating a patient's immune response, comprising administering an effective amount of a pharmaceutical composition of claim 39 to a subject in need thereof.

46. A method of stimulating proliferation of gamma delta T cells, comprising:
administering an effective amount of a pharmaceutical composition of claim 39 to a subject in need thereof thereby causing an in vivo proliferation of gamma delta T cells and/or
contacting an effective amount of a pharmaceutical composition of claim 39 with a cell derived from a subject in need thereof thereby causing an ex vivo proliferation of gamma delta T cells.

47. The method of any one of claims 44-46, wherein the subject's T cells are activated by the first domain.

48. The method of any one of claims 44-47 wherein the subject has a tumor and the gamma delta T cells modulate cells of the tumor.

49. A method of treating cancer, comprising administering to a subject in need thereof an effective amount of a pharmaceutical composition of claim 39 to a subject in need thereof.

50. The method of claim 49, wherein the cancer is a lymphoma.

51. The method of claim 49, wherein the cancer is a leukemia.

52. The method of any one of claims 49-51, wherein the cancer is a Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia.

53. The method of claim 49, wherein the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

54. The method of claim 49 or claim 53, wherein the cancer is prostate cancer.

55. The method of any one of claims 49, 53, or 54, wherein the cancer is an epithelial-derived carcinoma.

56. The method of any one of claims 49 to 55, wherein the cancer is known to express the antigenic target of the second domain of the heterodimeric protein.

57. The method of any one of claims 49 to 56, wherein the cancer has mutations which limit recognition by alpha beta T cells, optionally selected from mutations in MHC I, beta 2 microglobulin, and Transporter associated with antigen processing (TAP).

58. The method of any one of claims 49 to 57, wherein the subject is further administered autologous or allogeneic gamma delta T cells that were expanded ex vivo.

59. The method of claim 58, wherein the autologous or allogeneic gamma delta T cells express a Chimeric Antigen Receptor.

60. A tetrameric chimeric protein comprising two homodimeric chimeric proteins of any one of claims 1 to 34, the tetramer comprises two protein chains which homodimerize to form a tetramer unit comprising BTN2A1 and BTN3A1.

61. The tetrameric chimeric protein of claim 60, comprising a polypeptide having an amino acid sequence that has at least about 95% identity with an amino acid sequence selected from SEQ ID NOs: 43, 44 and 56-70.

62. The tetrameric chimeric protein as depicted in FIG. 11, optionally comprising a polypeptide having an amino acid sequence that has at least about 95% identity with an amino acid sequence selected from SEQ ID NOs: 43, 44 and 56-70.

63. A chimeric protein of a general structure of:
N terminus-(a)-(b)-(c)-C terminus,
wherein:
(a) is the first domain comprising the general structure of (a1)-SL-(a2), wherein
(a1) is an extracellular domain (ECD) of a butyrophilin family protein, or a fragment thereof,
(a2) is an extracellular domain (ECD) of a butyrophilin family protein, or a fragment thereof, and SL is a second linker adjoins (a1) and (a2) comprising a flexible amino acid sequence of about 4 to about 50 amino acids length, and
(c) is a second domain comprising a targeting domain, the targeting domain being selected from (i) an antibody, antibody-like molecule, or antigen binding fragment thereof, and (ii) an extracellular domain of a membrane protein, and
(b) is linker that adjoins the first and second domains, wherein the a linker comprises at least one cysteine residue capable of forming a disulfide bond.

64. The chimeric protein of claim 63, wherein the (a1) and (a2) are two of the same butyrophilin family proteins.

65. The chimeric protein of claim 63, wherein the (a1) and (a2) are different butyrophilin family proteins.

66. The chimeric protein of any one of claims 63 to 65, wherein the (a1) and/or (a2) is a fragment of the butyrophilin family protein comprising a variable domain.

67. The chimeric protein of any one of claims 63 to 66, wherein the (a1) and (a2) comprise butyrophilin family proteins independently selected from BTN1A1, BTN2A1, BTN2A2, BTN2A3, BTN3A1, BTN3A2, BTN3A3, BTNL2, BTNL3, BTNL8, BTNL9, BTNL10, and SKINTL.

68. The chimeric protein of claim 67, wherein the butyrophilin family proteins are independently selected from human BTN1A1, human BTN2A1, human BTN2A2, human BTN2A3, human BTN3A1, human BTN3A2, human BTN3A3, human BTNL2, human BTNL3, human BTNL8, human BTNL9, human BTNL10, and human SKINTL.

69. The chimeric protein of any one of claims 63 to 66, wherein the first domain comprises a polypeptide having
(a1) an amino acid sequence having at least 90%, or 95%, or 97%, or 98%, or 99% identity with SEQ ID NOs: 19, 35-36, 45, 71-72, 80-93; and
(a2) an amino acid sequence having at least 90%, or 95%, or 97%, or 98%, or 99% identity with SEQ ID NOs: 19, 35-36, 45, 71-72, 80-93.

70. The chimeric protein of claim 69, wherein the first domain comprises a polypeptide having an amino acid sequence of:
(a1) any one of SEQ ID NOs: 19, 35-36, 45, 71-72, 80-93; and
(a2) any one of SEQ ID NOs: 19, 35-36, 45, 71-72, 80-93.

71. The chimeric protein any one of claims 63 to 70, wherein the first domain comprises extracellular domains of:
(i) BTNL3 and BTNL8;
(ii) BTN2A1 and BTN3A1;
(iii) BTN3A1 and BTN3A2; or
(iv) BTN3A1 and BTN3A3.

72. The chimeric protein any one of claims 63 to 68, wherein the first domain comprises variable domains of:
(i) BTNL3 and BTNL8;
(ii) BTN2A1 and BTN3A1;
(iii) BTN3A1 and BTN3A2; or
(iv) BTN3A1 and BTN3A3.

73. The chimeric protein of any one of claims 63 to 72, wherein the second linker comprises an amino acid sequence of general formula G(G₃S)ₘ or GGGSₙ wherein m and n are integers in the range 1 to 16.

74. The chimeric protein of any one of claims 63 to 73, wherein the targeting domain is capable of binding an antigen on the surface of a cancer cell.

75. The chimeric protein of any one of claims 63 to 74, wherein the targeting domain comprises an extracellular domain of a membrane protein selected from LAG-3, PD-1, TIGIT, CD19, or PSMA.

76. The chimeric protein of any one of claims 63 to 74, wherein the targeting domain is an antibody, or an antigen binding fragment thereof.

77. The chimeric protein of claim 76, wherein the binding fragment comprises an Fv domain.

78. The chimeric protein of any one of claims 63 to 74, wherein the targeting domain is an antibody-like molecule, or antigen binding fragment thereof.

79. The chimeric protein of claim 78, wherein the binding fragment comprises an scFv domain.

80. The chimeric protein of any one of claims 76 to 79, wherein the targeting domain specifically binds one of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, CLL-1, and GD3.

81. The chimeric protein of claim 80, wherein the targeting domain specifically binds CD19.

82. The chimeric protein of claim 80, wherein the targeting domain specifically binds PSMA.

83. The chimeric protein of claim 80, wherein the targeting domain specifically binds CD33.

84. The chimeric protein of claim 80, wherein the targeting domain specifically binds CLL-1.

85. The chimeric protein of claim 80, wherein the targeting domain comprises a polypeptide having an amino acid sequence with at least 90%, or 95%, or 97%, or 98%, or 99% identity with a polypeptide selected from SEQ ID NOs: 20-27 and 94-126.

86. The chimeric protein of any one of claims 63-85, wherein the linker comprises the hinge-CH2-CH3 Fc domain.

87. The chimeric protein of claim 86, wherein the hinge-CH2-CH3 Fc domain is derived from IgG1, optionally human IgG1.

88. The chimeric protein of claim 86, wherein the hinge-CH2-CH3 Fc domain is derived from IgG4, optionally human IgG4.

89. The chimeric protein of claim 86, wherein the hinge-CH2-CH3 Fc domain comprises a polypeptide having an amino acid sequence with at least 90%, or 95%, or 97%, or 98%, or 99% identity with a polypeptide selected from SEQ ID NOs: 16-17, 28-32, and 52-55.

90. The chimeric protein of any one of claims 63-89, wherein the first domain and/or the chimeric protein modulates or is capable of modulating a γδ (gamma delta) T cell.

91. The chimeric protein of claim 90, wherein the gamma delta T cell expresses Vγ4 or Vγ9δ2.

92. The chimeric protein of claim 90 or claim 91, wherein the first domain comprises BTNL3 and BTNL8 and it modulates a Vγ4-expressing T cell.

93. The chimeric protein of claim 90 or claim 91, wherein the first domain modulates a Vγ9δ2-expressing T cell.

94. The chimeric protein of any one of claims 90, 91 and 93, wherein the first domain comprises:
(a) BTN2A1 and BTN3A1,
(b) BTN3A1 and BTN3A2, or
(c) BTN3A1 and BTNA3.

95. The chimeric protein of any one of claims 90-94, wherein the modulation of a gamma delta T cell is activation of a gamma delta T cell.

96. The chimeric protein of any one of claims 63 to 95, wherein the chimeric protein is capable of forming a synapse between a gamma delta T cell and a tumor cell and/or the chimeric protein is capable of contemporaneous activation and targeting of gamma delta T cells to tumor cells.

97. The chimeric protein of any one of claims 63 to 96, wherein the chimeric protein is a homodimer.

98. A pharmaceutical composition, comprising the chimeric protein of any one of claims 63 to 97.

99. An expression vector, comprising a nucleic acid encoding the first and/or second polypeptide chains of the chimeric protein of any one of claims 63 to 97.

100. The expression vector of claim 99, wherein the expression vector is a mammalian expression vector.

101. The expression vector of claim 99 or claim 100, wherein the expression vector comprises DNA or RNA.

102. A host cell, comprising the expression vector of any one of claims 99 to 101.

103. A method of contemporaneous activation and targeting of gamma delta T cells to tumor cells comprising administering to a subject in need thereof an effective amount of a pharmaceutical composition of claim 98 to a subject in need thereof.

104. A method of modulating a patient's immune response, comprising administering an effective amount of a pharmaceutical composition of claim 98 to a subject in need thereof.

105. A method of stimulating proliferation of gamma delta T cells, comprising:
administering an effective amount of a pharmaceutical composition of claim 98 to a subject in need thereof thereby causing an in vivo proliferation of gamma delta T cells and/or
contacting an effective amount of a pharmaceutical composition of claim 98 with a cell derived from a subject in need thereof thereby causing an ex vivo proliferation of gamma delta T cells.

106. The method of any one of claims 103-105, wherein the subject's T cells are activated by the first domain.

107. The method of any one of claims 103-106, wherein the subject has a tumor and the gamma delta T cells modulate cells of the tumor.

108. A method of treating cancer, comprising administering to a subject in need thereof an effective amount of a pharmaceutical composition of claim 98 to a subject in need thereof.

109. The method of claim 108, wherein the cancer is a lymphoma.

110. The method of claim 108, wherein the cancer is a leukemia.

111. The method of any one of claims 108-110, wherein the cancer is a Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia.

112. The method of claim 111, wherein the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

113. The method of claim 108 or claim 112, wherein the cancer is prostate cancer.

114. The method of any one of claims 108, 112, or 113, wherein the cancer is an epithelial-derived carcinoma.

115. The method of any one of claims 108 to 114, wherein the cancer is known to express the antigenic target of the second domain of the chimeric protein.

116. The method of any one of claims 108 to 115, wherein the cancer has mutations which limit recognition by alpha beta T cells, optionally selected from mutations in MHC I, beta 2 microglobulin, and Transporter associated with antigen processing (TAP).

117. The method of any one of claims 108 to 116, wherein the subject is further administered autologous or allogeneic gamma delta T cells that were expanded ex vivo.

118. The method of claim 117, wherein the autologous or allogeneic gamma delta T cells express a Chimeric Antigen Receptor.
